(19) United States
(12) Patent Application Publication
(10) Pub. No.: US 2016/0208170 A1
(43) Pub. Date: Jul. 21, 2016

(54) LIQUID-CRYSTALLINE MEDIUM

(71) Applicant: MERCK PATENT GMBH, Darmstadt (DE)

(72) Inventors: Harald Hirschmann, Darmstadt (DE); Martina Windhorst, I, Muenster (DE)

(73) Assignee: MERCK PATENT GMBH, Darmstadt (DE)

(21) Appl. No.: 15/001,772

(22) Filed: Jan. 20, 2016

Related U.S. Application Data
(60) Provisional application No. 62/105,578, filed on Jan. 20, 2015.

(51) Int. Cl. C09K 19/34; C09K 19/54; C07C 13/28; C09K 19/30

(52) U.S. Cl. CPC C09K 19/3491; C09K 19/3003; C09K 19/3405; C09K 19/3402; C09K 19/3066; C09K 19/54; C07C 13/28; C09K 2019/3009; C09K 2019/301; C09K 2019/3016; C09K 2019/3408; C09K 2019/3004; C09K 2019/3422; C07C 2101/14

(57) ABSTRACT

The invention relates to a liquid-crystalline medium which comprises the compound of the formula I,

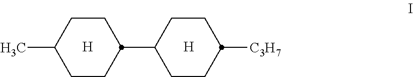

and to the use thereof for an active-matrix display, in particular based on the VA, PSA, PS-VA, PM-VA, SS-VA, PALC, IPS, PS-IPS, FFS or PS-FFS effect.

The invention relates to a liquid-crystalline medium which comprises the compound of the formula I,

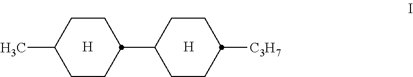

Media of this type can be used, in particular, for electro-optical displays having active-matrix addressing based on the ECB effect and for IPS (in-plane switching) displays or FFS (fringe field switching) displays.

The principle of electrically controlled birefringence, the ECB effect or also DAP (deformation of aligned phases) effect, was described for the first time in 1971 (M. F. Schieckel and K. Fahrenschon, “Deformation of nematic liquid crystals with vertical orientation in electrical fields”, Appl. Phys. Lett. 19 (1971), 3912). This was followed by papers by J. F. Kahn (Appl. Phys. Lett. 20 (1972), 1193) and G. Labrunie and J. Robert (J. Appl. Phys. 44 (1973), 4869).

The papers by J. Robert and F. Clerc (SID 80 Digest Techn. Papers (1980), 30), J. Duchene (Displays 7 (1986), 3) and H. Schad (SID 82 Digest Techn. Papers (1982), 244) showed that liquid-crystalline phases must have high values for the ratio of the elastic constants K₃/K₁, high values for the optical anisotropy Δn and values for the dielectric anisotropy of Δ∈≦−0.5 in order to be suitable for use in high-information display elements based on the ECB effect. Electro-optical display elements based on the ECB effect have a homeotropic edge alignment (VA technology=vertically aligned). Dielectrically negative liquid-crystal media can also be used in displays which use the so-called IPS or FFS effect.

Displays which use the ECB effect, as so-called VAN (vertically aligned nematic) displays, for example in the MVA (multi-domain vertical alignment, for example: Yoshide, H. et al., paper 3.1: “MVA LCD for Notebook or Mobile PCs . . . ”, SID 2004 International Symposium, Digest of Technical Papers, XXXV, Book I, pp. 6 to 9, and Liu, C. T. et al., paper 15.1: “A 46-inch TFT-LCD HDTV Technology . . . ”, SID 2004 International Symposium, Digest of Technical Papers, XXXV, Book II, pp. 750 to 753), PVA (patterned vertical alignment, for example: Kim, Sang Soo, paper 15.4: “Super PVA Sets New State-of-the-Art for LCD-TV”, SID 2004 International Symposium, Digest of Technical Papers, XXXV, Book II, pp. 760 to 763), ASV (advanced super view, for example: Shigeta, Mitzuhiro and Fukuoka, Hirofumi, paper 15.2: “Development of High Quality LCDTV”, SID 2004 International Symposium, Digest of Technical Papers, XXXV, Book II, pp. 754 to 757) modes, have established themselves as one of the three more recent types of liquid-crystal display that are currently the most important, in particular for television applications, besides IPS (in-plane switching) displays (for example: Yeo, S. D., paper 15.3: “An LC Display for the TV Application”, SID 2004 International Symposium, Digest of Technical Papers, XXXV, Book II, pp. 758 & 759) and the long-known TN (twisted nematic) displays. The technologies are compared in general form, for example, in Souk, Jun, SID Seminar 2004, seminar M-6: “Recent Advances in LCD Technology”, Seminar Lecture Notes, M-6/1 to M-6/26, and Miller, Ian, SID Seminar 2004, seminar M-7: “LCD-Television”, Seminar Lecture Notes, M-7/1 to M-7/32. Although the response times of modern ECB displays have already been significantly improved by addressing methods with overdrive, for example: Kim, Hyeon Kyeong et al., paper 9.1: “A 57-in. Wide UXGA TFT-LCD for HDTV Application”, SID 2004 International Symposium, Digest of Technical Papers, XXXV, Book I, pp. 106 to 109, the achievement of video-compatible response times, in particular on switching of grey shades, is still a problem which has not yet been satisfactorily solved.

Industrial application of this effect in electro-optical display elements requires LC phases, which have to satisfy a multiplicity of requirements. Particularly important here are chemical resistance to moisture, air and physical influences, such as heat, infrared, visible and ultraviolet radiation and direct and alternating electric fields.

Furthermore, industrially usable LC phases are required to have a liquid-crystalline mesophase in a suitable temperature range and low viscosity. None of the hitherto-disclosed series of compounds having a liquid-crystalline mesophase includes a single compound which meets all these requirements. Mixtures of two to 25, preferably three to 18, compounds are therefore generally prepared in order to obtain substances which can be used as LC phases. However, it has not been possible to prepare optimum phases easily in this way since no liquid-crystal materials having significantly negative dielectric anisotropy and adequate long-term stability were hitherto available.

Matrix liquid-crystal displays (MLC displays) are known. Non-linear elements which can be used for individual switching of the individual pixels are, for example, active elements (i.e. transistors). The term “active matrix” is then used, where a distinction can be made between two types:
1. MOS (metal oxide semiconductor) transistors on a silicon wafer as substrate
2. thin-film transistors (TFTs) on a glass plate as substrate.

In the case of type 1, the electro-optical effect used is usually dynamic scattering or the guest-host effect. The use of single-crystal silicon as substrate material restricts the display size, since even modular assembly of various part-displays results in problems at the joints.

In the case of the more promising type 2, which is preferred, the electro-optical effect used is usually the TN effect.

A distinction is made between two technologies: TFTs comprising compound semiconductors, such as, for example, CdSe, or TFTs based on polycrystalline or amorphous silicon. The latter technology is being worked on intensively worldwide.

The TFT matrix is applied to the inside of one glass plate of the display, while the other glass plate carries the transparent counter-electrode on its inside. Compared with the size of the pixel electrode, the TFT is very small and has virtually no adverse effect on the image. This technology can also be extended to fully color-capable displays, in which a mosaic of red, green and blue filters is arranged in such a way that a filter element is opposite each switchable pixel.

The term MLC displays here covers any matrix display with integrated non-linear elements, i.e. besides the active matrix, also displays with passive elements, such as varistors or diodes (MIM=metal-insulator-metal).

MLC displays of this type are particularly suitable for TV applications (for example pocket TVs) or for high-information displays in automobile or aircraft construction. Besides problems regarding the angle dependence of the contrast and the response times, difficulties also arise in MLC displays due to insufficiently high specific resistance of the liquid-crystal mixtures [TOGASHI, S., SEKIGUCHI, K., TANABE, H., YAMAMOTO, E., SORIMACHI, K., TAJIMA, E., WATANABE, H., SHIMIZU, H., Proc. Eurodisplay 84, September 1984: A 210-288 Matrix LCD Controlled by Double Stage Diode Rings, pp. 141 ff., Paris; STROMER, M., Proc. Eurodisplay 84, September 1984: Design of Thin Film Transistors for Matrix Addressing of Television Liquid Crystal Displays, pp. 145 ff., Paris]. With decreasing resistance, the contrast of an MLC display deteriorates. Since the specific resistance of the liquid-crystal mixture generally drops over the life of an MLC display owing to interaction with the inside surfaces of the display, a high (initial) resistance is very important for displays that have to have acceptable resistance values over a long operating period.

There is thus still a great demand for MLC displays having fast response times at the same time as a large working-temperature range and a low threshold voltage, with the aid of which various grey shades can be generated.

The disadvantage of the MLC-TN displays frequently used is due to their comparatively low contrast, the relatively high viewing-angle dependence and the difficulty of generating grey shades in these displays.

VA displays have significantly better viewing-angle dependences and are therefore principally used for televisions and monitors. However, there continues to be a need to improve the response times here, in particular in view of use for televisions having frame rates (image change frequency/refresh rate) of greater than 60 Hz. However, the properties, such as, for example, the low-temperature stability, must not be impaired at the same time.

The invention is based on the object of providing liquid-crystal mixtures, in particular for monitor and TV applications, based on the ECB effect or on the IPS or FFS effect, which do not have the disadvantages indicated above, or only do so to a reduced extent. In particular, it must be ensured for monitors and televisions that they also work at extremely high and extremely low temperatures and at the same time have very short response times and at the same time have improved reliability behavior, in particular exhibit no or significantly reduced image sticking after long operating times.

Surprisingly, it is possible to reduce the ratio of rotational viscosity λ₁ and the elastic constant K₃₃ (λ₁/K₃₃) and thus to improve the response times if the compound of the formula I is used in liquid-crystal mixtures, in particular in LC mixtures having negative dielectric anisotropy, preferably for VA, IPS and FFS displays, furthermore for PM (passive matrix)-VA displays.

Surprisingly, the compound of the formula I simultaneously has a very low rotational viscosity and a high absolute value of the dielectric anisotropy. It is therefore possible to prepare liquid-crystal mixtures, preferably VA, IPS and FFS mixtures, which have very short response times, at the same time good phase properties and good low-temperature behavior.

The invention thus relates to a liquid-crystalline medium which comprises a compound of the formula I.

The mixtures according to the invention preferably exhibit very broad nematic phase ranges with clearing points ≧68° C., preferably ≧70° C., very favorable values of the capacitive threshold, relatively high values of the holding ratio and at the same time very good low-temperature stabilities at −20° C. and −30° C., as well as low rotational viscosities and short response times. The mixtures according to the invention are furthermore distinguished by the fact that, in addition to the improvement in the rotational viscosity yi, relatively high values of the elastic constants K₃₃ for improving the response times can be observed.

Some preferred embodiments of the mixtures according to the invention are indicated below.

The compound of the formula I is preferably employed in the liquid-crystalline medium in amounts of ≧1% by weight, preferably ≧3% by weight, based on the mixture as a whole. Particular preference is given to liquid-crystalline media which comprise 5-30% by weight, very particularly preferably 5-15% by weight, of the compound of the formula I.

Preferred embodiments of the liquid-crystalline medium according to the invention are indicated below:
a) Liquid-crystalline medium which additionally comprises one or more compounds selected from the group of the compounds of the formulae IIA, IIB and IIC,

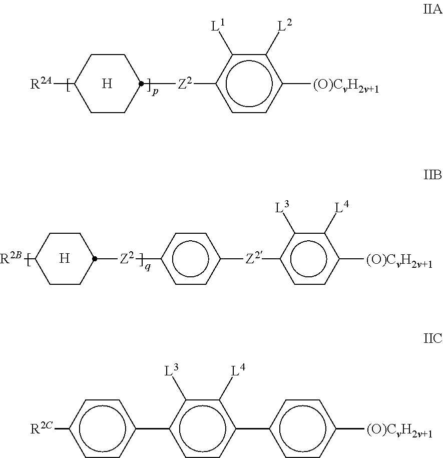

in which
R^{2A}, R^{2B} and R^{2C} each, independently of one another, denote H, an alkyl or alkenyl radical having up to 15 C atoms which is unsubstituted, monosubstituted by CN or CF₃ or at least monosubstituted by halogen, where, in addition, one or more CH₂ groups in these radicals are each optionally replaced by —O—, —S—,

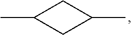

—C≡C—, —CF₂O—, —OCF₂—, —OC—O— or —O—CO— in such a way that O atoms are not linked directly to one another, or a cycloalkyl ring having 3 to 6 C atoms,
L¹⁻⁴ each, independently of one another, denote F, Cl, CF₃ or CHF₂,
Z² and Z² each, independently of one another, denote a single bond, —CH₂CH₂—, —CH═CH—, —CF₂O—, —OCF₂—, —CH₂O—, —OCH₂—, —COO—, —OCO—, —C₂F₄—, —CF═CF—, or —CH═CHCH₂O—,
(O) denotes O or a single bond,
p denotes 1 or 2,
q denotes 0 or 1, and
v denotes 1 to 6.
In the compounds of the formulae IIA and IIB, Z² may have identical or different meanings. In the compounds of the formula IIB, Z² and Z^{2′} may have identical or different meanings.
In the compounds of the formulae IIA, IIB and IIC, R^{2A}, R^{2B} and R^{2C} each preferably denote alkyl having 1-6 C atoms, in particular CH₃, C₂H₅, n-C₃H₇, n-C₄H₉, or n-C₅H₁₁.
In the compounds of the formulae IIA and IIB, L¹, L², L³ and L⁴ preferably denote L¹=L²=F and L³=L⁴=F, furthermore L¹=F and L²=Cl, L¹=Cl and L²=F, L³=F and L⁴=Cl, L³=Cl and L⁴=F. Z² and Z^{2′} in the formulae IIA and IIB preferably each, independently of one another, denote a single bond, furthermore a —C₂H₄— or —CH₂O—bridge.
If in the formula IIB Z²=—C₂H₄— or —CH₂O—, Z^{2′} is preferably a single bond or, if Z^{2′}=—C₂H₄— or —CH₂O—, Z² is preferably a single bond. In the compounds of the formulae IIA and IIB, (O)CᵥH₂ᵥ₊₁ preferably denotes OCᵥH₂ᵥ₊₁, furthermore CᵥH₂ᵥ₊₁. In the compounds of the formula IIC, (O)CᵥH₂ᵥ₊₁ preferably denotes CᵥH₂ᵥ₊₁. In the compounds of the formula IIC, L³ and L⁴ preferably each denote F.
Preferred compounds of the formulae IIA, IIB and IIC are indicated below:

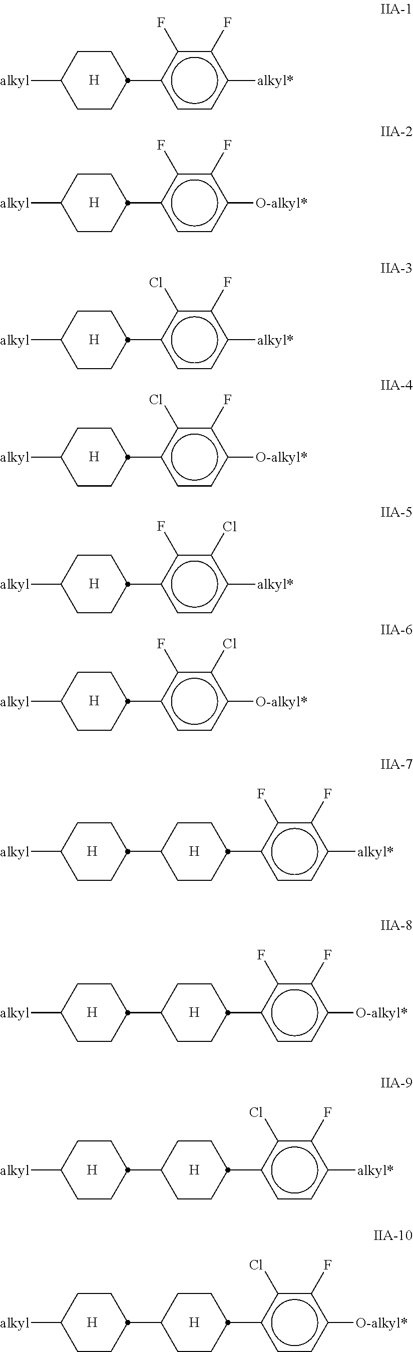

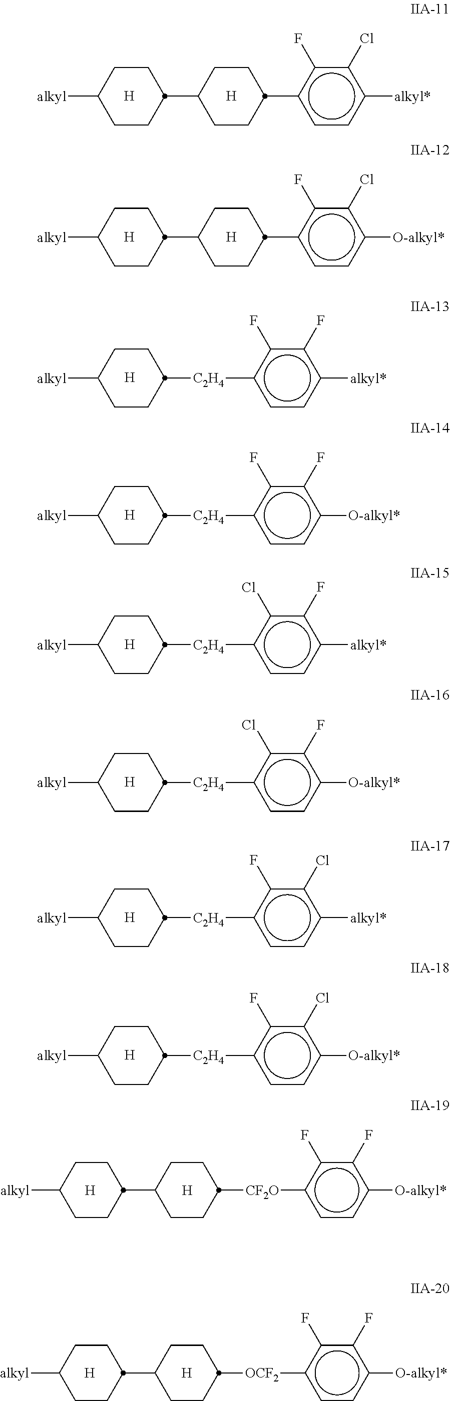

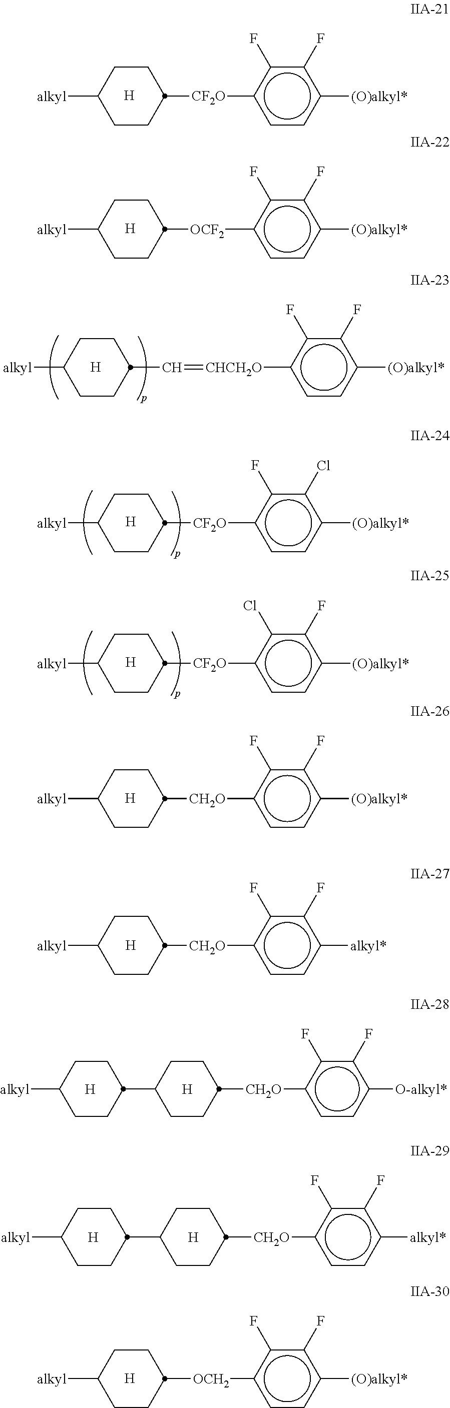

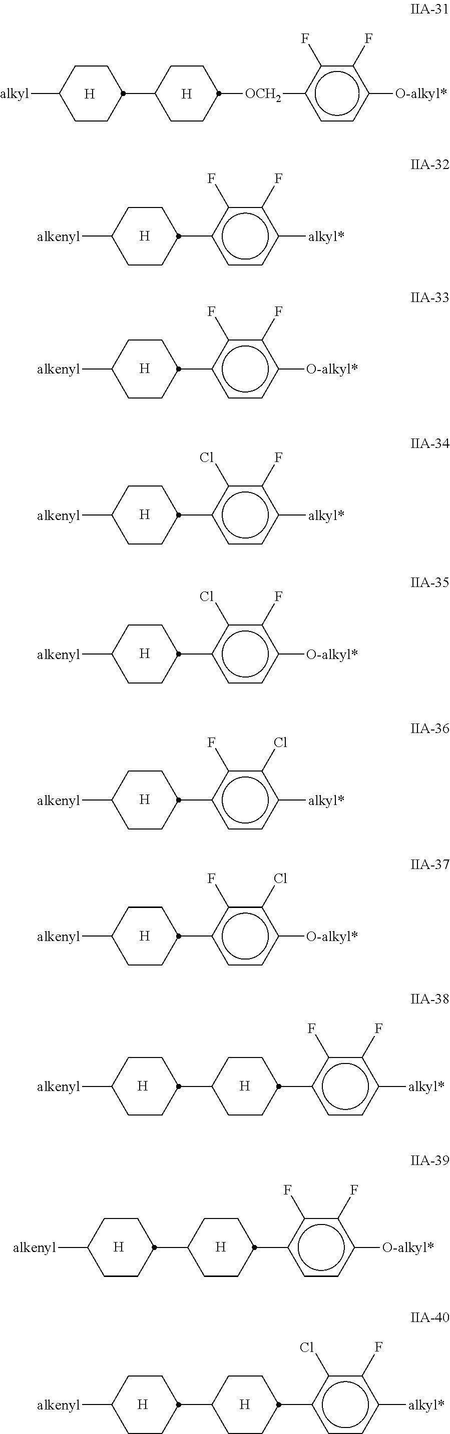

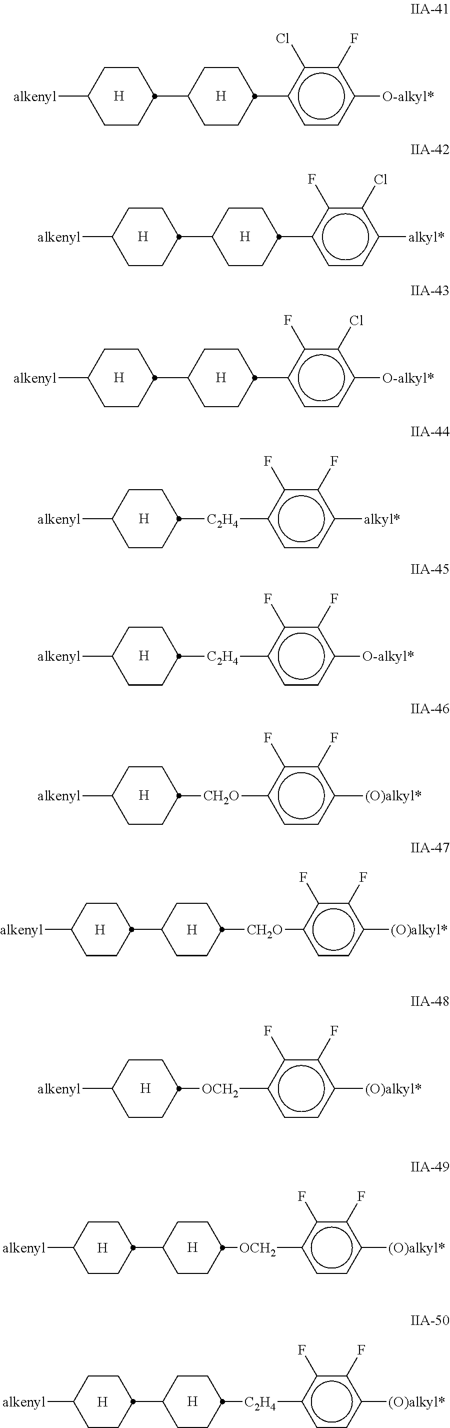

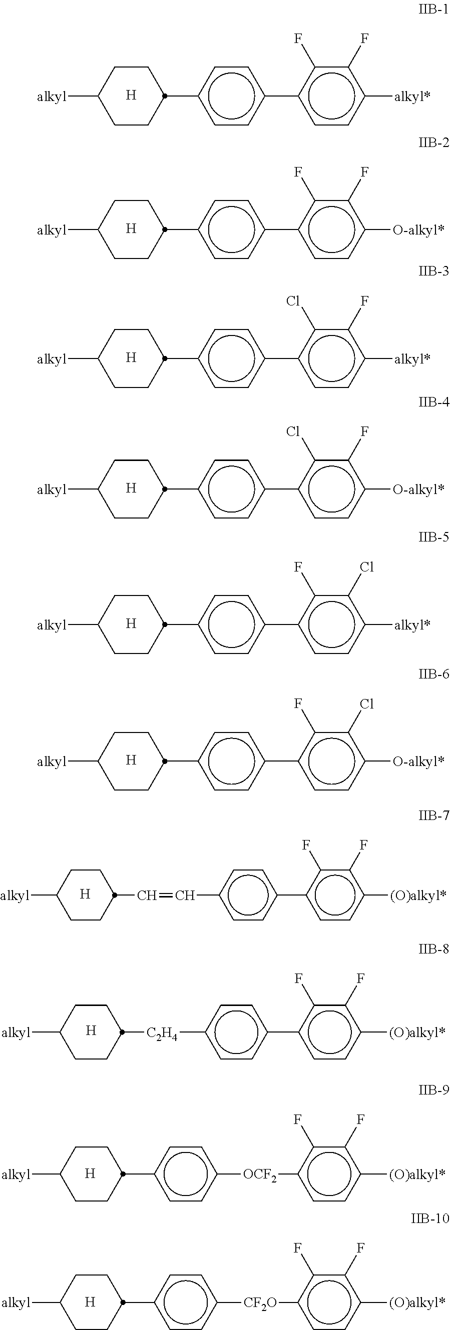

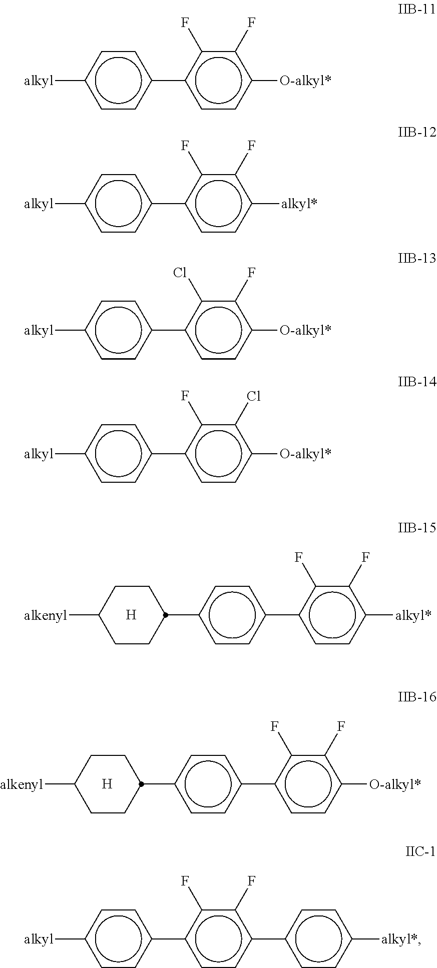

in which alkyl and alkyl* each, independently of one another, denote a straight-chain alkyl radical having 1-6 C atoms, alkenyl denotes an alkenyl radical having 2-6 C atoms, and (O) denotes O or a single bond.
Particularly preferred mixtures according to the invention comprise one or more compounds of the formulae IIA-2, IIA-8, IIA-14, IIA-26, IIA-28, IIA-33, IIA-39, IIA-45, IIA-46, IIA-47, IIB-2, IIB-11, IIB-16 and IIC-1.
The proportion of compounds of the formulae IIA and/or IIB in the mixture as a whole is preferably at least 20% by weight.
Particularly preferred media according to the invention comprise at least one compound of the formula IIC-1,

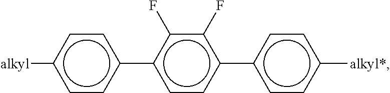

in which alkyl and alkyl* have the meanings indicated above, preferably in amounts of >3% by weight, in particular >5% by weight and particularly preferably 5-25% by weight.
b) Liquid-crystalline medium which additionally comprises one or more compounds of the formula III,

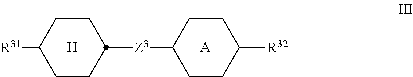

in which
R³¹ and R³² each, independently of one another, denote a straight-chain alkyl, alkoxyalkyl or alkoxy radical having up to 12 C atoms, and

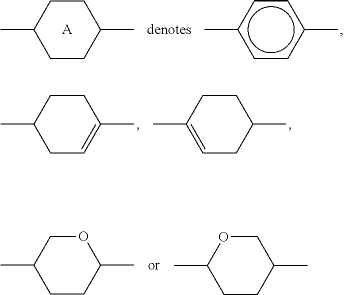

Z³ denotes a single bond, —CH₂CH₂—, —CH═CH—, —CF₂O—, —OCF₂—, —CH₂O—, —OCH₂—, —COO—, —OCO—, —C₂F₄—, —C₄H₈—, or —CF═CF—.
Preferred compounds of the formula III are indicated below:

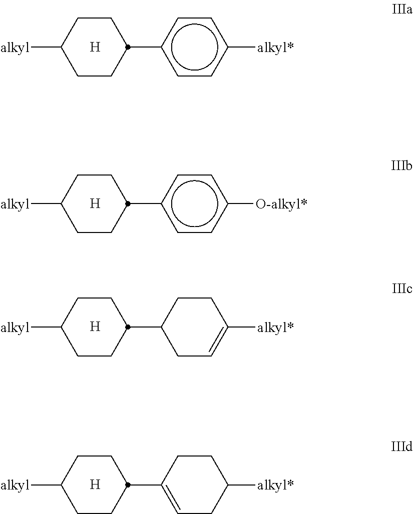

in which
alkyl and
alkyl* each, independently of one another, denote a straight-chain alkyl radical having 1-6 C atoms.
The medium according to the invention preferably comprises at least one compound of the formula IIIa and/or formula IIIb.
The proportion of compounds of the formula III in the mixture as a whole is preferably at least 5% by weight.
c) Liquid-crystalline medium which additionally comprises one or more tetracyclic compounds of the formulae

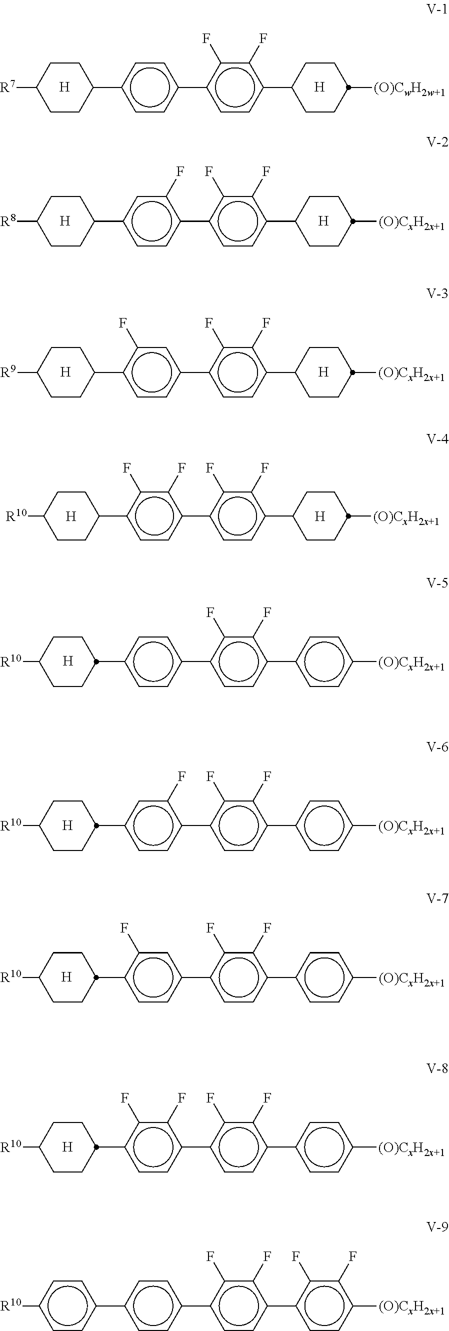

in which
R⁷⁻¹⁰ each, independently of one another, have one of the meanings indicated for R^{2A} in formula IIA and
w and x each, independently of one another, denote 1 to 6.
Particular preference is given to mixtures comprising at least one compound of the formula V-9.
d) Liquid-crystalline medium which additionally comprises one or more compounds of the formulae Y-1 to Y-6,

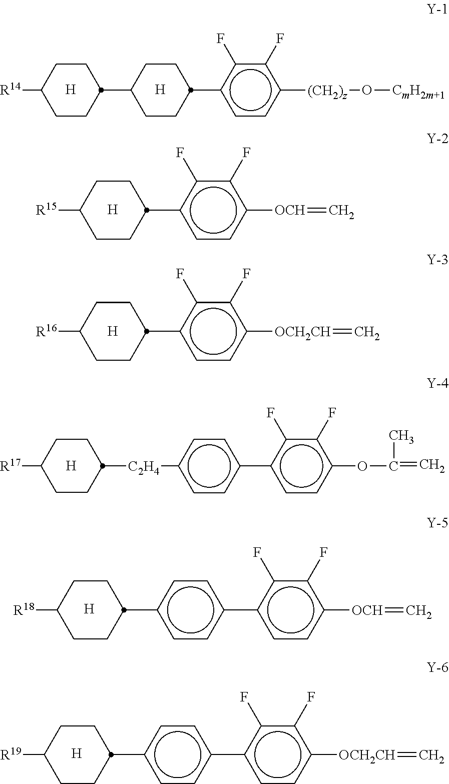

in which R¹⁴-R¹⁹ each, independently of one another, denotes an alkyl or alkoxy radical having 1-6 C atoms; and z and m each, independently of one another, denote 1-6.
The medium according to the invention particularly preferably comprises one or more compounds of the formulae Y-1 to Y-6, preferably in amounts of ≧5% by weight.
e) Liquid-crystalline medium additionally comprising one or more fluorinated terphenyls of the formulae T-1 to T-21,

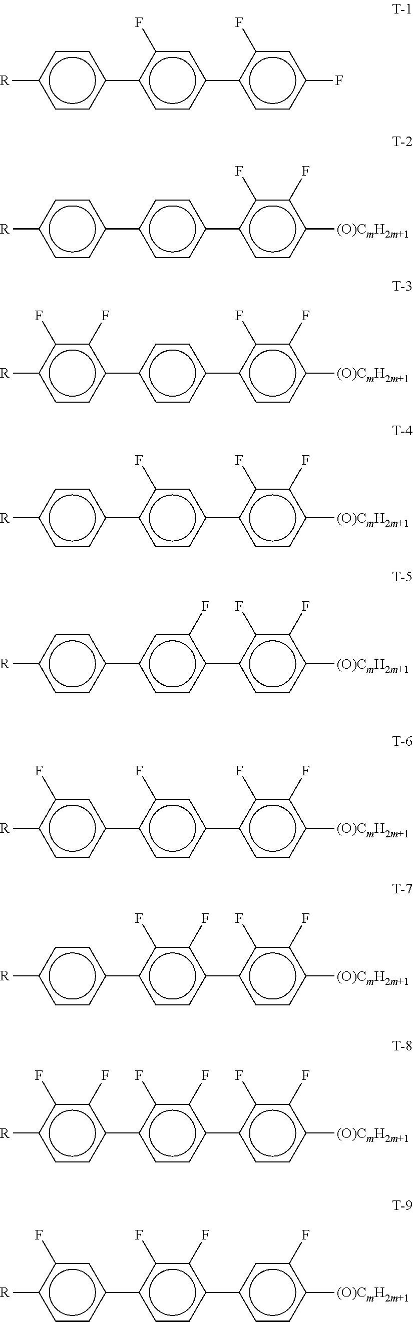

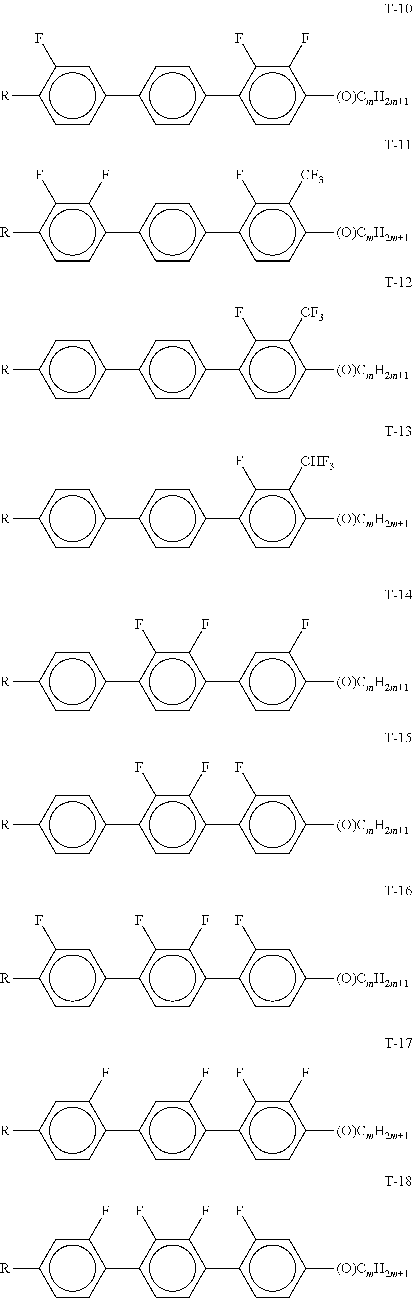

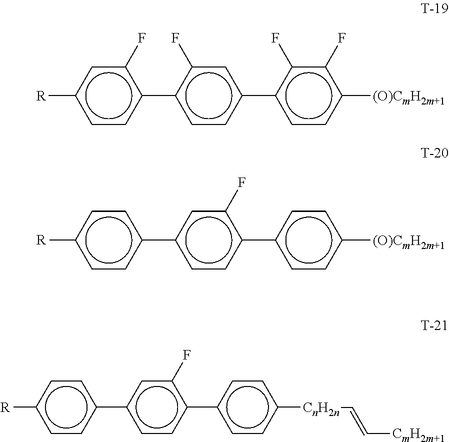

in which
R denotes a straight-chain alkyl or alkoxy radical having 1-7 C atoms or alkenyl having 2-7 C atoms,
(O) denotes O or a single bond,
m=0, 1, 2, 3, 4, 5 or 6, and
n denotes 0, 1, 2, 3 or 4.
R preferably denotes methyl, ethyl, propyl, butyl, pentyl, hexyl, methoxy, ethoxy, propoxy, butoxy, or pentoxy.
The medium according to the invention preferably comprises the terphenyls of the formulae T-1 to T-21 in amounts of 2-30% by weight, in particular 5-20% by weight.
Particular preference is given to compounds of the formulae T-1, T-2, T-20 and T-21. In these compounds, R preferably denotes alkyl, furthermore alkoxy, each having 1-5 C atoms. In the compounds of the formula T-20, R preferably denotes alkyl or alkenyl, in particular alkyl. In the compound of the formula T-21, R preferably denotes alkyl.
The terphenyls are preferably employed in the mixtures according to the invention if the Δn value of the mixture is to be ≧0.1. Preferred mixtures comprise 2-20% by weight of one or more terphenyl compounds selected from the group of the compounds T-1 to T-21.
f) Liquid-crystalline medium additionally comprising one or more biphenyls of the formulae B-1 to B-4,

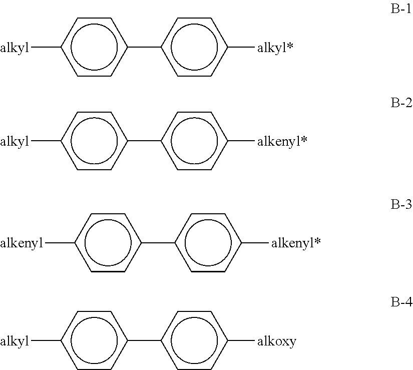

in which
alkyl and alkyl* each, independently of one another, denote a straight-chain alkyl radical having 1-6 C atoms,
alkenyl and alkenyl* each, independently of one another, denote a straight-chain alkenyl radical having 2-6 C atoms, and
alkoxy denotes a straight-chain alkoxy radical having 1-6 C atoms.
The proportion of the biphenyls of the formulae B-1 to B-4 in the mixture as a whole is preferably at least 3% by weight, in particular ≧5% by weight.
Of the compounds of the formulae B-1 to B-4, the compounds of the formula B-2 are particularly preferred.
Particularly preferred biphenyls are

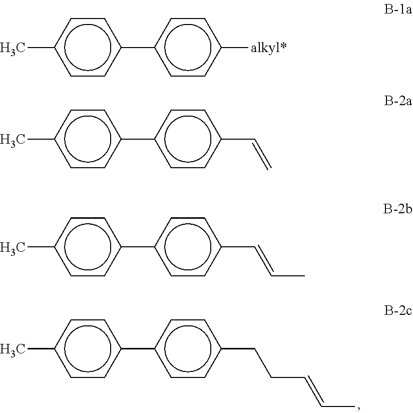

in which alkyl* denotes an alkyl radical having 1-6 C atoms. The medium according to the invention particularly preferably comprises one or more compounds of the formulae B-1a and/or B-2c.
g) Liquid-crystalline medium comprising at least one compound of the formulae Z-1 to Z-7,

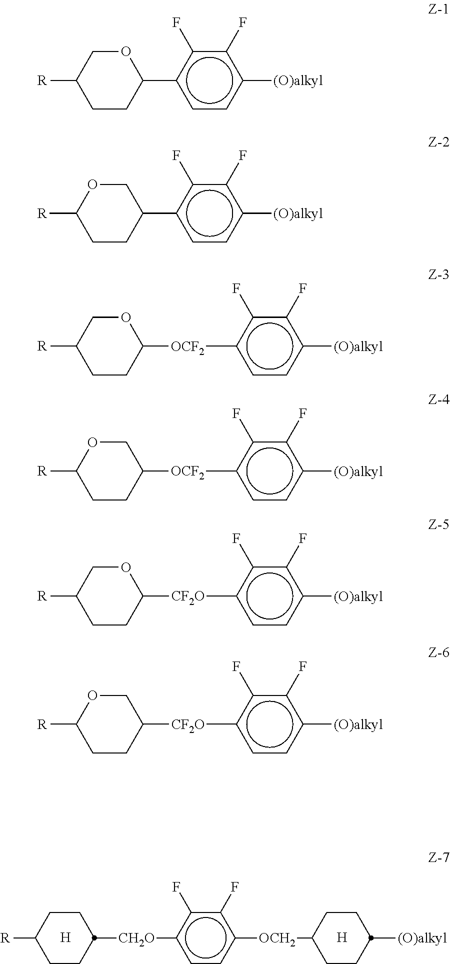

in which
R denotes a straight-chain alkyl or alkoxy radical having 1-7 C atoms or an alkenyl radical having 2-7 C atoms,
alkyl denotes an alkyl radical having 1-6 C atoms, and
(O)alkyl denotes alkyl or Oalkyl.
h) Liquid-crystalline medium comprising at least one compound of the formulae O-1 to O-17,

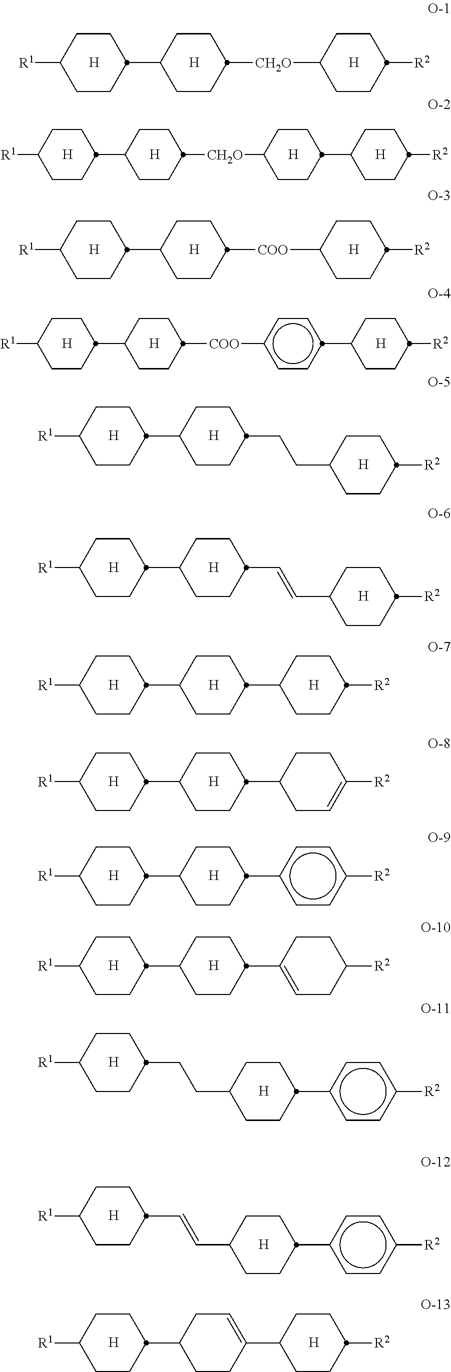

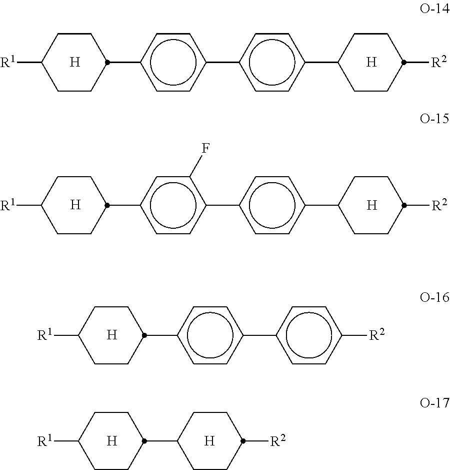

in which R¹ and R² have the meanings indicated for R^{2A} in formula IIA and the compound(s) of the formula O-17 is (are) not identical with the compound of the formula I. R¹ and R² preferably each, independently of one another, denote straight-chain alkyl having 1-6 C atoms or R¹ denotes straight-chain alkyl having 1-6 C atoms and R² denotes alkenyl having 2-6 C atoms.
Preferred media comprise one or more compounds of the formulae O-1, O-3, O-4, O-5, O-9, O-12, O-14, O-15, O-16 and/or O-17.
Mixtures according to the invention very particularly preferably comprise the compounds of the formulae O-9, O-12, O-16 and/or O-17, in particular in amounts of 5-30%.
Preferred compounds of the formulae O-9 and O-17 are indicated below:

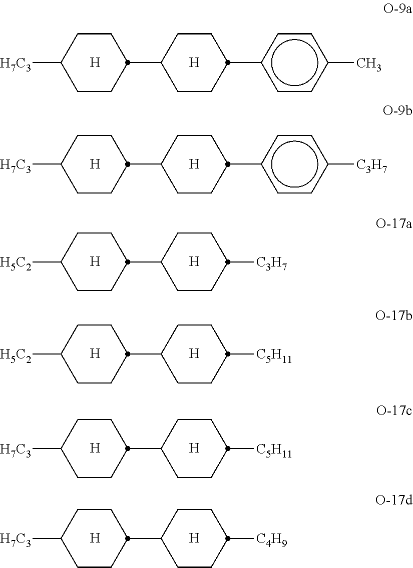

The medium according to the invention particularly preferably comprises the tricyclic compounds of the formula O-9a and/or of the formula O-9b in combination with one or more bicyclic compounds of the formulae O-17a to O-17d. The total proportion of the compounds of the formulae O-9a and/or O-9b in combination with one or more compounds selected from the bicyclic compounds of the formulae O-17a to O-17d is preferably 5-40%, very particularly preferably 15-35%.
Very particularly preferred mixtures comprise the compounds O-9a and O-17a:

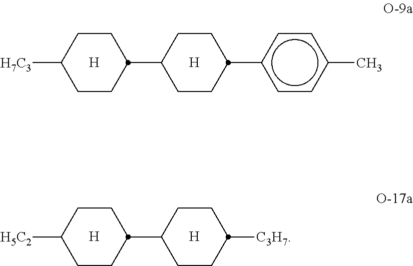

The compounds O-9a and O-17a are preferably present in the mixture in a concentration of 15-35%, particularly preferably 15-25% and especially preferably 18-22%, based on the mixture as a whole.
Very particularly preferred mixtures comprise the compounds O-9b and O-17a:

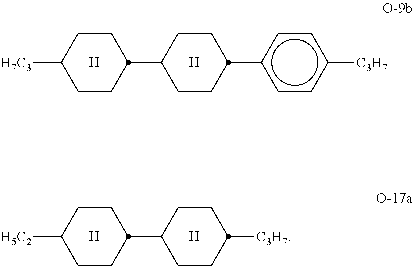

The compounds O-9b and O-17a are preferably present in the mixture in a concentration of 15-35%, particularly preferably 15-25% and especially preferably 18-22%, based on the mixture as a whole.
Very particularly preferred mixtures comprise the following three compounds:

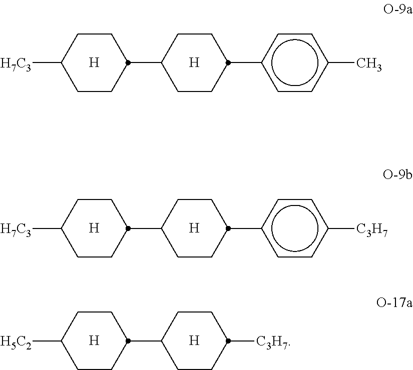

The compounds O-9a, O-9b and O-17a are preferably present in the mixture in a concentration of 15-35%, particularly preferably 15-25% and especially preferably 18-22%, based on the mixture as a whole.
Preferred compounds of the formula O-17 are furthermore the compounds selected from the group of the compounds of the formulae

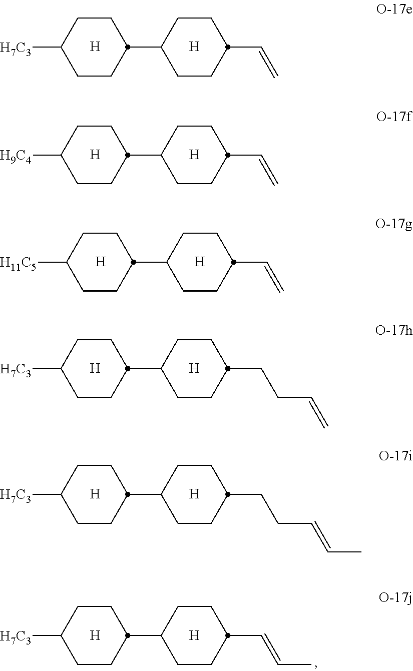

preferably in each case in amounts of ≧3% by weight, in particular ≧10% by weight.
Preferred mixtures comprise 5-60% by weight, preferably 10-55% by weight, in particular 20-50% by weight, of the compound of the formula O-17e

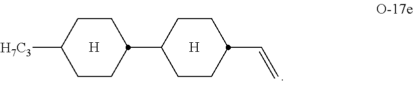

Preference is furthermore given to liquid-crystalline mixtures which comprise the compound O-17e

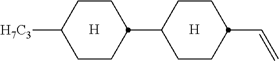

and the compound O-17i

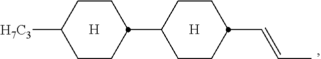

preferably in total amounts of 3-60% by weight.
i) Liquid-crystalline medium comprising one or more compounds of the formula BA

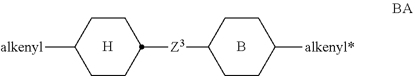

in which
alkenyl and alkenyl* each, independently of one another, denote a straight-chain alkenyl radical having 2-12 C atoms,

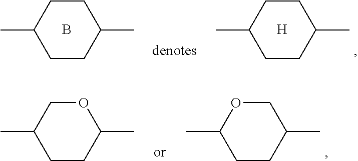

Z³ denotes a single bond, —CH₂CH₂—, —CH═CH—, —CF₂O—, —OCF₂—, —CH₂O—, —OCH₂—, —COO—, —OCO—, —C₂F₄—, —C₄H₈—, or —CF═CF—.
Preferred compounds of the formula BA are indicated below:

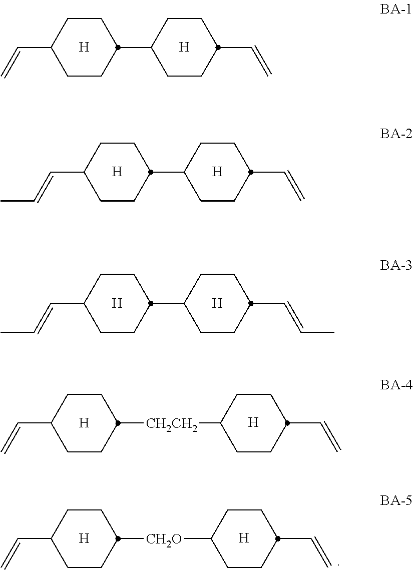

The compounds of formulae BA-1 to BA-5 are preferably present in the mixture in a concentration of 2-40% based on the mixture as a whole.
j) Preferred liquid-crystalline media according to the invention comprise one or more substances which contain a tetrahydronaphthyl or naphthyl unit, such as, for example, the compounds of the formulae N-1 to N-5,

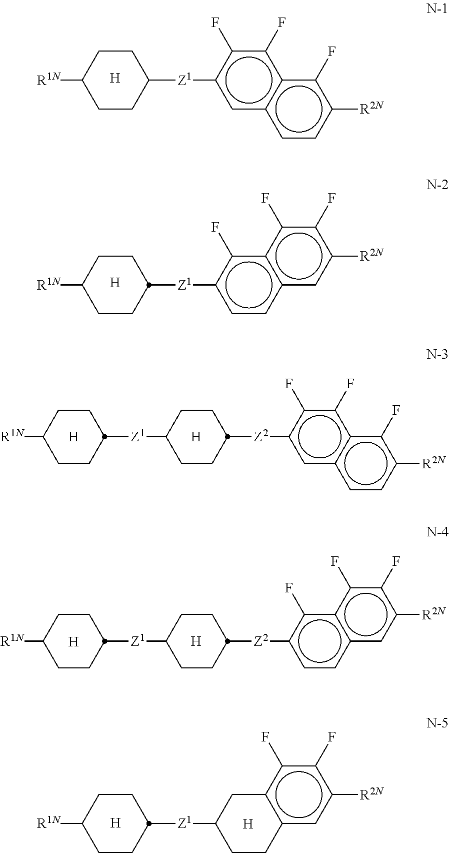

in which R^{1N} and R^{2N} each, independently of one another, have the meanings indicated for R^{2A} in formula IIA, preferably denote straight-chain alkyl, straight-chain alkoxy or straight-chain alkenyl, and
Z¹ and Z² each, independently of one another, denote —C₂H₄—, —CH═CH—, —(CH₂)₄—, —(CH₂)₃O—, —O(CH₂)₃—, —CH═CHCH₂CH₂—, —CH₂CH₂CH═CH—, —CH₂O—, —OCH₂—, —COO—, —OCO—, —C₂F₄—, —CF═CF—, —CF═CH—, —CH═CF—, —CF₂O—, —OCF₂—, —CH₂— or a single bond.
k) Preferred mixtures comprise one or more compounds selected from the group of the difluorodibenzochroman compounds of the formula BC, chromans of the formula CR, fluorinated phenanthrenes of the formulae PH-1 and PH-2, fluorinated dibenzofurans of the formulae BF-1 and BF-2, and fluorinated dibenzothiophenes of the formulae BS-1 and BS-2,

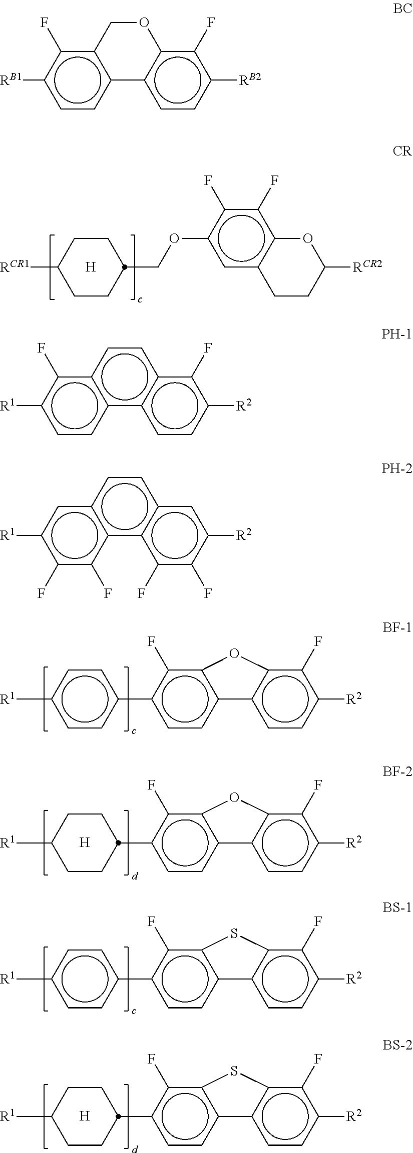

in which
R^{B1}, R^{B2}, R^{CR1}, R^{CR2}, R¹, R² each, independently of one another, have the meaning of R^{2A} in formula IIA. c is 0, 1 or 2. d is 1 or 2. R¹ and R² preferably, independently of one another, denote alkyl, alkoxy, alkenyl or alkenyloxy having 1 to 6 or 2 to 6 C atoms respectively.
The mixtures according to the invention preferably comprise the compounds of the formulae BC, CR, PH-1, PH-2, BF-1, BF-2, BS-1 and/or BS-2 in amounts of 3 to 20% by weight, in particular in amounts of 3 to 15% by weight.
Particularly preferred compounds of the formulae BC, CR, BF and BS are the compounds BC-1 to BC-7 and CR-1 to CR-5,

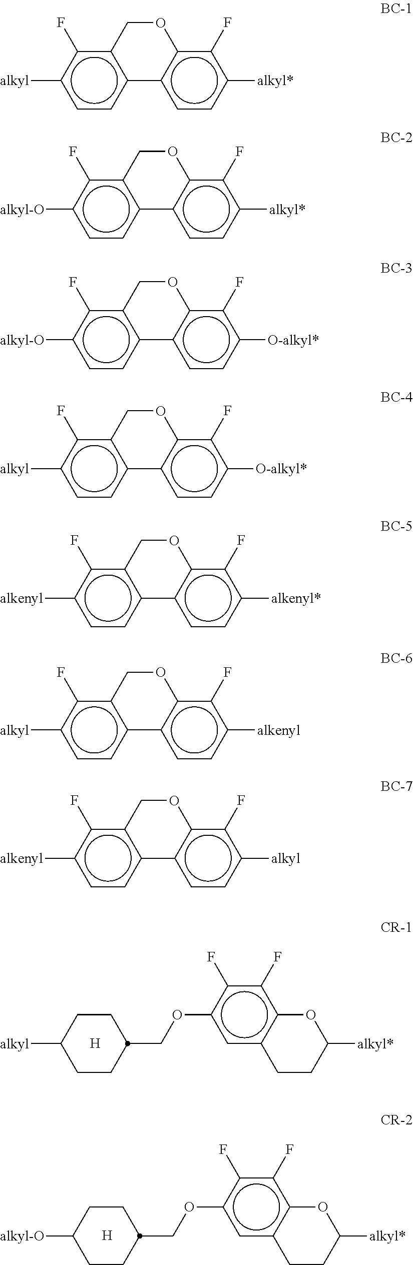

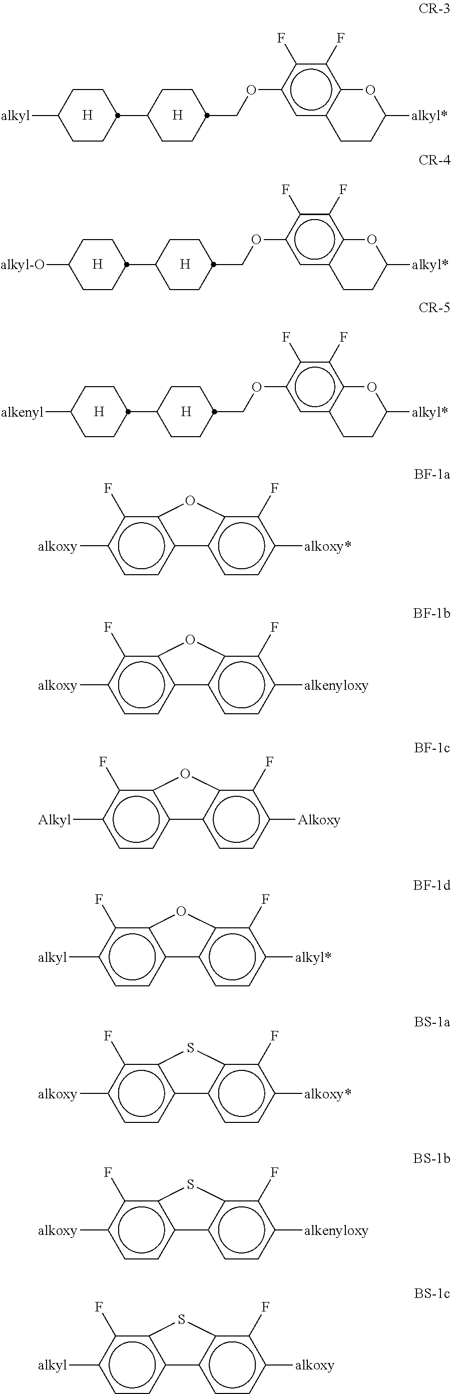

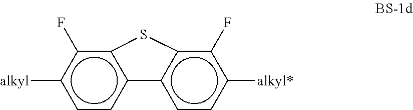

in which
alkyl and alkyl* each, independently of one another, denote a straight-chain alkyl radical having 1-6 C atoms,
alkoxy and
alkoxy* each, independently of one another, denote a straight-chain alkoxy radical having 1-6 C atoms,
alkenyl and
alkenyl* each, independently of one another, denote a straight-chain alkenyl radical having 2-6 C atoms, and
alkenyloxy denotes a straight-chain alkenyloxy radical having 2-6 C atoms.
Very particular preference is given to mixtures comprising one, two or three compounds of the formulae BC-2 and/or BF-1a.
I) Preferred mixtures comprise one or more indane compounds of the formula In,

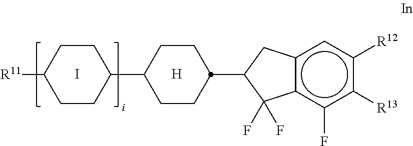

in which
R¹¹, R¹²,
R¹³ each, independently of one another, denote a straight-chain alkyl, alkoxy, alkoxyalkyl or alkenyl radical having 1-6 C atoms or 2-6 C atoms respectively,
R¹² and R¹³ additionally may denote H or halogen, preferably F,

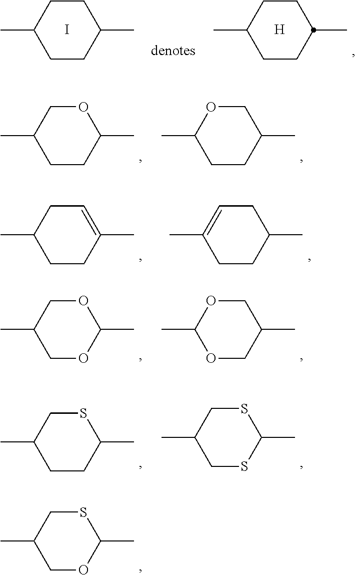

i denotes 0, 1 or 2.
Preferred compounds of the formula In are the compounds of the formulae In-1 to In-16 indicated below:

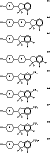

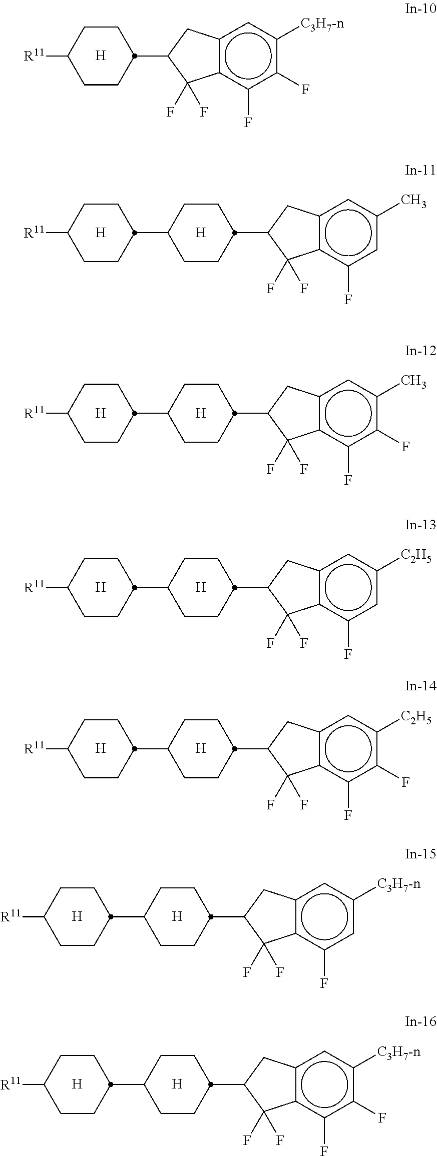

Particular preference is given to the compounds of the formulae In-1, In-2, In-3 and In-4.
The compounds of the formula In and the sub-formulae In-1 to In-16 are preferably employed in the mixtures according to the invention in concentrations ≧5% by weight, in particular 5-30% by weight and very particularly preferably 5-25% by weight.
m) Preferred mixtures additionally comprise one or more compounds of the formulae L-1 to L-11,

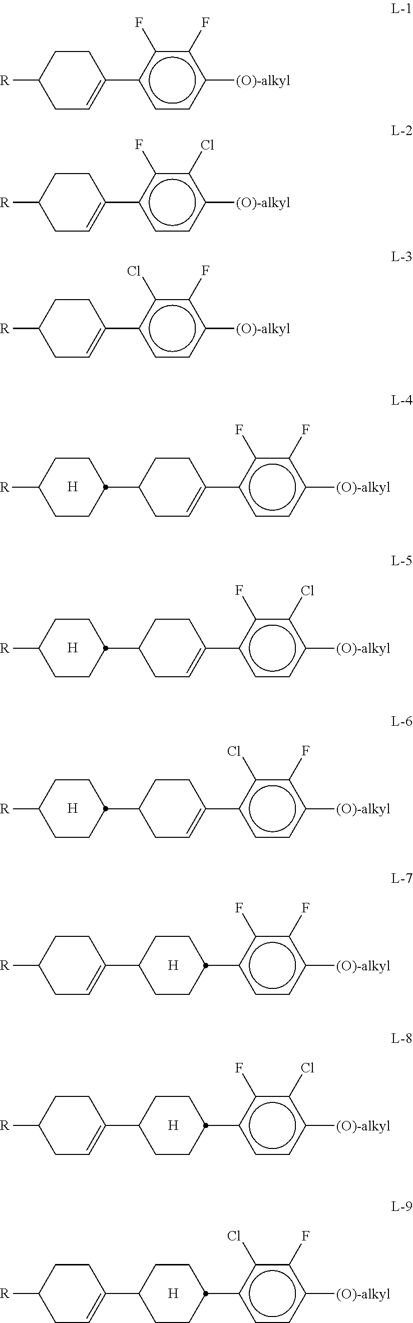

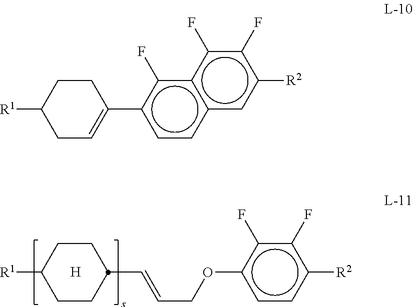

in which
R, R¹ and R² each, independently of one another, have the meanings indicated for R^{2A} in formula IIA, (O) denotes O or a single bond, and alkyl denotes an alkyl radical having 1-6 C atoms. s denotes 1 or 2.

Particular preference is given to the compounds of the formulae L-1 and L-4, in particular L-4.

The compounds of the formulae L-1 to L-11 are preferably employed in concentrations of 5-50% by weight, in particular 5-40% by weight and very particularly preferably 10-40% by weight.
n) The medium additionally comprises one or more compounds of the formula EY

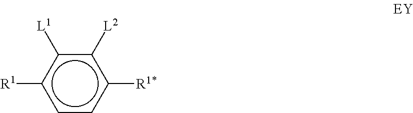

in which
R¹ and R^{1*} each, independently of one another, have the meanings indicated for R^{2A} in formula IIA and
L¹ and L² each, independently of one another, denote F, Cl, CF₃ or CHF₂.
In the compounds of the formula EY, R¹ and R^{1*} preferably denote alkoxy having ≧2 C atoms and L¹=L²=F. Particular preference is given to the compounds of the formulae

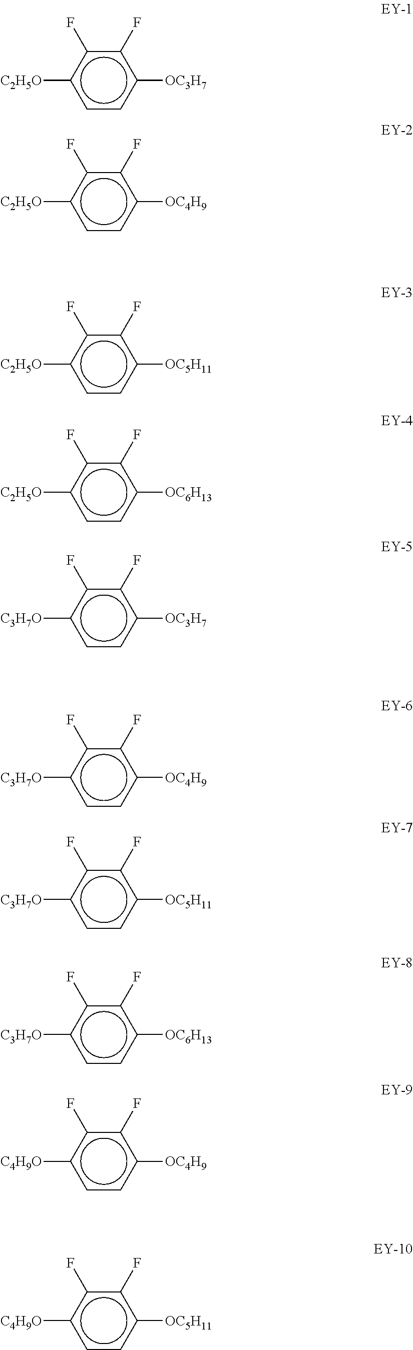

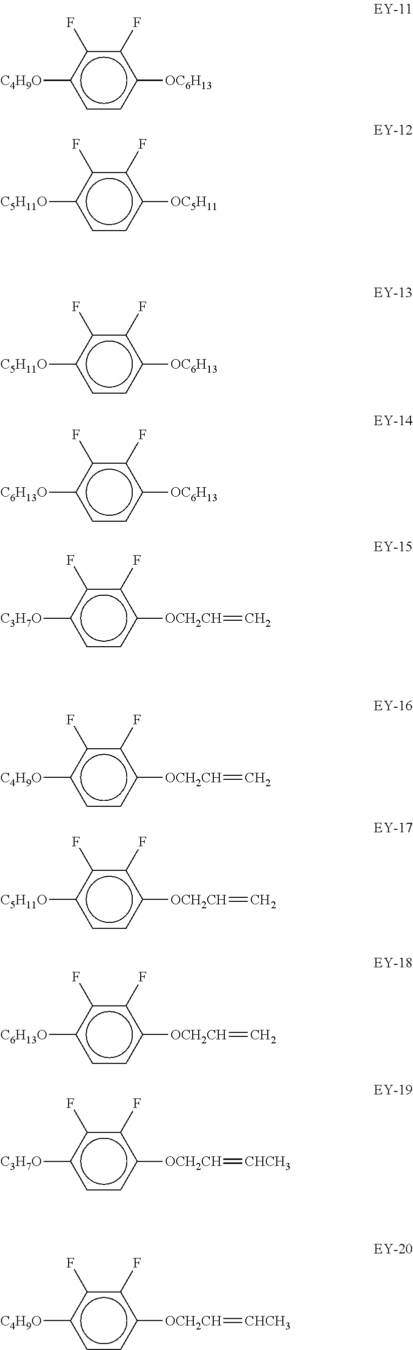

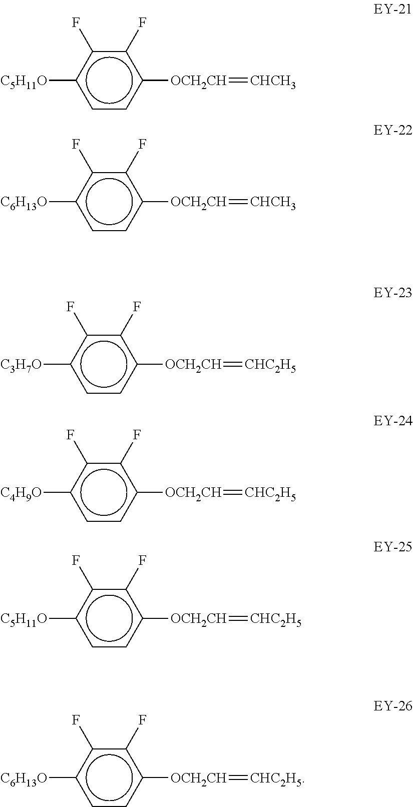

o) The medium additionally comprises one or more compounds selected from the following formulae:

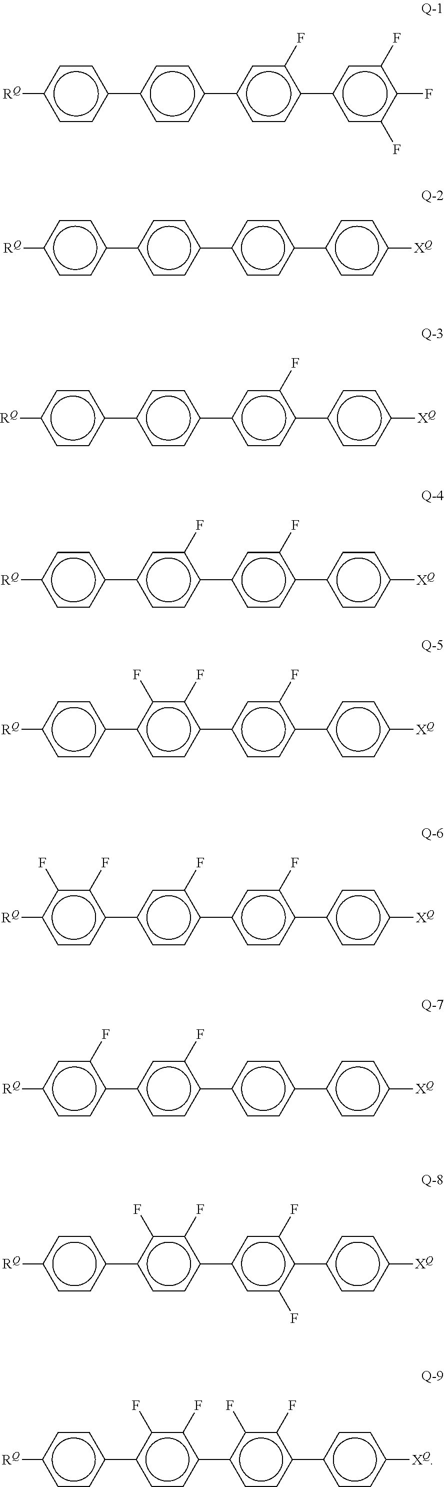

In the compounds of the formulae Q-1 to Q-9, R^{Q} and X^{Q} each, independently of one another, have the meanings of R^{2A} in formula IIA. R^{Q} and X^{Q} preferably denote a straight-chain alkyl radical having 1-6 C atoms, in particular having 2-5 C atoms.

Particularly preferred mixture concepts are indicated below: (the acronyms used are explained in Tables 1-3 and in Table A. n and m here each, independently of one another, denotes 1-6).

The mixtures according to the invention preferably comprise
the compound of the formula I in combination with one or more compounds of the formula O-17, preferably with the compound CCH-23,
   and/or
the compound of the formula I in combination with CCH-25, CCH-34 and/or CCH-35,
   and/or
CPY-n-Om, in particular CPY-2-O2, CPY-3-O2 and/or CPY-5-O2, preferably in concentrations >5%, in particular 10-30%, based on the mixture as a whole,
   and/or
B-nO-Om, preferably in concentrations of 1-15,
   and/or
CY-n-Om, preferably CY-3-O2, CY-3-O4, CY-5-O2 and/or CY-5-O4, preferably in concentrations >5%, in particular 15-50%, based on the mixture as a whole,
   and/or
CCY-n-Om, preferably CCY-4-O2, CCY-3-O2, CCY-3-O3, CCY-3-O1 and/or CCY-5-O2, preferably in concentrations >5%, in particular 10-30%, based on the mixture as a whole,
   and/or
CLY-n-Om, preferably CLY-2-O4, CLY-3-O2 and/or CLY-3-O3, preferably in concentrations >5%, in particular 10-30%, based on the mixture as a whole,
   and/or
CK-n-F, preferably CK-3-F, CK-4-F and/or CK-5-F, preferably >5%, in particular 5-25%, based on the mixture as a whole.

Preference is furthermore given to mixtures according to the invention which comprise the following mixture concepts:

(n and m each, independently of one another, denote 1-6.)
CPY-n-Om and CY-n-Om, preferably in concentrations of 10-80%, based on the mixture as a whole,
   and/or
CPY-n-Om and CK-n-F, preferably in concentrations of 10-70%, based on the mixture as a whole,
   and/or
CPY-n-Om and PY-n-Om, preferably CPY-2-O2 and/or CPY-3-O2 and PY-3-O2, preferably in concentrations of 10-40%, based on the mixture as a whole,
   and/or
CPY-n-Om and CLY-n-Om, preferably in concentrations of 10-80%, based on the mixture as a whole,
   and/or
CC-3-V1, preferably in amounts of 3-15%
   and/or
   CC-V-V, preferably in amounts of 5-60%
   and/or
   CC-3-V, preferably in amounts of 5-60%
   and/or
   PGIY-n-Om, preferably in amounts of 3-15%,
   and/or
   CC-n-2V1, preferably in amounts of 3-20%.

The invention furthermore relates to an electro-optical display having active-matrix addressing based on the ECB, VA, PS-VA, PVA, PM-VA, SS-VA, IPS, PS-IPS, FFS or PS-FFS effect, in particular UB-FFS, characterized in that it contains, as dielectric, a liquid-crystalline medium according to one or more of Claims 1 to 14.

The liquid-crystalline medium according to the invention preferably has a nematic phase from ≦−20° C. to ≧70° C., particularly preferably from ≦−30° C. to ≧80° C., very particularly preferably from ≦−40° C. to ≧90° C.

The expression “have a nematic phase” here means on the one hand that no smectic phase and no crystallization are observed at low temperatures at the corresponding temperature and on the other hand that clearing still does not occur on heating from the nematic phase. The investigation at low temperatures is carried out in a flow viscometer at the corresponding temperature and checked by storage in test cells having a layer thickness corresponding to the electro-optical use for at least 100 hours. If the storage stability at a temperature of −20° C. in a corresponding test cell is 1000 h or more, the medium is referred to as stable at this temperature. At temperatures of −30° C. and −40° C., the corresponding times are 500 h and 250 h respectively. At high temperatures, the clearing point is measured by conventional methods in capillaries.

The liquid-crystal mixture preferably has a nematic phase range of at least 60 K and a flow viscosity v₂₀ of at most 30 mm²·s⁻¹ at 20° C.

The values of the birefringence Δn in the liquid-crystal mixture are generally between 0.07 and 0.16, preferably between 0.08 and 0.13.

The liquid-crystal mixture according to the invention has a Δ∈ of −0.5 to −8.0, in particular −2.5 to −6.0, where Δ∈ denotes the dielectric anisotropy. The rotational viscosity γ₁ at 20° C. is preferably ≦150 mPa·s, in particular ≦130 mPa·s.

The liquid-crystal media according to the invention have relatively small values for the threshold voltage (V₀). They are preferably in the range from 1.7 V to 3.0 V, particularly preferably ≦2.5 V and very particularly preferably ≦2.3 V.

For the present invention, the term “threshold voltage” relates to the capacitive threshold (V₀), also known as the Freedericks threshold, unless explicitly indicated otherwise.

In addition, the liquid-crystal media according to the invention have high values for the voltage holding ratio in liquid-crystal cells.

In general, liquid-crystal media having a low addressing voltage or threshold voltage exhibit a lower voltage holding ratio than those having a higher addressing voltage or threshold voltage and vice versa.

For the present invention, the term “dielectrically positive compounds” denotes compounds having a Δ∈>1.5, the term “dielectrically neutral compounds” denotes those where −1.5≦Δ∈≦1.5 and the term “dielectrically negative compounds” denotes those having Δ∈<−1.5. The dielectric anisotropy of the compounds is determined here by dissolving 10% of the compounds in a liquid-crystalline host and determining the capacitance of the resultant mixture in at least one test cell in each case having a layer thickness of 20 μm with homeotropic and with homogeneous surface alignment at 1 kHz. The measurement voltage is typically 0.5 V to 1.0 V, but is always lower than the capacitive threshold of the respective liquid-crystal mixture investigated.

All temperature values indicated for the present invention are in ° C.

The mixtures according to the invention are suitable for all VA-TFT applications, such as, for example, VAN, MVA, (S)-PVA, ASV, PSA (polymer sustained VA), SS (surface-stabilized)-VA and PS-VA (polymer stabilized VA). They are furthermore suitable for IPS (in-plane switching) and FFS (fringe field switching), in particular UB-FFS, having negative Δ∈.

The nematic liquid-crystal mixtures in the displays according to the invention generally comprise two components A and B, which themselves consist of one or more individual compounds.

Component A has significantly negative dielectric anisotropy and gives the nematic phase a dielectric anisotropy of ≦−0.5. Besides one or more compounds of the formula I, it preferably comprises the compounds of the formulae IIA, IIB and/or IIC, furthermore compounds of the formula Ill.

The proportion of component A is preferably between 45 and 100%, in particular between 60 and 100%.

For component A, one (or more) individual compound(s) which has (have) a value of Δ∈≦−0.8 is (are) preferably selected. This value must be more negative, the smaller the proportion A in the mixture as a whole.

Component B has pronounced nematogeneity and a flow viscosity of not greater than 30 mm²·s⁻¹, preferably not greater than 25 mm²·s⁻¹, at 20° C.

A multiplicity of suitable materials is known to the person skilled in the art from the literature. Particular preference is given to compounds of the formula Ill.

Particularly preferred individual compounds in component B are extremely low-viscosity nematic liquid crystals having a flow viscosity of not greater than 18 mm²·s⁻¹, preferably not greater than 12 mm²·s⁻¹, at 20° C.

Component B is monotropically or enantiotropically nematic, has no smectic phases and is able to prevent the occurrence of smectic phases down to very low temperatures in liquid-crystal mixtures. For example, if various materials of high nematogeneity are in each case added to a smectic liquid-crystal mixture, the nematogeneity of these materials can be compared through the degree of suppression of smectic phases that is achieved.

The mixture may optionally also comprise a component C, comprising compounds having a dielectric anisotropy of Δ∈≧1.5. These so-called positive compounds are generally present in a mixture of negative dielectric anisotropy in amounts of ≦20% by weight, based on the mixture as a whole.

If the mixture according to the invention comprises one or more compounds having a dielectric anisotropy of Δ∈≧1.5, these are preferably one or more compounds of the formulae P-1 and/or P-2,

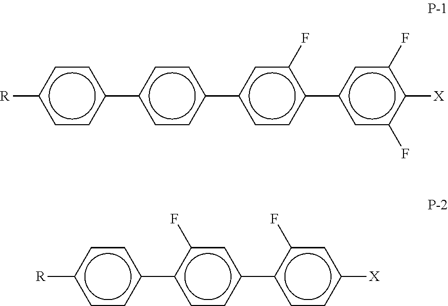

in which
R denotes straight-chain alkyl, alkoxy or alkenyl, each having 1 to 6 C atoms or 2 to 6 C atoms respectively, and
X denotes F, Cl, CF₃, OCF₃, OCHFCF₃ or CCF₂CHFCF₃, preferably F or OCF₃.

The compounds of the formulae P-1 and/or P-2 are preferably employed in the mixtures according to the invention in concentrations of 0.5-10% by weight, in particular 0.5-8% by weight.

Particular preference is given to the compound of the formula

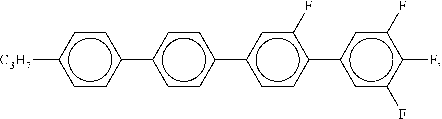

which is preferably employed in an amount of 0.5-3% by weight.

In addition, these liquid-crystal phases may also comprise more than 18 components, preferably 18 to 25 components.

Besides one or more compounds of the formula I, the phases preferably comprise 4 to 15, in particular 5 to 12, and particularly preferably <10, compounds of the formulae IIA, IIB and/or IIC and optionally Ill.

Besides compounds of the formula I and the compounds of the formulae IIA, IIB and/or IIC and optionally III, other constituents may also be present, for example in an amount of up to 45% of the mixture as a whole, but preferably up to 35%, in particular up to 10%.

The other constituents are preferably selected from nematic or nematogenic substances, in particular known substances, from the classes of the azoxybenzenes, benzylideneanilines, biphenyls, terphenyls, phenyl or cyclohexyl benzoates, phenyl or cyclohexyl cyclohexanecarboxylates, phenylcyclohexanes, cyclohexylbiphenyls, cyclohexylcyclohexanes, cyclohexylnaphthalenes, 1,4-biscyclohexylbiphenyls or cyclohexylpyrimidines, phenyl- or cyclohexyldioxanes, optionally halogenated stilbenes, benzyl phenyl ethers, tolans and substituted cinnamic acid esters.

The most important compounds which are suitable as constituents of liquid-crystal phases of this type can be characterized by the formula IV,

R²⁰-L-G-E-R²¹ IV

in which L and E each denote a carbo- or heterocyclic ring system from the group formed by 1,4-disubstituted benzene and cyclohexane rings, 4,4′-disubstituted biphenyl, phenylcyclohexane and cyclohexylcyclohexane systems, 2,5-disubstituted pyrimidine and 1,3-dioxane rings, 2,6-disubstituted naphthalene, di- and tetrahydronaphthalene, quinazoline and tetrahydroquinazoline,
G denotes —CH═CH— —N(O)═N—
   —CH═CQ— —CH═N(O)—
   —C≡C— —CH₂—CH₂—
   —CO—O— —CH₂—O—
   —CO—S— —CH₂—S—
   —CH═N— —COO-Phe-COO—
   —CF₂O— —CF═CF—
   —OCF₂— —OCH₂—
   —(CH₂)₄— —(CH₂)₃O—
      or a C—C single bond, Q denotes halogen, preferably chlorine, or —CN, and R²⁰ and R²¹ each denote alkyl, alkenyl, alkoxy, alkoxyalkyl or alkoxycarbonyloxy having up to 18, preferably up to 8, carbon atoms, or one of these radicals alternatively denotes CN, NC, NO₂, NCS, CF₃, SF₅, OCF₃, F, Cl or Br.

In most of these compounds, R²⁰ and R²¹ are different from one another, one of these radicals usually being an alkyl or alkoxy group. Other variants of the proposed substituents are also common. Many such substances or also mixtures thereof are commercially available. All these substances can be prepared by methods known from the literature.

It goes without saying for the person skilled in the art that the VA, IPS or FFS mixture according to the invention may also comprise compounds in which, for example, H, N, O, CI and F have been replaced by the corresponding isotopes.

Polymerizable compounds, so-called reactive mesogens (RMs), for example as disclosed in U.S. Pat. No. 6,861,107, may furthermore be added to the mixture s according to the invention in concentrations of preferably 0.01-5% by weight, particularly preferably 0.2-2% by weight, based on the mixture. These mixtures may optionally also comprise an initiator, as described, for example, in U.S. Pat. No. 6,781,665. The initiator, for example Irganox-1076 from BASF, is preferably added to the mixture comprising polymerizable compounds in amounts of O-1%. Mixtures of this type can be used for so-called polymer-stabilized VA modes (PS-VA) or PSA (polymer sustained VA), in which polymerization of the reactive mesogens is intended to take place in the liquid-crystalline mixture. The prerequisite for this is that the liquid-crystal mixture itself comprises no polymerizable components which likewise polymerize under the conditions where the compounds of the formula M polymerize.

The polymerization is preferably carried out under the following conditions: the polymerizable components are polymerized in a cell using a UV-A lamp of defined intensity for a defined period and applied voltage (typically 10 to 30 V alternating voltage, frequencies in the range from 60 Hz to 1 kHz). The UV-A light source employed is typically a metal-halide vapor lamp or high-pressure mercury lamp having an intensity of 50 mW/cm². These are conditions where, for example, liquid-crystalline compounds containing an alkenyl or alkenyloxy side chain, such as, for example, the compounds of the formula

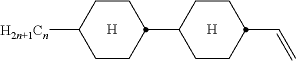

where n=2, 3, 4, 5 or 6,
do not polymerize.

In a preferred embodiment of the invention, the polymerizable compounds are selected from the compounds of the formula M

R^{Ma}-A^{M1}-(Z^{M1}-A^{M2})ₘ₁-R^{Mb} M

in which the individual radicals have the following meaning:
R^{Ma} and R^{Mb} each, independently of one another, denote P, P-Sp-, H, F, Cl, Br, I, —CN, —NO₂, —NCO, —NCS, —OCN, —SCN, SF₅ or straight-chain or branched alkyl, alkenyl, or alkynyl having up to 25 C atoms, in which, in addition, one or more non-adjacent CH₂ groups may each be replaced, independently of one another, by —C(R⁰)═C(R⁰⁰)—, —C≡C—, —N(R⁰⁰)—, —O—, —S—, —CO—, —CO—O—, —O—CO—, or —O—CO—O— in such a way that O and/or S atoms are not linked directly to one another, and in which, in addition, one or more H atoms are each optionally replaced by F, Cl, Br, I, CN, P or P-Sp-, where at least one of the radicals R^{Ma} and R^{Mb} preferably denotes or contains a group P or P-Sp-,
P denotes a polymerizable group,
Sp denotes a spacer group or a single bond,
A^{M1} and A^{M2} each, independently of one another, denote an aromatic, heteroaromatic, alicyclic or heterocyclic group, preferably having 4 to 25 ring atoms, preferably C atoms, which also includes or may contain annellated rings, and which may optionally be mono- or polysubstituted by L,
L denotes P, P-Sp-, OH, CH₂OH, F, Cl, Br, I, —CN, —NO₂, —NCO, —NCS, —OCN, —SCN, —C(═O)N(R^{x})₂, —C(═O)Y¹, —C(═O)R^{x},
   —N(R^{x})₂, optionally substituted silyl, optionally substituted aryl having 6 to 20 C atoms, or straight-chain or branched alkyl, alkoxy, alkylcarbonyl, alkoxycarbonyl, alkylcarbonyloxy or alkoxycarbonyloxy having 1 to 25 C atoms, in which, in addition, one or more H atoms are each optionally replaced by F, Cl, P or P-Sp-, preferably P, P-Sp-, H, OH, CH₂OH, halogen, SF₅, NO₂, an alkyl, alkenyl or alkynyl group,
Y¹ denotes halogen,
Z^{M1} denotes —O—, —S—, —CO—, —CO—O—, —OCO—, —O—CO—O—, —OCH₂—,
   —CH₂O—, —SCH₂—, —CH₂S—, —CF₂O—, —OCF₂—, —CF₂S—, —SCF₂—, —(CH₂)ₙ₁—, —CF₂CH₂—, —CH₂CF₂—, —(CF₂)ₙ₁—, —CH═CH—, —CF═CF—, —C≡C—, —CH═CH—, —COO—, —OCO—CH═CH—, CR⁰R⁰⁰ or a single bond,
R⁰ and R⁰⁰ each, independently of one another, denote H or alkyl having 1 to 12 C atoms,
R^{x} denotes P, P-Sp-, H, halogen, straight-chain, branched or cyclic alkyl having 1 to 25 C atoms, in which, in addition, one or more non-adjacent CH₂ groups are each optionally replaced by —O—,—S—, —CO—, —CO—O—, —O—CO—, or —O—CO—O— in such a way that O and/or S atoms are not linked directly to one another, and in which, in addition, one or more H atoms are each optionally replaced by F, Cl, P or P-Sp-, an optionally substituted aryl or aryloxy group having 6 to 40 C atoms, or an optionally substituted heteroaryl or heteroaryloxy group having 2 to 40 C atoms,
m1 denotes 0, 1, 2, 3 or 4, and
n1 denotes 1, 2, 3 or 4,
   where at least one, preferably one, two or three, particularly preferably one or two, from the group R^{Ma}, R^{Mb} and the substituents L present denotes a group P or P-Sp- or contains at least one group P or P-Sp-.

Particularly preferred compounds of the formula M are those in which
R^{Ma} and R^{Mb} each, independently of one another, denote P, P-Sp-, H, F, Cl, Br, I, —CN, —NO₂, —NCO, —NCS, —OCN, —SCN, SF₅ or straight-chain or branched alkyl having 1 to 25 C atoms, in which, in addition, one or more non-adjacent CH₂ groups may each be replaced, independently of one another, by —C(R⁰)═C(R⁰⁰)—, —C≡C—, —N(R⁰⁰)—, —O—, —S—, —CO—, —CO—O—, —O—CO—, or —O—CO—O— in such a way that O and/or S atoms are not linked directly to one another, and in which, in addition, one or more H atoms are each optionally replaced by F, Cl, Br, I, CN, P or P-Sp-, where at least one of the radicals R^{Ma} and R^{Mb} preferably denotes or contains a group P or P-Sp-,
A^{M1} and A^{M2} each, independently of one another, denote 1,4-phenylene, naphthalene-1,4-diyl, naphthalene-2,6-diyl, phenanthrene-2,7-diyl, anthracene-2,7-diyl, fluorene-2,7-diyl, coumarine, flavone, where, in addition, one or more CH groups in these groups are each optionally replaced by N, cyclohexane-1,4-diyl, in which, in addition, one or more non-adjacent CH₂ groups are each optionally replaced by O or S, 1,4-cyclohexenylene, bicyclo[1.1.1]pentane-1,3-diyl, bicyclo[2.2.2]octane-1,4-diyl, spiro[3.3]heptane-2,6-diyl, piperidine-1,4-diyl, decahydronaphthalene-2,6-diyl, 1,2,3,4-tetrahydronaphthalene-2,6-diyl, indane-2,5-diyl or octahydro-4,7-methanoindane-2,5-diyl, where all these groups are unsubstituted or mono- or polysubstituted by L,
L denotes P, P-Sp-, OH, CH₂OH, F, Cl, Br, I, —CN, —NO₂, —NCO, —NCS, —OCN, —SCN, —C(═O)N(R^{x})₂, —C(═O)Y¹, —C(═O)R^{x},
   —N(R^{x})₂, optionally substituted silyl, optionally substituted aryl having 6 to 20 C atoms, or straight-chain or branched alkyl, alkoxy, alkylcarbonyl, alkoxycarbonyl, alkylcarbonyloxy or alkoxycarbonyloxy having 1 to 25 C atoms, in which, in addition, one or more H atoms are each optionally replaced by F, Cl, P or P-Sp-,
P denotes a polymerizable group,
Y¹ denotes halogen,
R^{x} denotes P, P-Sp-, H, halogen, straight-chain, branched or cyclic alkyl having 1 to 25 C atoms, in which, in addition, one or more non-adjacent CH₂ groups are each optionally replaced by —O—, —S—, —CO—, —CO—O—, —O—CO—, or —O—CO—O— in such a way that O and/or S atoms are not linked directly to one another, and in which, in addition, one or more H atoms are each optionally replaced by F, Cl, P or P-Sp-, an optionally substituted aryl or aryloxy group having 6 to 40 C atoms, or an optionally substituted heteroaryl or heteroaryloxy group having 2 to 40 C atoms.

Very particular preference is given to compounds of the formula M in which one of R^{Ma} and R^{Mb} or both denote P or P-Sp-.

Suitable and preferred RMs for use in liquid-crystalline media and PS-VA displays or PSA displays according to the invention are selected, for example, from the following formulae:

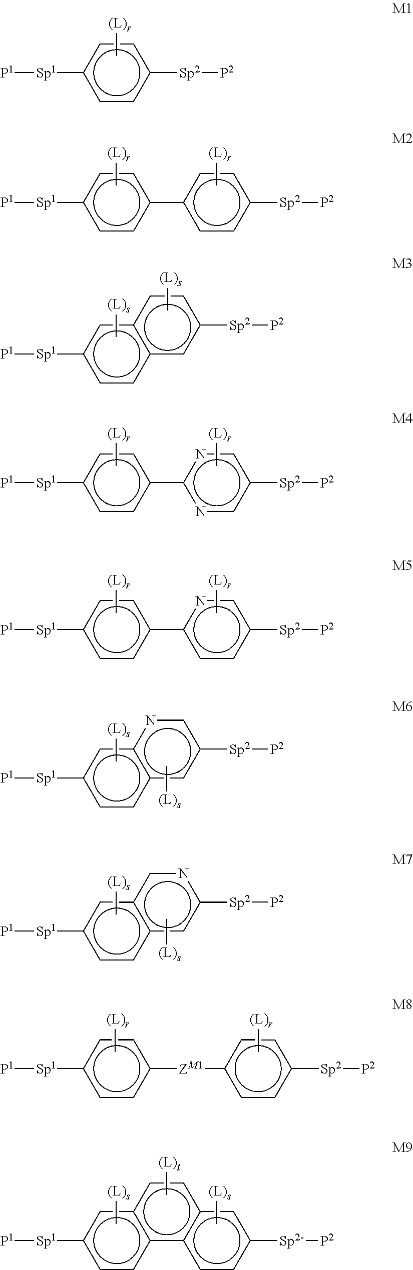

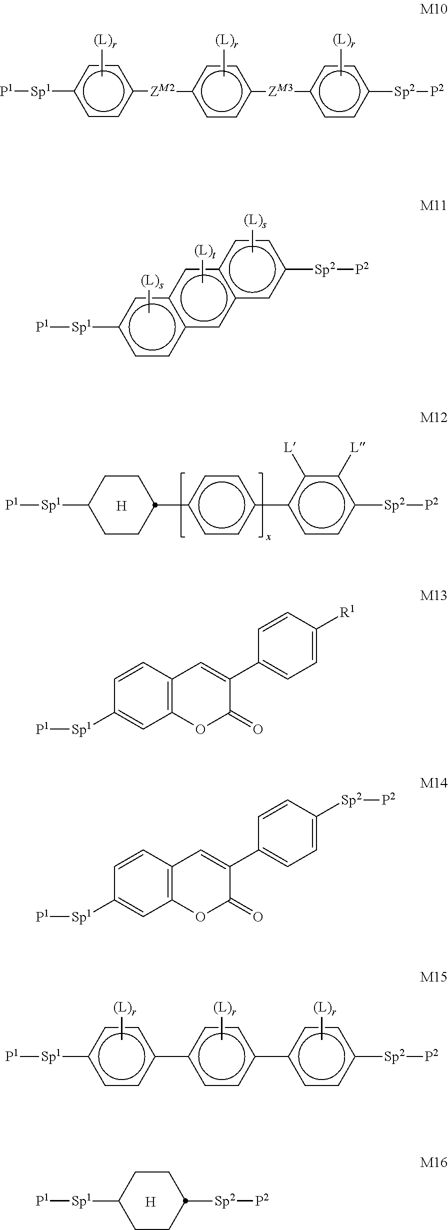

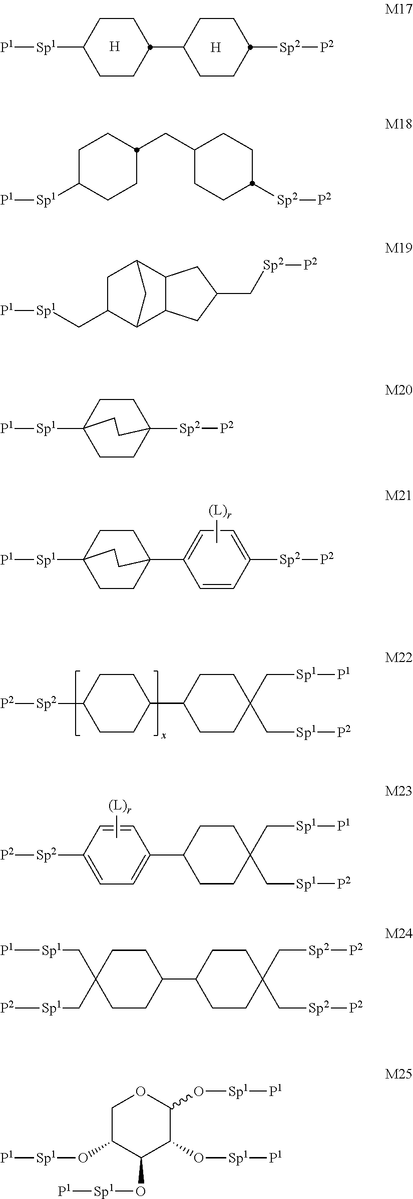

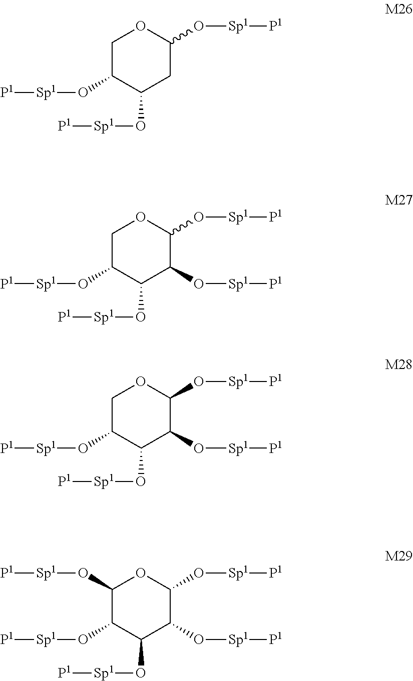

in which the individual radicals have the following meaning:
P¹ and P² each, independently of one another, denote a polymerizable group, preferably having one of the meanings indicated above and below for P, particularly preferably an acrylate, methacrylate, fluoroacrylate, oxetane, vinyloxy or epoxy group,
Sp¹ and Sp² each, independently of one another, denote a single bond or a spacer group, preferably having one of the meanings indicated above and below for Sp, and particularly preferably —(CH₂)ₚ₁—, —(CH₂)ₚ₁—O—, —(CH₂)ₚ₁—CO—O— or —(CH₂)ₚ₁—O—CO—O—, in which p1 is an integer from 1 to 12, and where in the last-mentioned groups the linking to the adjacent ring takes place via the O atom, where one of the radicals P¹-Sp¹- and P²-Sp²- may also denote R^{aa},
R^{aa} denotes H, F, Cl, CN or straight-chain or branched alkyl having 1 to 25 C atoms, in which, in addition, one or more non-adjacent CH₂ groups may each be replaced, independently of one another, by —C(R⁰)═C(R⁰⁰)—, —C≡C—, —N(R⁰)—, —O—, —S—, —CO—, —CO—O—, —O—CO—, —O—CO—O— in such a way that O and/or S atoms are not linked directly to one another, and in which, in addition, one or more H atoms are each optionally replaced by F, Cl, CN or P¹-Sp¹-, particularly preferably straight-chain or branched, optionally mono- or polyfluorinated, alkyl, alkoxy, alkenyl, alkynyl, alkylcarbonyl, alkoxycarbonyl or alkylcarbonyloxy having 1 to 12 C atoms (where the alkenyl and alkynyl radicals have at least two and the branched radicals at least three C atoms),
R⁰, R⁰⁰ each, independently of one another and on each occurrence identically or differently, denote H or alkyl having 1 to 12 C atoms,
Z^{M1} denotes —O—, —CO—, —C(R^{y}R^{Z})— or —CF₂CF₂—,
Z^{M2} and Z^{M3} each, independently of one another, denote —CO—O—, —O—CO—, —CH₂O—, —OCH₂—, —CF₂O—, —OCF₂— or —(CH₂)ₙ—, where n is 2, 3 or 4,
R^{y} and R^{z} each, independently of one another, denote H, F, CH₃ or CF₃,
L on each occurrence, identically or differently, denotes F, Cl, CN, or straight-chain or branched, optionally mono- or polyfluorinated, alkyl, alkoxy, alkenyl, alkynyl, alkylcarbonyl, alkoxycarbonyl or alkylcarbonyloxy having 1 to 12 C atoms, preferably F,
L′ and L″ each, independently of one another, denote H, F or Cl,
r denotes 0, 1, 2, 3 or 4,
s denotes 0, 1, 2 or 3,
t denotes 0, 1 or 2, and
x denotes 0 or 1.

Suitable polymerizable compounds are listed, for example, in Table D.

The liquid-crystalline media in accordance with the present application preferably comprise in total 0.1 to 10%, preferably 0.2 to 4.0%, particularly preferably 0.2 to 2.0%, of polymerizable compounds.

Particular preference is given to the polymerizable compounds of the formula M and of the formulae RM-1 to RM-102.

The mixtures according to the invention may furthermore comprise conventional additives, such as, for example, stabilizers, antioxidants, UV absorbers, nanoparticles, microparticles, etc.

The structure of the liquid-crystal displays according to the invention corresponds to the usual geometry, as described, for example, in EP-A 0 240 379, hereby incorporated by reference.

The following examples are intended to explain the invention without limiting it. Above and below, percent data denote percent by weight; all temperatures are indicated in degrees Celsius.

Throughout the patent application, 1,4-cyclohexylene rings and 1,4-phenylene rings are depicted as follows:

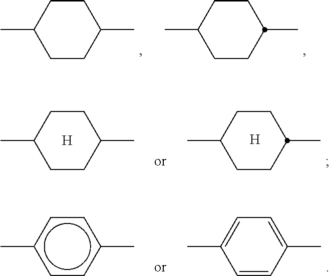

The cyclohexylene rings are trans-1,4-cyclohexylene rings.

Throughout the patent application and in the working examples, the structures of the liquid-crystal compounds are indicated by means of acronyms. Unless indicated otherwise, the transformation into chemical formulae is carried out in accordance with Tables 1-3. All radicals CₙH₂ₙ₊₁, CₘH₂ₘ₊₁ and C_{m′}H_{2m′+1} or CₙH₂ₙ and CₘH₂ₘ are straight-chain alkyl radicals or alkylene radicals, in each case having n, m, m′ or z C atoms respectively. n, m, m′ and z each, independently of one another, denote 1, 2, 3, 4, 5, 6, 7, 8, 9, 10, 11 or 12, preferably 1, 2, 3, 4, 5 or 6. In Table 1 the ring elements of the respective compound are coded, in Table 2 the bridging members are listed and in Table 3 the meanings of the symbols for the lefthand or right-hand side chains of the compounds are indicated.

TABLE 1
Ring elements

| | |
|---|---|
| 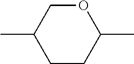 | A |
| 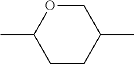 | AI |
| 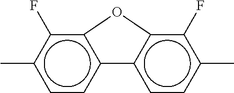 | B |
| 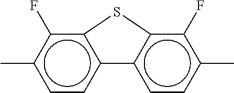 | B(S) |
| 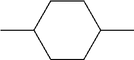 | C |
| 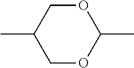 | D |
| 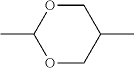 | DI |
| 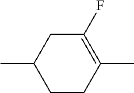 | F |
| 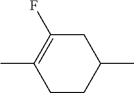 | FI |
| 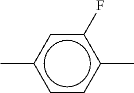 | G |
| 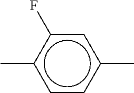 | GI |
| 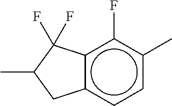 | K |
| 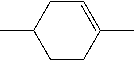 | L |
| 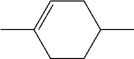 | LI |
| 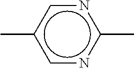 | M |
| 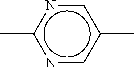 | MI |
| 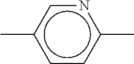 | N |
| 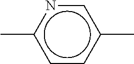 | NI |
| 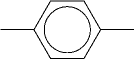 | P |
| 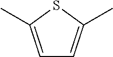 | S |
| 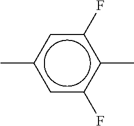 | U |
| 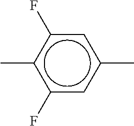 | UI |
| 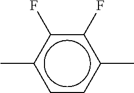 | Y |
| 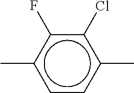 | Y(F,Cl) |
| 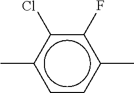 | Y(Cl,F) |

TABLE 2
Bridging members

| | | | |
|---|---|---|---|
| E | —CH₂CH₂— | | |
| V | —CH═CH— | | |
| T | —C≡C— | | |
| W | —CF₂CF₂— | | |
| Z | —COO— | ZI | —OCO— |
| O | —CH₂O— | OI | —OCH₂— |
| Q | —CF₂O— | QI | —OCF₂— |

TABLE 3
Side chains

| Left-hand side chain | | Right-hand side chain | |
|---|---|---|---|
| n- | CₙH₂ₙ₊₁— | -n | —CₙH₂ₙ₊₁ |
| nO- | CₙH₂ₙ₊₁—O— | -On | —O—CₙH₂ₙ₊₁ |
| V- | CH₂═CH— | -V | —CH═CH₂ |
| nV- | CₙH₂ₙ₊₁—CH═CH— | -nV | —CₙH₂ₙ—CH═CH₂ |
| Vn- | CH₂═CH—CₙH₂ₙ— | -Vn | —CH═CH—CₙH₂ₙ₊₁ |
| nVm- | CₙH₂ₙ₊₁—CH═CH—CₘH₂ₘ— | -nVm | —CₙH₂ₙ—CH═CH—CₘH₂ₘ₊₁ |
| N- | N≡C— | -N | —C≡N |
| F- | F— | -F | —F |
| Cl- | Cl— | -Cl | —Cl |
| M- | CFH₂— | -M | —CFH₂ |
| D- | CF₂H— | -D | —CF₂H |
| T- | CF₃— | -T | —CF₃ |
| MO- | CFH₂O— | -OM | —OCFH₂ |
| DO- | CF₂HO— | -OD | —OCF₂H |
| TO- | CF₃O— | -OT | —OCF₃ |
| T- | CF₃— | -T | —CF₃ |
| A- | H—C≡C— | -A | —C≡C—H |

Besides the compounds of the formulae IIA and/or IIB and/or IIC and one or more compounds of the formula I, the mixtures according to the invention preferably comprise one or more of the compounds from Table A indicated below.

TABLE A

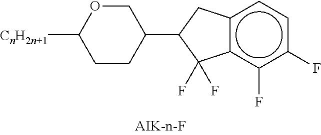

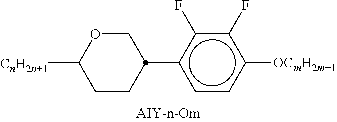

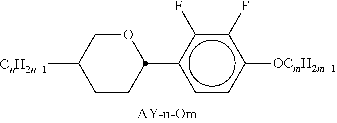

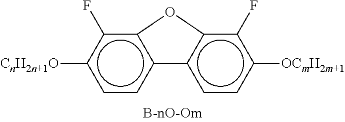

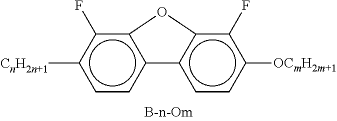

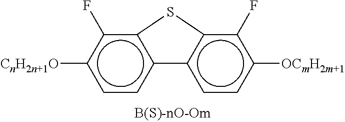

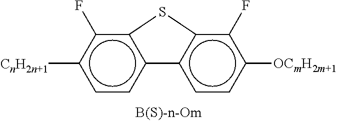

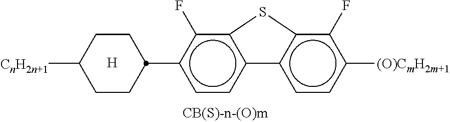

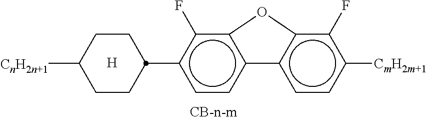

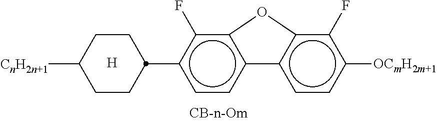

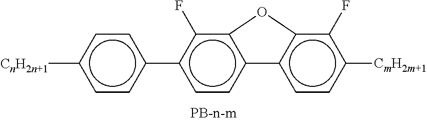

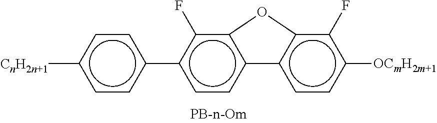

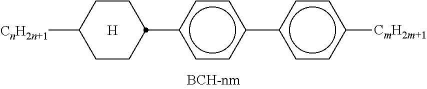

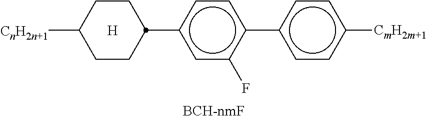

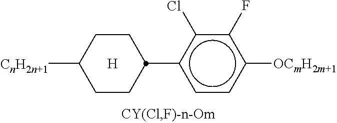

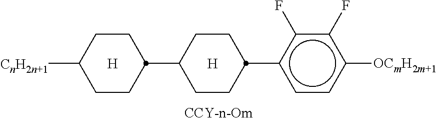

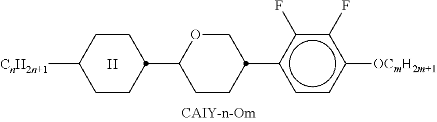

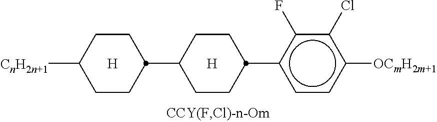

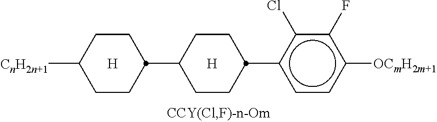

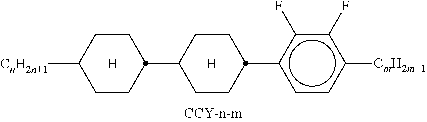

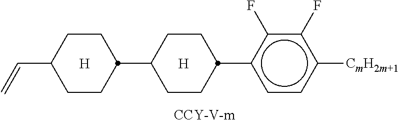

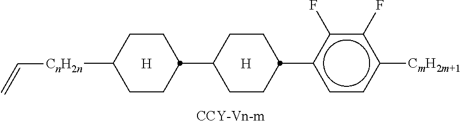

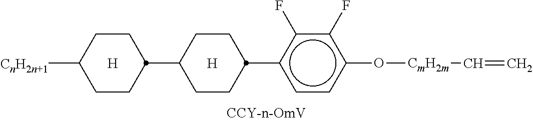

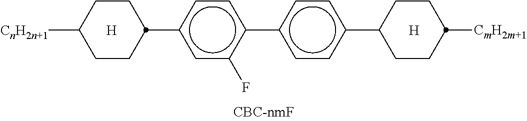

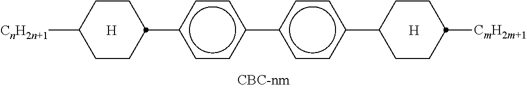

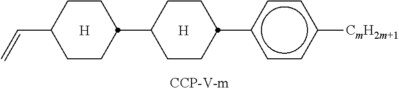

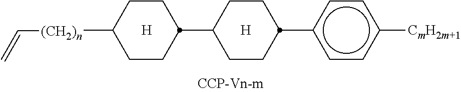

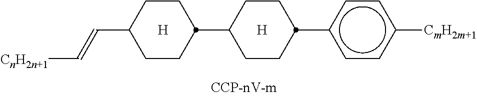

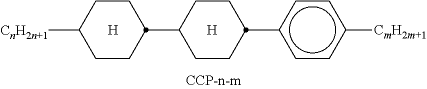

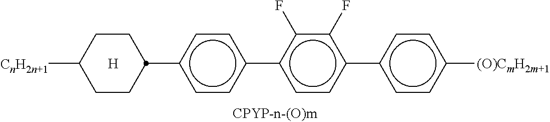

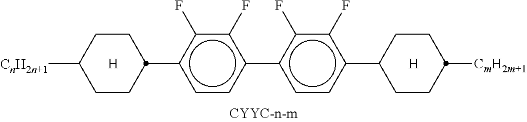

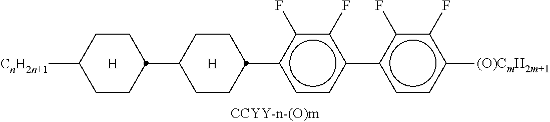

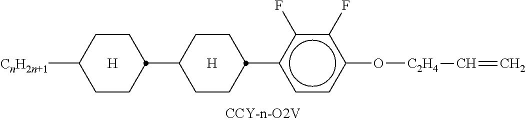

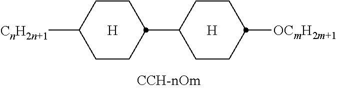

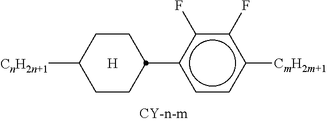

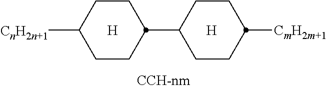

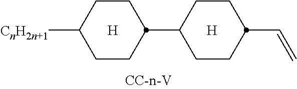

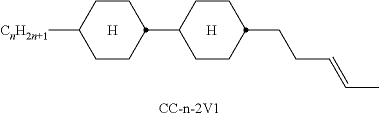

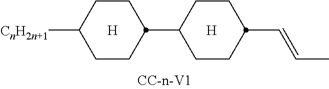

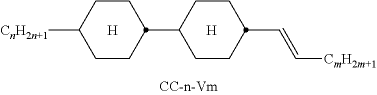

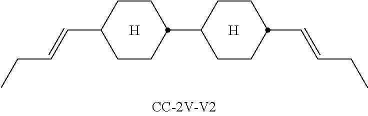

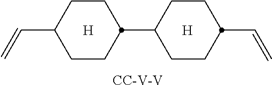

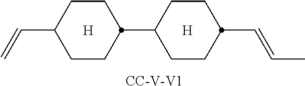

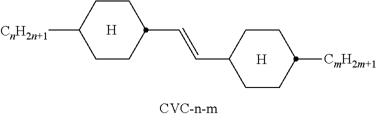

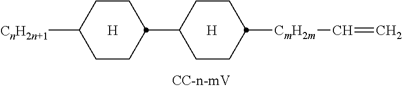

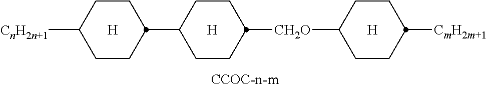

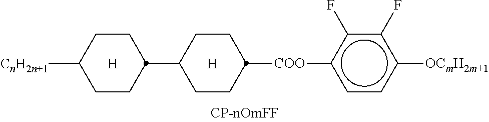

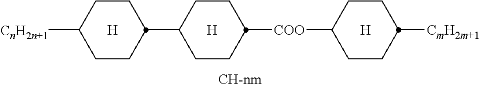

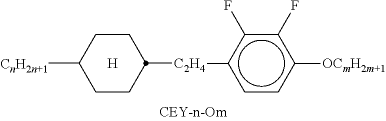

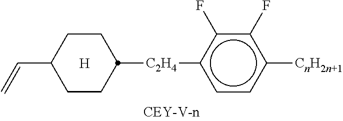

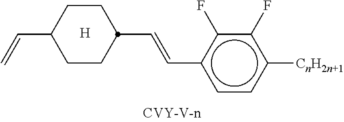

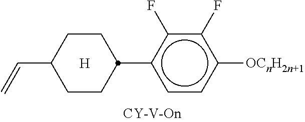

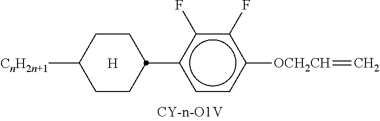

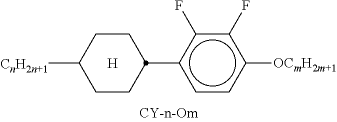

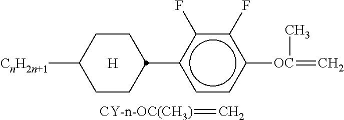

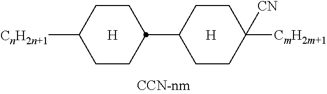

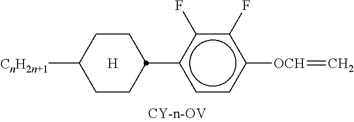

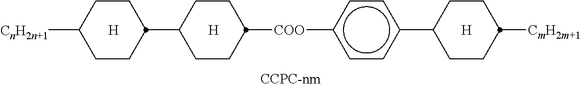

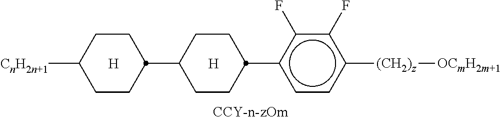

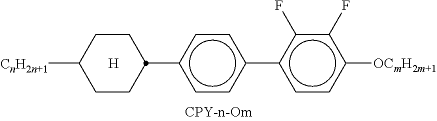

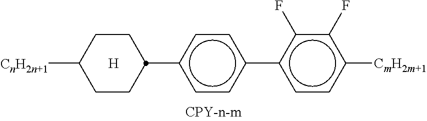

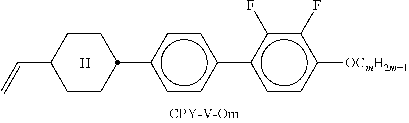

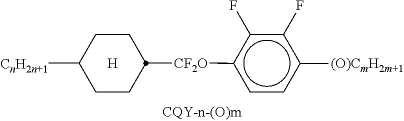

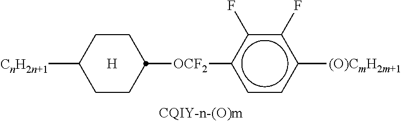

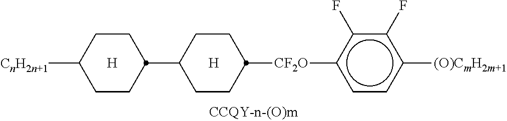

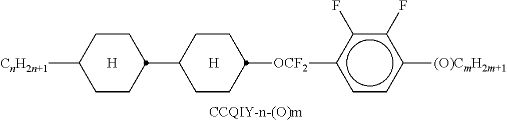

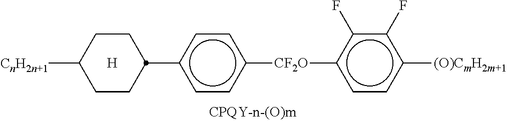

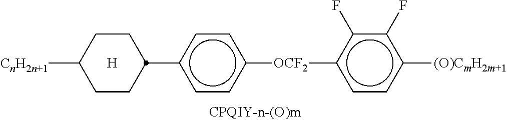

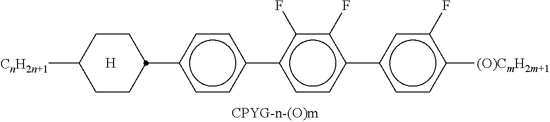

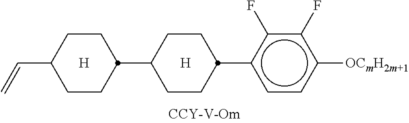

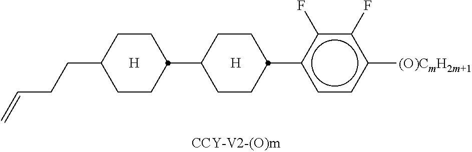

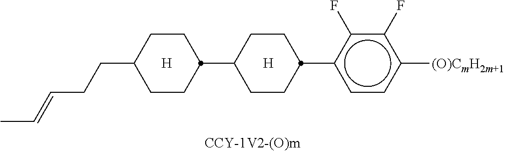

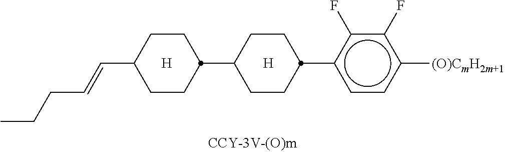

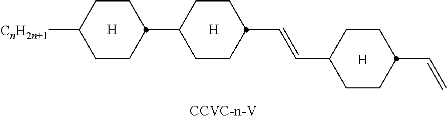

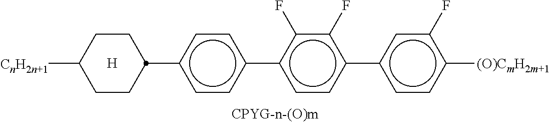

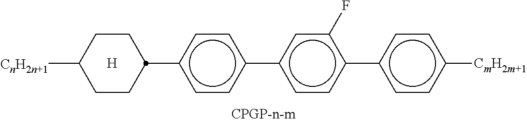

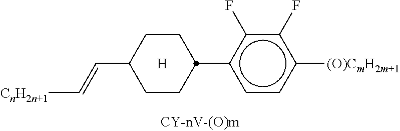

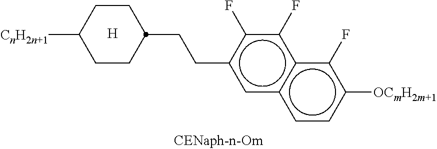

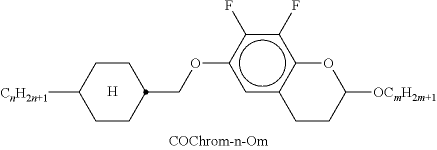

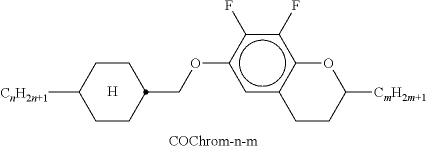

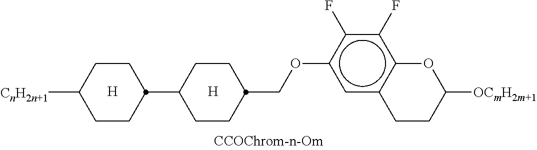

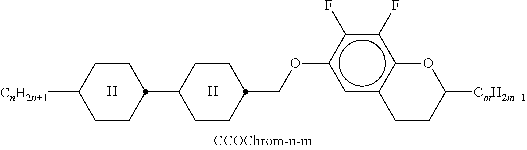

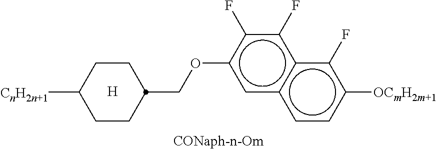

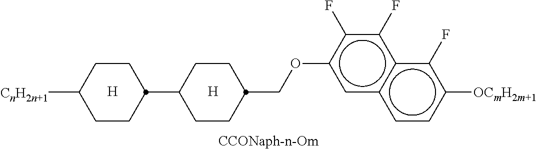

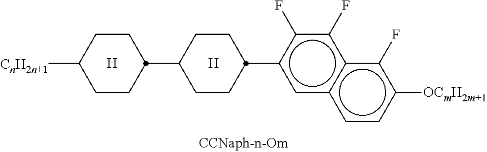

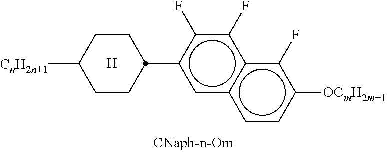

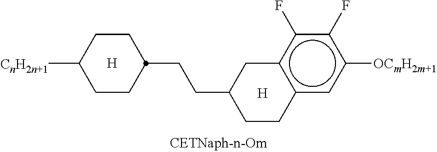

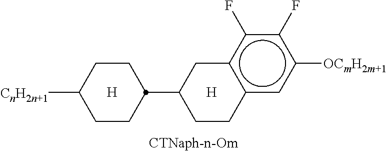

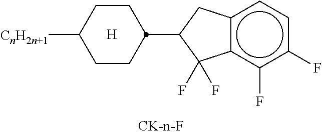

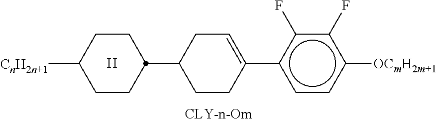

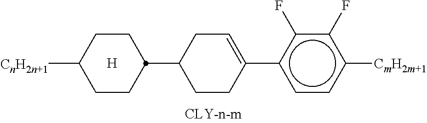

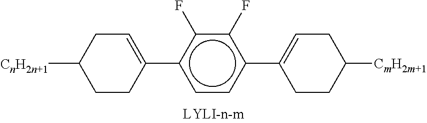

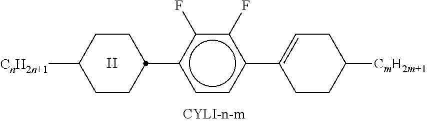

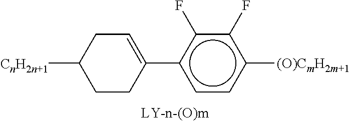

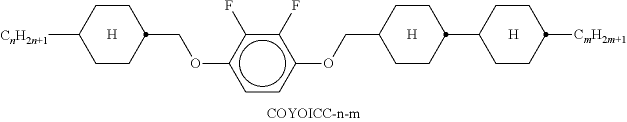

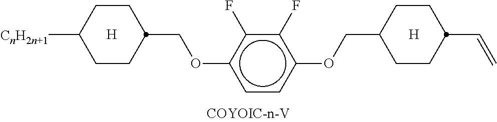

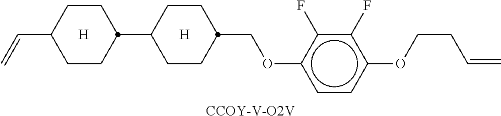

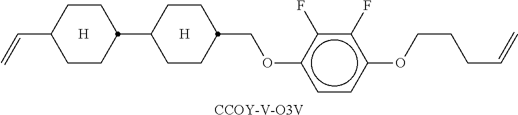

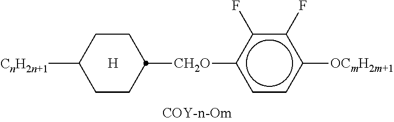

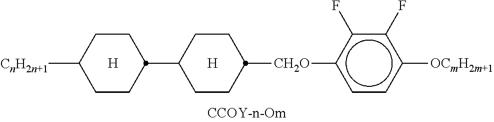

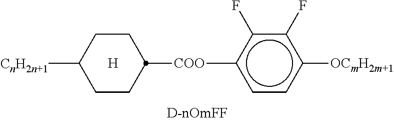

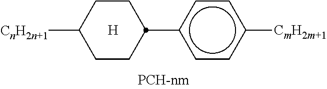

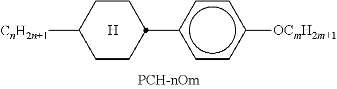

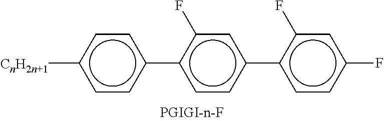

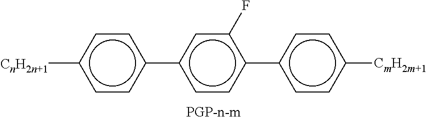

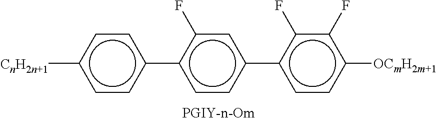

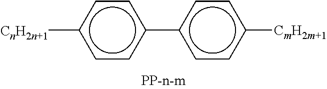

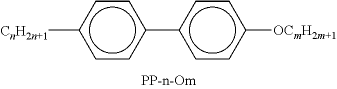

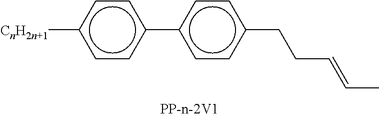

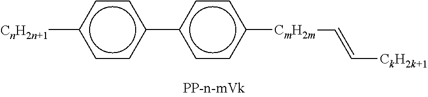

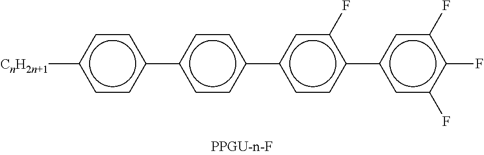

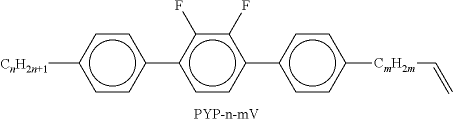

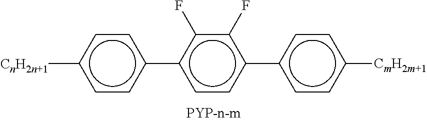

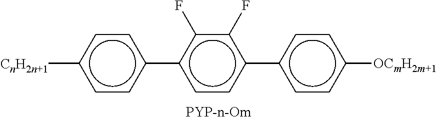

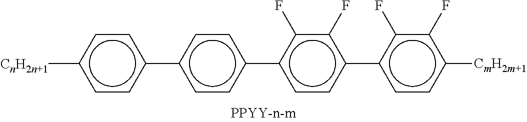

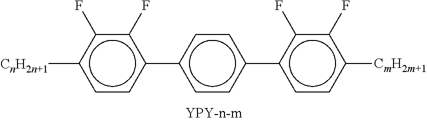

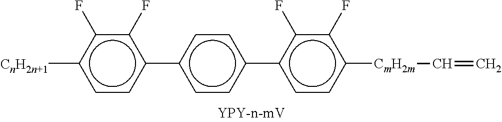

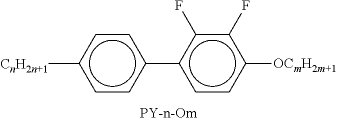

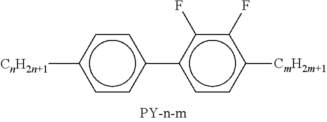

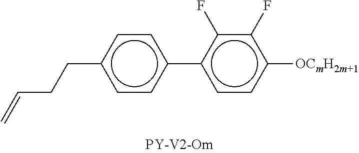

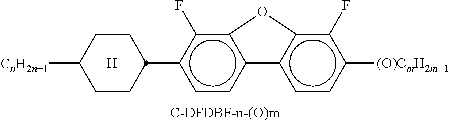

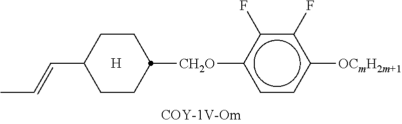

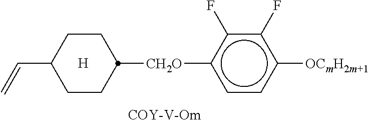

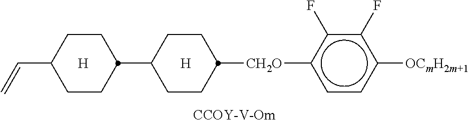

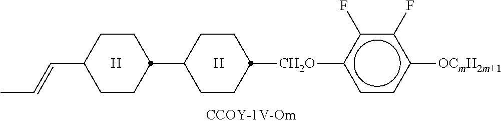

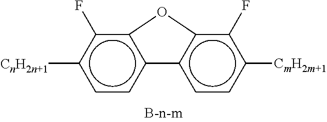

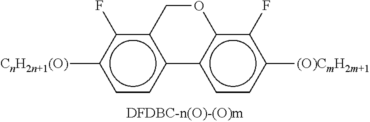

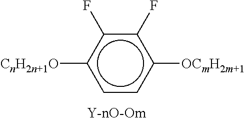

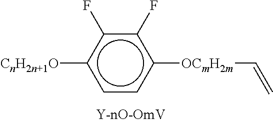

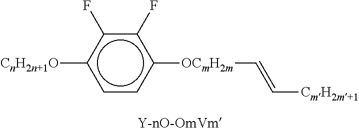

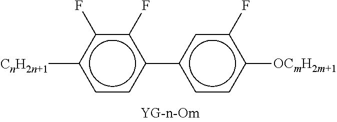

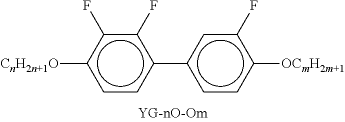

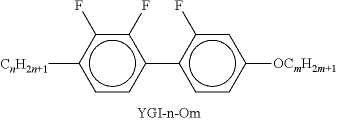

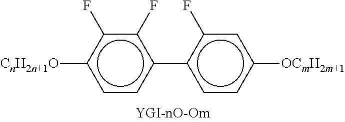

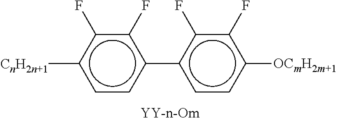

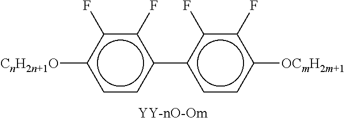

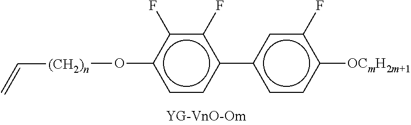

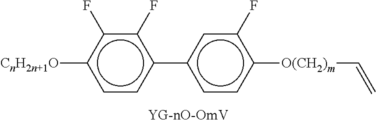

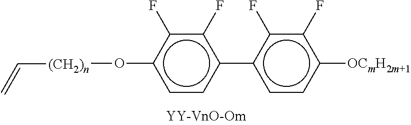

The following abbreviations are used:
(n, m, m′, z: each, independently of one another, 1, 2, 3, 4, 5 or 6;
(O)CₘH₂ₘ₊₁ means OCₘH₂ₘ₊₁ or CₘH₂ₘ₊₁)

The liquid-crystal mixtures which can be used in accordance with the invention are prepared in a manner which is conventional per se. In general, the desired amount of the components used in lesser amount is dissolved in the components making up the principal constituent, advantageously at elevated temperature. It is also possible to mix solutions of the components in an organic solvent, for example in acetone, chloroform or methanol, and to remove the solvent again, for example by distillation, after thorough mixing.

By means of suitable additives, the liquid-crystal phases according to the invention can be modified in such a way that they can be employed in any type of, for example, ECB, VAN, IPS, GH or ASM-VA LCD display that has been disclosed to date.

The dielectrics may also comprise further additives known to the person skilled in the art and described in the literature, such as, for example, UV absorbers, antioxidants, nanoparticles and free-radical scavengers. For example, 0-15% of pleochroic dyes, stabilizers or chiral dopants may be added. Suitable dopants for the mixtures according to the invention are, in particular, those listed in Table B. Suitable stabilizers for the mixtures according to the invention are, in particular, those listed in Table C.

For example, 0-15% of pleochroic dyes, furthermore conductive salts, preferably ethyldimethyldodecylammonium 4-hexoxybenzoate, tetrabutylammonium tetraphenylboranate or complex salts of crown ethers (cf., for example, Haller et al., Mol. Cryst. Liq. Cryst., Volume 24, pages 249-258 (1973)), may be added in order to improve the conductivity or substances may be added in order to modify the dielectric anisotropy, the viscosity and/or the alignment of the nematic phases. Substances of this type are described, for example, in DE-A 22 09 127, 22 40 864, 23 21 632, 23 38 281, 24 50 088, 26 37 430 and 28 53 728.

TABLE B

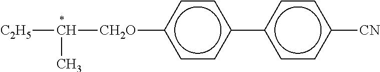

C 15

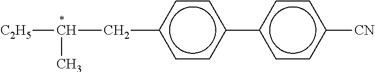

CB 15

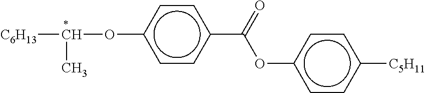

CM 21

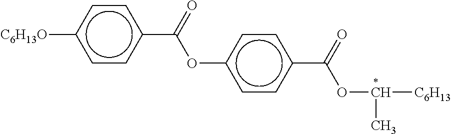

R/S-811

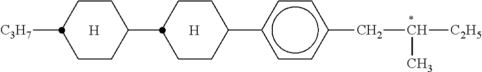

CM 44

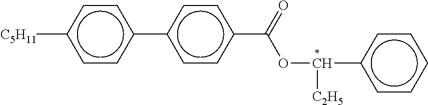

CM 45

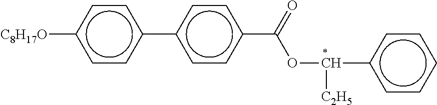

CM 47

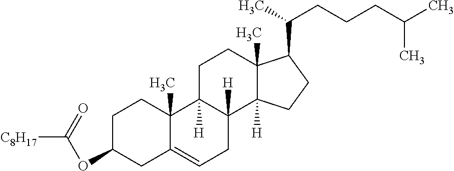

CN

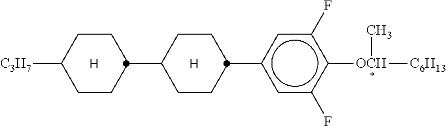

R/S-2011

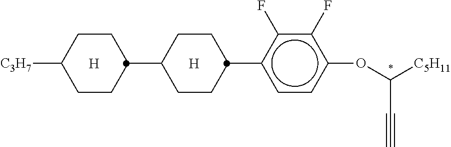

R/S-3011

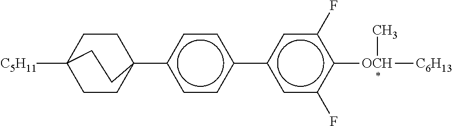

R/S-4011

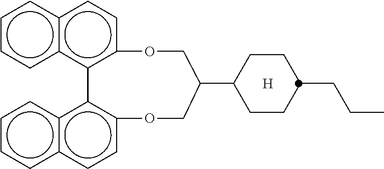

R/S-5011

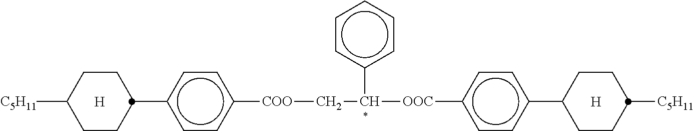

R/S-1011

Table B shows possible dopants which are generally added to the mixtures according to the invention. The mixtures preferably comprise 0-10% by weight, in particular 0.01-5% by weight and particularly preferably 0.01-3% by weight of dopants. If the mixtures comprise only one dopant, it is employed in amounts of 0.01-4% by weight, preferably 0.1-1.0% by weight.

TABLE C
Stabilizers which can be added, for example, to the mixtures according to
the invention in amounts of 0-10% by weight are shown below.

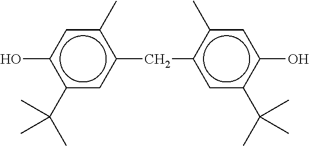

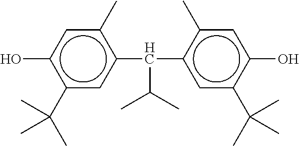

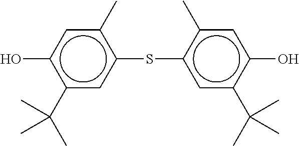

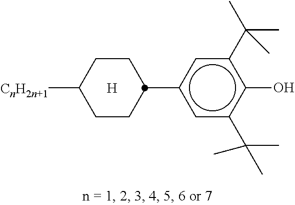

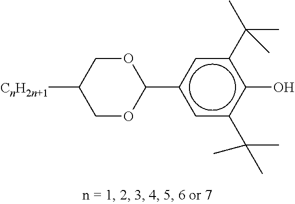

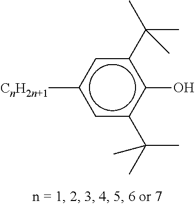

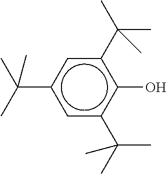

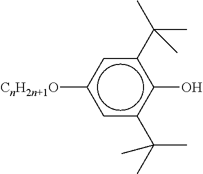

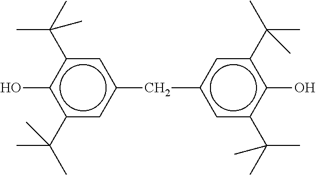

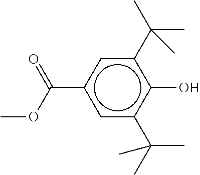

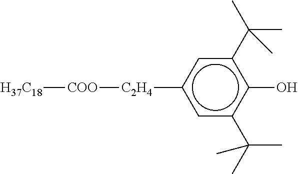

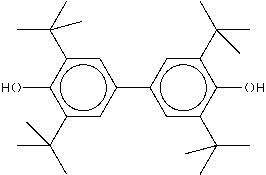

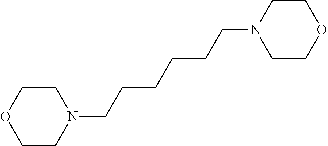 [SMILES: C(CCCN1CCOCC1)CCN1CCOCC1]

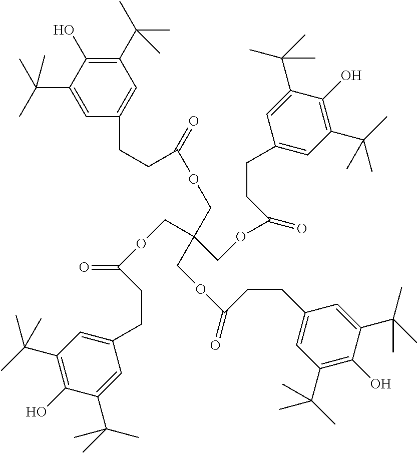 [SMILES: CC(C)(C)c1cc(CCC(=O)OCC(COC(=O)CCc2cc(C(C)(C)C)c(O)c(C(C)(C)C)c2)(COC(=O)CCc2cc(C(C)(C)C)c(O)c(C(C)(C)C)c2)COC(=O)CCc2cc(C(C)(C)C)c(O)c(C(C)(C)C)c2)cc(C(C)(C)C)c1O]

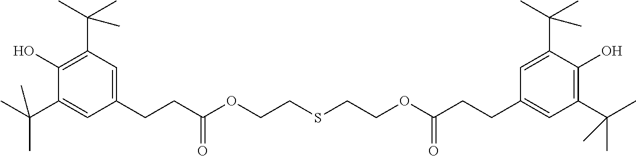 [SMILES: CC(C)(C)c1cc(CCC(=O)OCCSCCOC(=O)CCc2cc(C(C)(C)C)c(O)c(C(C)(C)C)c2)cc(C(C)(C)C)c1O]

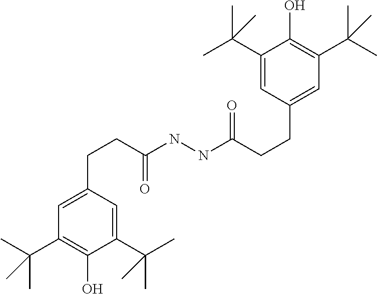 [SMILES: CC(C)(C)c1cc(CCC(=O)NNC(=O)CCc2cc(C(C)(C)C)c(O)c(C(C)(C)C)c2)cc(C(C)(C)C)c1O]

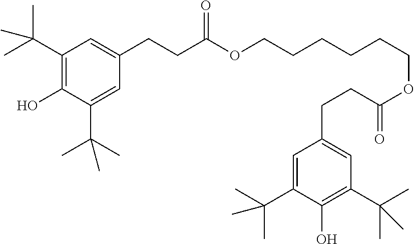 [SMILES: CC(C)(C)c1cc(CCC(=O)OCCCCCCOC(=O)CCc2cc(C(C)(C)C)c(O)c(C(C)(C)C)c2)cc(C(C)(C)C)c1O]

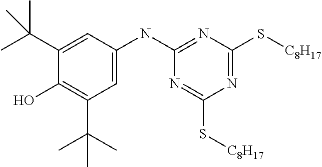

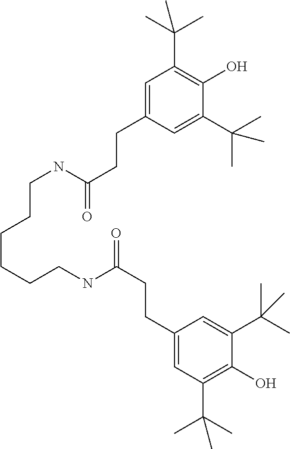 [SMILES: CC(C)(C)c1cc(CCC(=O)NCCCCCCNC(=O)CCc2cc(C(C)(C)C)c(O)c(C(C)(C)C)c2)cc(C(C)(C)C)c1O]

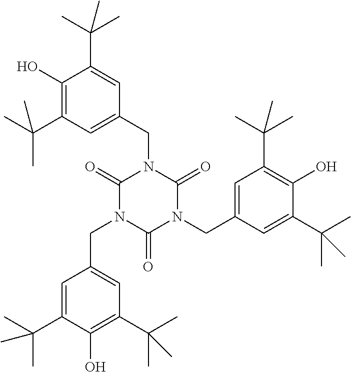 [SMILES: CC(C)(C)c1cc(Cn2c(=O)n(Cc3cc(C(C)(C)C)c(O)c(C(C)(C)C)c3)c(=O)n(Cc3cc(C(C)(C)C)c(O)c(C(C)(C)C)c3)c2=O)cc(C(C)(C)C)c1O]

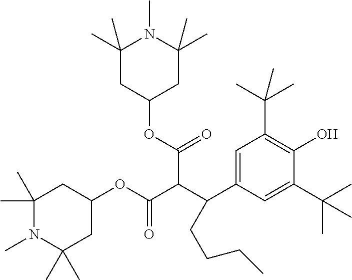 [SMILES: CCCCC(c1cc(C(C)(C)C)c(O)c(C(C)(C)C)c1)C(C(=O)OC1CC(C)(C)N(C)C(C)(C)C1)C(=O)OC1CC(C)(C)N(C)C(C)(C)C1]

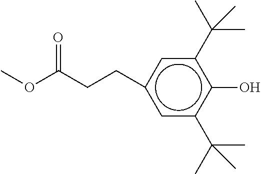

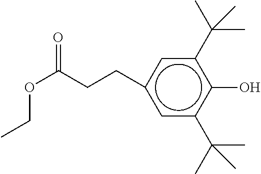

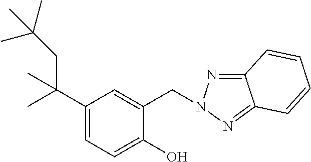 [SMILES: CC(C)(C)CC(C)(C)c1ccc(O)c(Cn2nc3ccccc3n2)c1]

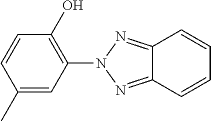 [SMILES: Cc1ccc(O)c(-n2nc3ccccc3n2)c1]

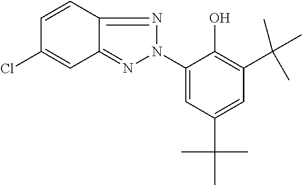 [SMILES: CC(C)(C)c1cc(-n2nc3ccc(Cl)cc3n2)c(O)c(C(C)(C)C)c1]

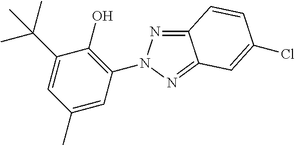 [SMILES: Cc1cc(-n2nc3ccc(Cl)cc3n2)c(O)c(C(C)(C)C)c1]

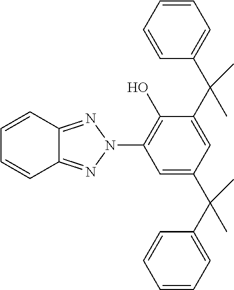 [SMILES: CC(C)(c1ccccc1)c1cc(-n2nc3ccccc3n2)c(O)c(C(C)(C)c2ccccc2)c1]

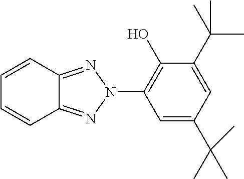 [SMILES: CC(C)(C)c1cc(-n2nc3ccccc3n2)c(O)c(C(C)(C)C)c1]

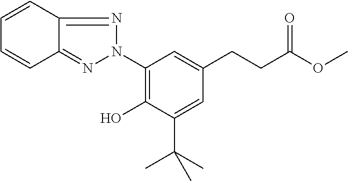 [SMILES: COC(=O)CCc1cc(-n2nc3ccccc3n2)c(O)c(C(C)(C)C)c1]

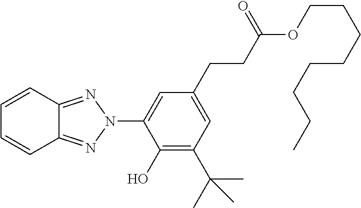 [SMILES: CCCCCCCCOC(=O)CCc1cc(-n2nc3ccccc3n2)c(O)c(C(C)(C)C)c1]

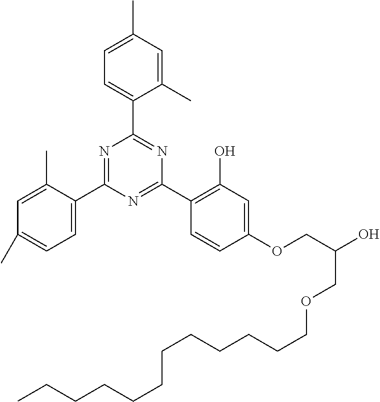 [SMILES: CCCCCCCCCCCCOCC(O)COc1ccc(-c2nc(-c3ccc(C)cc3C)nc(-c3ccc(C)cc3C)n2)c(O)c1]

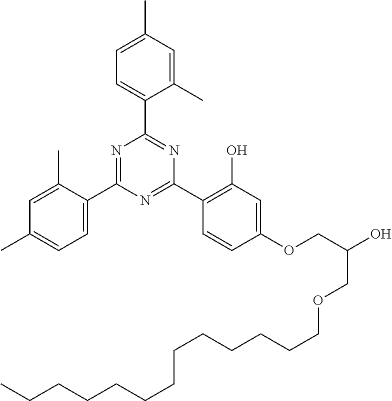 [SMILES: CCCCCCCCCCCCCOCC(O)COc1ccc(-c2nc(-c3ccc(C)cc3C)nc(-c3ccc(C)cc3C)n2)c(O)c1]

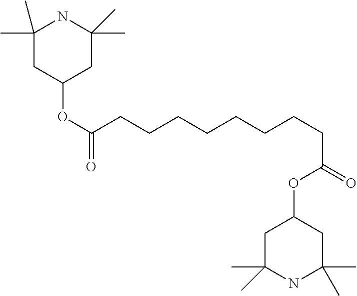

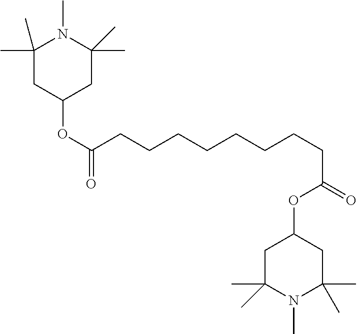

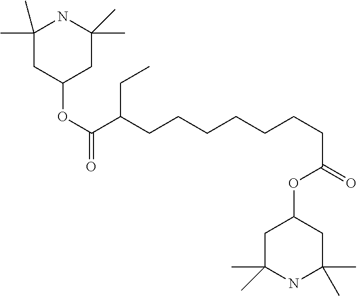

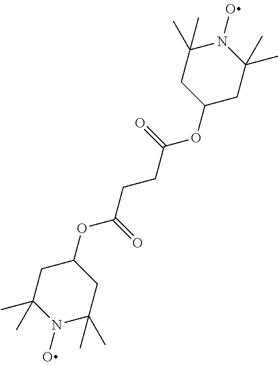

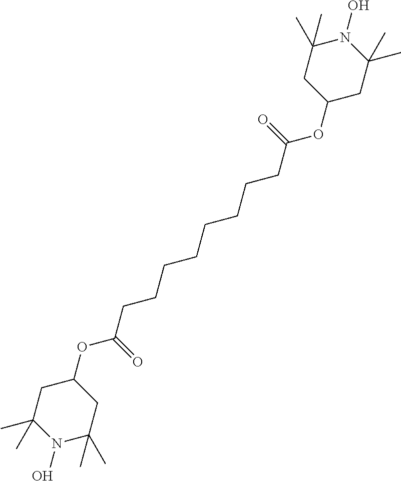

The medium according to the invention particularly preferably comprises Tinuvin® 770 (bis(2,2,6,6-tetramethyl-4-piperidyl) sebacate), preferably in amounts of 0.001-5% by weight, based on the liquid-crystalline medium.

TABLE D

| | |
|---|---|
| 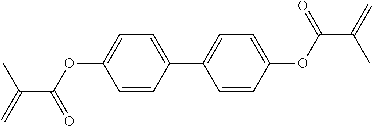 [SMILES: C=C(C)C(=O)Oc1ccc(-c2ccc(OC(=O)C(=C)C)cc2)cc1] | RM-1 |
| 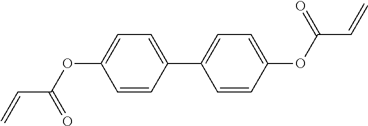 [SMILES: C=CC(=O)Oc1ccc(-c2ccc(OC(=O)C=C)cc2)cc1] | RM-2 |
| 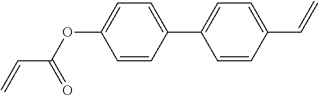 [SMILES: C=CC(=O)Oc1ccc(-c2ccc(C=C)cc2)cc1] | RM-3 |
| 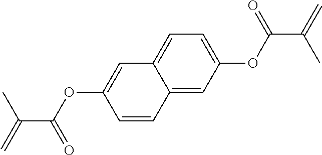 [SMILES: C=C(C)C(=O)Oc1ccc2cc(OC(=O)C(=C)C)ccc2c1] | RM-4 |
| 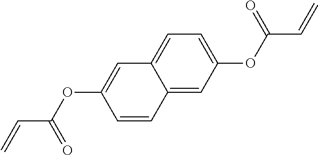 [SMILES: C=CC(=O)Oc1ccc2cc(OC(=O)C=C)ccc2c1] | RM-5 |
| 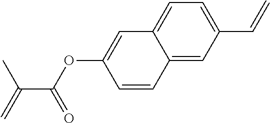 [SMILES: C=Cc1ccc2cc(OC(=O)C(=C)C)ccc2c1] | RM-6 |
| 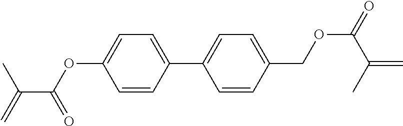 [SMILES: C=C(C)C(=O)OCc1ccc(-c2ccc(OC(=O)C(=C)C)cc2)cc1] | RM-7 |
| 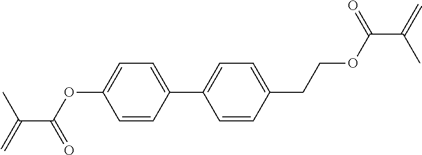 [SMILES: C=C(C)C(=O)OCCc1ccc(-c2ccc(OC(=O)C(=C)C)cc2)cc1] | RM-8 |
| 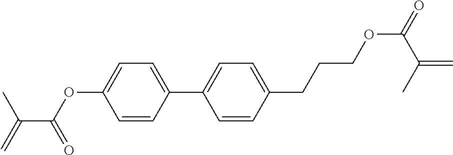 [SMILES: C=C(C)C(=O)OCCCc1ccc(-c2ccc(OC(=O)C(=C)C)cc2)cc1] | RM-9 |
| 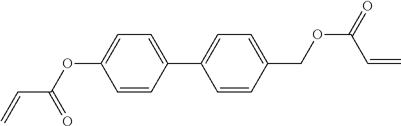 [SMILES: C=CC(=O)OCc1ccc(-c2ccc(OC(=O)C=C)cc2)cc1] | RM-10 |
| 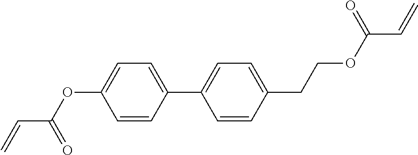 [SMILES: C=CC(=O)OCCc1ccc(-c2ccc(OC(=O)C=C)cc2)cc1] | RM-11 |
| 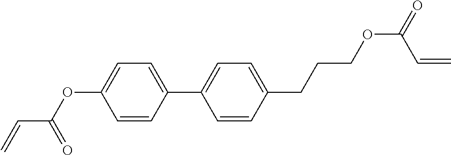 [SMILES: C=CC(=O)OCCCc1ccc(-c2ccc(OC(=O)C=C)cc2)cc1] | RM-12 |
| 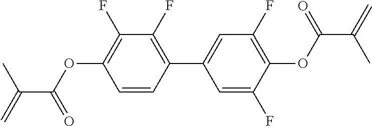 [SMILES: C=C(C)C(=O)Oc1ccc(-c2cc(F)c(OC(=O)C(=C)C)c(F)c2)c(F)c1F] | RM-13 |
| 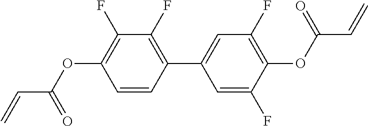 [SMILES: C=CC(=O)Oc1ccc(-c2cc(F)c(OC(=O)C=C)c(F)c2)c(F)c1F] | RM-14 |
| 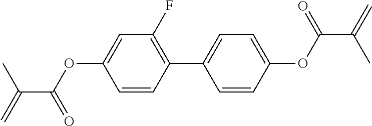 [SMILES: C=C(C)C(=O)Oc1ccc(-c2ccc(OC(=O)C(=C)C)cc2F)cc1] | RM-15 |
| 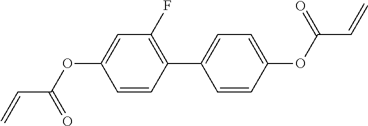 [SMILES: C=CC(=O)Oc1ccc(-c2ccc(OC(=O)C=C)cc2F)cc1] | RM-16 |
| 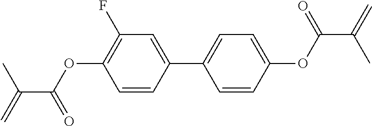 [SMILES: C=C(C)C(=O)Oc1ccc(-c2ccc(OC(=O)C(=C)C)c(F)c2)cc1] | RM-17 |
| 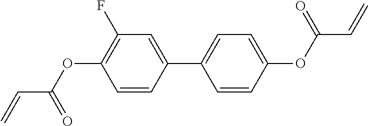 [SMILES: C=CC(=O)Oc1ccc(-c2ccc(OC(=O)C=C)c(F)c2)cc1] | RM-18 |
| 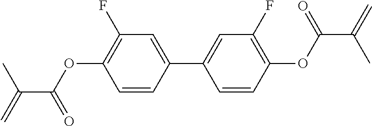 [SMILES: C=C(C)C(=O)Oc1ccc(-c2ccc(OC(=O)C(=C)C)c(F)c2)cc1F] | RM-19 |
| 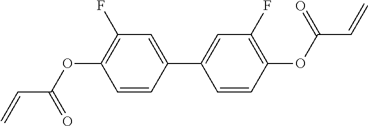 [SMILES: C=CC(=O)Oc1ccc(-c2ccc(OC(=O)C=C)c(F)c2)cc1F] | RM-20 |
| 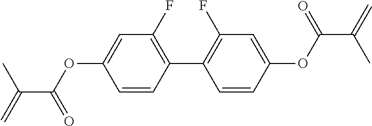 [SMILES: C=C(C)C(=O)Oc1ccc(-c2ccc(OC(=O)C(=C)C)cc2F)c(F)c1] | RM-21 |
| 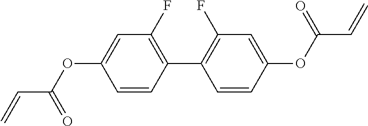 [SMILES: C=CC(=O)Oc1ccc(-c2ccc(OC(=O)C=C)cc2F)c(F)c1] | RM-22 |
| 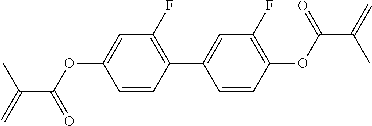 [SMILES: C=C(C)C(=O)Oc1ccc(-c2ccc(OC(=O)C(=C)C)c(F)c2)c(F)c1] | RM-23 |
| 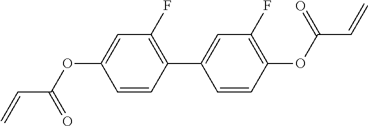 [SMILES: C=CC(=O)Oc1ccc(-c2ccc(OC(=O)C=C)c(F)c2)c(F)c1] | RM-24 |
| 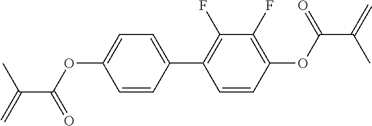 [SMILES: C=C(C)C(=O)Oc1ccc(-c2ccc(OC(=O)C(=C)C)c(F)c2F)cc1] | RM-25 |
| 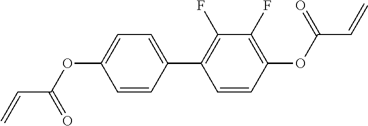 [SMILES: C=CC(=O)Oc1ccc(-c2ccc(OC(=O)C=C)c(F)c2F)cc1] | RM-26 |
| 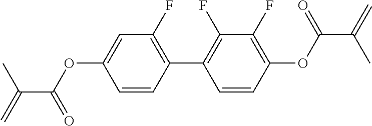 [SMILES: C=C(C)C(=O)Oc1ccc(-c2ccc(OC(=O)C(=C)C)c(F)c2F)c(F)c1] | RM-27 |
| 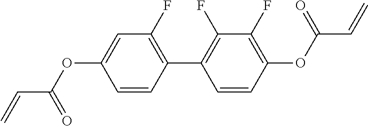 [SMILES: C=CC(=O)Oc1ccc(-c2ccc(OC(=O)C=C)c(F)c2F)c(F)c1] | RM-28 |
| 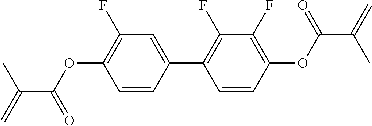 [SMILES: C=C(C)C(=O)Oc1ccc(-c2ccc(OC(=O)C(=C)C)c(F)c2F)cc1F] | RM-29 |
| 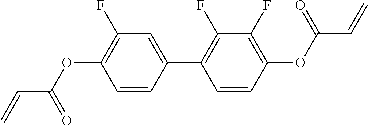 [SMILES: C=CC(=O)Oc1ccc(-c2ccc(OC(=O)C=C)c(F)c2F)cc1F] | RM-30 |
| 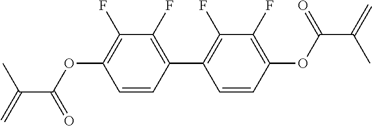 [SMILES: C=C(C)C(=O)Oc1ccc(-c2ccc(OC(=O)C(=C)C)c(F)c2F)c(F)c1F] | RM-31 |
| 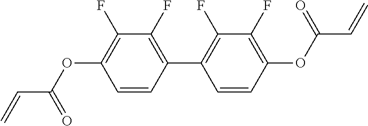 [SMILES: C=CC(=O)Oc1ccc(-c2ccc(OC(=O)C=C)c(F)c2F)c(F)c1F] | RM-32 |
| 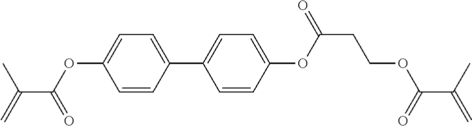 [SMILES: C=C(C)C(=O)OCCC(=O)Oc1ccc(-c2ccc(OC(=O)C(=C)C)cc2)cc1] | RM-33 |
| 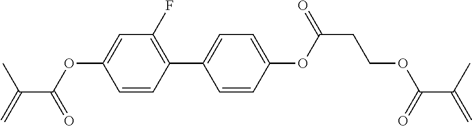 [SMILES: C=C(C)C(=O)OCCC(=O)Oc1ccc(-c2ccc(OC(=O)C(=C)C)cc2F)cc1] | RM-34 |
| 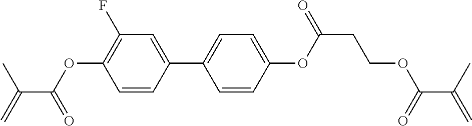 [SMILES: C=C(C)C(=O)OCCC(=O)Oc1ccc(-c2ccc(OC(=O)C(=C)C)c(F)c2)cc1] | RM-35 |
| 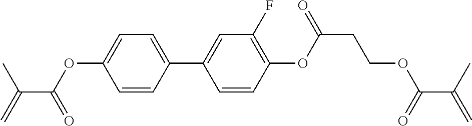 [SMILES: C=C(C)C(=O)OCCC(=O)Oc1ccc(-c2ccc(OC(=O)C(=C)C)cc2)cc1F] | RM-36 |
| 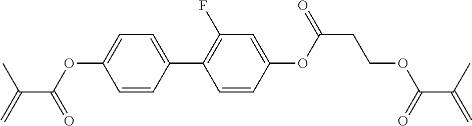 [SMILES: C=C(C)C(=O)OCCC(=O)Oc1ccc(-c2ccc(OC(=O)C(=C)C)cc2)c(F)c1] | RM-37 |
| 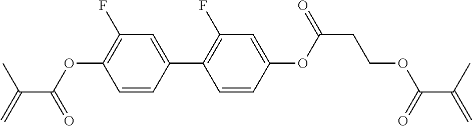 [SMILES: C=C(C)C(=O)OCCC(=O)Oc1ccc(-c2ccc(OC(=O)C(=C)C)c(F)c2)c(F)c1] | RM-38 |
| 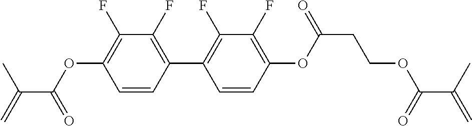 [SMILES: C=C(C)C(=O)OCCC(=O)Oc1ccc(-c2ccc(OC(=O)C(=C)C)c(F)c2F)c(F)c1F] | RM-39 |
| 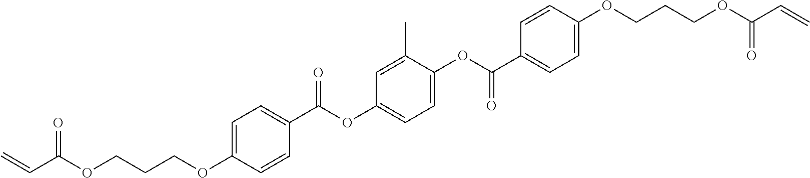 [SMILES: C=CC(=O)OCCCOc1ccc(C(=O)Oc2ccc(OC(=O)c3ccc(OCCCOC(=O)C=C)cc3)c(C)c2)cc1] | RM-40 |
| 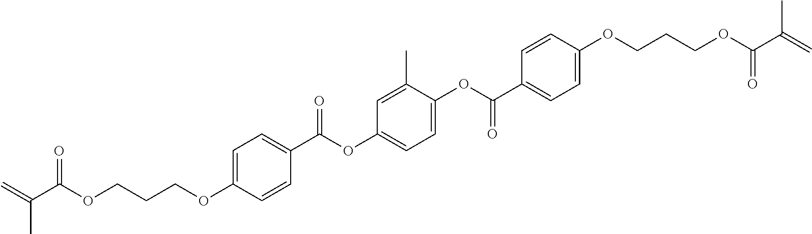 [SMILES: C=C(C)C(=O)OCCCOc1ccc(C(=O)Oc2ccc(OC(=O)c3ccc(OCCCOC(=O)C(=C)C)cc3)c(C)c2)cc1] | RM-41 |
| 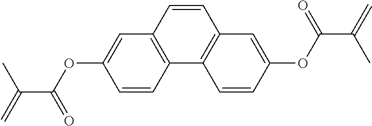 [SMILES: C=C(C)C(=O)Oc1ccc2c(ccc3cc(OC(=O)C(=C)C)ccc32)c1] | RM-42 |
| 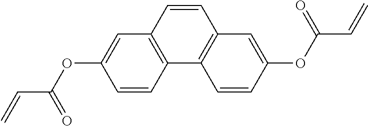 [SMILES: C=CC(=O)Oc1ccc2c(ccc3cc(OC(=O)C=C)ccc32)c1] | RM-43 |
| 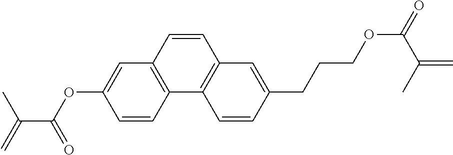 [SMILES: C=C(C)C(=O)OCCCc1ccc2c(ccc3cc(OC(=O)C(=C)C)ccc32)c1] | RM-44 |
| 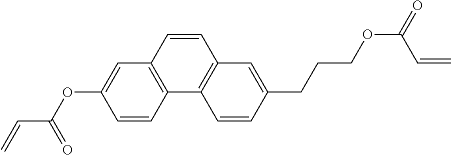 [SMILES: C=CC(=O)OCCCc1ccc2c(ccc3cc(OC(=O)C=C)ccc32)c1] | RM-45 |
| 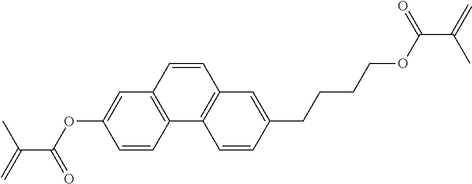 [SMILES: C=C(C)C(=O)OCCCCc1ccc2c(ccc3cc(OC(=O)C(=C)C)ccc32)c1] | RM-46 |
| 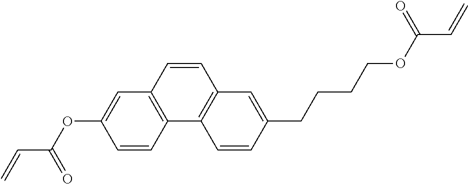 [SMILES: C=CC(=O)OCCCCc1ccc2c(ccc3cc(OC(=O)C=C)ccc32)c1] | RM-47 |
| 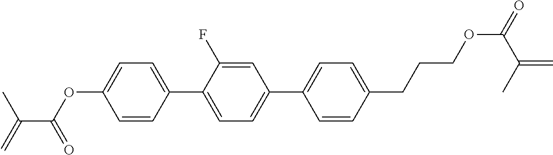 [SMILES: C=C(C)C(=O)OCCCc1ccc(-c2ccc(-c3ccc(OC(=O)C(=C)C)cc3)c(F)c2)cc1] | RM-48 |
| 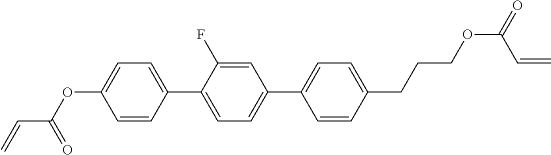 [SMILES: C=CC(=O)OCCCc1ccc(-c2ccc(-c3ccc(OC(=O)C=C)cc3)c(F)c2)cc1] | RM-49 |
| 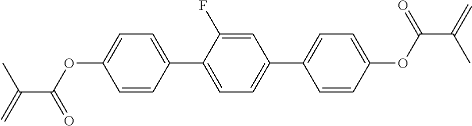 [SMILES: C=C(C)C(=O)Oc1ccc(-c2ccc(-c3ccc(OC(=O)C(=C)C)cc3)c(F)c2)cc1] | RM-50 |
| 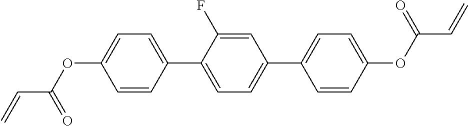 [SMILES: C=CC(=O)Oc1ccc(-c2ccc(-c3ccc(OC(=O)C=C)cc3)c(F)c2)cc1] | RM-51 |
| 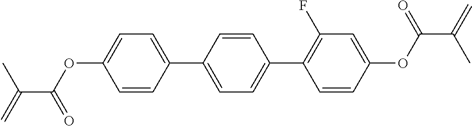 [SMILES: C=C(C)C(=O)Oc1ccc(-c2ccc(-c3ccc(OC(=O)C(=C)C)cc3F)cc2)cc1] | RM-52 |
| 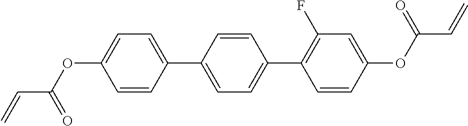 [SMILES: C=CC(=O)Oc1ccc(-c2ccc(-c3ccc(OC(=O)C=C)cc3F)cc2)cc1] | RM-53 |
| 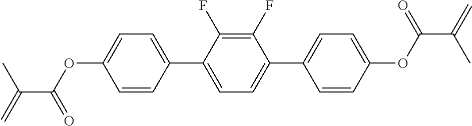 [SMILES: C=C(C)C(=O)Oc1ccc(-c2ccc(-c3ccc(OC(=O)C(=C)C)cc3)c(F)c2F)cc1] | RM-54 |
| 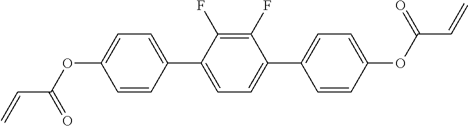 [SMILES: C=CC(=O)Oc1ccc(-c2ccc(-c3ccc(OC(=O)C=C)cc3)c(F)c2F)cc1] | RM-55 |
| 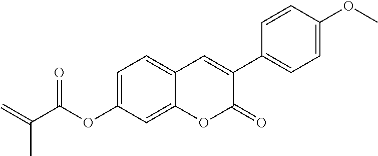 [SMILES: C=C(C)C(=O)Oc1ccc2cc(-c3ccc(OC)cc3)c(=O)oc2c1] | RM-56 |
| 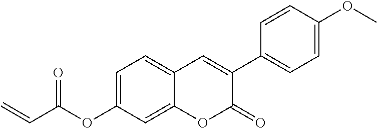 [SMILES: C=CC(=O)Oc1ccc2cc(-c3ccc(OC)cc3)c(=O)oc2c1] | RM-57 |
| 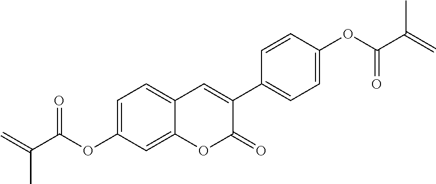 [SMILES: C=C(C)C(=O)Oc1ccc(-c2cc3ccc(OC(=O)C(=C)C)cc3oc2=O)cc1] | RM-58 |
| 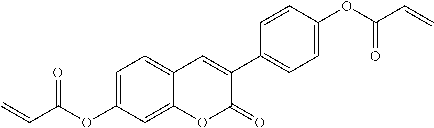 [SMILES: C=CC(=O)Oc1ccc(-c2cc3ccc(OC(=O)C=C)cc3oc2=O)cc1] | RM-59 |
| 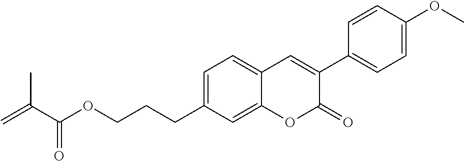 [SMILES: C=C(C)C(=O)OCCCc1ccc2cc(-c3ccc(OC)cc3)c(=O)oc2c1] | RM-60 |
| 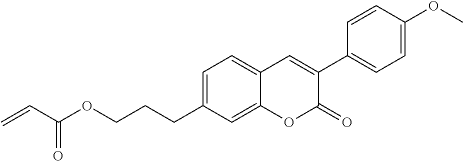 [SMILES: C=CC(=O)OCCCc1ccc2cc(-c3ccc(OC)cc3)c(=O)oc2c1] | RM-61 |
| 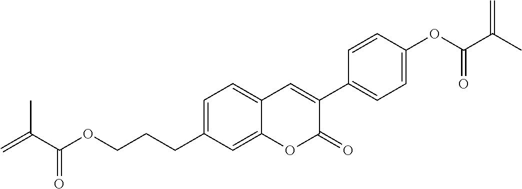 [SMILES: C=C(C)C(=O)OCCCc1ccc2cc(-c3ccc(OC(=O)C(=C)C)cc3)c(=O)oc2c1] | RM-62 |
| 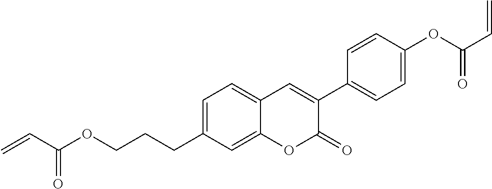 [SMILES: C=CC(=O)OCCCc1ccc2cc(-c3ccc(OC(=O)C=C)cc3)c(=O)oc2c1] | RM-63 |
| 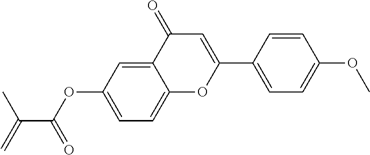 [SMILES: C=C(C)C(=O)Oc1ccc2oc(-c3ccc(OC)cc3)cc(=O)c2c1] | RM-64 |
| 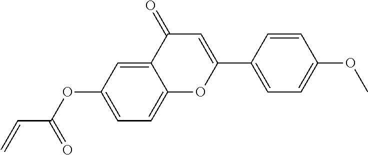 [SMILES: C=CC(=O)Oc1ccc2oc(-c3ccc(OC)cc3)cc(=O)c2c1] | RM-65 |
| 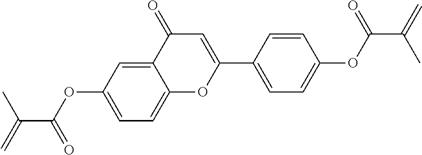 [SMILES: C=C(C)C(=O)Oc1ccc(-c2cc(=O)c3cc(OC(=O)C(=C)C)ccc3o2)cc1] | RM-66 |
| 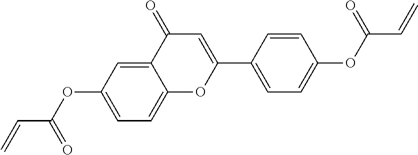 [SMILES: C=CC(=O)Oc1ccc(-c2cc(=O)c3cc(OC(=O)C=C)ccc3o2)cc1] | RM-67 |
| 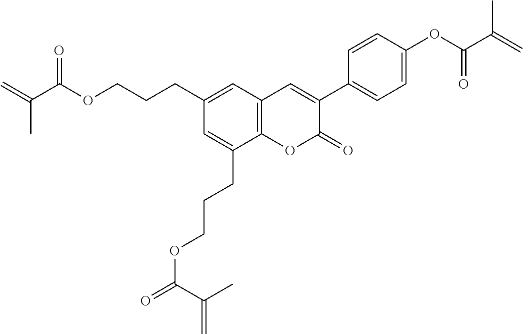 [SMILES: C=C(C)C(=O)OCCCc1cc(CCCOC(=O)C(=C)C)c2oc(=O)c(-c3ccc(OC(=O)C(=C)C)cc3)cc2c1] | RM-68 |
| 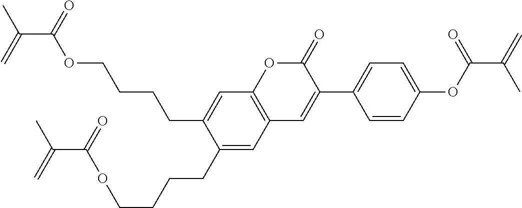 [SMILES: C=C(C)C(=O)OCCCCc1cc2cc(-c3ccc(OC(=O)C(=C)C)cc3)c(=O)oc2cc1CCCCOC(=O)C(=C)C] | RM-69 |
| 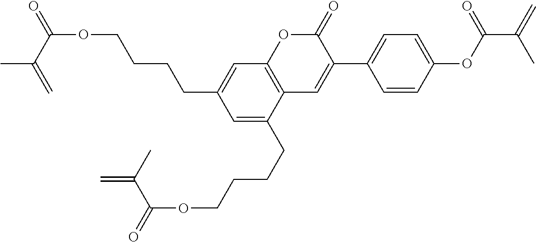 [SMILES: C=C(C)C(=O)OCCCCc1cc(CCCCOC(=O)C(=C)C)c2cc(-c3ccc(OC(=O)C(=C)C)cc3)c(=O)oc2c1] | RM-70 |
| 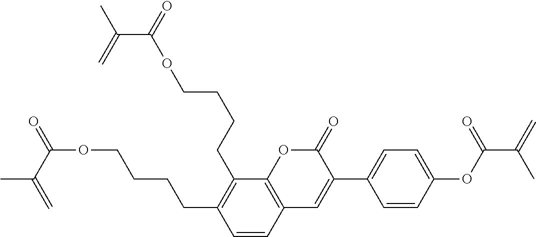 [SMILES: C=C(C)C(=O)OCCCCc1ccc2cc(-c3ccc(OC(=O)C(=C)C)cc3)c(=O)oc2c1CCCCOC(=O)C(=C)C] | RM-71 |
| 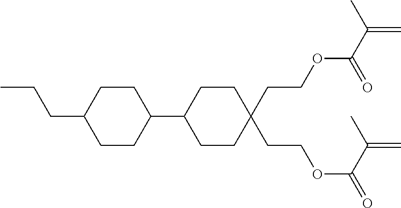 [SMILES: C=C(C)C(=O)OCCC1(CCOC(=O)C(=C)C)CCC(C2CCC(CCC)CC2)CC1] | RM-72 |
| 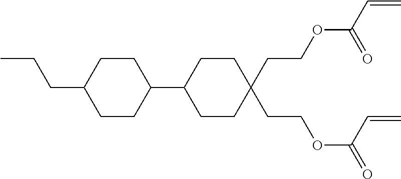 [SMILES: C=CC(=O)OCCC1(CCOC(=O)C=C)CCC(C2CCC(CCC)CC2)CC1] | RM-73 |
| 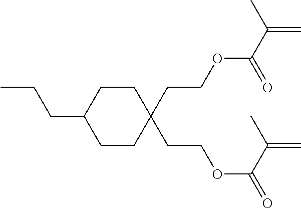 [SMILES: C=C(C)C(=O)OCCC1(CCOC(=O)C(=C)C)CCC(CCC)CC1] | RM-74 |
| 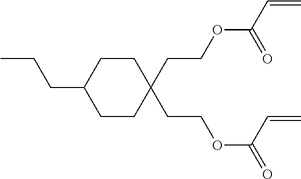 [SMILES: C=CC(=O)OCCC1(CCOC(=O)C=C)CCC(CCC)CC1] | RM-75 |
| 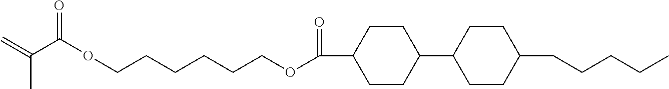 [SMILES: C=C(C)C(=O)OCCCCCCOC(=O)C1CCC(C2CCC(CCCCC)CC2)CC1] | RM-76 |
| 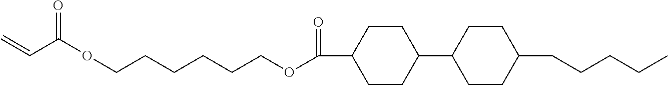 [SMILES: C=CC(=O)OCCCCCCOC(=O)C1CCC(C2CCC(CCCCC)CC2)CC1] | RM-77 |
| 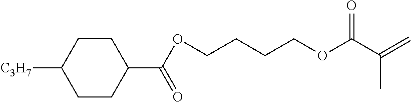 | RM-78 |
| 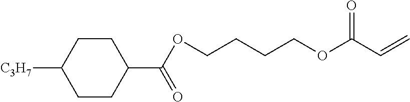 | RM-79 |
| 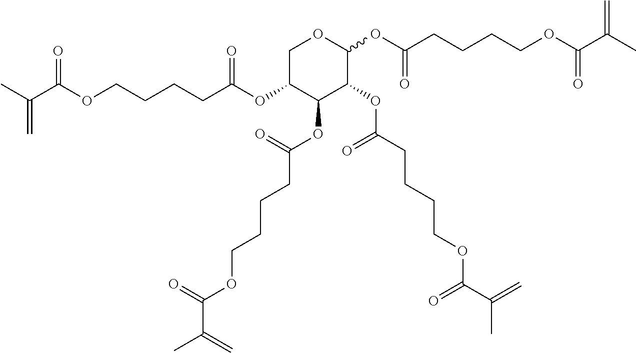 [SMILES: C=C(C)C(=O)OCCCCC(=O)OC1OC[C@@H](OC(=O)CCCCOC(=O)C(=C)C)[C@H](OC(=O)CCCCOC(=O)C(=C)C)[C@H]1OC(=O)CCCCOC(=O)C(=C)C] | RM-80 |
| 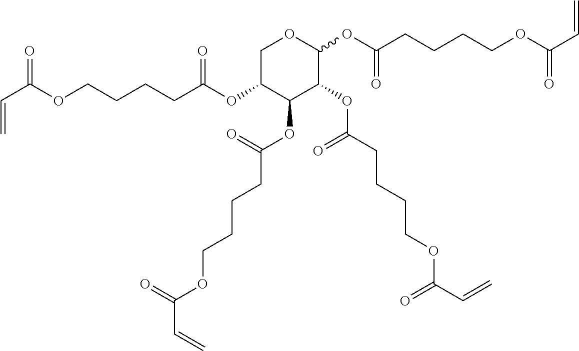 [SMILES: C=CC(=O)OCCCCC(=O)OC1OC[C@@H](OC(=O)CCCCOC(=O)C=C)[C@H](OC(=O)CCCCOC(=O)C=C)[C@H]1OC(=O)CCCCOC(=O)C=C] | RM-81 |
| 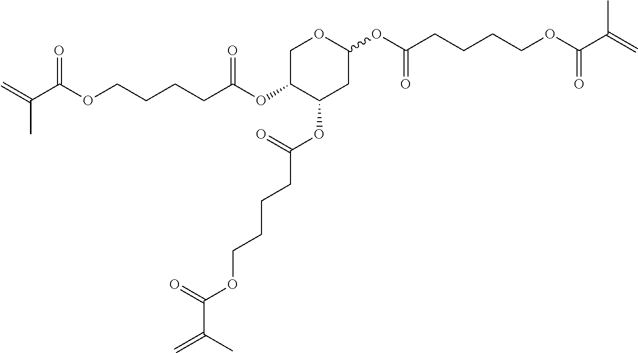 [SMILES: C=C(C)C(=O)OCCCCC(=O)OC1C[C@H](OC(=O)CCCCOC(=O)C(=C)C)[C@H](OC(=O)CCCCOC(=O)C(=C)C)CO1] | RM-82 |
| 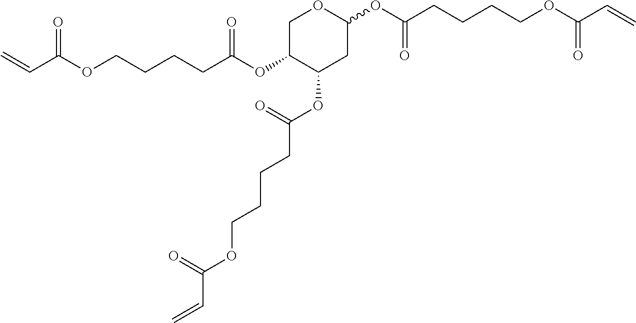 [SMILES: C=CC(=O)OCCCCC(=O)OC1C[C@H](OC(=O)CCCCOC(=O)C=C)[C@H](OC(=O)CCCCOC(=O)C=C)CO1] | RM-83 |
| 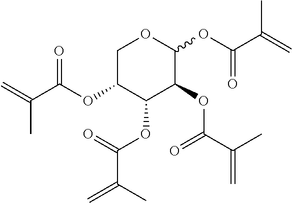 [SMILES: C=C(C)C(=O)OC1OC[C@@H](OC(=O)C(=C)C)[C@@H](OC(=O)C(=C)C)[C@@H]1OC(=O)C(=C)C] | RM-84 |
| 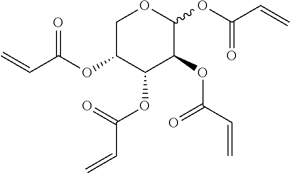 [SMILES: C=CC(=O)OC1OC[C@@H](OC(=O)C=C)[C@@H](OC(=O)C=C)[C@@H]1OC(=O)C=C] | RM-85 |
| 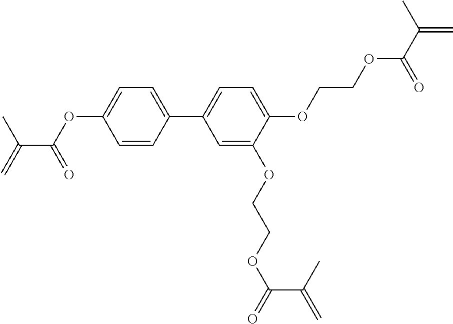 [SMILES: C=C(C)C(=O)OCCOc1ccc(-c2ccc(OC(=O)C(=C)C)cc2)cc1OCCOC(=O)C(=C)C] | RM-86 |
| 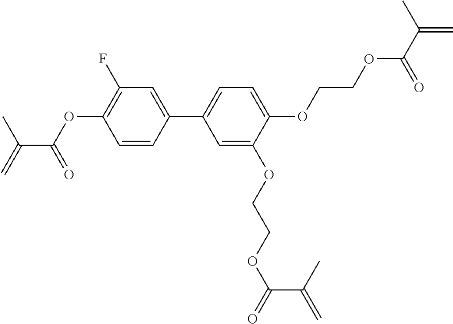 [SMILES: C=C(C)C(=O)OCCOc1ccc(-c2ccc(OC(=O)C(=C)C)c(F)c2)cc1OCCOC(=O)C(=C)C] | RM-87 |
| 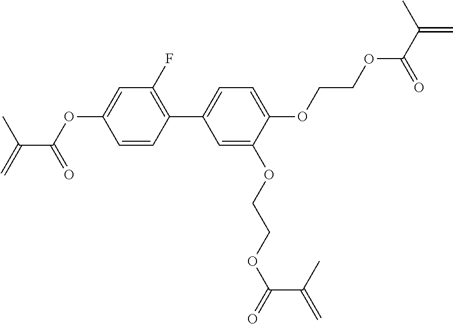 [SMILES: C=C(C)C(=O)OCCOc1ccc(-c2ccc(OC(=O)C(=C)C)cc2F)cc1OCCOC(=O)C(=C)C] | RM-88 |
| 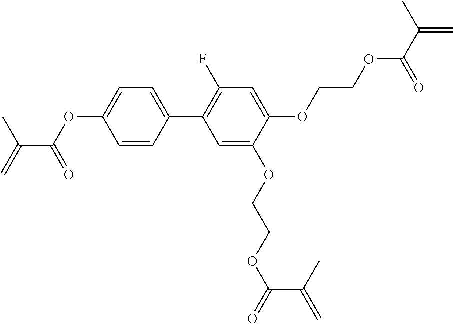 [SMILES: C=C(C)C(=O)OCCOc1cc(F)c(-c2ccc(OC(=O)C(=C)C)cc2)cc1OCCOC(=O)C(=C)C] | RM-89 |
| 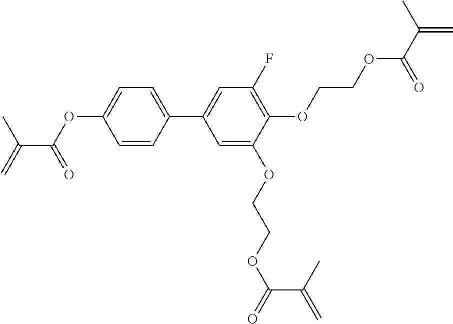 [SMILES: C=C(C)C(=O)OCCOc1cc(-c2ccc(OC(=O)C(=C)C)cc2)cc(F)c1OCCOC(=O)C(=C)C] | RM-90 |
| 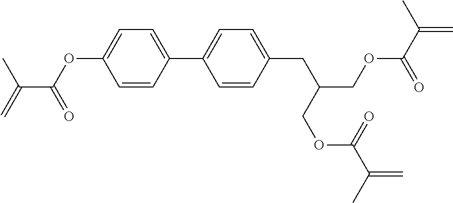 [SMILES: C=C(C)C(=O)OCC(COC(=O)C(=C)C)Cc1ccc(-c2ccc(OC(=O)C(=C)C)cc2)cc1] | RM-91 |
| 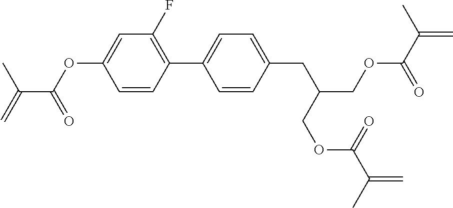 [SMILES: C=C(C)C(=O)OCC(COC(=O)C(=C)C)Cc1ccc(-c2ccc(OC(=O)C(=C)C)cc2F)cc1] | RM-92 |
| 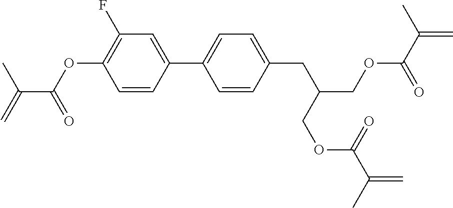 [SMILES: C=C(C)C(=O)OCC(COC(=O)C(=C)C)Cc1ccc(-c2ccc(OC(=O)C(=C)C)c(F)c2)cc1] | RM-93 |
| 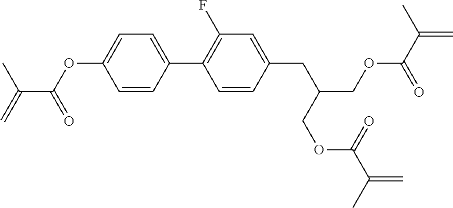 [SMILES: C=C(C)C(=O)OCC(COC(=O)C(=C)C)Cc1ccc(-c2ccc(OC(=O)C(=C)C)cc2)c(F)c1] | RM-94 |
| 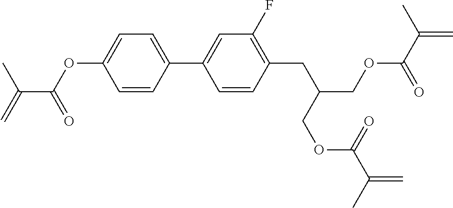 [SMILES: C=C(C)C(=O)OCC(COC(=O)C(=C)C)Cc1ccc(-c2ccc(OC(=O)C(=C)C)cc2)cc1F] | RM-95 |
| 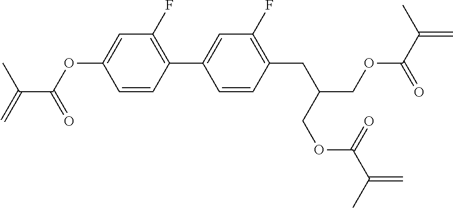 [SMILES: C=C(C)C(=O)OCC(COC(=O)C(=C)C)Cc1ccc(-c2ccc(OC(=O)C(=C)C)cc2F)cc1F] | RM-96 |
| 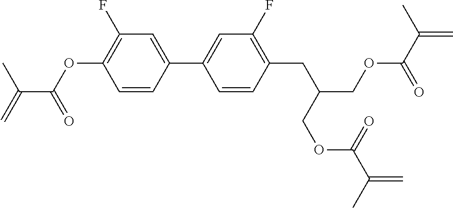 [SMILES: C=C(C)C(=O)OCC(COC(=O)C(=C)C)Cc1ccc(-c2ccc(OC(=O)C(=C)C)c(F)c2)cc1F] | RM-97 |
| 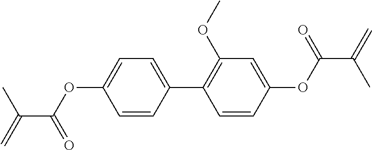 [SMILES: C=C(C)C(=O)Oc1ccc(-c2ccc(OC(=O)C(=C)C)cc2OC)cc1] | RM-98 |
| 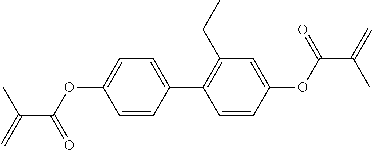 [SMILES: C=C(C)C(=O)Oc1ccc(-c2ccc(OC(=O)C(=C)C)cc2CC)cc1] | RM-99 |
| 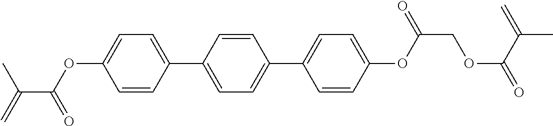 [SMILES: C=C(C)C(=O)OCC(=O)Oc1ccc(-c2ccc(-c3ccc(OC(=O)C(=C)C)cc3)cc2)cc1] | RM-100 |
| 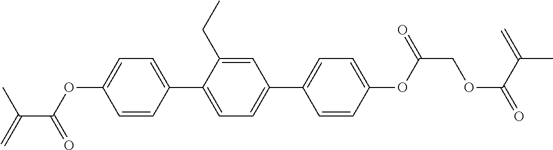 [SMILES: C=C(C)C(=O)OCC(=O)Oc1ccc(-c2ccc(-c3ccc(OC(=O)C(=C)C)cc3)c(CC)c2)cc1] | RM-101 |
| 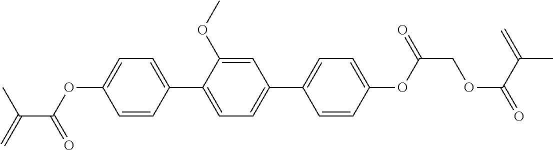 [SMILES: C=C(C)C(=O)OCC(=O)Oc1ccc(-c2ccc(-c3ccc(OC(=O)C(=C)C)cc3)c(OC)c2)cc1] | RM-102 |

Table D shows example compounds which can preferably be used as reactive mesogenic compounds in the LC media in accordance with the present invention. If the mixtures according to the invention comprise one or more reactive compounds, they are preferably employed in amounts of 0.01-5% by weight. It may also be necessary to add an initiator or a mixture of two or more initiators for the polymerization. The initiator or initiator mixture is preferably added in amounts of 0.001-2% by weight, based on the mixture. A suitable initiator is, for example, Irgacure (BASF) or Irganox (BASF).

In a preferred embodiment, the mixtures according to the invention comprise one or more polymerizable compounds, preferably selected from the polymerizable compounds of the formulae RM-1 to RM-102. Media of this type are suitable, in particular, for PS-FFS and PS-IPS applications. Of the reactive mesogens shown in Table D, compounds RM-1, RM-2, RM-3, RM-4, RM-5, RM-9, RM-17, RM-42, RM-48, RM-68, RM-87, RM-91, RM-98, RM-99 and RM-101 are particularly preferred.

The reactive mesogens or the polymerizable compounds of the formula M and of the formulae RM-1 to RM-102 are furthermore suitable as stabilizers. In this case, the polymerizable compounds are not polymerized, but instead are added to the liquid-crystalline medium in concentrations >1%.

### WORKING EXAMPLES

The following examples are intended to explain the invention without limiting it. In the examples, m.p. denotes the melting point and C denotes the clearing point of a liquid-crystalline substance in degrees Celsius; boiling temperatures are denoted by b.p. Furthermore:

C denotes crystalline solid state, S denotes smectic phase (the index denotes the phase type), N denotes nematic state, Ch denotes cholesteric phase, I denotes isotropic phase, T_{g} denotes glass-transition temperature. The number between two symbols indicates the conversion temperature in degrees Celsius.

The host mixture used for determination of the optical anisotropy Δn of the compounds of the formula I is the commercial mixture ZLI-4792 (Merck KGaA). The dielectric anisotropy Δ∈ is determined using commercial mixture ZLI-2857. The physical data of the compound to be investigated are obtained from the change in the dielectric constants of the host mixture after addition of the compound to be investigated and extrapolation to 100% of the compound employed. In general, 10% of the compound to be investigated are dissolved in the host mixture, depending on the solubility.

Unless indicated otherwise, parts or percent data denote parts by weight or percent by weight.

Above and below, the symbols and abbreviations have the following meanings:
Vₒ threshold voltage, capacitive [V] at 20° C.
Δn the optical anisotropy measured at 20° C. and 589 nm
Δ∈ the dielectric anisotropy at 20° C. and 1 kHz
cl.p. clearing point [° C.]
K₁ elastic constant, “splay” deformation at 20° C. [pN]
K₃ elastic constant, “bend” deformation at 20° C. [pN]
Y₁ rotational viscosity measured at 20° C. [mPa·s], determined by the rotation method in a magnetic field
LTS low-temperature stability (nematic phase), determined in test cells.

The display used for measurement of the threshold voltage has two plane-parallel outer plates at a separation of 20 μm and electrode layers with alignment layers comprising SE-1211 (Nissan Chemicals) on top on the insides of the outer plates, which effect a homeotropic alignment of the liquid crystals.

All concentrations in this application, unless explicitly indicated otherwise, relate to the corresponding mixture or mixture component. All physical properties are determined in accordance with “Merck Liquid Crystals, Physical Properties of Liquid Crystals”, status November 1997, Merck KGaA, Germany, and apply to a temperature of 20° C., unless explicitly indicated otherwise.

### MIXTURE EXAMPLES

### Example M1

| | | | |
|---|---|---|---|
| CY-3-O2 | 16.50% | Clearing point [° C.]: | 74.0 |
| CCY-4-O2 | 10.50% | Δn [589 nm, 20° C.]: | 0.1069 |
| CCY-5-O2 | 6.00% | Δε [1 kHz, 20° C.]: | −3.2 |
| CPY-2-O2 | 9.00% | ε_{\|\|} [1 kHz, 20° C.]: | 3.5 |
| CPY-3-O2 | 9.00% | K₁ [pN, 20° C.]: | 12.9 |
| CCH-34 | 9.00% | K₃ [pN, 20° C.]: | 13.7 |
| CCH-13 | 20.00% | γ₁ [mPa · s, 20° C.]: | 117 |
| CCP-3-1 | 2.00% | V₀ [20° C., V]: | 2.18 |
| PYP-2-3 | 6.50% | | |
| PYP-2-4 | 6.50% | | |
| PCH-3O1 | 5.00% | | |

### Example M2

| | | | |
|---|---|---|---|
| CY-3-O2 | 16.50% | Clearing point [° C.]: | 74.5 |
| CCY-4-O2 | 9.50% | Δn [589 nm, 20° C.]: | 0.1070 |
| CCY-5-O2 | 4.00% | Δε [1 kHz, 20° C.]: | −3.2 |
| CPY-2-O2 | 9.00% | ε_{\|\|} [1 kHz, 20° C.]: | 3.5 |
| CPY-3-O2 | 9.00% | K₁ [pN, 20° C.]: | 13.1 |
| CCH-34 | 9.00% | K₃ [pN, 20° C.]: | 13.9 |
| CCH-13 | 20.00% | γ₁ [mPa · s, 20° C.]: | 117 |
| CCP-3-1 | 5.00% | V₀ [20° C., V]: | 2.19 |
| PYP-2-3 | 4.00% | | |
| PYP-2-4 | 4.00% | | |
| PCH-3O1 | 5.00% | | |
| PGIY-2-O4 | 5.00% | | |

### Example M3

| | | | |
|---|---|---|---|
| CY-3-O2 | 16.00% | Clearing point [° C.]: | 75.0 |
| CCY-3-O1 | 4.50% | Δn [589 nm, 20° C.]: | 0.1081 |
| CCY-3-O2 | 10.00% | Δε [1 kHz, 20° C.]: | −3.4 |
| CPY-2-O2 | 8.00% | ε_{\|\|} [1 kHz, 20° C.]: | 3.5 |
| CPY-3-O2 | 10.50% | K₁ [pN, 20° C.]: | 13.3 |
| CCH-23 | 9.00% | K₃ [pN, 20° C.]: | 14.6 |
| CCH-13 | 20.00% | γ₁ [mPa · s, 20° C.]: | 118 |
| CCP-3-1 | 6.00% | V₀ [20° C., V]: | 2.20 |
| PYP-2-3 | 4.00% | | |
| PYP-2-4 | 4.00% | | |
| PCH-3O1 | 3.00% | | |
| PGIY-2-O4 | 5.00% | | |

### Example M4

| | | | |
|---|---|---|---|
| CY-3-O2 | 17.00% | Clearing point [° C.]: | 75.5 |
| CCY-3-O2 | 11.50% | Δn [589 nm, 20° C.]: | 0.1082 |
| CPY-2-O2 | 8.00% | Δε [1 kHz, 20° C.]: | −3.3 |
| CPY-3-O2 | 11.50% | ε_{\|\|} [1 kHz, 20° C.]: | 3.5 |
| CCH-23 | 20.00% | K₁ [pN, 20° C.]: | 13.2 |
| CCH-13 | 10.00% | K₃ [pN, 20° C.]: | 14.5 |
| CCP-3-1 | 6.00% | γ₁ [mPa · s, 20° C.]: | 115 |
| PYP-2-3 | 4.00% | V₀ [20° C., V]: | 2.21 |
| PYP-2-4 | 4.00% | | |
| PCH-3O1 | 3.00% | | |
| PGIY-2-O4 | 5.00% | | |

### Example M5

| | | | |
|---|---|---|---|
| BCH-32 | 4.25% | Clearing point [° C.]: | 75.1 |
| CC-3-V1 | 10.00% | Δn [589 nm, 20° C.]: | 0.0975 |
| CCH-13 | 12.00% | Δε [1 kHz, 20° C.]: | −3.6 |
| CCH-35 | 3.75% | ε_{\|\|} [1 kHz, 20° C.]: | 3.5 |
| CCP-3-1 | 10.00% | K₁ [pN, 20° C.]: | 14.4 |
| CCY-3-O1 | 5.00% | K₃ [pN, 20° C.]: | 16.5 |
| CCY-3-O2 | 3.25% | γ₁ [mPa · s, 20° C.]: | 127 |
| CPY-2-O2 | 7.00% | V₀ [20° C., V]: | 2.26 |
| CPY-3-O2 | 11.75% | | |
| CY-3-O2 | 15.50% | | |
| CY-3-O4 | 3.50% | | |
| CY-5-O2 | 14.00% | | |

### Example M6

| | | | |
|---|---|---|---|
| BCH-32 | 2.50% | Clearing point [° C.]: | 75.7 |
| CCH-13 | 16.75% | Δn [589 nm, 20° C.]: | 0.0974 |
| CCH-34 | 4.50% | Δε [1 kHz, 20° C.]: | −3.5 |
| CCH-35 | 7.25% | ε_{\|\|} [1 kHz, 20° C.]: | 3.5 |
| CCP-3-1 | 10.00% | K₁ [pN, 20° C.]: | 14.7 |
| CCY-3-O1 | 11.50% | K₃ [pN, 20° C.]: | 16.1 |
| CCY-3-O2 | 9.75% | γ₁ [mPa · s, 20° C.]: | 123 |
| CPY-3-O2 | 9.25% | V₀ [20° C., V]: | 2.28 |
| CY-3-O2 | 15.00% | | |
| PY-3-O2 | 13.50% | | |

### Example M7

| | | | |
|---|---|---|---|
| CC-3-V1 | 10.00% | Clearing point [° C.]: | 75.1 |
| CCH-13 | 14.50% | Δn [589 nm, 20° C.]: | 0.0979 |
| CCH-34 | 4.00% | Δε [1 kHz, 20° C.]: | −3.5 |
| CCH-35 | 7.00% | ε_{\|\|} [1 kHz, 20° C.]: | 3.5 |
| CCP-3-1 | 3.50% | K₁ [pN, 20° C.]: | 14.7 |
| CCY-3-O1 | 12.00% | K₃ [pN, 20° C.]: | 16.0 |
| CCY-3-O2 | 7.00% | γ₁ [mPa · s, 20° C.]: | 122 |
| CPY-2-O2 | 4.25% | V₀ [20° C., V]: | 2.24 |
| CPY-3-O2 | 12.50% | | |
| CY-3-O4 | 12.75% | | |
| PY-3-O2 | 12.50% | | |

### Example M8

For the preparation of a PS-VA mixture, 99.7% of the mixture according to Example M1 are mixed with 0.3% of the polymerizable compound of the formula

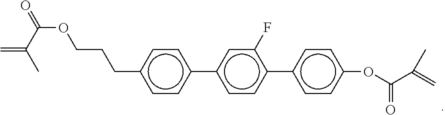

### Example M9

For the preparation of a PS-VA mixture, 99.75% of the mixture according to Example M1 are mixed with 0.25% of the polymerizable compound of the formula

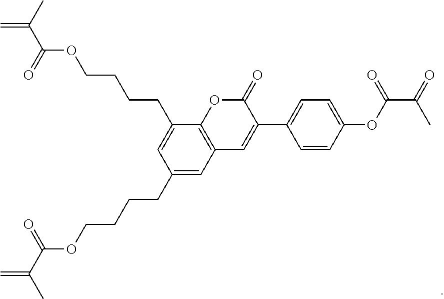 [SMILES: C=C(C)C(=O)OCCCCc1cc(CCCCOC(=O)C(=C)C)c2oc(=O)c(-c3ccc(OC(=O)C(C)=O)cc3)cc2c1]

### Example M10

For the preparation of a PS-VA mixture, 99.8% of the mixture according to Example M1 are mixed with 0.2% of the polymerizable compound of the formula

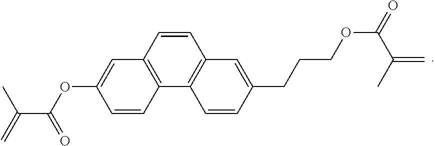 [SMILES: C=C(C)C(=O)OCCCc1ccc2c(ccc3cc(OC(=O)C(=C)C)ccc32)c1]

### Example M11

For the preparation of a PS-VA mixture, 99.75% of the mixture according to Example M1 are mixed with 0.25% of the polymerizable compound of the formula

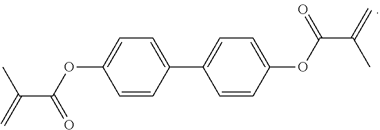 [SMILES: C=C(C)C(=O)Oc1ccc(-c2ccc(OC(=O)C(=C)C)cc2)cc1]

### Example M12

For the preparation of a PS-VA mixture, 99.75% of the mixture according to Example M1 are mixed with 0.25% of the polymerizable compound of the formula

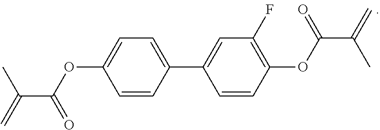 [SMILES: C=C(C)C(=O)Oc1ccc(-c2ccc(OC(=O)C(=C)C)c(F)c2)cc1]

### Example M13

For the preparation of a PS-VA mixture, 99.75% of the mixture according to Example M1 are mixed with 0.25% of the polymerizable compound of the formula

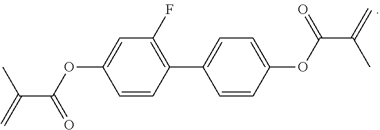 [SMILES: C=C(C)C(=O)Oc1ccc(-c2ccc(OC(=O)C(=C)C)cc2F)cc1]

### Example M14

For the preparation of a PS-VA mixture, 99.8% of the mixture according to Example M1 are mixed with 0.2% of the polymerizable compound of the formula

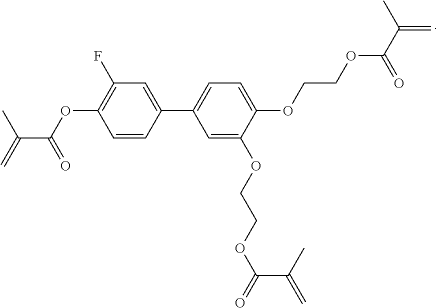 [SMILES: C=C(C)C(=O)OCCOc1ccc(-c2ccc(OC(=O)C(=C)C)c(F)c2)cc1OCCOC(=O)C(=C)C]

### Example M15

For the preparation of a PS-VA mixture, 99.8% of the mixture according to Example M1 are mixed with 0.2% of the polymerizable compound of the formula

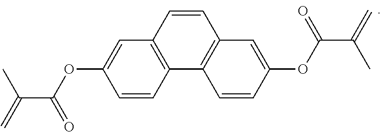 [SMILES: C=C(C)C(=O)Oc1ccc2c(ccc3cc(OC(=O)C(=C)C)ccc32)c1]

### Example M16

For the preparation of a PS-VA mixture, 99.75% of the mixture according to Example M2 are mixed with 0.25% of the polymerizable compound of the formula

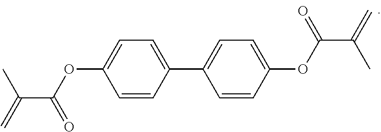 [SMILES: C=C(C)C(=O)Oc1ccc(-c2ccc(OC(=O)C(=C)C)cc2)cc1]

### Example M17

For the preparation of a PS-VA mixture, 99.7% of the mixture according to Example M2 are mixed with 0.3% of the polymerizable compound of the formula

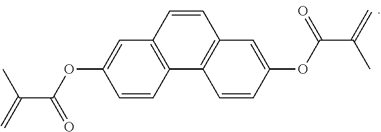 [SMILES: C=C(C)C(=O)Oc1ccc2c(ccc3cc(OC(=O)C(=C)C)ccc32)c1]

### Example M18

For the preparation of a PS-VA mixture, 99.7% of the mixture according to Example M2 is mixed with 0.3% of the polymerizable compound of the formula

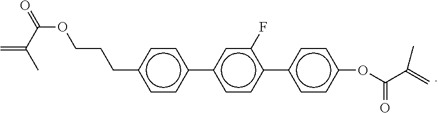

### Example M19

For the preparation of a PS-VA mixture, 99.75% of the mixture according to Example M2 is mixed with 0.25% of the polymerizable compound of the formula

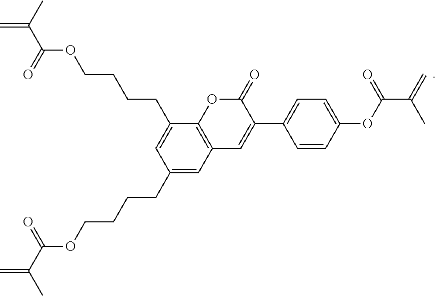 [SMILES: C=C(C)C(=O)OCCCCc1cc(CCCCOC(=O)C(=C)C)c2oc(=O)c(-c3ccc(OC(=O)C(=C)C)cc3)cc2c1]

### Example M20

For the preparation of a PS-VA mixture, 99.7% of the mixture according to Example M2 is mixed with 0.3% of the polymerizable compound of the formula

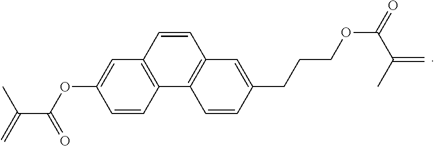 [SMILES: C=C(C)C(=O)OCCCc1ccc2c(ccc3cc(OC(=O)C(=C)C)ccc32)c1]

### Example M21

For the preparation of a PS-VA mixture, 99.75% of the mixture according to Example M2 is mixed with 0.25% of the polymerizable compound of the formula

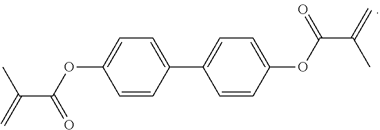 [SMILES: C=C(C)C(=O)Oc1ccc(-c2ccc(OC(=O)C(=C)C)cc2)cc1]

### Example M22

For the preparation of a PS-VA mixture, 99.7% of the mixture according to Example M2 is mixed with 0.3% of the polymerizable compound of the formula

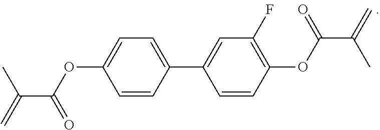 [SMILES: C=C(C)C(=O)Oc1ccc(-c2ccc(OC(=O)C(=C)C)c(F)c2)cc1]

### Example M23

For the preparation of a PS-VA mixture, 99.75% of the mixture according to Example M2 is mixed with 0.25% of the polymerizable compound of the formula

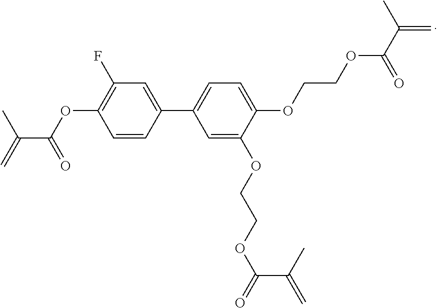 [SMILES: C=C(C)C(=O)OCCOc1ccc(-c2ccc(OC(=O)C(=C)C)c(F)c2)cc1OCCOC(=O)C(=C)C]

### Example M24

For the preparation of a PS-VA mixture, 99.75% of the mixture according to Example M3 is mixed with 0.25% of the polymerizable compound of the formula

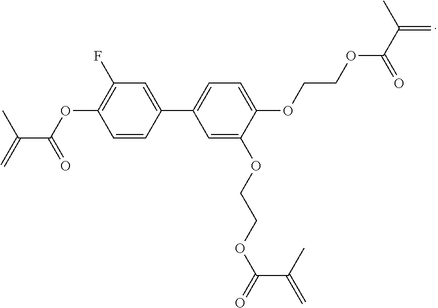 [SMILES: C=C(C)C(=O)OCCOc1ccc(-c2ccc(OC(=O)C(=C)C)c(F)c2)cc1OCCOC(=O)C(=C)C]

### Example M25

For the preparation of a PS-VA mixture, 99.8% of the mixture according to Example M3 is mixed with 0.2% of the polymerizable compound of the formula

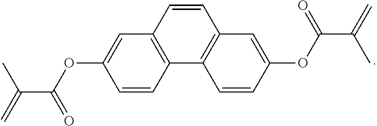 [SMILES: C=C(C)C(=O)Oc1ccc2c(ccc3cc(OC(=O)C(=C)C)ccc32)c1]

### Example M26

For the preparation of a PS-VA mixture, 99.7% of the mixture according to Example M3 is mixed with 0.3% of the polymerizable compound of the formula

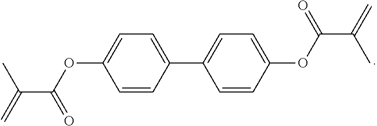 [SMILES: C=C(C)C(=O)Oc1ccc(-c2ccc(OC(=O)C(=C)C)cc2)cc1]

### Example M27

For the preparation of a PS-VA mixture, 99.7% of the mixture according to Example M3 is mixed with 0.3% of the polymerizable compound of the formula

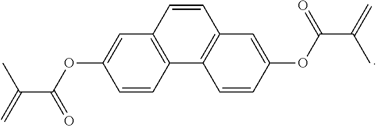 [SMILES: C=C(C)C(=O)Oc1ccc2c(ccc3cc(OC(=O)C(=C)C)ccc32)c1]

### Example M28

For the preparation of a PS-VA mixture, 99.7% of the mixture according to Example M3 is mixed with 0.3% of the polymerizable compound of the formula

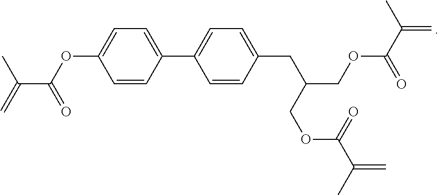 [SMILES: C=C(C)C(=O)OCC(COC(=O)C(=C)C)Cc1ccc(-c2ccc(OC(=O)C(=C)C)cc2)cc1]

### Example M29

For the preparation of a PS-VA mixture, 99.7% of the mixture according to Example M3 is mixed with 0.3% of the polymerizable compound of the formula

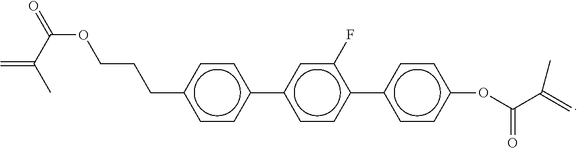

### Example M30

For the preparation of a PS-VA mixture, 99.7% of the mixture according to Example M4 is mixed with 0.3% of the polymerizable compound of the formula

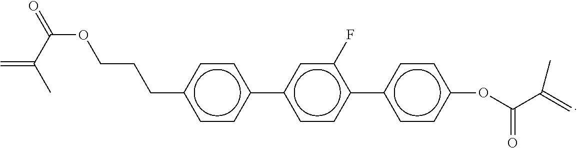

### Example M31

For the preparation of a PS-VA mixture, 99.75% of the mixture according to Example M4 is mixed with 0.25% of the polymerizable compound of the formula

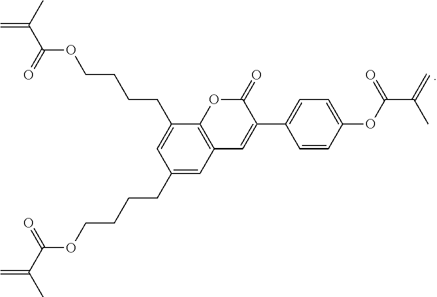 [SMILES: C=C(C)C(=O)OCCCCc1cc(CCCCOC(=O)C(=C)C)c2oc(=O)c(-c3ccc(OC(=O)C(=C)C)cc3)cc2c1]

### Example M32

For the preparation of a PS-VA mixture, 99.75% of the mixture according to Example M4 is mixed with 0.25% of the polymerizable compound of the formula

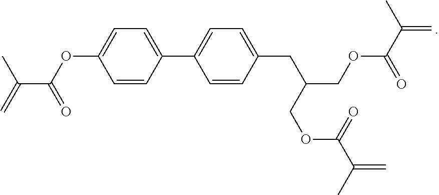 [SMILES: C=C(C)C(=O)OCC(COC(=O)C(=C)C)Cc1ccc(-c2ccc(OC(=O)C(=C)C)cc2)cc1]

### Example M33

For the preparation of a PS-VA mixture, 99.7% of the mixture according to Example M4 is mixed with 0.3% of the polymerizable compound of the formula

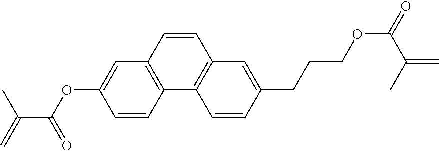 [SMILES: C=C(C)C(=O)OCCCc1ccc2c(ccc3cc(OC(=O)C(=C)C)ccc32)c1]

### Example M34

For the preparation of a PS-VA mixture, 99.75% of the mixture according to Example M4 is mixed with 0.25% of the polymerizable compound of the formula

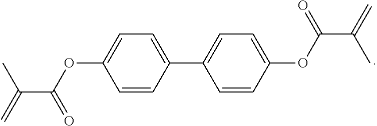 [SMILES: C=C(C)C(=O)Oc1ccc(-c2ccc(OC(=O)C(=C)C)cc2)cc1]

### Example M35

For the preparation of a PS-VA mixture, 99.75% of the mixture according to Example M4 is mixed with 0.25% of the polymerizable compound of the formula

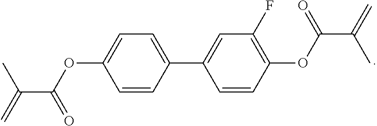 [SMILES: C=C(C)C(=O)Oc1ccc(-c2ccc(OC(=O)C(=C)C)c(F)c2)cc1]

### Example M36

For the preparation of a PS-VA mixture, 99.7% of the mixture according to Example M5 is mixed with 0.3% of the polymerizable compound of the formula

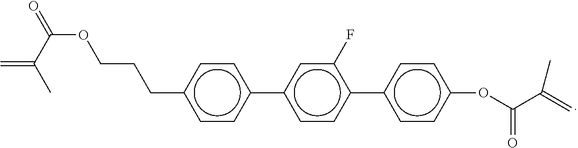

### Example M37

For the preparation of a PS-VA mixture, 99.75% of the mixture according to Example M5 is mixed with 0.25% of the polymerizable compound of the formula

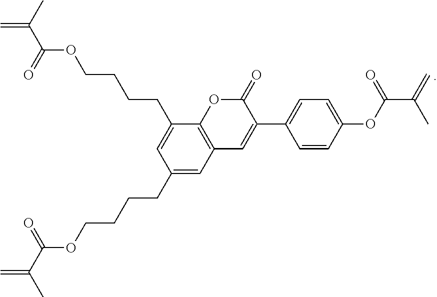 [SMILES: C=C(C)C(=O)OCCCCc1cc(CCCCOC(=O)C(=C)C)c2oc(=O)c(-c3ccc(OC(=O)C(=C)C)cc3)cc2c1]

### Example M38

For the preparation of a PS-VA mixture, 99.8% of the mixture according to Example M5 is mixed with 0.2% of the polymerizable compound of the formula

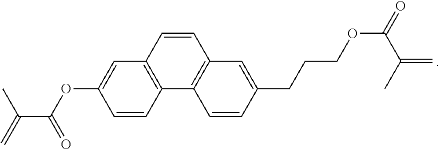 [SMILES: C=C(C)C(=O)OCCCc1ccc2c(ccc3cc(OC(=O)C(=C)C)ccc32)c1]

### Example M39

For the preparation of a PS-VA mixture, 99.75% of the mixture according to Example M5 is mixed with 0.25% of the polymerizable compound of the formula

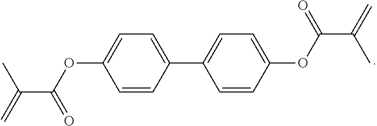 [SMILES: C=C(C)C(=O)Oc1ccc(-c2ccc(OC(=O)C(=C)C)cc2)cc1]

### Example M40

For the preparation of a PS-VA mixture, 99.75% of the mixture according to Example M5 is mixed with 0.25% of the polymerizable compound of the formula

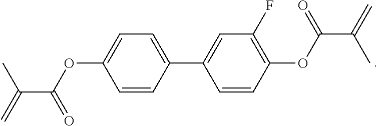 [SMILES: C=C(C)C(=O)Oc1ccc(-c2ccc(OC(=O)C(=C)C)c(F)c2)cc1]

### Example M41

For the preparation of a PS-VA mixture, 99.75% of the mixture according to Example M5 is mixed with 0.25% of the polymerizable compound of the formula

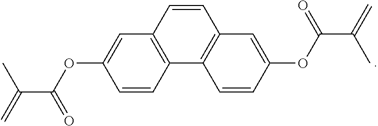 [SMILES: C=C(C)C(=O)Oc1ccc2c(ccc3cc(OC(=O)C(=C)C)ccc32)c1]

### Example M42

For the preparation of a PS-VA mixture, 99.75% of the mixture according to Example M5 is mixed with 0.25% of the polymerizable compound of the formula

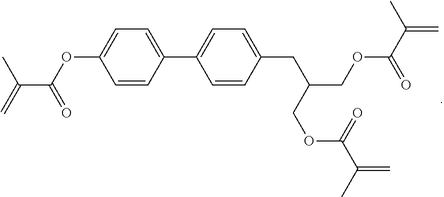 [SMILES: C=C(C)C(=O)OCC(COC(=O)C(=C)C)Cc1ccc(-c2ccc(OC(=O)C(=C)C)cc2)cc1]

### Example M43

For the preparation of a PS-VA mixture, 99.75% of the mixture according to Example M6 is mixed with 0.25% of the polymerizable compound of the formula

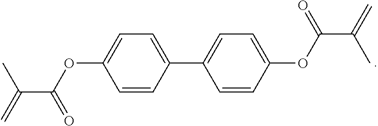 [SMILES: C=C(C)C(=O)Oc1ccc(-c2ccc(OC(=O)C(=C)C)cc2)cc1]

### Example M44

For the preparation of a PS-VA mixture, 99.7% of the mixture according to Example M6 is mixed with 0.3% of the polymerizable compound of the formula

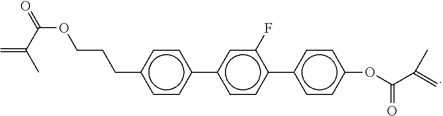

### Example M45

For the preparation of a PS-VA mixture, 99.75% of the mixture according to Example M6 is mixed with 0.25% of the polymerizable compound of the formula

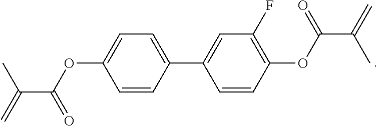 [SMILES: C=C(C)C(=O)Oc1ccc(-c2ccc(OC(=O)C(=C)C)c(F)c2)cc1]

### Example M46

For the preparation of a PS-VA mixture, 99.75% of the mixture according to Example M7 is mixed with 0.25% of the polymerizable compound of the formula

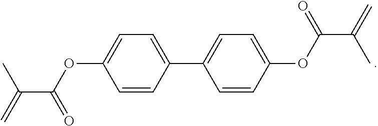 [SMILES: C=C(C)C(=O)Oc1ccc(-c2ccc(OC(=O)C(=C)C)cc2)cc1]

### Example M47

For the preparation of a PS-VA mixture, 99.7% of the mixture according to Example M7 is mixed with 0.3% of the polymerizable compound of the formula

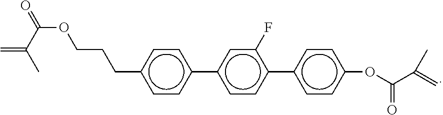

### Example M48

For the preparation of a PS-VA mixture, 99.75% of the mixture according to Example M7 is mixed with 0.25% of the polymerizable compound of the formula

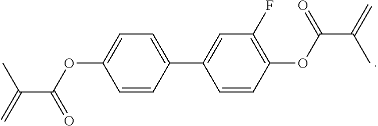 [SMILES: C=C(C)C(=O)Oc1ccc(-c2ccc(OC(=O)C(=C)C)c(F)c2)cc1]

### Example M49

For the preparation of a PS-VA mixture, 99.75% of the mixture according to Example M7 is mixed with 0.25% of the polymerizable compound of the formula

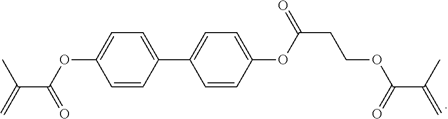 [SMILES: C=C(C)C(=O)OCCC(=O)Oc1ccc(-c2ccc(OC(=O)C(=C)C)cc2)cc1]

### Example M50

| | | | |
|---|---|---|---|
| CC-3-V | 15.00% | Clearing point [° C.]: | 74 |
| CC-3-V1 | 9.00% | Δn [589 nm, 20° C.]: | 0.1085 |
| CCH-23 | 8.00% | Δε [1 kHz, 20° C.]: | −3.3 |
| CCH-13 | 6.00% | ε_{\|\|} [1 kHz, 20° C.]: | 3.5 |
| CCY-3-O2 | 12.00% | ε_{⊥} [1 kHz, 20° C.]: | 6.8 |
| CCY-5-O2 | 8.00% | K₁ [pN, 20° C.]: | 14.1 |
| CPY-2-O2 | 3.00% | K₃ [pN, 20° C.]: | 15.8 |
| CPY-3-O2 | 9.50% | γ₁ [mPa · s, 20° C.]: | 102 |
| CY-3-O2 | 7.50% | V₀ [20° C., V]: | 2.31 |
| PY-3-O2 | 14.00% | | |
| PYP-2-3 | 8.00% | | |

### Example M51

| | | | |
|---|---|---|---|
| CY-3-O2 | 16.50% | Clearing point [° C.]: | 74.5 |
| CCY-3-O2 | 11.50% | Δn [589 nm, 20° C.]: | 0.1079 |
| CPY-2-O2 | 3.00% | Δε [1 kHz, 20° C.]: | −3.5 |
| CPY-3-O2 | 11.00% | ε_{\|\|} [1 kHz, 20° C.]: | 3.6 |
| CCH-34 | 10.00% | ε_{⊥} [1 kHz, 20° C.]: | 7.1 |
| CCH-13 | 20.00% | K₁ [pN, 20° C.]: | 13.9 |
| CCP-3-1 | 9.00% | K₃ [pN, 20° C.]: | 15.2 |
| PYP-2-3 | 7.00% | γ₁ [mPa · s, 20° C.]: | 115 |
| PCH-3O1 | 3.00% | V₀ [20° C., V]: | 2.20 |
| PGIY-2-O4 | 5.00% | | |
| B-2O-O5 | 4.00% | | |

### Example M52

For the preparation of a PS-VA mixture, 99.75% of the mixture according to Example M51 are mixed with 0.25% of the polymerizable compound of the formula

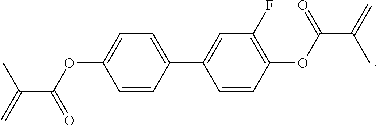 [SMILES: C=C(C)C(=O)Oc1ccc(-c2ccc(OC(=O)C(=C)C)c(F)c2)cc1]

### Example M53

| | | | |
|---|---|---|---|
| CY-3-O2 | 18.00% | Clearing point [° C.]: | 75 |
| CCY-3-O2 | 10.50% | Δn [589 nm, 20° C.]: | 0.1076 |
| CPY-2-O2 | 1.50% | Δε [1 kHz, 20° C.]: | −3.5 |
| CPY-3-O2 | 11.00% | ε_{\|\|} [1 kHz, 20° C.]: | 3.6 |
| CCH-23 | 20.00% | ε_{⊥} [1 kHz, 20° C.]: | 7.0 |
| CCH-13 | 8.00% | K₁ [pN, 20° C.]: | 13.7 |
| CCP-3-1 | 12.00% | K₃ [pN, 20° C.]: | 15.1 |
| PYP-2-3 | 7.00% | γ₁ [mPa · s, 20° C.]: | 113 |
| PCH-3O1 | 3.00% | V₀ [20° C., V]: | 2.21 |
| PGIY-2-O4 | 5.00% | LTS [bulk −20° C.]: | >1000 h |
| B-2O-O5 | 4.00% | | |

### Example M54

| | | | |
|---|---|---|---|
| CY-3-O2 | 15.00% | Clearing point [° C.]: | 74.5 |
| CY-5-O2 | 5.00% | Δn [589 nm, 20° C.]: | 0.1071 |
| CCP-3-1 | 1.00% | Δε [1 kHz, 20° C.]: | −2.9 |
| CCY-3-O2 | 11.00% | ε_{\|\|} [1 kHz, 20° C.]: | 3.5 |
| CPY-2-O2 | 5.50% | ε_{⊥} [1 kHz, 20° C.]: | 6.4 |
| CPY-3-O2 | 11.00% | K₁ [pN, 20° C.]: | 14.0 |
| CC-5-V | 19.00% | K₃ [pN, 20° C.]: | 15.6 |
| CC-3-V1 | 8.50% | γ₁ [mPa · s, 20° C.]: | 103 |
| CCH-13 | 11.00% | V₀ [20° C., V]: | 2.41 |
| PYP-2-3 | 12.50% | | |
| PPGU-3-F | 0.50% | | |

### Example M55

For the preparation of a PS-VA mixture, 99.75% of the mixture according to Example M54 are mixed with 0.25% of the polymerizable compound of the formula

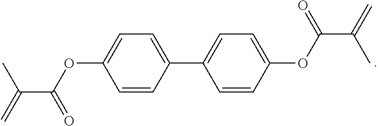 [SMILES: C=C(C)C(=O)Oc1ccc(-c2ccc(OC(=O)C(=C)C)cc2)cc1]

### Example M56

| | | | |
|---|---|---|---|
| CC-5-V | 15.00% | Clearing point [° C.]: | 74.5 |
| CC-3-V1 | 9.00% | Δn [589 nm, 20° C.]: | 0.1087 |
| CCH-13 | 8.00% | Δε [1 kHz, 20° C.]: | −2.9 |
| CCH-34 | 7.50% | ε_{\|\|} [1 kHz, 20° C.]: | 3.5 |
| CCY-3-O2 | 10.00% | ε_{⊥} [1 kHz, 20° C.]: | 6.7 |
| CCY-5-O2 | 8.00% | K₁ [pN, 20° C.]: | 14.9 |
| CPY-2-O2 | 3.00% | K₃ [pN, 20° C.]: | 15.6 |
| CPY-3-O2 | 8.50% | γ₁ [mPa · s, 20° C.]: | 105 |
| CY-3-O2 | 7.00% | V₀ [20° C., V]: | 2.33 |
| PY-3-O2 | 16.00% | | |
| PYP-2-3 | 8.00% | | |

### Example M57

| | | | |
|---|---|---|---|
| CC-5-V | 20.00% | Clearing point [° C.]: | 75 |
| CCH-13 | 14.50% | Δn [589 nm, 20° C.]: | 0.1078 |
| CC-3-V1 | 8.00% | Δε [1 kHz, 20° C.]: | −2.9 |
| CCP-3-1 | 2.00% | ε_{\|\|} [1 kHz, 20° C.]: | 3.4 |
| CCY-3-O1 | 6.00% | ε_{⊥} [1 kHz, 20° C.]: | 6.3 |
| CCY-3-O2 | 10.00% | K₁ [pN, 20° C.]: | 15.0 |
| CPY-2-O2 | 5.50% | K₃ [pN, 20° C.]: | 15.6 |
| CPY-3-O2 | 11.50% | γ₁ [mPa · s, 20° C.]: | 98 |
| PY-3-O2 | 17.50% | V₀ [20° C., V]: | 2.45 |
| PYP-2-3 | 5.00% | LTS [bulk −20° C.]: | >1000 h |

### Example M58

| | | | |
|---|---|---|---|
| CC-3-V1 | 9.00% | Clearing point [° C.]: | 74.5 |
| CCH-13 | 18.00% | Δn [589 nm, 20° C.]: | 0.0987 |
| CCH-34 | 9.00% | Δε [1 kHz, 20° C.]: | −3.5 |
| CCP-3-1 | 5.50% | ε_{\|\|} [1 kHz, 20° C.]: | 3.5 |
| CCY-3-O1 | 3.50% | ε_{⊥} [1 kHz, 20° C.]: | 6.9 |
| CCY-3-O2 | 11.50% | K₁ [pN, 20° C.]: | 14.5 |
| CPY-2-O2 | 9.00% | K₃ [pN, 20° C.]: | 16.1 |
| CPY-3-O2 | 11.00% | γ₁ [mPa · s, 20° C.]: | 107 |
| CY-3-O2 | 13.50% | V₀ [20° C., V]: | 2.28 |
| PY-3-O2 | 10.00% | | |

### Example M59

For the preparation of a PS-VA mixture, 99.7% of the mixture according to Example M58 are mixed with 0.3% of the polymerizable compound of the formula

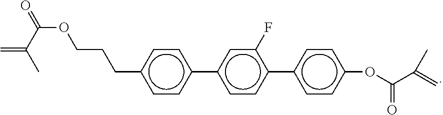

### Example M60

| | | | |
|---|---|---|---|
| CC-3-V | 25.50% | Clearing point [° C.]: | 66.5 |
| CCH-13 | 12.00% | Δn [589 nm, 20° C.]: | 0.0879 |
| PY-3-O2 | 9.00% | Δε [1 kHz, 20° C.]: | −3.6 |
| CEY-3-O2 | 9.00% | K₁ [pN, 20° C.]: | 12.1 |
| PY-5-O2 | 5.00% | K₃ [pN, 20° C.]: | 14.5 |
| CCP-3-1 | 5.00% | γ₁ [mPa · s, 20° C.]: | 92 |
| CCY-V-O1 | 5.00% | V₀ [20° C., V]: | 2.13 |
| CCY-V-O2 | 11.00% | | |
| CCP-3O1 | 2.00% | | |
| CCP-3-3 | 2.00% | | |
| CAIY-3-O2 | 7.00% | | |
| CCY-V-O4 | 7.50% | | |

### Example M61

For the preparation of a PS-VA mixture, 99.75% of the mixture according to Example M60 are mixed with 0.25% of the polymerizable compound of the formula

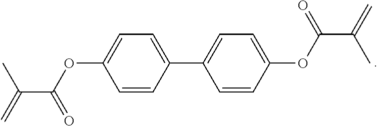 [SMILES: C=C(C)C(=O)Oc1ccc(-c2ccc(OC(=O)C(=C)C)cc2)cc1]

### Example M62

| | | | |
|---|---|---|---|
| CC-5-V | 20.00% | Clearing point [° C.]: | 74.5 |
| CCH-13 | 14.50% | Δn [589 nm, 20° C.]: | 0.1083 |
| CC-3-V1 | 8.00% | Δε [1 kHz, 20° C.]: | −3.0 |
| CCP-3-1 | 2.00% | ε_{\|\|} [1 kHz, 20° C.]: | 3.4 |
| CCY-3-O1 | 4.00% | ε_{⊥} [1 kHz, 20° C.]: | 6.4 |
| CCY-3-O2 | 11.00% | K₁ [pN, 20° C.]: | 15.6 |
| CPY-2-O2 | 7.50% | K₃ [pN, 20° C.]: | 15.9 |
| CPY-3-O2 | 11.50% | γ₁ [mPa · s, 20° C.]: | 97 |
| PY-3-O2 | 12.00% | V₀ [20° C., V]: | 2.44 |
| PYP-2-3 | 1.00% | | |
| B-2O-O5 | 4.00% | | |
| PP-1-2V1 | 4.50% | | |

### Example M63

| | | | |
|---|---|---|---|
| CC-5-V | 20.00% | Clearing point [° C.]: | 74.5 |
| CC-3-V1 | 8.00% | Δn [589 nm, 20° C.]: | 0.1083 |
| CCH-13 | 10.00% | Δε [1 kHz, 20° C.]: | −3.1 |
| CCH-34 | 4.00% | ε_{\|\|} [1 kHz, 20° C.]: | 3.5 |
| CCY-3-O2 | 11.50% | ε_{⊥} [1 kHz, 20° C.]: | 6.6 |
| CCY-5-O2 | 3.00% | K₁ [pN, 20° C.]: | 14.9 |
| CPY-2-O2 | 7.50% | K₃ [pN, 20° C.]: | 15.4 |
| CPY-3-O2 | 11.00% | γ₁ [mPa · s, 20° C.]: | 102 |
| CY-3-O2 | 3.50% | V₀ [20° C., V]: | 2.35 |
| PY-3-O2 | 16.50% | | |
| PYP-2-3 | 5.00% | | |

### Example M64

| | | | |
|---|---|---|---|
| CC-5-V | 20.00% | Clearing point [° C.]: | 74.5 |
| CCH-13 | 14.50% | Δn [589 nm, 20° C.]: | 0.1080 |
| CC-3-V1 | 8.00% | Δε [1 kHz, 20° C.]: | −3.2 |
| CCY-3-O1 | 4.00% | ε_{\|\|} [1 kHz, 20° C.]: | 3.5 |
| CCY-3-O2 | 11.00% | ε_{⊥} [1 kHz, 20° C.]: | 6.6 |
| CPY-2-O2 | 3.50% | K₁ [pN, 20° C.]: | 15.8 |
| CPY-3-O2 | 11.50% | K₃ [pN, 20° C.]: | 16.3 |
| CLY-3-O2 | 5.00% | γ₁ [mPa · s, 20° C.]: | 99 |
| PY-3-O2 | 13.00% | V₀ [20° C., V]: | 2.39 |
| PYP-2-3 | 1.00% | | |
| B-2O-O5 | 4.00% | | |
| PP-1-2V1 | 4.50% | | |

### Example M65

For the preparation of a PS-VA mixture, 99.7% of the mixture according to Example M62 are mixed with 0.3% of the polymerizable compound of the formula

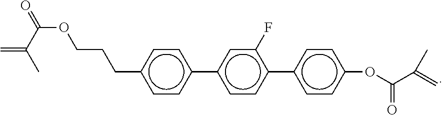

### Example M66

| | | | |
|---|---|---|---|
| CC-5-V | 20.00% | Clearing point [° C.]: | 75 |
| CCH-13 | 14.50% | Δn [589 nm, 20° C.]: | 0.1083 |
| CC-3-V1 | 8.00% | Δε [1 kHz, 20° C.]: | −3.1 |
| CCY-3-O1 | 4.00% | ε_{\|\|} [1 kHz, 20° C.]: | 3.5 |
| CCY-3-O2 | 11.00% | ε_{⊥} [1 kHz, 20° C.]: | 6.6 |
| CPY-2-O2 | 3.50% | K₁ [pN, 20° C.]: | 15.8 |
| CPY-3-O2 | 11.50% | K₃ [pN, 20° C.]: | 16.1 |
| CLY-3-O2 | 5.00% | γ₁ [mPa · s, 20° C.]: | 98 |
| PY-3-O2 | 13.00% | V₀ [20° C., V]: | 2.41 |
| PYP-2-3 | 1.00% | | |
| B-2O-O5 | 4.00% | | |
| PP-1-2V1 | 4.50% | | |

### Example M67

| | | | |
|---|---|---|---|
| CC-4-V | 16.00% | Clearing point [° C.]: | 75 |
| CCH-13 | 12.00% | Δn [589 nm, 20° C.]: | 0.1080 |
| CC-3-V1 | 8.00% | Δε [1 kHz, 20° C.]: | −3.1 |
| CCP-3-1 | 12.50% | ε_{\|\|} [1 kHz, 20° C.]: | 3.5 |
| CCY-3-O1 | 3.50% | ε_{⊥} [1 kHz, 20° C.]: | 6.7 |
| CCY-3-O2 | 11.00% | K₁ [pN, 20° C.]: | 15.0 |
| CPY-3-O2 | 10.50% | K₃ [pN, 20° C.]: | 15.8 |
| PY-3-O2 | 20.00% | γ₁ [mPa · s, 20° C.]: | 98 |
| PYP-2-3 | 2.50% | V₀ [20° C., V]: | 2.37 |
| B-2O-O5 | 4.00% | | |

### Example M68

| | | | |
|---|---|---|---|
| CC-3-V | 20.00% | Clearing point [° C.]: | 72.5 |
| CC-3-V1 | 8.00% | Δn [589 nm, 20° C.]: | 0.1078 |
| CCH-13 | 7.50% | Δε [1 kHz, 20° C.]: | −3.0 |
| CCP-3-1 | 14.50% | ε_{\|\|} [1 kHz, 20° C.]: | 3.5 |
| CCP-3-3 | 5.00% | ε_{⊥} [1 kHz, 20° C.]: | 6.5 |
| CCY-3-O2 | 10.00% | K₁ [pN, 20° C.]: | 15.0 |
| CY-3-O2 | 4.00% | K₃ [pN, 20° C.]: | 15.8 |
| PY-3-O2 | 16.00% | γ₁ [mPa · s, 20° C.]: | 83 |
| PYP-2-3 | 5.00% | V₀ [20° C., V]: | 2.44 |
| B-2O-O5 | 3.00% | | |
| B(S)-2O-O5 | 3.00% | | |
| B(S)-2O-O4 | 2.00% | | |
| B(S)-2O-O6 | 2.00% | | |

### Example M69

For the preparation of a PS-VA mixture, 99.7% of the mixture according to Example M68 are mixed with 0.3% of the polymerizable compound of the formula

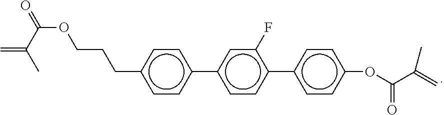

### Example M70

| | | | |
|---|---|---|---|
| CC-3-V1 | 10.00% | Clearing point [° C.]: | 74.6 |
| CCH-13 | 16.00% | Δn [589 nm, 20° C.]: | 0.0985 |
| CCH-34 | 6.00% | Δε [1 kHz, 20° C.]: | −3.4 |
| CCH-35 | 2.50% | ε_{\|\|} [1 kHz, 20° C.]: | 3.5 |
| CCOC-3-3 | 3.50% | ε_{⊥} [1 kHz, 20° C.]: | 6.9 |
| CCOC-4-3 | 3.50% | K₁ [pN, 20° C.]: | 14.8 |
| CCP-3-1 | 1.00% | K₃ [pN, 20° C.]: | 16.2 |
| CCY-3-O2 | 12.50% | γ₁ [mPa · s, 20° C.]: | 120 |
| CPY-2-O2 | 6.50% | V₀ [20° C., V]: | 2.29 |
| CPY-3-O2 | 13.00% | | |
| CY-3-O2 | 11.50% | | |
| PY-3-O2 | 14.00% | | |

### Example M71

For the preparation of a polymer-stabilized LC mixture, 99.75% of the mixture according to Example M70 are mixed with

0.25% of the polymerizable compound of the formula

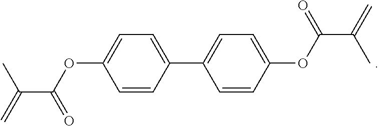 [SMILES: C=C(C)C(=O)Oc1ccc(-c2ccc(OC(=O)C(=C)C)cc2)cc1]

### Example M72

For the preparation of a polymer-stabilized LC mixture, 99.7% of the mixture according to Example M70 are mixed with

0.3% of the polymerizable compound of the formula

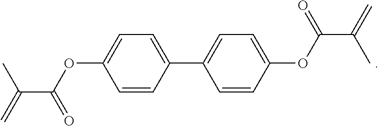 [SMILES: C=C(C)C(=O)Oc1ccc(-c2ccc(OC(=O)C(=C)C)cc2)cc1]

### Example M73

For the preparation of a polymer-stabilized LC mixture, 99.7% of the mixture according to Example M70 are mixed with

0.3% of the polymerizable compound of the formula

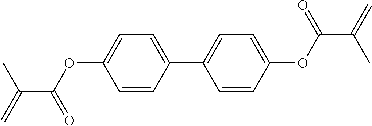 [SMILES: C=C(C)C(=O)Oc1ccc(-c2ccc(OC(=O)C(=C)C)cc2)cc1]

### and 0.001% of Irganox-1076 (BASF).

The mixture according to Example M73 is preferably suitable for PS-VA applications, in particular 2D and 3D TV applications.

The above-mentioned mixture examples for PS-VA applications are of course also suitable for PS-IPS and PS-FFS applications.

In order to improve the reliability, the mixtures according to Examples M1 to M69 may additionally be stabilized with one or two stabilizers selected from the group of compounds a) to h) mentioned below:

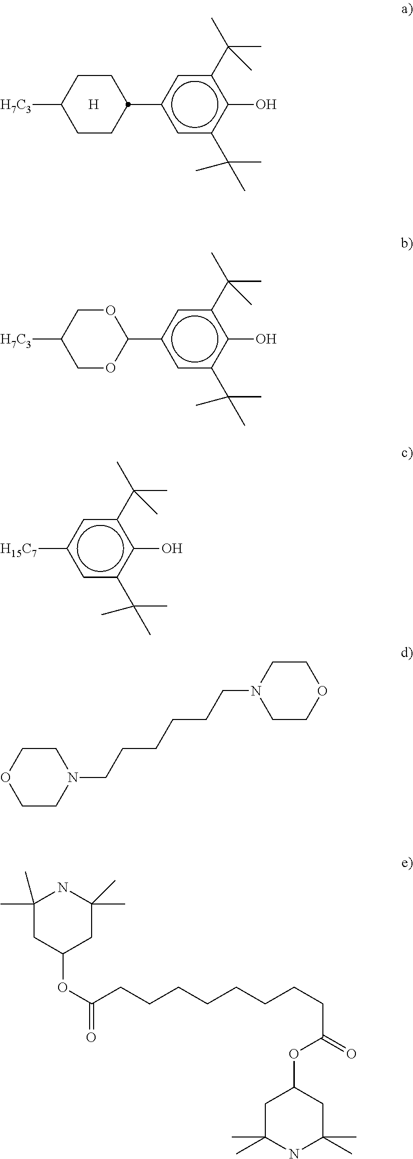

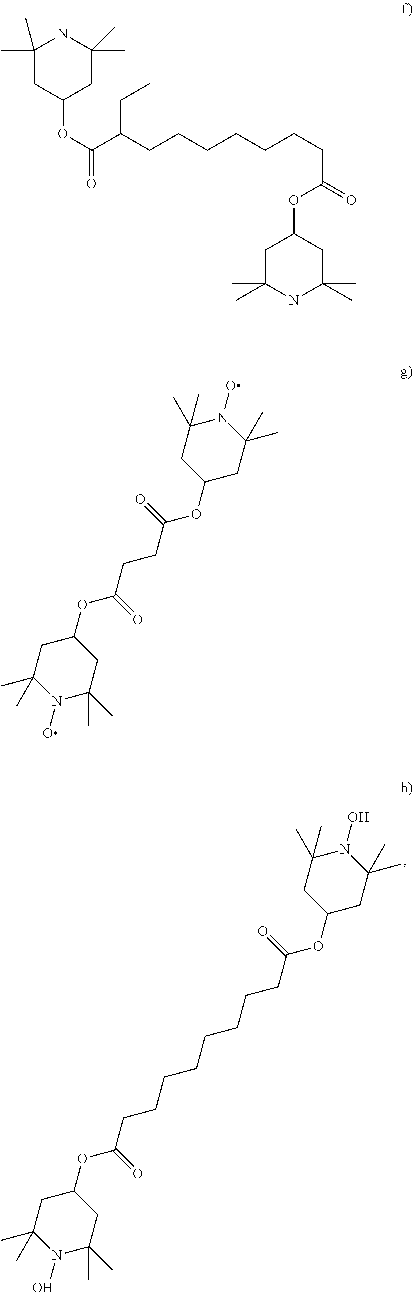

where the stabilizer is in each case added in amounts of 0.01-0.04%, based on the mixture.

### Example M74

| | | | |
|---|---|---|---|
| CCY-3-O2 | 4.50% | Clearing point [° C.]: | 74.5 |
| CPY-3-O2 | 11.00% | Δn [589 nm, 20° C.]: | 0.1284 |
| PYP-2-3 | 9.50% | Δε [1 kHz, 20° C.]: | −2.0 |
| PY-3-O2 | 15.00% | ε_{\|\|} [1 kHz, 20° C.]: | 3.4 |
| Y-4O-O4 | 4.00% | ε_{⊥} [1 kHz, 20° C.]: | 5.4 |
| CCH-23 | 15.00% | K₁ [pN, 20° C.]: | 14.7 |
| CCH-13 | 8.00% | K₃ [pN, 20° C.]: | 14.5 |
| CCP-3-1 | 11.00% | γ₁ [mPa · s, 20° C.]: | 95 |
| CCP-3-3 | 2.00% | V₀ [20° C., V]: | 2.81 |
| BCH-32 | 15.00% | LTS [bulk −20° C.]: | >1000 h |
| PP-1-3 | 5.00% | | |

### Example M75

| | | | |
|---|---|---|---|
| CC-3-V1 | 8.50% | Clearing point [° C.]: | 74.5 |
| CCH-13 | 7.50% | Δn [589 nm, 20° C.]: | 0.0998 |
| CCH-23 | 16.50% | Δε [1 kHz, 20° C.]: | −3.4 |
| CCP-3-1 | 16.00% | ε_{⊥} [1 kHz, 20° C.]: | 7.0 |
| CCY-3-O2 | 11.00% | K₁ [pN, 20° C.]: | 14.7 |
| CCY-3-O1 | 6.00% | K₃ [pN, 20° C.]: | 16.0 |
| CPY-3-O2 | 11.50% | γ₁ [mPa · s, 20° C.]: | 98 |
| B-2O-O5 | 3.00% | V₀ [20° C., V]: | 2.30 |
| Y-4O-O4 | 5.00% | | |
| PY-2-O2 | 8.00% | | |
| PY-1-O2 | 7.00% | | |

### Example M76

| | | | |
|---|---|---|---|
| CC-3-V1 | 4.00% | Clearing point [° C.]: | 73.5 |
| CCH-23 | 14.00% | Δn [589 nm, 20° C.]: | 0.0977 |
| CCH-34 | 3.00% | Δε [1 kHz, 20° C.]: | −3.5 |
| CCH-35 | 4.00% | ε_{∥} [1 kHz, 20° C.]: | 3.6 |
| CCH-13 | 4.00% | ε_{⊥} [1 kHz, 20° C.]: | 7.1 |
| CCP-3-1 | 14.00% | K₁ [pN, 20° C.]: | 14.6 |
| CCY-3-O2 | 11.00% | K₃ [pN, 20° C.]: | 16.1 |
| CCY-3-O1 | 5.50% | γ₁ [mPa · s, 20° C.]: | 107 |
| CPY-3-O2 | 11.00% | V₀ [20° C., V]: | 2.26 |
| CY-3-O2 | 12.00% | | |
| PY-3-O2 | 14.50% | | |
| Y-4O-O4 | 3.00% | | |

### Example M77

| | | | |
|---|---|---|---|
| BCH-32 | 4.25% | Clearing point [° C.]: | 75.1 |
| CC-3-V1 | 10.00% | Δn [589 nm, 20° C.]: | 0.0975 |
| CCH-13 | 12.00% | Δε [1 kHz, 20° C.]: | −3.6 |
| CCH-35 | 3.75% | ε_{∥} [1 kHz, 20° C.]: | 3.5 |
| CCP-3-1 | 10.00% | ε_{⊥} [1 kHz, 20° C.]: | 7.1 |
| CCY-3-O1 | 5.00% | K₁ [pN, 20° C.]: | 14.4 |
| CCY-3-O2 | 3.25% | K₃ [pN, 20° C.]: | 16.5 |
| CPY-2-O2 | 7.00% | γ₁ [mPa · s, 20° C.]: | |
| CPY-3-O2 | 11.75% | V₀ [20° C., V]: | 2.26 |
| CY-3-O2 | 15.50% | | |
| CY-3-O4 | 3.50% | | |
| CY-5-O2 | 14.00% | | |

### Example M78

| | | | |
|---|---|---|---|
| BCH-32 | 6.50% | Clearing point [° C.]: | 75.1 |
| CC-3-V1 | 1.00% | Δn [589 nm, 20° C.]: | 0.0888 |
| CCH-13 | 20.50% | Δε [1 kHz, 20° C.]: | −3.0 |
| CCH-3O1 | 3.00% | ε_{∥} [1 kHz, 20° C.]: | 3.4 |
| CCH-34 | 6.00% | ε_{⊥} [1 kHz, 20° C.]: | 6.4 |
| CCH-35 | 6.00% | K₁ [pN, 20° C.]: | 13.4 |
| CCY-3-O2 | 5.00% | K₃ [pN, 20° C.]: | 13.6 |
| CCY-3-O2 | 10.00% | V₀ [20° C., V]: | 2.22 |
| CCY-4-O2 | 5.00% | | |
| CPY-2-O2 | 9.00% | | |
| CPY-3-O2 | 7.50% | | |
| CY-3-O2 | 12.00% | | |
| CY-5-O2 | 8.50% | | |

### Example M79

| | | | |
|---|---|---|---|
| BCH-52 | 1.50% | Clearing point [° C.]: | 74.8 |
| CC-3-V | 15.50% | Δn [589 nm, 20° C.]: | 0.0980 |
| CC-3-V1 | 10.00% | Δε [1 kHz, 20° C.]: | −3.4 |
| CCH-13 | 4.50% | ε_{∥} [1 kHz, 20° C.]: | 3.5 |
| CCH-34 | 5.00% | ε_{⊥} [1 kHz, 20° C.]: | 6.9 |
| CCH-35 | 5.00% | K₁ [pN, 20° C.]: | 14.7 |
| CCY-3-O1 | 10.00% | K₃ [pN, 20° C.]: | 15.4 |
| CCY-3-O2 | 14.00% | γ₁ [mPa · s, 20° C.]: | 101 |
| CCY-4-O2 | 8.50% | V₀ [20° C., V]: | 2.27 |
| PY-1-O4 | 8.00% | | |
| PY-3-O2 | 17.00% | | |
| PYP-3-O2 | 1.50% | | |

### Example M80

For the preparation of a PS-VA mixture, 99.75% of the mixture according to Example M60 are mixed with 0.25% of the polymerizable compound of the formula

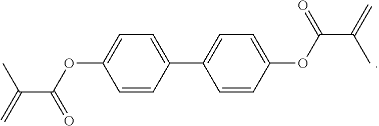 [SMILES: C=C(C)C(=O)Oc1ccc(-c2ccc(OC(=O)C(=C)C)cc2)cc1]

### Example M81

| | | | |
|---|---|---|---|
| BCH-32 | 2.00% | Clearing point [° C.]: | 74.8 |
| CC-3-V | 16.00% | Δn [589 nm, 20° C.]: | 0.0980 |
| CC-3-V1 | 9.50% | Δε [1 kHz, 20° C.]: | −3.3 |
| CCH-13 | 2.00% | ε_{∥} [1 kHz, 20° C.]: | 3.5 |
| CCH-3O1 | 3.00% | ε_{⊥} [1 kHz, 20° C.]: | 6.8 |
| CCH-34 | 6.00% | K₁ [pN, 20° C.]: | 14.4 |
| CCH-35 | 4.00% | K₃ [pN, 20° C.]: | 15.0 |
| CCY-3-O1 | 10.00% | γ₁ [mPa · s, 20° C.]: | 99 |
| CCY-3-O2 | 13.50% | V₀ [20° C., V]: | 2.28 |
| CCY-4-O2 | 8.00% | | |
| PY-1-O4 | 7.50% | | |
| PY-3-O2 | 17.00% | | |
| PYP-2-3 | 1.50% | | |

### Example M82

| | | | |
|---|---|---|---|
| BCH-52 | 2.00% | Clearing point [° C.]: | 75.1 |
| CC-3-V | 16.00% | Δn [589 nm, 20° C.]: | 0.0980 |
| CC-3-V1 | 9.50% | Δε [1 kHz, 20° C.]: | −3.3 |
| CCH-13 | 2.00% | ε_{∥} [1 kHz, 20° C.]: | 3.5 |
| CCH-34 | 3.00% | ε_{⊥} [1 kHz, 20° C.]: | 6.8 |
| CCH-35 | 6.00% | K₁ [pN, 20° C.]: | 14.8 |
| CCY-3-O1 | 4.00% | K₃ [pN, 20° C.]: | 15.5 |
| CCY-3-O2 | 10.00% | γ₁ [mPa · s, 20° C.]: | 103 |
| CCY-3-O3 | 13.50% | V₀ [20° C., V]: | 2.29 |
| CCY-4-O2 | 8.00% | | |
| PY-1-O4 | 7.50% | | |
| PY-3-O2 | 17.00% | | |
| PYP-2-3 | 1.50% | | |

### Example M83

| | | | |
|---|---|---|---|
| CC-3-V1 | 10.00% | Clearing point [° C.]: | 74.6 |
| CCH-13 | 16.00% | Δn [589 nm, 20° C.]: | 0.0985 |
| CCH-34 | 6.00% | Δε [1 kHz, 20° C.]: | −3.4 |
| CCH-35 | 2.50% | ε_{∥} [1 kHz, 20° C.]: | 3.5 |
| CCOC-3-3 | 3.50% | ε_{⊥} [1 kHz, 20° C.]: | 6.9 |
| CCOC-4-3 | 3.50% | K₁ [pN, 20° C.]: | 14.8 |
| CCP-3-1 | 1.00% | K₃ [pN, 20° C.]: | 16.2 |
| CCY-3-O2 | 12.50% | V₀ [20° C., V]: | 2.29 |
| CPY-2-O2 | 6.50% | | |
| CPY-3-O2 | 13.00% | | |
| CY-3-O2 | 11.50% | | |
| PY-3-O2 | 14.00% | | |

### Example M84

| | | | |
|---|---|---|---|
| BCH-52 | 1.00% | Clearing point [° C.]: | 74.9 |
| CC-3-V | 15.00% | Δn [589 nm, 20° C.]: | 0.0980 |
| CC-3-V1 | 10.00% | Δε [1 kHz, 20° C.]: | −3.4 |
| CCH-13 | 4.50% | ε_{⊥} [1 kHz, 20° C.]: | 7.0 |
| CCH-34 | 5.00% | K₁ [pN, 20° C.]: | 14.8 |
| CCH-35 | 5.00% | K₃ [pN, 20° C.]: | 15.5 |
| CCY-3-O1 | 8.50% | γ₁ [mPa · s, 20° C.]: | 104 |
| CCY-3-O2 | 14.50% | V₀ [20° C., V]: | 2.25 |
| CCY-3-O3 | 1.00% | | |
| CCY-4-O2 | 9.00% | | |
| PY-1-O4 | 8.50% | | |
| PY-3-O2 | 17.00% | | |
| PYP-2-3 | 1.00% | | |

### Example M85

| | | | |
|---|---|---|---|
| B-2O-O5 | 4.50% | Clearing point [° C.]: | 74.8 |
| BCH-32 | 2.50% | Δn [589 nm, 20° C.]: | 0.0989 |
| CC-3-V1 | 13.00% | Δε [1 kHz, 20° C.]: | −3.4 |
| CCH-13 | 13.00% | ε_{⊥} [1 kHz, 20° C.]: | 6.9 |
| CCH-34 | 6.00% | K₁ [pN, 20° C.]: | 15.7 |
| CCH-35 | 3.00% | K₃ [pN, 20° C.]: | 15.8 |
| CCP-V2-1 | 2.50% | γ₁ [mPa · s, 20° C.]: | 105 |
| CCY-3-O1 | 5.00% | V₀ [20° C., V]: | 2.29 |
| CCY-3-O2 | 8.00% | | |
| CCY-3-O3 | 3.00% | | |
| CCY-4-O2 | 12.00% | | |
| PY-1-O4 | 5.50% | | |
| PY-3-O2 | 15.00% | | |
| PYP-2-3 | 1.00% | | |

### Example M86

| | | | |
|---|---|---|---|
| B-2O-O5 | 4.00% | Clearing point [° C.]: | 75 |
| BCH-32 | 6.50% | Δn [589 nm, 20° C.]: | 0.1028 |
| CC-3-V | 22.50% | Δε [1 kHz, 20° C.]: | −3.3 |
| CC-3-V1 | 9.00% | ε_{∥} [1 kHz, 20° C.]: | 3.6 |
| CCH-13 | 7.50% | ε_{⊥} [1 kHz, 20° C.]: | 6.9 |
| CCY-3-O1 | 7.00% | K₁ [pN, 20° C.]: | 14.4 |
| CCY-3-O2 | 12.50% | K₃ [pN, 20° C.]: | 15.0 |
| CCY-4-O2 | 10.00% | γ₁ [mPa · s, 20° C.]: | 97 |
| PY-1-O4 | 3.00% | V₀ [20° C., V]: | 2.27 |
| PY-3-O2 | 16.00% | | |
| PYP-2-3 | 2.00% | | |

### Example M87

| | | | |
|---|---|---|---|
| B-2O-O5 | 4.50% | Clearing point [° C.]: | 74.5 |
| BCH-32 | 2.50% | Δn [589 nm, 20° C.]: | 0.0978 |
| CC-3-V1 | 18.50% | Δε [1 kHz, 20° C.]: | −3.4 |
| CCH-13 | 13.00% | ε_{∥} [1 kHz, 20° C.]: | 3.5 |
| CCH-34 | 6.00% | ε_{⊥} [1 kHz, 20° C.]: | 6.9 |
| CCH-35 | 4.00% | K₁ [pN, 20° C.]: | 15.6 |
| CCP-V2-1 | 2.50% | K₃ [pN, 20° C.]: | 15.8 |
| CCY-3-O1 | 6.00% | γ₁ [mPa · s, 20° C.]: | 106 |
| CCY-3-O2 | 8.00% | V₀ [20° C., V]: | 2.29 |
| CCY-3-O3 | 4.50% | | |
| CCY-4-O2 | 9.50% | | |
| PY-1-O4 | 5.50% | | |
| PY-3-O2 | 15.0% | | |
| PYP-2-3 | 0.50% | | |

### Example M88

For the preparation of a PS-VA mixture, 99.7% of the mixture according to Example M87 are mixed with 0.3% of the polymerizable compound of the formula

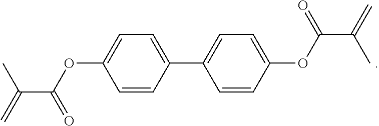 [SMILES: C=C(C)C(=O)Oc1ccc(-c2ccc(OC(=O)C(=C)C)cc2)cc1]

### Example M89

| | | | |
|---|---|---|---|
| B-2O-O5 | 4.00% | Clearing point [° C.]: | 74.6 |
| BCH-52 | 1.00% | Δn [589 nm, 20° C.]: | 0.0985 |
| CC-3-V | 15.00% | Δε [1 kHz, 20° C.]: | −3.5 |
| CC-3-V1 | 10.00% | ε_{∥} [1 kHz, 20° C.]: | 3.6 |
| CCH-13 | 4.00% | ε_{⊥} [1 kHz, 20° C.]: | 7.1 |
| CCH-34 | 6.00% | K₁ [pN, 20° C.]: | 14.7 |
| CCH-35 | 7.50% | K₃ [pN, 20° C.]: | 15.3 |
| CCY-3-O1 | 9.50% | γ₁ [mPa · s, 20° C.]: | 100 |
| CCY-3-O2 | 14.50% | V₀ [20° C., V]: | 2.22 |
| CCY-4-O2 | 5.50% | | |
| PY-1-O4 | 5.00% | | |
| PY-3-O2 | 16.00% | | |
| PYP-2-3 | 2.0% | | |

### Example M90

For the preparation of a PS-VA mixture, 99.75% of the mixture according to Example M89 are mixed with 0.25% of the polymerizable compound of the formula

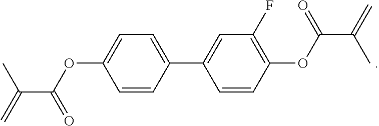 [SMILES: C=C(C)C(=O)Oc1ccc(-c2ccc(OC(=O)C(=C)C)c(F)c2)cc1]

### Example M91

For the preparation of a PS-VA mixture, 99.75% of the mixture according to Example M89 are mixed with 0.25% of the polymerizable compound of the formula

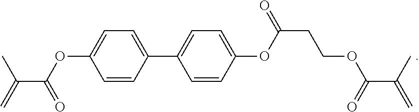 [SMILES: C=C(C)C(=O)OCCC(=O)Oc1ccc(-c2ccc(OC(=O)C(=C)C)cc2)cc1]

### Example M92

| | | | |
|---|---|---|---|
| B-2O-O5 | 4.00% | Clearing point [° C.]: | 85.2 |
| CC-3-V | 31.50% | Δn [589 nm, 20° C.]: | 0.0976 |
| CCH-13 | 6.00% | Δε [1 kHz, 20° C.]: | −3.5 |
| CCH-34 | 6.00% | ε_{∥} [1 kHz, 20° C.]: | 3.5 |
| CCY-3-O1 | 5.50% | ε_{⊥} [1 kHz, 20° C.]: | 6.9 |
| CCY-3-O2 | 3.00% | K₁ [pN, 20° C.]: | 14.7 |
| CCY-4-O2 | 4.00% | K₃ [pN, 20° C.]: | 15.1 |
| CLY-2-O4 | 6.00% | γ₁ [mPa · s, 20° C.]: | 100 |
| CLY-3-O2 | 4.00% | V₀ [20° C., V]: | 2.21 |
| CPY-2-O2 | 10.00% | | |
| CPY-3-O2 | 10.00% | | |
| CY-3-O2 | 7.00% | | |
| PGIY-2-O4 | 16.00% | | |

### Example M93

For the preparation of a PS-VA mixture, 99.7% of the mixture according to Example M6 are mixed with 0.3% of the polymerizable compound of the formula

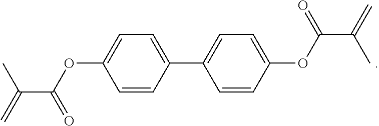 [SMILES: C=C(C)C(=O)Oc1ccc(-c2ccc(OC(=O)C(=C)C)cc2)cc1]

### Example M94

| | | | |
|---|---|---|---|
| CC-3-V | 20.50% | Clearing point [° C.]: | 74.8 |
| CC-3-V1 | 10.00% | Δn [589 nm, 20° C.]: | 0.1024 |
| CCH-13 | 10.50% | Δε [1 kHz, 20° C.]: | −3.2 |
| CCY-3-O1 | 7.00% | ε_{∥} [1 kHz, 20° C.]: | 3.5 |
| CCY-3-O2 | 13.00% | ε_{⊥} [1 kHz, 20° C.]: | 6.7 |
| CCY-4-O2 | 4.50% | K₁ [pN, 20° C.]: | 13.7 |
| CPY-2-O2 | 5.00% | K₃ [pN, 20° C.]: | 15.4 |
| CPY-3-O2 | 8.00% | γ₁ [mPa · s, 20° C.]: | 97 |
| PY-3-O2 | 16.00% | V₀ [20° C., V]: | 2.31 |
| PY-4-O2 | 3.50% | | |
| PYP-2-3 | 2.00% | | |

### Example M95

| | | | |
|---|---|---|---|
| CC-3-V | 20.50% | Clearing point [° C.]: | 74.5 |
| CC-3-V1 | 10.00% | Δn [589 nm, 20° C.]: | 0.1018 |
| CCH-13 | 10.50% | Δε [1 kHz, 20° C.]: | −3.2 |
| CCY-3-O1 | 7.00% | ε_{\|\|} [1 kHz, 20° C.]: | 3.6 |
| CCY-3-O2 | 13.00% | ε_{⊥} [1 kHz, 20° C.]: | 6.8 |
| CCY-4-O2 | 4.50% | K₁ [pN, 20° C.]: | 14.0 |
| CPY-2-O2 | 5.00% | K₃ [pN, 20° C.]: | 14.8 |
| CPY-3-O2 | 8.00% | γ₁ [mPa · s, 20° C.]: | 95 |
| PY-3-O2 | 16.00% | V₀ [20° C., V]: | 2.26 |
| PY-4-O2 | 3.50% | | |
| PYP-2-3 | 2.00% | | |

### Example M96

The mixture of Example M95 additionally comprises 0.001% of the compound of the formula

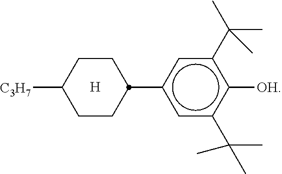

### Example M97

| | | | |
|---|---|---|---|
| BCH-32 | 0.50% | Clearing point [° C.]: | 74.6 |
| CC-3-V | 22.50% | Δn [589 nm, 20° C.]: | 0.1032 |
| CC-3-V1 | 9.00% | Δε [1 kHz, 20° C.]: | −3.2 |
| CCH-13 | 1.50% | ε_{\|\|} [1 kHz, 20° C.]: | 3.5 |
| CCH-34 | 5.50% | ε_{⊥} [1 kHz, 20° C.]: | 6.7 |
| CCH-35 | 4.00% | K₁ [pN, 20° C.]: | 14.2 |
| CCY-3-O1 | 8.50% | K₃ [pN, 20° C.]: | 14.8 |
| CCY-3-O2 | 12.00% | γ₁ [mPa · s, 20° C.]: | 95 |
| CPY-2-O2 | 12.00% | V₀ [20° C., V]: | 2.28 |
| CPY-3-O2 | 2.00% | | |
| PY-1-O4 | 3.00% | | |
| PY-3-O2 | 17.00% | | |
| PY-4-O2 | 1.50% | | |
| PYP-2-3 | 1.00% | | |

### Example M98

The mixture according to Example M97 is additionally stabilized with 0.01% of the compound of the formula

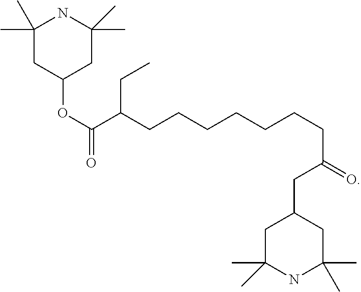

### Example M99

| | | | |
|---|---|---|---|
| BCH-52 | 0.50% | Clearing point [° C.]: | 75 |
| CC-3-V | 22.50% | Δn [589 nm, 20° C.]: | 0.1030 |
| CC-3-V1 | 9.00% | Δε [1 kHz, 20° C.]: | −3.2 |
| CCH-13 | 1.00% | ε_{\|\|} [1 kHz, 20° C.]: | 3.5 |
| CCH-34 | 5.50% | ε_{⊥} [1 kHz, 20° C.]: | 6.8 |
| CCH-35 | 4.00% | K₁ [pN, 20° C.]: | 14.2 |
| CCY-3-O1 | 9.50% | K₃ [pN, 20° C.]: | 15.0 |
| CCY-3-O2 | 12.50% | γ₁ [mPa · s, 20° C.]: | 95 |
| CPY-2-O2 | 10.00% | V₀ [20° C., V]: | 2.29 |
| CPY-3-O2 | 2.00% | | |
| CY-3-O2 | 0.50% | | |
| PY-1-O4 | 4.50% | | |
| PY-3-O2 | 17.00% | | |
| PYP-2-3 | 1.50% | | |

### Example M100

For the preparation of a PS-VA mixture, 99.7% of the mixture according to Example M99 are mixed with 0.3% of the polymerizable compound of the formula

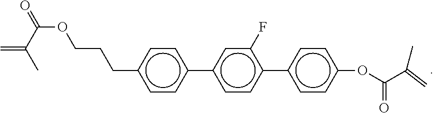

### Example M101

For the preparation of a PS-VA mixture, 99.7% of the mixture according to Example M77 are mixed with 0.3% of the polymerizable compound of the formula

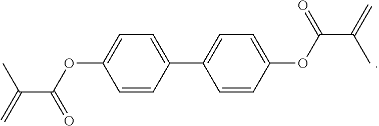 [SMILES: C=C(C)C(=O)Oc1ccc(-c2ccc(OC(=O)C(=C)C)cc2)cc1]

### Example M102

| | | | |
|---|---|---|---|
| CC-3-V | 22.50% | Clearing point [° C.]: | 74.8 |
| CC-3-V1 | 9.75% | Δn [589 nm, 20° C.]: | 0.1027 |
| CCH-13 | 0.75% | Δε [1 kHz, 20° C.]: | −3.2 |
| CCH-34 | 5.50% | ε_{\|\|} [1 kHz, 20° C.]: | 3.5 |
| CCH-35 | 4.00% | ε_{⊥} [1 kHz, 20° C.]: | 6.8 |
| CCY-3-O1 | 10.00% | K₁ [pN, 20° C.]: | 14.4 |
| CCY-3-O2 | 12.00% | K₃ [pN, 20° C.]: | 15.2 |
| CPY-2-O2 | 10.00% | γ₁ [mPa · s, 20° C.]: | 94 |
| CPY-3-O2 | 2.00% | V₀ [20° C., V]: | 2.29 |
| CY-3-O2 | 0.50% | | |
| PP-1-2V1 | 0.25% | | |
| PY-1-O4 | 4.25% | | |
| PY-3-O2 | 17.00% | | |
| PYP-2-3 | 1.50% | | |

### Example M103

For the preparation of a PS-VA mixture, 99.7% of the mixture according to Example M102 are mixed with 0.3% of the polymerizable compound of the formula

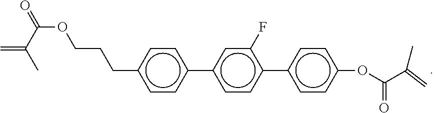

### Example M104

| | | | |
|---|---|---|---|
| CC-3-V | 0.50% | Clearing point [° C.]: | 75 |
| CC-3-V1 | 22.50% | Δn [589 nm, 20° C.]: | 0.1023 |
| CCH-13 | 9.75% | Δε [1 kHz, 20° C.]: | −3.2 |
| CCH-34 | 0.50% | ε_{\|\|} [1 kHz, 20° C.]: | 3.5 |
| CCH-35 | 5.75% | ε_{⊥} [1 kHz, 20° C.]: | 6.7 |
| CCY-3-O1 | 4.00% | K₁ [pN, 20° C.]: | 14.4 |
| CCY-3-O2 | 10.00% | K₃ [pN, 20° C.]: | 15.8 |
| CPY-2-O2 | 11.50% | γ₁ [mPa · s, 20° C.]: | 95 |
| CPY-3-O2 | 10.00% | V₀ [20° C., V]: | 2.29 |
| CY-3-O2 | 2.50% | | |
| PP-1-2V1 | 0.50% | | |
| PY-1-O4 | 4.40% | | |
| PY-3-O2 | 17.00% | | |
| PYP-2-3 | 1.00% | | |

### Example M105

For the preparation of a PS-VA mixture, 99.7% of the mixture according to Example M102 are mixed with 0.3% of the polymerizable compound of the formula

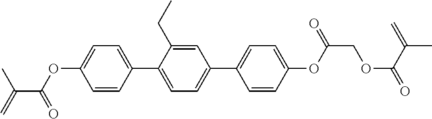 [SMILES: C=C(C)C(=O)OCC(=O)Oc1ccc(-c2ccc(-c3ccc(OC(=O)C(=C)C)cc3)c(CC)c2)cc1]

### and 0.001% of Irganox-1076 (BASF).

### Example M106

For the preparation of a PS-VA mixture, 99.7% of the mixture according to Example M7 are mixed with 0.3% of the polymerizable compound of the formula

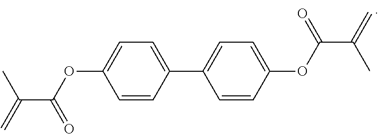 [SMILES: C=C(C)C(=O)Oc1ccc(-c2ccc(OC(=O)C(=C)C)cc2)cc1]

### Example M107

| | | | |
|---|---|---|---|
| CC-3-V | 22.50% | Clearing point [° C.]: | 74.8 |
| CC-3-V1 | 9.75% | Δn [589 nm, 20° C.]: | 0.1031 |
| CCH-13 | 0.50% | Δε [1 kHz, 20° C.]: | −3.2 |
| CCH-34 | 5.75% | ε_{\|\|} [1 kHz, 20° C.]: | 3.5 |
| CCH-35 | 4.00% | ε_{⊥} [1 kHz, 20° C.]: | 6.8 |
| CCY-3-O1 | 10.00% | K₁ [pN, 20° C.]: | 14.4 |
| CCY-3-O2 | 11.00% | K₃ [pN, 20° C.]: | 15.0 |
| CPY-2-O2 | 10.00% | γ₁ [mPa · s, 20° C.]: | 95 |
| CPY-3-O2 | 3.00% | V₀ [20° C., V]: | 2.28 |
| PY-1-O4 | 5.50% | | |
| PY-3-O2 | 16.50% | | |
| PYP-2-3 | 1.50% | | |

### Example M108

| | | | |
|---|---|---|---|
| BCH-52 | 0.75% | Clearing point [° C.]: | 74.5 |
| CC-3-V | 22.50% | Δn [589 nm, 20° C.]: | 0.1030 |
| CC-3-V1 | 10.00% | Δε [1 kHz, 20° C.]: | −3.2 |
| CCH-13 | 0.50% | ε_{\|\|} [1 kHz, 20° C.]: | 3.6 |
| CCH-34 | 5.75% | ε_{⊥} [1 kHz, 20° C.]: | 6.7 |
| CCH-35 | 3.50% | K₁ [pN, 20° C.]: | 14.2 |
| CCY-3-O1 | 9.50% | K₃ [pN, 20° C.]: | 14.9 |
| CCY-3-O2 | 11.50% | γ₁ [mPa · s, 20° C.]: | 95 |
| CPY-2-O2 | 10.00% | V₀ [20° C., V]: | 2.28 |
| CPY-3-O2 | 2.50% | | |
| PY-1-O4 | 5.50% | | |
| PY-3-O2 | 17.00% | | |
| PYP-2-3 | 1.00% | | |

### Example M109

The mixture of Example M108 additionally comprises 0.001% of the compound of the formula

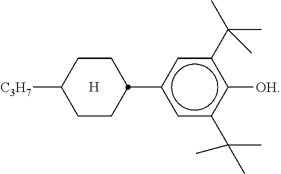

### Example M110

For the preparation of a PS-VA mixture, 99.7% of the mixture according to Example M109 are mixed with 0.3% of the polymerizable compound of the formula

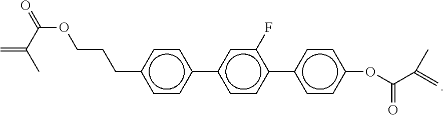

### Example M111

For the preparation of a PS-VA mixture, 99.75% of the mixture according to Example M108 are mixed with 0.25% of the polymerizable compound of the formula

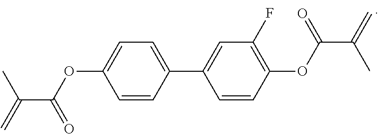 [SMILES: C=C(C)C(=O)Oc1ccc(-c2ccc(OC(=O)C(=C)C)c(F)c2)cc1]

### Example M112

| | | | |
|---|---|---|---|
| CC-3-V | 22.50% | Clearing point [° C.]: | 74.8 |
| CC-3-V1 | 8.00% | Δn [589 nm, 20° C.]: | 0.1031 |
| CCH-13 | 1.00% | Δε [1 kHz, 20° C.]: | −3.2 |
| CCH-34 | 6.00% | ε_{\|\|} [1 kHz, 20° C.]: | 3.5 |
| CCH-35 | 5.25% | ε_{⊥} [1 kHz, 20° C.]: | 6.8 |
| CCY-3-O1 | 6.00% | K₁ [pN, 20° C.]: | 14.5 |
| CCY-3-O2 | 13.25% | K₃ [pN, 20° C.]: | 15.0 |
| CPY-2-O2 | 11.00% | γ₁ [mPa · s, 20° C.]: | 96 |
| CPY-3-O2 | 4.00% | V₀ [20° C., V]: | 2.28 |
| CY-3-O2 | 1.00% | | |
| PY-1-O4 | 4.00% | | |
| PY-3-O2 | 17.00% | | |
| PYP-2-3 | 1.00% | | |

### Example M113

| | | | |
|---|---|---|---|
| CC-3-V | 22.50% | Clearing point [° C.]: | 74.5 |
| CC-3-V1 | 10.50% | Δn [589 nm, 20° C.]: | 0.1030 |
| CCH-13 | 1.50% | Δε [1 kHz, 20° C.]: | −3.2 |
| CCH-34 | 6.00% | ε_{\|\|} [1 kHz, 20° C.]: | 3.5 |
| CCH-35 | 3.00% | ε_{⊥} [1 kHz, 20° C.]: | 6.7 |
| CCY-3-O1 | 7.00% | K₁ [pN, 20° C.]: | 14.2 |
| CCY-3-O2 | 9.50% | K₃ [pN, 20° C.]: | 16.0 |
| CPY-2-O2 | 12.50% | γ₁ [mPa · s, 20° C.]: | 94 |
| CPY-3-O2 | 5.50% | V₀ [20° C., V]: | 2.30 |
| CY-3-O2 | 2.50% | | |
| PY-1-O4 | 6.00% | | |
| PY-3-O2 | 12.50% | | |
| PYP-2-3 | 1.00% | | |

### Example M114

For the preparation of a PS-VA mixture, 99.75% of the mixture according to Example M113 are mixed with 0.25% of the polymerizable compound of the formula

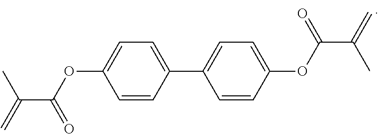 [SMILES: C=C(C)C(=O)Oc1ccc(-c2ccc(OC(=O)C(=C)C)cc2)cc1]

### Example M115

For the preparation of a polymer-stabilized LC mixture, 99.7% of the mixture according to Example M70 are mixed with

0.3% of the polymerizable compound of the formula

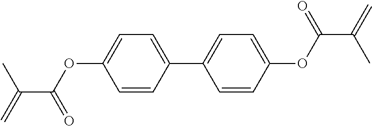 [SMILES: C=C(C)C(=O)Oc1ccc(-c2ccc(OC(=O)C(=C)C)cc2)cc1]

### and 0.001% of Irganox-1076 (BASF).

### Example M116

For the preparation of a PS-VA mixture, 99.75% of the mixture according to Example M113 are mixed with 0.25% of the polymerizable compound of the formula

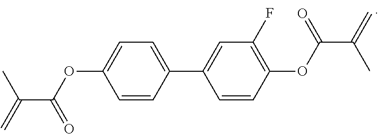 [SMILES: C=C(C)C(=O)Oc1ccc(-c2ccc(OC(=O)C(=C)C)c(F)c2)cc1]

### Example M117

| | | | |
|---|---|---|---|
| BCH-32 | 3.50% | Clearing point [° C.]: | 75.1 |
| CC-3-V1 | 10.00% | Δn [589 nm, 20° C.]: | 0.1122 |
| CCH-13 | 15.50% | Δε [1 kHz, 20° C.]: | −3.4 |
| CCH-34 | 6.00% | ε_{\|\|} [1 kHz, 20° C.]: | 3.6 |
| CCOC-3-3 | 3.50% | ε_{⊥} [1 kHz, 20° C.]: | 7.0 |
| CCOC-3-4 | 3.50% | K₁ [pN, 20° C.]: | 15.0 |
| CCY-3-O2 | 11.75% | K₃ [pN, 20° C.]: | 15.6 |
| CPY-2-O2 | 10.00% | V₀ [20° C., V]: | 2.26 |
| CPY-3-O2 | 11.00% | | |
| PY-1-O4 | 8.75% | | |
| PY-3-O2 | 14.00% | | |
| PY-4-O2 | 2.50% | | |

### Example M118

For the preparation of a PS-VA mixture, 99.7% of the mixture according to Example M117 are mixed with 0.3% of the polymerizable compound of the formula

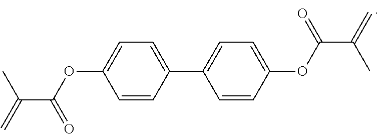 [SMILES: C=C(C)C(=O)Oc1ccc(-c2ccc(OC(=O)C(=C)C)cc2)cc1]

### Example M119

For the preparation of a PS-VA mixture, 99.7% of the mixture according to Example M117 are mixed with 0.3% of the polymerizable compound of the formula

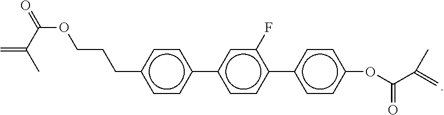

### Example M120

| | | | |
|---|---|---|---|
| BCH-32 | 7.00% | Clearing point [° C.]: | 74 |
| CC-3-V1 | 10.00% | Δn [589 nm, 20° C.]: | 0.1118 |
| CCH-13 | 15.00% | Δε [1 kHz, 20° C.]: | −3.4 |
| CCH-34 | 3.25% | ε_{\|\|} [1 kHz, 20° C.]: | 3.6 |
| CCOC-3-3 | 3.50% | ε_{⊥} [1 kHz, 20° C.]: | 7.0 |
| CCOC-4-3 | 3.50% | K₁ [pN, 20° C.]: | 14.7 |
| CCY-3-O2 | 12.50% | K₃ [pN, 20° C.]: | 15.8 |
| CPY-2-O2 | 4.50% | V₀ [20° C., V]: | 2.27 |
| CPY-3-O2 | 12.00% | | |
| CY-3-O4 | 5.25% | | |
| PY-1-O4 | 9.50% | | |
| PY-3-O2 | 14.00% | | |

### Example M121

For the preparation of a PS-VA mixture, 99.75% of the mixture according to Example M120 are mixed with 0.25% of the polymerizable compound of the formula

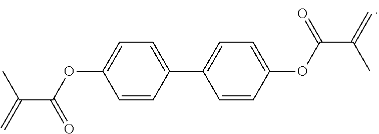 [SMILES: C=C(C)C(=O)Oc1ccc(-c2ccc(OC(=O)C(=C)C)cc2)cc1]

### Example M122

| | | | |
|---|---|---|---|
| BCH-32 | 5.00% | Clearing point [° C.]: | 74 |
| CCH-13 | 17.00% | Δn [589 nm, 20° C.]: | 0.1355 |
| CCH-34 | 6.50% | Δε [1 kHz, 20° C.]: | −3.1 |
| CCH-35 | 6.00% | ε_{\|\|} [1 kHz, 20° C.]: | 3.7 |
| CCY-3-O2 | 7.00% | ε_{⊥} [1 kHz, 20° C.]: | 6.6 |
| CPY-2-O2 | 10.00% | K₁ [pN, 20° C.]: | 14.1 |
| CPY-3-O2 | 10.00% | K₃ [pN, 20° C.]: | 13.8 |
| PY-1-O4 | 8.00% | V₀ [20° C., V]: | 2.24 |
| PY-3-O2 | 11.00% | | |
| PYP-2-3 | 10.00% | | |
| PYP-2-4 | 9.50% | | |

### Example M123

For the preparation of a PS-VA mixture, 99.7% of the mixture according to Example M122 are mixed with 0.3% of the polymerizable compound of the formula

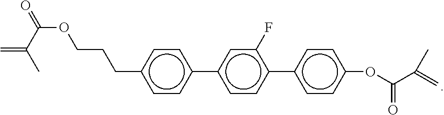

### Example M124

For the preparation of a PS-VA mixture, 99.7% of the mixture according to Example M122 are mixed with 0.3% of the polymerizable compound of the formula

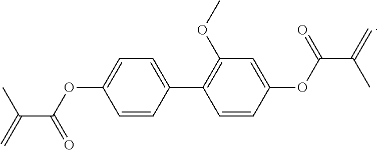 [SMILES: C=C(C)C(=O)Oc1ccc(-c2ccc(OC(=O)C(=C)C)cc2OC)cc1]

### Example M125

| | |
|---|---|
| CC-3-V | 15.00% |
| CC-3-V1 | 9.00% |
| CCH-23 | 8.00% |
| CCH-13 | 6.00% |
| CCOY-3-O2 | 12.00% |
| CCY-5-O2 | 8.00% |
| CPY-2-O2 | 3.00% |
| CPY-3-O2 | 9.50% |
| CY-3-O2 | 7.50% |
| PY-3-O2 | 14.00% |
| PYP-2-3 | 8.00% |

### Example M126

For the preparation of a PS-VA mixture, 99.7% of the mixture according to Example M125 are mixed with 0.3% of the polymerizable compound of the formula

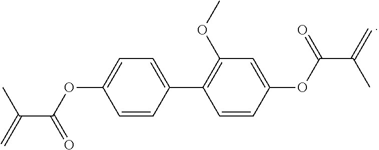 [SMILES: C=C(C)C(=O)Oc1ccc(-c2ccc(OC(=O)C(=C)C)cc2OC)cc1]

### Example M127

| | | | |
|---|---|---|---|
| BCH-32 | 5.00% | Clearing point [° C.]: | 74.2 |
| CCH-13 | 17.00% | Δn [589 nm, 20° C.]: | 0.1349 |
| CCH-34 | 7.00% | Δε [1 kHz, 20° C.]: | −2.8 |
| CCH-35 | 5.00% | ε_{\|\|} [1 kHz, 20° C.]: | 3.5 |
| CCP-3-1 | 6.50% | ε_{⊥} [1 kHz, 20° C.]: | 6.2 |
| CPY-2-O2 | 11.00% | K₁ [pN, 20° C.]: | 14.2 |
| CPY-3-O2 | 11.00% | K₃ [pN, 20° C.]: | 13.8 |
| PY-1-O4 | 7.50% | V₀ [20° C., V]: | 2.40 |
| PY-3-O2 | 12.00% | | |
| PYP-2-3 | 9.00% | | |
| PYP-2-4 | 9.00% | | |

### Example M128

For the preparation of a polymer-stabilized LC mixture, 99.7% of the mixture according to Example M127 are mixed with

0.3% of the polymerizable compound of the formula

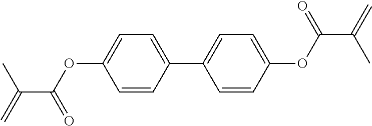 [SMILES: C=C(C)C(=O)Oc1ccc(-c2ccc(OC(=O)C(=C)C)cc2)cc1]

### and 0.001% of Irganox-1076 (BASF).

### Example M129

For the preparation of a PS-VA mixture, 99.7% of the mixture according to Example M127 are mixed with 0.3% of the polymerizable compound of the formula

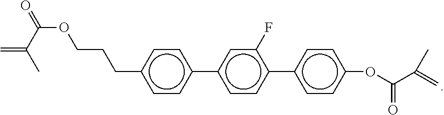

### Example M130

| | | | |
|---|---|---|---|
| BCH-32 | 8.00% | Clearing point [° C.]: | 74.3 |
| BCH-52 | 2.50% | Δn [589 nm, 20° C.]: | 0.1116 |
| CC-3-V1 | 10.00% | Δε [1 kHz, 20° C.]: | −3.4 |
| CCH-13 | 15.50% | ε_{\|\|} [1 kHz, 20° C.]: | 3.6 |
| CCOC-3-3 | 3.50% | ε_{⊥} [1 kHz, 20° C.]: | 7.0 |
| CCOC-4-3 | 3.50% | K₁ [pN, 20° C.]: | 14.1 |
| CCY-3-O2 | 6.75% | K₃ [pN, 20° C.]: | 15.4 |
| CPY-2-O2 | 10.00% | V₀ [20° C., V]: | 2.26 |
| CPY-3-O2 | 11.00% | | |
| CY-3-O2 | 14.00% | | |
| CY-3-O4 | 1.25% | | |
| PY-3-O2 | 14.00% | | |

### Example M131

For the preparation of a polymer-stabilized LC mixture, 99.99% of the mixture according to Example M130 are mixed with

0.01% of the polymerizable compound of the formula

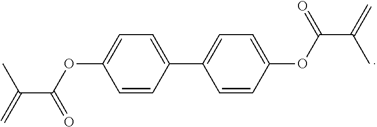 [SMILES: C=C(C)C(=O)Oc1ccc(-c2ccc(OC(=O)C(=C)C)cc2)cc1]

### Example M132

| | | | |
|---|---|---|---|
| BCH-32 | 6.00% | Clearing point [° C.]: | 74.8 |
| CC-3-V1 | 10.00% | | |
| CCH-13 | 15.50% | | |
| CCH-34 | 6.00% | | |
| CCY-3-O1 | 5.50% | | |
| CCOY-3-O2 | 10.00% | | |
| CPY-2-O2 | 10.00% | | |
| CPY-3-O2 | 11.00% | | |
| CY-3-O4 | 9.00% | | |
| PP-1-2V1 | 2.00% | | |
| PY-3-O2 | 15.00% | | |

### Example M133

| | | | |
|---|---|---|---|
| BCH-32 | 6.50% | Clearing point [° C.]: | 75.5 |
| CC-3-V1 | 9.00% | Δn [589 nm, 20° C.]: | 0.1120 |
| CCH-13 | 15.00% | Δε [1 kHz, 20° C.]: | −3.5 |
| CCOC-3-3 | 3.50% | ε_{\|\|} [1 kHz, 20° C.]: | 3.6 |
| CCOC-4-3 | 3.50% | K₁ [pN, 20° C.]: | 14.4 |
| CCY-3-O1 | 2.50% | K₃ [pN, 20° C.]: | 15.9 |
| CCY-3-O2 | 9.00% | V₀ [20° C., V]: | 2.24 |
| CPY-2-O2 | 11.00% | | |
| CPY-3-O2 | 11.00% | | |
| CY-3-O2 | 14.50% | | |
| PP-1-3 | 3.50% | | |
| PY-3-O2 | 11.00% | | |

### Example M134

| | | | |
|---|---|---|---|
| BCH-32 | 4.50% | Clearing point [° C.]: | 74.5 |
| BCH-52 | 3.25% | Δn [589 nm, 20° C.]: | 0.1123 |
| CC-3-V1 | 10.00% | Δε [1 kHz, 20° C.]: | −3.4 |
| CCH-13 | 14.25% | ε_{\|\|} [1 kHz, 20° C.]: | 3.6 |
| CCOC-3-3 | 3.50% | ε_{⊥} [1 kHz, 20° C.]: | 7.0 |
| CCOC-4-3 | 3.50% | K₁ [pN, 20° C.]: | 14.2 |
| CCY-3-O1 | 2.50% | K₃ [pN, 20° C.]: | 15.6 |
| CCY-3-O2 | 7.50% | V₀ [20° C., V]: | 2.26 |
| CPY-2-O2 | 10.00% | | |
| CPY-3-O2 | 11.00% | | |
| CY-3-O2 | 14.75% | | |
| PP-1-3 | 3.25% | | |
| PY-3-O2 | 12.00% | | |

### Example M135

For the preparation of a PS-VA mixture, 99.7% of the mixture according to Example M134 are mixed with 0.3% of the polymerizable compound of the formula

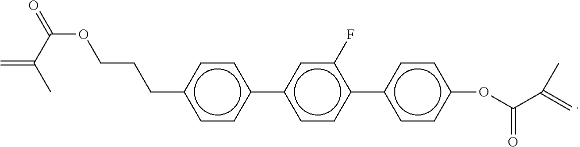

### Example M136

| | | | |
|---|---|---|---|
| B-2O-O5 | 4.00% | Clearing point [° C.]: | 85.5 |
| CC-3-V | 32.00% | | |
| CCH-13 | 6.00% | | |
| CCH-34 | 5.00% | | |
| CCY-3-O1 | 7.00% | | |
| CCOY-3-O2 | 4.00% | | |
| CCOY-4-O2 | 1.50% | | |
| CLY-2-O4 | 5.50% | | |
| CLY-3-O2 | 4.00% | | |
| CPY-2-O2 | 10.00% | | |
| CPY-3-O2 | 10.00% | | |
| CY-3-O2 | 7.00% | | |
| PGIY-2-O4 | 4.00% | | |

### Example M137

For the preparation of a PS-VA mixture, 99.7% of the mixture according to Example M136 are mixed with 0.3% of the polymerizable compound of the formula

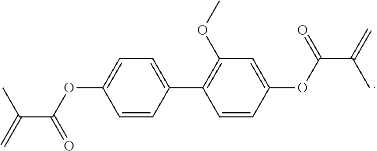 [SMILES: C=C(C)C(=O)Oc1ccc(-c2ccc(OC(=O)C(=C)C)cc2OC)cc1]

### Example M138

For the preparation of a PS-VA mixture, 99.75% of the mixture according to Example M136 are mixed with 0.25% of the polymerizable compound of the formula

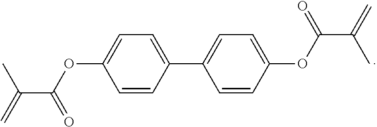 [SMILES: C=C(C)C(=O)Oc1ccc(-c2ccc(OC(=O)C(=C)C)cc2)cc1]

### Example M139

For the preparation of a PS-VA mixture, 99.7% of the mixture according to Example M136 are mixed with 0.3% of the polymerizable compound of the formula

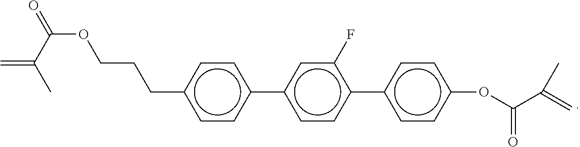

### Example M140

| | | | |
|---|---|---|---|
| BCH-32 | 4.50% | Clearing point [° C.]: | 74.2 |
| BCH-52 | 2.50% | Δn [589 nm, 20° C.]: | 0.1121 |
| CC-3-V1 | 10.00% | Δε [1 kHz, 20° C.]: | −3.4 |
| CCH-13 | 13.75% | ε_{\|\|} [1 kHz, 20° C.]: | 3.7 |
| CCOC-3-3 | 3.50% | ε_{⊥} [1 kHz, 20° C.]: | 7.1 |
| CCOC-4-3 | 3.50% | K₁ [pN, 20° C.]: | 14.3 |
| CCY-3-O1 | 2.50% | K₃ [pN, 20° C.]: | 15.7 |
| CCY-3-O2 | 7.50% | V₀ [20° C., V]: | 2.26 |
| CPY-2-O2 | 10.00% | | |
| CPY-3-O2 | 11.00% | | |
| CY-3-O2 | 15.50% | | |
| PP-1-3 | 3.25% | | |
| PY-3-O2 | 12.00% | | |
| PPGU-3-F | 0.50% | | |

### Example M141

The mixture according to Example M140 additionally comprises 0.03% of

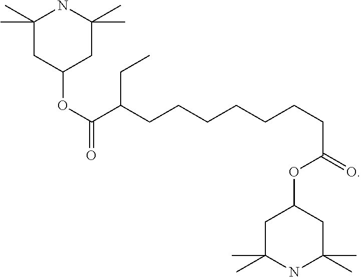

### Example M142

| | | | |
|---|---|---|---|
| B-2O-O5 | 4.00% | Clearing point [° C.]: | 85.1 |
| CC-3-V | 32.00% | Δn [589 nm, 20° C.]: | 0.0998 |
| CCH-13 | 6.00% | Δε [1 kHz, 20° C.]: | −3.5 |
| CCH-34 | 5.00% | ε_{\|\|} [1 kHz, 20° C.]: | 3.5 |
| CCY-3-O1 | 7.00% | ε_{⊥} [1 kHz, 20° C.]: | 7.0 |
| CCY-3-O2 | 4.00% | K₁ [pN, 20° C.]: | 14.8 |
| CCY-4-O2 | 1.50% | K₃ [pN, 20° C.]: | 15.2 |
| CLY-2-O4 | 5.50% | V₀ [20° C., V]: | 2.20 |
| CLY-3-O2 | 4.00% | | |
| CPY-2-O2 | 10.00% | | |
| CPY-3-O2 | 10.00% | | |
| CY-3-O2 | 7.00% | | |
| PGIY-2-O4 | 4.00% | | |

### Example M143

| | | | |
|---|---|---|---|
| CC-3-V | 22.50% | Clearing point [° C.]: | 74.7 |
| CC-3-V1 | 10.50% | Δn [589 nm, 20° C.]: | 0.0984 |
| CCH-13 | 1.00% | Δε [1 kHz, 20° C.]: | −3.1 |
| CCH-34 | 6.50% | ε_{\|\|} [1 kHz, 20° C.]: | 3.7 |
| CCY-3-O1 | 7.00% | ε_{⊥} [1 kHz, 20° C.]: | 6.6 |
| CCY-3-O2 | 13.00% | K₁ [pN, 20° C.]: | 14.0 |
| CPY-2-O2 | 2.00% | K₃ [pN, 20° C.]: | 13.7 |
| CPY-3-O2 | 10.50% | γ₁ [mPa · s, 20° C.]: | 98 |
| CY-3-O2 | 12.00% | V₀ [20° C., V]: | 2.24 |
| PCH-3O1 | 0.50% | | |
| PY-3-O2 | 11.50% | | |
| PYP-2-4 | 3.00% | | |

### Example M144

| | | | |
|---|---|---|---|
| B-2O-O5 | 4.00% | Clearing point [° C.]: | 84.9 |
| BCH-32 | 4.50% | Δn [589 nm, 20° C.]: | 0.1066 |
| CC-3-V | 29.00% | Δε [1 kHz, 20° C.]: | −4.0 |
| CCH-13 | 6.00% | ε_{\|\|} [1 kHz, 20° C.]: | 3.6 |
| CCY-3-O1 | 6.00% | ε_{⊥} [1 kHz, 20° C.]: | 7.6 |
| CCY-4-O2 | 5.50% | K₁ [pN, 20° C.]: | 15.3 |
| CLY-2-O4 | 6.00% | K₃ [pN, 20° C.]: | 15.6 |
| CLY-3-O2 | 3.00% | V₀ [20° C., V]: | 2.09 |
| CPY-2-O2 | 10.00% | | |
| CPY-3-O2 | 10.00% | | |
| CY-3-O2 | 6.00% | | |
| CY-5-O2 | 6.00% | | |
| PGIY-2-O4 | 4.00% | | |

### Example M145

For the preparation of a PS-VA mixture, 99.7% of the mixture according to Example M144 are mixed with 0.3% of the polymerizable compound of the formula

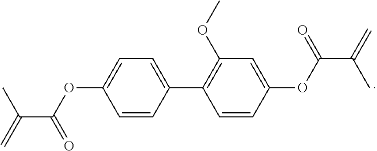 [SMILES: C=C(C)C(=O)Oc1ccc(-c2ccc(OC(=O)C(=C)C)cc2OC)cc1]

### Example M146

For the preparation of a PS-VA mixture, 99.7% of the mixture according to Example M144 are mixed with 0.3% of the polymerizable compound of the formula

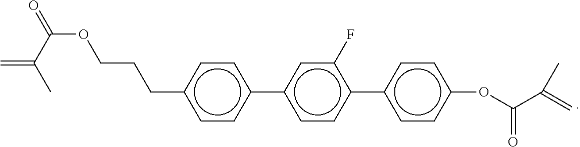

### Example M147

| | | | |
|---|---|---|---|
| CC-3-V1 | 10.00% | Clearing point [° C.]: | 75.5 |
| CCH-13 | 16.50% | Δn [589 nm, 20° C.]: | 0.1110 |
| CCP-3-1 | 6.50% | Δε [1 kHz, 20° C.]: | −3.7 |
| CCY-3-O1 | 9.00% | ε_{\|\|} [1 kHz, 20° C.]: | 3.6 |
| CCY-3-O2 | 9.50% | | |
| CPY-2-O2 | 10.00% | | |
| CPY-3-O2 | 10.00% | | |
| CY-3-O4 | 10.50% | | |
| PP-1-2V1 | 3.50% | | |
| PY-V2-O2 | 14.50% | | |

### Example M148

For the preparation of a PS-VA mixture, 99.7% of the mixture according to Example M147 are mixed with 0.3% of the polymerizable compound of the formula

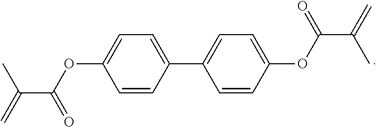 [SMILES: C=C(C)C(=O)Oc1ccc(-c2ccc(OC(=O)C(=C)C)cc2)cc1]

### Example M149

| | | | |
|---|---|---|---|
| B-2O-O5 | 5.00% | Clearing point [° C.]: | 74.5 |
| BCH-52 | 8.00% | Δn [589 nm, 20° C.]: | 0.1033 |
| CC-3-V | 22.50% | Δε [1 kHz, 20° C.]: | −3.0 |
| CCH-13 | 9.40% | ε_{\|\|} [1 kHz, 20° C.]: | 3.6 |
| CCH-34 | 5.50% | ε_{⊥} [1 kHz, 20° C.]: | 6.6 |
| CCY-3-O1 | 8.20% | K₁ [pN, 20° C.]: | 13.4 |
| CCY-3-O2 | 8.80% | K₃ [pN, 20° C.]: | 13.5 |
| CCY-3-O2 | 11.60% | γ₁ [mPa · s, 20° C.]: | 96 |
| PP-1-2V1 | 2.00% | V₀ [20° C., V]: | 2.26 |
| PY-3-O2 | 15.00% | | |
| PY-4-O2 | 1.00% | | |
| PYP-2-3 | 3.00% | | |

### Example M150

For the preparation of a PS-VA mixture, 99.7% of the mixture according to Example M149 are mixed with 0.3% of the polymerizable compound of the formula

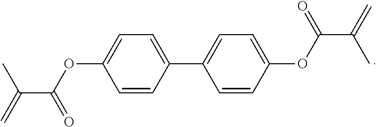 [SMILES: C=C(C)C(=O)Oc1ccc(-c2ccc(OC(=O)C(=C)C)cc2)cc1]

### Example M151

| | | | |
|---|---|---|---|
| B-2O-O5 | 5.00% | Clearing point [° C.]: | 74.5 |
| BCH-52 | 7.25% | Δn [589 nm, 20° C.]: | 0.1033 |
| CC-3-V | 22.50% | Δε [1 kHz, 20° C.]: | −3.1 |
| CCH-13 | 5.75% | ε_{\|\|} [1 kHz, 20° C.]: | 3.5 |
| CCH-3O1 | 1.00% | ε_{⊥} [1 kHz, 20° C.]: | 6.6 |
| CCH-34 | 5.50% | K₁ [pN, 20° C.]: | 14.4 |
| CCH-35 | 3.50% | K₃ [pN, 20° C.]: | 13.9 |
| CCY-3-O1 | 8.00% | γ₁ [mPa · s, 20° C.]: | 95 |
| CCY-3-O2 | 10.00% | V₀ [20° C., V]: | 2.26 |
| CCY-4-O2 | 9.50% | | |
| PP-1-2V1 | 2.50% | | |
| PY-3-O2 | 11.00% | | |
| PY-4-O2 | 5.50% | | |
| PYP-2-3 | 3.00% | | |

### Example M152

| | | | |
|---|---|---|---|
| B-2O-O5 | 4.00% | Clearing point [° C.]: | 84.9 |
| BCH-52 | 4.50% | Δn [589 nm, 20° C.]: | 0.1066 |
| CC-3-V | 28.50% | Δε [1 kHz, 20° C.]: | −4.1 |
| CCH-13 | 5.00% | ε_{\|\|} [1 kHz, 20° C.]: | 3.6 |
| CCY-3-O1 | 6.00% | ε_{⊥} [1 kHz, 20° C.]: | 7.7 |
| CCY-4-O2 | 5.50% | K₁ [pN, 20° C.]: | 14.9 |
| CLY-2-O4 | 6.50% | K₃ [pN, 20° C.]: | 15.4 |
| CLY-3-O2 | 3.00% | V₀ [20° C., V]: | 2.05 |
| CPY-2-O2 | 10.00% | | |
| CPY-3-O2 | 10.00% | | |
| CY-3-O2 | 6.50% | | |
| CY-5-O2 | 7.00% | | |
| PGIY-2-O4 | 3.50% | | |

### Example M153

For the preparation of a PS-FFS mixture, 99.7% of the mixture according to Example M152 are mixed with 0.3% of the polymerizable compound of the formula

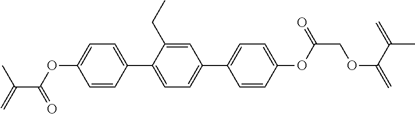 [SMILES: C=C(C)C(=C)OCC(=O)Oc1ccc(-c2ccc(-c3ccc(OC(=O)C(=C)C)cc3)c(CC)c2)cc1]

### and 0.001% of Irganox-1076 (BASF).

### Example M154

| | | | |
|---|---|---|---|
| CC-3-V1 | 10.00% | Clearing point [° C.]: | 75.1 |
| CCH-13 | 16.50% | Δn [589 nm, 20° C.]: | 0.1109 |
| CCP-3-1 | 6.50% | Δε [1 kHz, 20° C.]: | −3.8 |
| CCY-3-O1 | 9.00% | ε_{\|\|} [1 kHz, 20° C.]: | 3.6 |
| CCY-3-O2 | 9.50% | ε_{⊥} [1 kHz, 20° C.]: | 7.4 |
| CPY-2-O2 | 10.00% | K₁ [pN, 20° C.]: | 14.7 |
| CPY-3-O2 | 10.00% | K₃ [pN, 20° C.]: | 16.7 |
| CY-3-O4 | 10.50% | V₀ [20° C., V]: | 2.21 |
| PP-1-2V1 | 3.50% | | |
| PY-3-O2 | 14.50% | | |

### Example M155

The mixture according to Example M152 additionally comprises 0.04% of the compound of the formula

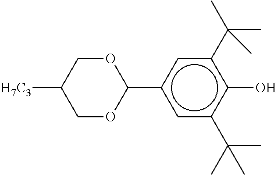

and 0.02% of the compound of the formula

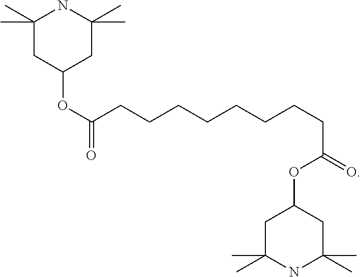

### Example M156

| | | | |
|---|---|---|---|
| BCH-32 | 5.00% | Clearing point [° C.]: | 74.6 |
| CC-3-V1 | 10.00% | Δn [589 nm, 20° C.]: | 0.1110 |
| CCH-13 | 16.00% | Δε [1 kHz, 20° C.]: | −3.6 |
| CCH-34 | 6.00% | ε_{\|\|} [1 kHz, 20° C.]: | 3.6 |
| CCY-3-O1 | 6.50% | ε_{⊥} [1 kHz, 20° C.]: | 7.2 |
| CCY-3-O2 | 10.00% | K₁ [pN, 20° C.]: | 14.4 |
| CPY-2-O2 | 10.00% | K₃ [pN, 20° C.]: | 15.8 |
| CPY-3-O2 | 11.00% | V₀ [20° C., V]: | 2.21 |
| CY-3-O2 | 9.00% | | |
| PP-1-2V1 | 1.50% | | |
| PY-3-O2 | 15.00% | | |

### Example M157

The mixture according to Example M156 additionally comprises 0.03% of the compound of the formula

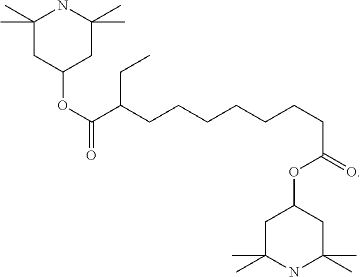

### Example M158

For the preparation of a PS-VA mixture, 99.7% of the mixture according to Example M156 are mixed with 0.3% of the polymerizable compound of the formula

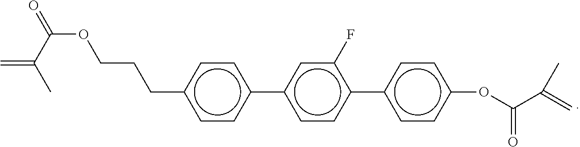

### Example M159

| | | | |
|---|---|---|---|
| CC-3-V1 | 8.50% | Clearing point [° C.]: | 74 |
| CCH-13 | 15.50% | Δn [589 nm, 20° C.]: | 0.1052 |
| CCH-34 | 7.00% | Δε [1 kHz, 20° C.]: | −2.8 |
| CCP-3-1 | 13.50% | ε_{\|\|} [1 kHz, 20° C.]: | 3.3 |
| CCY-3-O1 | 3.50% | ε_{⊥} [1 kHz, 20° C.]: | 6.2 |
| CCY-3-O2 | 10.00% | K₁ [pN, 20° C.]: | 15.7 |
| CPY-3-O2 | 12.00% | K₃ [pN, 20° C.]: | 17.9 |
| CY-3-O2 | 10.00% | V₀ [20° C., V]: | 2.66 |
| PP-1-2V1 | 5.50% | | |
| PY-3-O2 | 14.50% | | |

### Example M160

| | | | |
|---|---|---|---|
| BCH-32 | 6.00% | Clearing point [° C.]: | 74.6 |
| CC-3-V1 | 10.00% | Δn [589 nm, 20° C.]: | 0.1117 |
| CCH-13 | 15.50% | Δε [1 kHz, 20° C.]: | −3.5 |
| CCH-34 | 6.00% | ε_{\|\|} [1 kHz, 20° C.]: | 3.5 |
| CCY-3-O1 | 5.50% | ε_{⊥} [1 kHz, 20° C.]: | 7.0 |
| CCY-3-O2 | 10.00% | K₁ [pN, 20° C.]: | 14.7 |
| CPY-2-O2 | 10.00% | K₃ [pN, 20° C.]: | 15.7 |
| CPY-3-O2 | 11.00% | V₀ [20° C., V]: | 2.23 |
| CY-3-O4 | 9.00% | | |
| PP-1-2V1 | 2.00% | | |
| PY-3-O2 | 15.00% | | |

### Example M161

The mixture according to Example M160 additionally comprises 0.03% of the compound of the formula

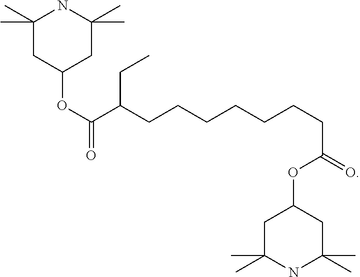

### Example M162

For the preparation of a polymer-stabilized LC mixture, 99.7% of the mixture according to Example M160 are mixed with

0.3% of the polymerizable compound of the formula

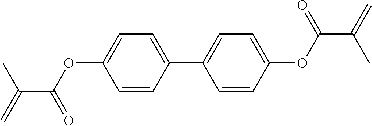 [SMILES: C=C(C)C(=O)Oc1ccc(-c2ccc(OC(=O)C(=C)C)cc2)cc1]

### and 0.001% of Irganox-1076 (BASF).

### Example M163

| | | | |
|---|---|---|---|
| BCH-32 | 6.00% | Clearing point [° C.]: | 74.6 |
| CC-3-V1 | 10.00% | Δn [589 nm, 20° C.]: | 0.1117 |
| CCH-13 | 15.50% | Δε [1 kHz, 20° C.]: | −3.4 |
| CCH-34 | 6.00% | ε_{\|\|} [1 kHz, 20° C.]: | 3.5 |
| CCY-3-O1 | 5.50% | ε_{⊥} [1 kHz, 20° C.]: | 6.9 |
| CCY-3-O2 | 10.00% | K₁ [pN, 20° C.]: | 14.7 |
| CPY-2-O2 | 10.00% | K₃ [pN, 20° C.]: | 15.8 |
| CPY-3-O2 | 11.00% | V₀ [20° C., V]: | 2.27 |
| CY-3-O4 | 9.00% | | |
| PP-1-2V1 | 3.00% | | |
| PY-3-O2 | 14.00% | | |

### Example M164

For the preparation of a polymer-stabilized LC mixture, 99.7% of the mixture according to Example M163 are mixed with

0.3% of the polymerizable compound of the formula

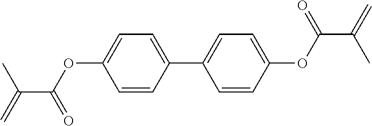 [SMILES: C=C(C)C(=O)Oc1ccc(-c2ccc(OC(=O)C(=C)C)cc2)cc1]

### and 0.001% of Irganox-1076 (BASF).

### Example M165

The mixture according to Example M163 additionally comprises 0.03% of the compound of the formula

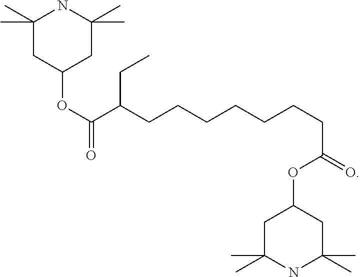

### Example M166

| | | | |
|---|---|---|---|
| B-2O-O5 | 4.00% | Clearing point [° C.]: | 85.2 |
| CC-3-V | 31.50% | Δn [589 nm, 20° C.]: | 0.0976 |
| CCH-13 | 6.00% | Δε [1 kHz, 20° C.]: | −3.5 |
| CCH-34 | 6.00% | ε_{\|\|} [1 kHz, 20° C.]: | 3.5 |
| CCY-3-O1 | 5.50% | ε_{⊥} [1 kHz, 20° C.]: | 6.9 |
| CCY-3-O2 | 3.00% | K₁ [pN, 20° C.]: | 14.7 |
| CCY-4-O2 | 4.00% | K₃ [pN, 20° C.]: | 15.1 |
| CLY-2-O4 | 6.00% | V₀ [20° C., V]: | 2.21 |
| CLY-3-O2 | 4.00% | | |
| CPY-2-O2 | 10.00% | | |
| CPY-3-O2 | 10.00% | | |
| CY-3-O2 | 7.00% | | |
| PGIY-2-O4 | 3.00% | | |

### Example M167

For the preparation of a PS-VA mixture, 99.7% of the mixture according to Example M166 are mixed with 0.3% of the polymerizable compound of the formula

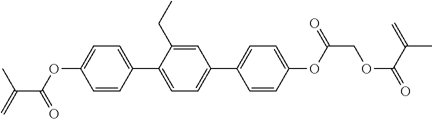 [SMILES: C=C(C)C(=O)OCC(=O)Oc1ccc(-c2ccc(-c3ccc(OC(=O)C(=C)C)cc3)c(CC)c2)cc1]

### and 0.001% of Irganox-1076 (BASF).

### Example M168

| | | | |
|---|---|---|---|
| B-2O-O5 | 4.00% | Clearing point [° C.]: | 85.3 |
| CC-3-V | 31.50% | | |
| CCH-13 | 6.00% | | |
| CCH-34 | 6.00% | | |
| CCY-3-O1 | 5.50% | | |
| CCOY-3-O2 | 3.00% | | |
| CCOY-4-O2 | 4.00% | | |
| CLY-2-O4 | 6.00% | | |
| CLY-3-O2 | 4.00% | | |
| CPY-2-O2 | 10.00% | | |
| CPY-3-O2 | 10.00% | | |
| CY-3-O2 | 7.00% | | |
| PGIY-2-O4 | 3.00% | | |

### Example M168a

For the preparation of a polymer-stabilized LC mixture, 99.99% of the mixture according to Example M168 are mixed with

0.01% of the polymerizable compound of the formula

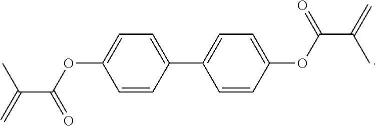 [SMILES: C=C(C)C(=O)Oc1ccc(-c2ccc(OC(=O)C(=C)C)cc2)cc1]

### Example M169

For the preparation of a PS-VA mixture, 99.7% of the mixture according to Example M168 are mixed with 0.3% of the polymerizable compound of the formula

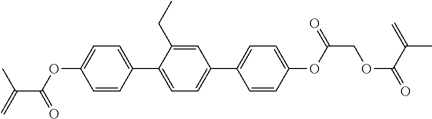 [SMILES: C=C(C)C(=O)OCC(=O)Oc1ccc(-c2ccc(-c3ccc(OC(=O)C(=C)C)cc3)c(CC)c2)cc1]

### and 0.001% of Irganox-1076 (BASF).

### Example M170

| | | | |
|---|---|---|---|
| BCH-32 | 3.00% | Clearing point [° C.]: | 110.4 |
| CCH-13 | 16.50% | Δn [589 nm, 20° C.]: | 0.0981 |
| CCH-34 | 4.50% | Δε [1 kHz, 20° C.]: | −3.7 |
| CCH-35 | 6.00% | ε_{\|\|} [1 kHz, 20° C.]: | 3.3 |
| CCP-3-1 | 5.50% | ε_{⊥} [1 kHz, 20° C.]: | 7.0 |
| CCY-3-1 | 4.00% | K₁ [pN, 20° C.]: | 19.7 |
| CCY-3-O1 | 11.00% | K₃ [pN, 20° C.]: | 20.5 |
| CCY-3-O2 | 13.00% | γ₁ [mPa · s, 20° C.]: | 212 |
| CCY-4-O2 | 11.00% | V₀ [20° C., V]: | 2.48 |
| CPY-2-O2 | 5.50% | | |
| CPY-3-O2 | 11.50% | | |
| CY-3-O2 | 8.00% | | |
| CY-5-O2 | 0.50% | | |

### Example M171

| | | | |
|---|---|---|---|
| BCH-32 | 3.00% | Clearing point [° C.]: | 110.3 |
| CCH-13 | 16.50% | Δn [589 nm, 20° C.]: | 0.0983 |
| CCH-34 | 4.50% | Δε [1 kHz, 20° C.]: | −3.7 |
| CCH-35 | 6.00% | ε_{\|\|} [1 kHz, 20° C.]: | 3.3 |
| CCP-3-1 | 5.50% | ε_{⊥} [1 kHz, 20° C.]: | 7.0 |
| CCY-3-1 | 4.00% | K₁ [pN, 20° C.]: | 19.6 |
| CCY-3-O1 | 11.00% | K₃ [pN, 20° C.]: | 20.5 |
| CCY-3-O2 | 11.50% | γ₁ [mPa · s, 20° C.]: | 213 |
| CCY-4-O2 | 11.00% | V₀ [20° C., V]: | 2.49 |
| CCY-5-O2 | 1.50% | | |
| CPY-2-O2 | 5.50% | | |
| CPY-3-O2 | 11.50% | | |
| CY-3-O2 | 8.50% | | |

### Example M172

For the preparation of a polymer-stabilized LC mixture, 99.9% of the mixture according to Example M171 are mixed with

0.1% of the polymerizable compound of the formula

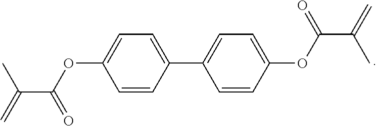 [SMILES: C=C(C)C(=O)Oc1ccc(-c2ccc(OC(=O)C(=C)C)cc2)cc1]

### Example M173

| | | | |
|---|---|---|---|
| BCH-32 | 3.00% | Clearing point [° C.]: | 110.1 |
| CCH-13 | 14.25% | Δn [589 nm, 20° C.]: | 0.0980 |
| CCH-34 | 5.00% | Δε [1 kHz, 20° C.]: | −3.7 |
| CCH-35 | 6.50% | ε_{\|\|} [1 kHz, 20° C.]: | 3.3 |
| CCP-3-1 | 4.50% | ε_{⊥} [1 kHz, 20° C.]: | 7.0 |
| CCY-3-1 | 6.50% | K₁ [pN, 20° C.]: | 19.2 |
| CCY-3-O1 | 9.00% | K₃ [pN, 20° C.]: | 20.3 |
| CCY-3-O2 | 14.50% | γ₁ [mPa · s, 20° C.]: | |
| CCY-4-O2 | 10.00% | V₀ [20° C., V]: | 2.48 |
| CPY-2-O2 | 92.75% | | |
| CPY-3-O2 | 14.00% | | |
| CY-3-O4 | 9.50% | | |

### Example M174

| | | | |
|---|---|---|---|
| BCH-32 | 5.50% | Clearing point [° C.]: | 75.1 |
| CC-3-V1 | 8.50% | Δn [589 nm, 20° C.]: | 0.984 |
| CCH-13 | 19.00% | Δε [1 kHz, 20° C.]: | −3.3 |
| CCH-3O1 | 4.00% | ε_{\|\|} [1 kHz, 20° C.]: | 3.5 |
| CCH-34 | 5.50% | ε_{⊥} [1 kHz, 20° C.]: | 6.8 |
| CCY-3-O1 | 9.00% | K₁ [pN, 20° C.]: | 14.1 |
| CCY-3-O2 | 9.00% | K₃ [pN, 20° C.]: | 15.1 |
| CPY-2-O2 | 10.00% | γ₁ [mPa · s, 20° C.]: | |
| CPY-3-O2 | 10.00% | V₀ [20° C., V]: | 2.25 |
| CY-3-O2 | 13.00% | | |
| PY-3-O2 | 6.50% | | |

### Example M175

| | | | |
|---|---|---|---|
| B-2O-O5 | 4.00% | Clearing point [° C.]: | 74.1 |
| BCH-32 | 8.00% | Δn [589 nm, 20° C.]: | 0.1084 |
| CC-3-V1 | 9.00% | Δε [1 kHz, 20° C.]: | −3.2 |
| CCH-13 | 5.00% | ε_{\|\|} [1 kHz, 20° C.]: | 3.6 |
| CCH-34 | 8.00% | ε_{⊥} [1 kHz, 20° C.]: | 6.8 |
| CCH-35 | 8.00% | K₁ [pN, 20° C.]: | 14.6 |
| CCP-3-1 | 8.00% | K₃ [pN, 20° C.]: | 16.0 |
| CCY-3-O2 | 4.50% | γ₁ [mPa · s, 20° C.]: | |
| CLY-3-O2 | 10.00% | V₀ [20° C., V]: | 2.35 |
| CY-3-O2 | 14.00% | | |
| PCH-3O1 | 5.00% | | |
| PGIY-2-O4 | 5.00% | | |
| PY-3-O2 | 11.50% | | |

### Example M176

| | | | |
|---|---|---|---|
| B-2O-O5 | 4.00% | Clearing point [° C.]: | 74.5 |
| BCH-32 | 8.00% | Δn [589 nm, 20° C.]: | 0.1090 |
| CC-3-V1 | 9.00% | Δε [1 kHz, 20° C.]: | −3.1 |
| CCH-13 | 10.00% | ε_{\|\|} [1 kHz, 20° C.]: | 3.5 |
| CCH-34 | 8.00% | ε_{⊥} [1 kHz, 20° C.]: | 6.6 |
| CCH-35 | 4.00% | K₁ [pN, 20° C.]: | 14.7 |
| CCP-3-1 | 8.00% | K₃ [pN, 20° C.]: | 16.5 |
| CCY-3-O2 | 11.00% | γ₁ [mPa · s, 20° C.]: | |
| CCY-4-O2 | 3.00% | V₀ [20° C., V]: | 2.43 |
| CPY-3-O2 | 7.00% | | |
| CY-3-O2 | 6.00% | | |
| PCH-3O1 | 4.00% | | |
| PY-3-O2 | 18.00% | | |

### Example M177

| | | | |
|---|---|---|---|
| B-2O-O5 | 4.00% | Clearing point [° C.]: | 74.5 |
| BCH-32 | 8.00% | Δn [589 nm, 20° C.]: | 0.1089 |
| CC-3-V1 | 9.00% | Δε [1 kHz, 20° C.]: | −3.1 |
| CCH-13 | 6.00% | ε_{\|\|} [1 kHz, 20° C.]: | 3.5 |
| CCH-34 | 8.00% | ε_{⊥} [1 kHz, 20° C.]: | 6.6 |
| CCH-35 | 7.00% | K₁ [pN, 20° C.]: | 14.7 |
| CCP-3-1 | 8.00% | K₃ [pN, 20° C.]: | 16.6 |
| CCY-3-O2 | 11.00% | γ₁ [mPa · s, 20° C.]: | |
| CCY-4-O2 | 2.50% | V₀ [20° C., V]: | 2.42 |
| CPY-3-O2 | 6.00% | | |
| CY-3-O2 | 7.50% | | |
| PCH-3O1 | 5.00% | | |
| PY-3-O2 | 18.00% | | |

### Example M178

| | | | |
|---|---|---|---|
| B-2O-O5 | 4.00% | Clearing point [° C.]: | 74.3 |
| BCH-32 | 8.00% | Δn [589 nm, 20° C.]: | 0.1088 |
| CC-3-V1 | 9.00% | Δε [1 kHz, 20° C.]: | −3.2 |
| CCH-13 | 10.00% | ε_{\|\|} [1 kHz, 20° C.]: | 3.5 |
| CCH-34 | 8.00% | ε_{⊥} [1 kHz, 20° C.]: | 6.7 |
| CCH-35 | 4.00% | K₁ [pN, 20° C.]: | 14.9 |
| CCP-3-1 | 8.00% | K₃ [pN, 20° C.]: | 16.6 |
| CCY-3-O2 | 11.00% | γ₁ [mPa · s, 20° C.]: | 102 |
| CCY-4-O2 | 3.00% | V₀ [20° C., V]: | 2.39 |
| CPY-3-O2 | 7.00% | | |
| CY-3-O2 | 7.50% | | |
| PCH-3O1 | 2.50% | | |
| PY-3-O2 | 18.00% | | |

### Example M179

For the preparation of a polymer-stabilized LC mixture, 99.7% of the mixture according to Example M173 are mixed with

0.3% of the polymerizable compound of the formula

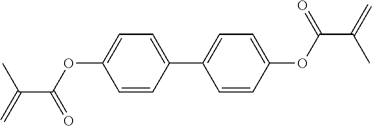 [SMILES: C=C(C)C(=O)Oc1ccc(-c2ccc(OC(=O)C(=C)C)cc2)cc1]

### and 0.001% of Irganox-1076 (BASF).

### Example M180

| | | | |
|---|---|---|---|
| CC-5-V | 20.00% | Clearing point [° C.]: | 75.0 |
| CCH-13 | 14.50% | Δn [589 nm, 20° C.]: | 0.1078 |
| CC-3-V1 | 8.00% | Δε [1 kHz, 20° C.]: | −2.9 |
| CCP-3-1 | 2.00% | ε_{\|\|} [1 kHz, 20° C.]: | 3.4 |
| CCY-3-O1 | 6.00% | ε_{⊥} [1 kHz, 20° C.]: | 6.3 |
| CCY-3-O2 | 10.00% | K₁ [pN, 20° C.]: | 15.0 |
| CPY-2-O2 | 5.50% | K₃ [pN, 20° C.]: | 15.6 |
| CCY-3-O2 | 11.50% | γ₁ [mPa · s, 20° C.]: | 98 |
| PY-3-O2 | 17.50% | V₀ [20° C., V]: | 2.45 |
| PYP-2-3 | 5.00% | | |

### Example M181

| | | | |
|---|---|---|---|
| CC-3-V1 | 8.50% | Clearing point [° C.]: | 74.5 |
| CCH-13 | 11.00% | Δn [589 nm, 20° C.]: | 0.0983 |
| CCH-35 | 7.00% | Δε [1 kHz, 20° C.]: | −3.5 |
| CCH-34 | 5.00% | ε_{\|\|} [1 kHz, 20° C.]: | 3.6 |
| CCP-3-1 | 13.50% | ε_{⊥} [1 kHz, 20° C.]: | 7.1 |
| CCY-3-O1 | 6.00% | K₁ [pN, 20° C.]: | 15.5 |
| CCY-3-O2 | 9.00% | K₃ [pN, 20° C.]: | 16.5 |
| CPY-3-O2 | 10.50% | γ₁ [mPa · s, 20° C.]: | 101 |
| CY-3-O2 | 11.50% | V₀ [20° C., V]: | 2.30 |
| Y-4O-O4 | 6.00% | LTS [bulk −20° C.]: | >1000 h |
| B(S)-2O-O5 | 4.00% | | |
| PP-1-3 | 5.00% | | |
| B(S)-2O-O4 | 3.00% | | |

### Example M182

| | | | |
|---|---|---|---|
| CC-V-V | 10.00% | Clearing point [° C.]: | 73.5 |
| CCH-13 | 10.00% | Δn [589 nm, 20° C.]: | 0.1067 |
| CCP-V-1 | 10.00% | Δε [1 kHz, 20° C.]: | −2.8 |
| CCY-2-1 | 10.00% | ε_{\|\|} [1 kHz, 20° C.]: | 3.5 |
| CCY-3-1 | 10.00% | ε_{⊥} [1 kHz, 20° C.]: | 6.2 |
| CCY-3-O1 | 10.00% | K₁ [pN, 20° C.]: | 12.8 |
| CCY-3-O2 | 7.00% | K₃ [pN, 20° C.]: | 14.5 |
| CPY-2-O2 | 10.00% | γ₁ [mPa · s, 20° C.]: | |
| CY-3-O2 | 4.00% | V₀ [20° C., V]: | 2.42 |
| PP-1-3 | 10.00% | | |
| PY-3-O2 | 9.00% | | |

### Example M183

| | | | |
|---|---|---|---|
| CC-3-V1 | 11.00% | Clearing point [° C.]: | 75 |
| CCH-13 | 9.00% | Δn [589 nm, 20° C.]: | 0.0983 |
| CCH-1-4 | 8.00% | Δε [1 kHz, 20° C.]: | −3.4 |
| CCH-34 | 10.00% | ε_{\|\|} [1 kHz, 20° C.]: | 3.4 |
| CCP-V-1 | 5.00% | ε_{⊥} [1 kHz, 20° C.]: | 6.8 |
| CCY-3-O1 | 1.50% | K₁ [pN, 20° C.]: | 14.8 |
| CCY-3-O2 | 9.50% | K₃ [pN, 20° C.]: | 15.8 |
| CLY-3-O2 | 5.00% | γ₁ [mPa · s, 20° C.]: | 104 |
| CLY-3-O3 | 5.00% | V₀ [20° C., V]: | 2.29 |
| CPY-3-O2 | 10.00% | | |
| CY-3-O2 | 11.50% | | |
| PGIY-2-O4 | 2.50% | | |
| PY-3-O2 | 12.00% | | |

### Example M184

| | | | |
|---|---|---|---|
| BCH-32 | 15.00% | Clearing point [° C.]: | 75.2 |
| CCH-13 | 15.00% | Δn [589 nm, 20° C.]: | 0.1101 |
| CCH-23 | 10.00% | Δε [1 kHz, 20° C.]: | −2.5 |
| CCH-34 | 5.00% | ε_{\|\|} [1 kHz, 20° C.]: | 3.5 |
| CCP-3-1 | 11.50% | ε_{⊥} [1 kHz, 20° C.]: | 6.0 |
| CLY-3-O2 | 8.00% | K₁ [pN, 20° C.]: | 14.6 |
| CPY-2-O2 | 4.50% | K₃ [pN, 20° C.]: | 13.9 |
| CPY-3-O2 | 10.00% | γ₁ [mPa · s, 20° C.]: | |
| PGIY-2-O4 | 6.00% | V₀ [20° C., V]: | 2.39 |
| PY-3-2 | 5.00% | LTS [bulk −20° C.]: | >1000 h |
| Y-4O-O4 | 10.00% | | |

### Example M185

| | | | |
|---|---|---|---|
| BCH-32 | 13.00% | Clearing point [° C.]: | 71.2 |
| BCH-52 | 10.00% | Δn [589 nm, 20° C.]: | 0.1092 |
| CCH-13 | 15.00% | Δε [1 kHz, 20° C.]: | −2.2 |
| CCH-23 | 9.00% | ε_{\|\|} [1 kHz, 20° C.]: | 3.5 |
| CCH-3O1 | 7.00% | ε_{⊥} [1 kHz, 20° C.]: | 5.7 |
| CCH-34 | 5.00% | K₁ [pN, 20° C.]: | 12.8 |
| CLY-3-O2 | 8.00% | K₃ [pN, 20° C.]: | 11.9 |
| CPY-2-O2 | 8.00% | γ₁ [mPa · s, 20° C.]: | 104 |
| CPY-3-O2 | 6.50% | V₀ [20° C., V]: | 2.47 |
| PGIY-2-O4 | 6.00% | | |
| PYP-2-4 | 2.50% | | |
| Y-4O-O4 | 10.00% | | |

### Example M186

| | | | |
|---|---|---|---|
| CC-3-V1 | 10.00% | Clearing point [° C.]: | 75.5 |
| CCH-13 | 11.50% | Δn [589 nm, 20° C.]: | 0.0984 |
| CCH-34 | 5.50% | Δε [1 kHz, 20° C.]: | −3.5 |
| CCH-35 | 9.00% | ε_{\|\|} [1 kHz, 20° C.]: | 3.5 |
| CCP-3-1 | 5.50% | ε_{⊥} [1 kHz, 20° C.]: | 7.0 |
| CCY-3-O2 | 12.50% | K₁ [pN, 20° C.]: | 15.2 |
| CPY-2-O2 | 8.00% | K₃ [pN, 20° C.]: | 16.9 |
| CPY-3-O2 | 11.00% | γ₁ [mPa · s, 20° C.]: | 112 |
| CY-3-O2 | 15.50% | V₀ [20° C., V]: | 2.31 |
| PGIY-2-O4 | 2.00% | | |
| PY-3-O2 | 9.50% | | |

### Example M187

For the preparation of a polymer-stabilized LC mixture, 99.7% of the mixture according to Example M186 are mixed with

0.3% of the polymerizable compound of the formula

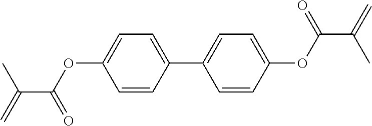 [SMILES: C=C(C)C(=O)Oc1ccc(-c2ccc(OC(=O)C(=C)C)cc2)cc1]

### and 0.001% of Irganox-1076 (BASF).

### Example M188

| | | | |
|---|---|---|---|
| BCH-32 | 2.50% | Clearing point [° C.]: | 75 |
| CC-3-V1 | 8.00% | Δn [589 nm, 20° C.]: | 0.01057 |
| CCH-13 | 15.00% | Δε [1 kHz, 20° C.]: | −2.8 |
| CCH-34 | 2.50% | ε_{\|\|} [1 kHz, 20° C.]: | 3.3 |
| CCH-35 | 5.00% | ε_{⊥} [1 kHz, 20° C.]: | 6.2 |
| CCP-3-1 | 11.00% | K₁ [pN, 20° C.]: | 15.9 |
| CCY-3-O1 | 6.00% | K₃ [pN, 20° C.]: | 18.2 |
| CCY-3-O2 | 10.00% | γ₁ [mPa · s, 20° C.]: | 106 |
| CPY-3-O2 | 10.00% | V₀ [20° C., V]: | 2.68 |
| CPY-3-O2 | 10.00% | | |
| CY-3-O2 | 12.00% | | |
| PP-1-2V1 | 6.00% | | |
| PY-3-O2 | 12.00% | | |

### Example M189

| | | | |
|---|---|---|---|
| BCH-32 | 4.50% | Clearing point [° C.]: | 75 |
| CC-3-V1 | 8.50% | Δn [589 nm, 20° C.]: | 0.1055 |
| CCH-13 | 14.50% | Δε [1 kHz, 20° C.]: | −2.8 |
| CCH-34 | 3.00% | ε_{\|\|} [1 kHz, 20° C.]: | 3.3 |
| CCH-35 | 4.50% | ε_{⊥} [1 kHz, 20° C.]: | 6.1 |
| CCP-3-1 | 11.00% | K₁ [pN, 20° C.]: | 15.6 |
| CCY-3-O1 | 4.50% | K₃ [pN, 20° C.]: | 17.6 |
| CCY-3-O2 | 10.00% | γ₁ [mPa · s, 20° C.]: | 102 |
| CPY-3-O2 | 9.00% | V₀ [20° C., V]: | 2.67 |
| CY-3-O2 | 12.00% | | |
| PP-1-2V1 | 5.00% | | |
| PY-3-O2 | 11.00% | | |
| PY-4-O2 | 2.00% | | |

### Example M190

| | | | |
|---|---|---|---|
| B-2O-O5 | 5.00% | Clearing point [° C.]: | 74.5 |
| BCH-52 | 8.00% | Δn [589 nm, 20° C.]: | 0.1033 |
| CC-3-V | 22.50% | Δε [1 kHz, 20° C.]: | −3.0 |
| CCH-13 | 9.40% | ε_{\|\|} [1 kHz, 20° C.]: | 3.6 |
| CCH-34 | 5.50% | K₁ [pN, 20° C.]: | 13.4 |
| CCY-3-O1 | 8.20% | K₃ [pN, 20° C.]: | 13.5 |
| CCY-3-O2 | 8.80% | γ₁ [mPa · s, 20° C.]: | 96 |
| CCY-4-O2 | 11.60% | V₀ [20° C., V]: | 2.26 |
| PP-1-2V1 | 2.00% | | |
| PY-3-O2 | 15.00% | | |
| PY-4-O2 | 1.00% | | |
| PYP-2-3 | 3.00% | | |

### Example M191

For the preparation of a polymer-stabilized LC mixture, 99.7% of the mixture according to Example M190 are mixed with

0.3% of the polymerizable compound of the formula

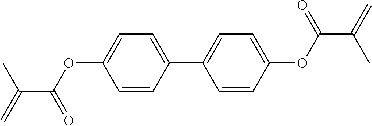 [SMILES: C=C(C)C(=O)Oc1ccc(-c2ccc(OC(=O)C(=C)C)cc2)cc1]

### and 0.001% of Irganox-1076 (BASF).

### Example M192

| | |
|---|---|
| CC-3-V1 | 12.50% |
| CCH-13 | 19.00% |
| CCH-34 | 6.50% |
| CCY-3-O1 | 12.00% |
| CCY-3-O2 | 12.00% |
| CCY-4-O2 | 9.00% |
| CPY-3-O2 | 1.00% |
| CY-3-O2 | 7.00% |
| PY-3-O2 | 13.00% |
| PYP-2-3 | 5.50% |
| PYP-2-4 | 2.50% |

### Example M193

For the preparation of a polymer-stabilized LC mixture, 99.7% of the mixture according to Example M192 are mixed with

0.3% of the polymerizable compound of the formula

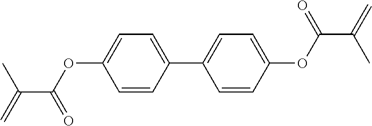 [SMILES: C=C(C)C(=O)Oc1ccc(-c2ccc(OC(=O)C(=C)C)cc2)cc1]

### and 0.001% of Irganox-1076 (BASF).

### Example M194

| | |
|---|---|
| CC-3-V1 | 11.25% |
| CCH-13 | 16.00% |
| CCH-3O1 | 0.75% |
| CCH-34 | 6.50% |
| CCH-35 | 5.00% |
| CCY-3-O1 | 11.50% |
| CCY-3-O2 | 13.50% |
| CCY-4-O2 | 6.25% |
| CPY-3-O2 | 0.50% |
| CY-3-O2 | 6.50% |
| PY-3-O2 | 14.50% |
| PYP-2-3 | 7.25% |
| PYP-2-4 | 0.50% |

### Example M195

For the preparation of a polymer-stabilized LC mixture, 99.7% of the mixture according to Example M194 are mixed with

0.3% of the polymerizable compound of the formula

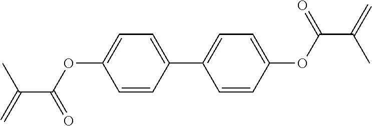 [SMILES: C=C(C)C(=O)Oc1ccc(-c2ccc(OC(=O)C(=C)C)cc2)cc1]

### and 0.001% of Irganox-1076 (BASF).

### Example M196

For the preparation of a polymer-stabilized LC mixture, 99.8% of the mixture according to Example M194 is mixed with

0.2% of the polymerizable compound of the formula

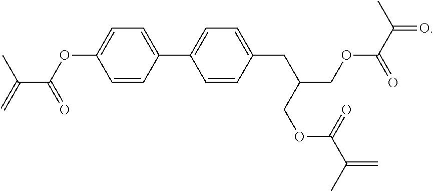 [SMILES: C=C(C)C(=O)OCC(COC(=O)C(C)=O)Cc1ccc(-c2ccc(OC(=O)C(=C)C)cc2)cc1]

### Example M197

| | | | |
|---|---|---|---|
| B-2O-O5 | 5.00% | Clearing point [° C.]: | 75 |
| BCH-52 | 7.25% | Δn [589 nm, 20° C.]: | 0.1034 |
| CC-3-V | 22.50% | | |
| CCH-13 | 5.75% | | |
| CCH-3O1 | 1.00% | | |
| CCH-34 | 5.50% | | |
| CCH-35 | 3.50% | | |
| CCY-3-O1 | 8.00% | | |
| CCOY-3-O2 | 10.00% | | |
| CCOY-4-O2 | 9.50% | | |
| PP-1-2V1 | 2.50% | | |
| PY-V2-O2 | 11.00% | | |
| PY-4-O2 | 5.50% | | |
| PYP-2-3 | 3.00% | | |

### Example M198

For the preparation of a polymer-stabilized LC mixture, 99.7% of the mixture according to Example M197 is mixed with

0.3% of the polymerizable compound of the formula

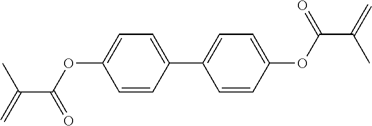 [SMILES: C=C(C)C(=O)Oc1ccc(-c2ccc(OC(=O)C(=C)C)cc2)cc1]

### and 0.001% of Irganox-1076 (BASF).

### Example M199

For the preparation of a polymer-stabilized LC mixture, 99.8% of the mixture according to Example M197 is mixed with

0.2% of the polymerizable compound of the formula

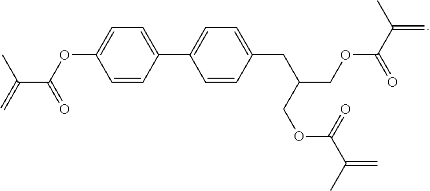 [SMILES: C=C(C)C(=O)OCC(COC(=O)C(=C)C)Cc1ccc(-c2ccc(OC(=O)C(=C)C)cc2)cc1]

### Example M200

The mixture according to Example M188 additionally comprises 0.03% of the compound of the formula

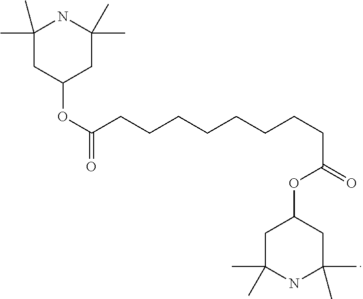

### Example M201

The mixture according to Example M188 additionally comprises

0.04% of the compound of the formula

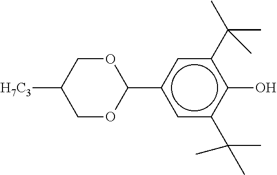

and 0.02% of the compound of the formula

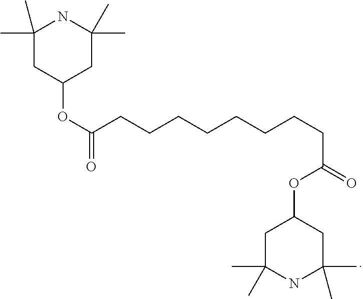

### Example M202

The mixture according to Example M99 additionally comprises 0.03% of the compound of the formula

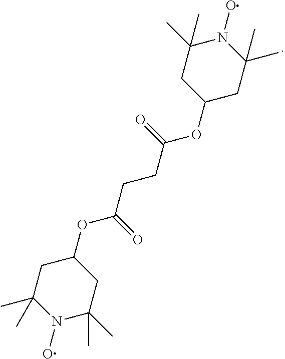

### Example M203

The mixture according to Example M99 additionally comprises

0.04% of the compound of the formula

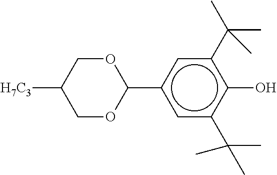

and 0.02% of the compound of the formula

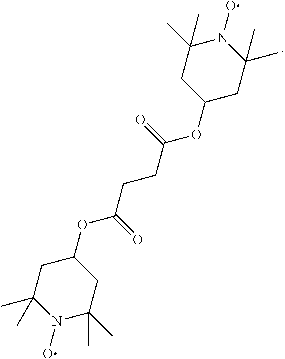

### Example M204

| | | | |
|---|---|---|---|
| CC-3-V1 | 9.00% | Clearing point [° C.]: | 74.8 |
| CCH-13 | 18.00% | | |
| CCH-34 | 9.00% | | |
| CCP-3-1 | 5.50% | | |
| CCY-3-O1 | 3.50% | | |
| CCY-3-O2 | 11.50% | | |
| CPY-V-O2 | 9.00% | | |
| CPY-3-O2 | 11.00% | | |
| CY-3-O2 | 13.50% | | |
| PY-3-O2 | 10.00% | | |

### Example M205

The mixture according to Example M204 additionally comprises 0.03% of the compound of the formula

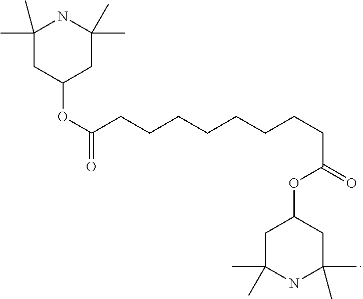

### Example M206

| | | | |
|---|---|---|---|
| CC-3-V1 | 10.00% | Clearing point [° C.]: | 77 |
| CCH-13 | 11.50% | | |
| CCH-34 | 5.50% | | |
| CCH-35 | 9.00% | | |
| CCP-3-1 | 5.50% | | |
| CCY-3-O2 | 12.50% | | |
| CPY-2-O2 | 8.00% | | |
| CPY-3-O2 | 11.00% | | |
| CY-3-O2 | 15.50% | | |
| CK-3-F | 2.00% | | |
| PY-3-O2 | 9.50% | | |

### Example M207

| | | | |
|---|---|---|---|
| CC-3-V1 | 12.50% | Clearing point [° C.]: | 74.9 |
| CCH-13 | 19.00% | Δn [589 nm, 20° C.]: | 0.0980 |
| CCH-34 | 6.50% | Δε [1 kHz, 20° C.]: | −3.3 |
| CCY-3-O1 | 12.00% | ε_{\|\|} [1 kHz, 20° C.]: | 3.4 |
| CCY-3-O2 | 12.00% | K₁ [pN, 20° C.]: | 14.7 |
| CCY-4-O2 | 9.00% | K₃ [pN, 20° C.]: | 15.6 |
| CPY-3-O2 | 1.00% | γ₁ [mPa · s, 20° C.]: | 109 |
| CY-3-O2 | 7.00% | V₀ [20° C., V]: | 2.29 |
| PY-3-O2 | 13.00% | | |
| PYP-2-3 | 5.50% | | |
| PYP-2-4 | 2.50% | | |

### Example M208

For the preparation of a PS-VA mixture, 99.7% of the mixture according to Example M207 is mixed with

0.3% of the polymerizable compound of the formula

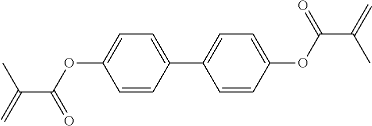 [SMILES: C=C(C)C(=O)Oc1ccc(-c2ccc(OC(=O)C(=C)C)cc2)cc1]

### and 0.001% of Irganox-1076 (BASF).

### Example M209

| | | | |
|---|---|---|---|
| CC-3-V1 | 11.25% | Clearing point [° C.]: | 74.7 |
| CCH-13 | 16.00% | Δn [589 nm, 20° C.]: | 0.0977 |
| CCH-3O1 | 0.75% | Δε [1 kHz, 20° C.]: | −3.2 |
| CCH-34 | 6.50% | ε_{\|\|} [1 kHz, 20° C.]: | 3.4 |
| CCH-35 | 5.00% | K₁ [pN, 20° C.]: | 14.7 |
| CCY-3-O1 | 11.50% | K₃ [pN, 20° C.]: | 15.6 |
| CCY-3-O2 | 13.50% | γ₁ [mPa · s, 20° C.]: | 106 |
| CCY-4-O2 | 6.25% | V₀ [20° C., V]: | 2.33 |
| CPY-3-O2 | 0.50% | | |
| CY-3-O2 | 6.50% | | |
| PY-3-O2 | 14.50% | | |
| PYP-2-3 | 7.25% | | |
| PYP-2-4 | 0.50% | | |

### Example M210

For the preparation of a PS-VA mixture, 99.7% of the mixture according to Example M209 is mixed with

0.3% of the polymerizable compound of the formula

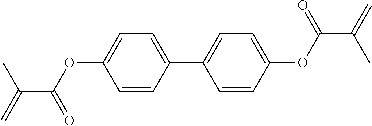 [SMILES: C=C(C)C(=O)Oc1ccc(-c2ccc(OC(=O)C(=C)C)cc2)cc1]

### and 0.001% of Irganox-1076 (BASF).

### Example M211

For the preparation of a PS-VA mixture, 99.7% of the mixture according to Example M151 is mixed with

0.3% of the polymerizable compound of the formula

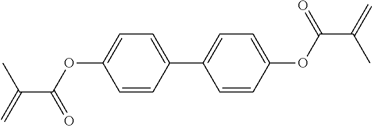 [SMILES: C=C(C)C(=O)Oc1ccc(-c2ccc(OC(=O)C(=C)C)cc2)cc1]

### and 0.001% of Irganox-1076 (BASF).

### Example M212

| | | | |
|---|---|---|---|
| BCH-52 | 5.00% | Clearing point [° C.]: | 74.2 |
| CCH-13 | 17.00% | Δn [589 nm, 20° C.]: | 0.1349 |
| CCH-34 | 7.00% | Δε [1 kHz, 20° C.]: | −2.7 |
| CCH-35 | 5.00% | ε_{\|\|} [1 kHz, 20° C.]: | 3.5 |
| CCP-3-1 | 6.50% | K₁ [pN, 20° C.]: | 14.2 |
| CPY-2-O2 | 11.00% | K₃ [pN, 20° C.]: | 13.8 |
| CPY-3-O2 | 11.00% | γ₁ [mPa · s, 20° C.]: | 119 |
| PY-1-O4 | 7.50% | V₀ [20° C., V]: | 2.40 |
| PY-3-O2 | 12.00% | | |
| PYP-2-3 | 9.00% | | |
| PYP-2-4 | 9.00% | | |

### Example M213

For the preparation of a PS-VA mixture, 99.7% of the mixture according to Example M212 is mixed with

0.3% of the polymerizable compound of the formula

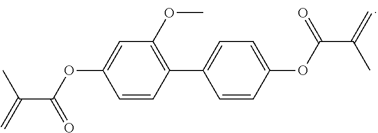 [SMILES: C=C(C)C(=O)Oc1ccc(-c2ccc(OC(=O)C(=C)C)cc2OC)cc1]

Without further elaboration, it is believed that one skilled in the art can, using the preceding description, utilize the present invention to its fullest extent. The preceding preferred specific embodiments and examples are, therefore, to be construed as merely illustrative, and not limitative of the remainder of the disclosure in any way whatsoever.

From the foregoing description, one skilled in the art can easily ascertain the essential characteristics of this invention and, without departing from the spirit and scope thereof, can make various changes and modifications of the invention to adapt it to various usages and conditions.

The entire disclosures of all applications, patents and publications, cited herein and of corresponding U.S. Provisional Application No. 62/105,578 filed Jan. 20, 2015, are incorporated by reference herein.

1. A liquid-crystalline medium based on a mixture of polar compounds, wherein said medium comprises the compound of formula I,

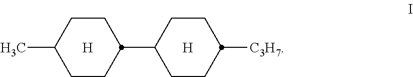

2. The liquid-crystalline medium according to claim 1, wherein said medium additionally comprises one or more compounds selected from the group of compounds of formulae IIA, IIB and IIC, 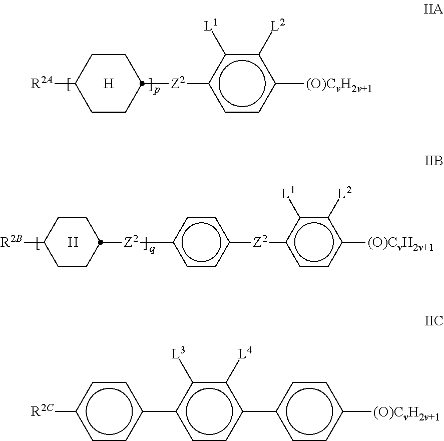
in which
R^{2A}, R^{2B} and R^{2C} each, independently of one another, denote H, an alkyl or alkenyl radical having up to 15 C atoms which is unsubstituted, monosubstituted by CN or CF₃ or at least monosubstituted by halogen, where, in addition, one or more CH₂ groups in these radicals are each optionally replaced by —O—, —S—, 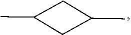
—C≡C—, —CF₂O—, —OCF₂—, —OC—O— or —O—CO— in such a way that O atoms are not linked directly to one another,
L¹⁻⁴ each, independently of one another, denote F or Cl,
Z² and Z^{2′} each, independently of one another, denote a single bond, —CH₂CH₂—, —CH═CH—, —CF₂O—, —OCF₂—, —CH₂O—, —OCH₂—, —COO—, —OCO—, —C₂F₄—, —CF═CF—, —CH═CHCH₂O—,
(O) denotes O or a single bond,
p denotes 1 or 2,
q denotes 0 or 1, and
v denotes 1 to 6.

3. The liquid-crystalline medium according to claim 1, wherein said medium additionally comprises one or more compounds of formula III, 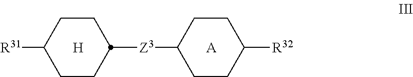
in which
R³¹ and R³² each, independently of one another, denote a straight-chain alkyl, alkoxyalkyl or alkoxy radical having up to 12 C atoms, 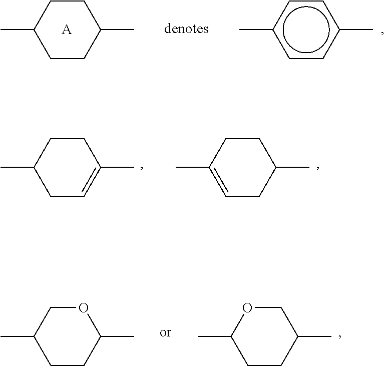
and
Z³ denotes a single bond, —CH₂CH₂—, —CH═CH—, —CF₂O—, —OCF₂—, —CH₂O—, —OCH₂—, —COO—, —OCO—, —C₂F₄—, —C₄H₉—, —CF═CF—.

4. The liquid-crystalline medium according to claim 1, wherein said medium additionally comprises one or more compounds of formulae L-1 to L-11, 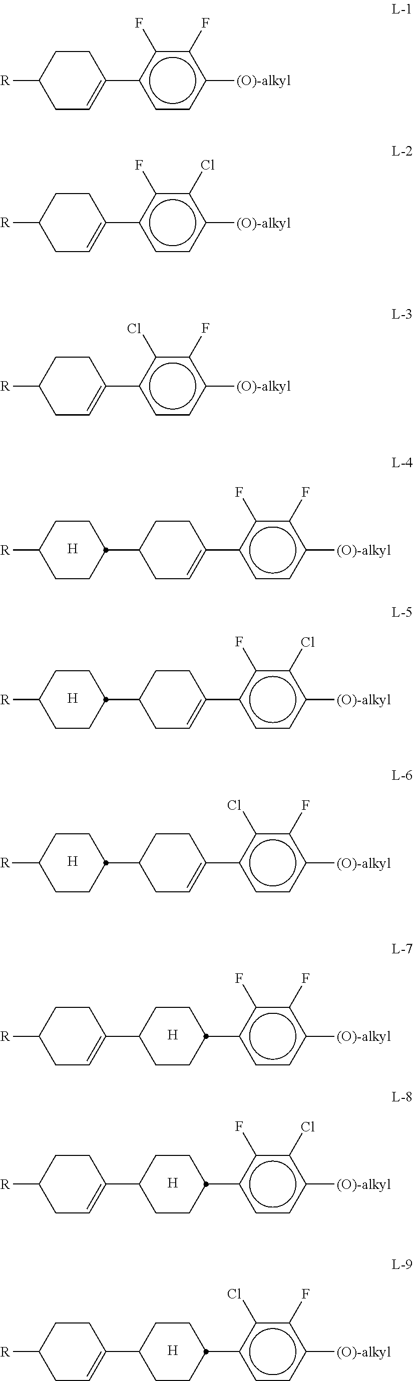 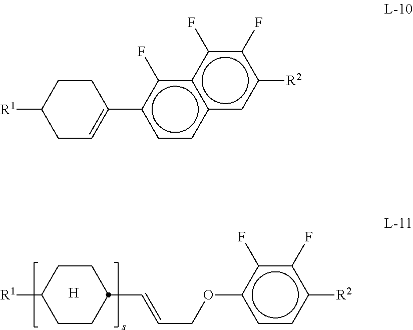
in which
R, R¹ and R² each, independently of one another, denote H, an alkyl or alkenyl radical having up to 15 C atoms which is unsubstituted, monosubstituted by CN or CF₃ or at least monosubstituted by halogen, where, in addition, one or more CH₂ groups in these radicals are each optionally replaced by —O—, —S—, 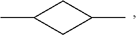
—C≡C—, —CF₂O—, —OCF₂—, —OC—O— or —O—CO— in such a way that O atoms are not linked directly to one another,
(O) denotes O or a single bond,
alkyl denotes an alkyl radical having 1-6 C atoms, and
s denotes 1 or 2.

5. The liquid-crystalline medium according to claim 1, wherein said medium additionally comprises one or more terphenyls of formulae T-1 to T-21, 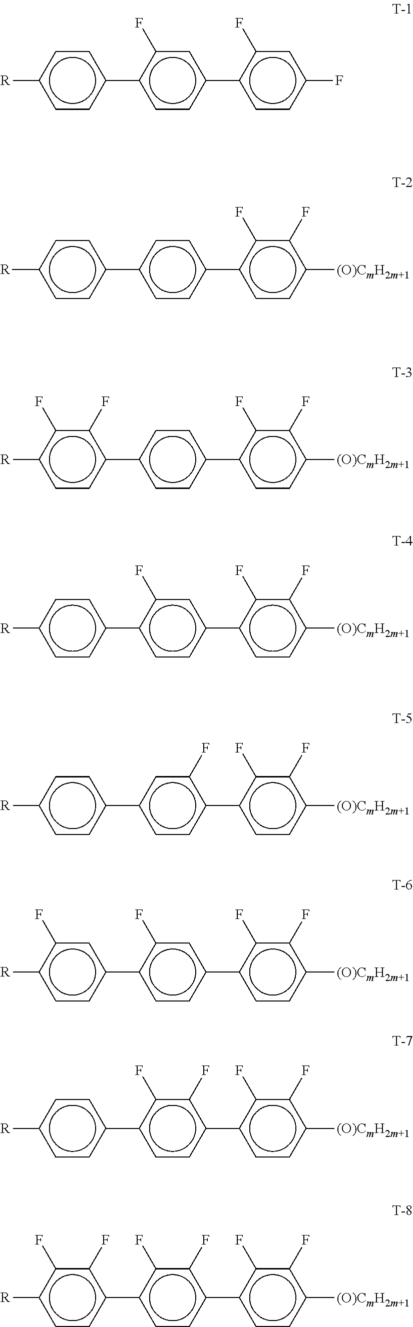 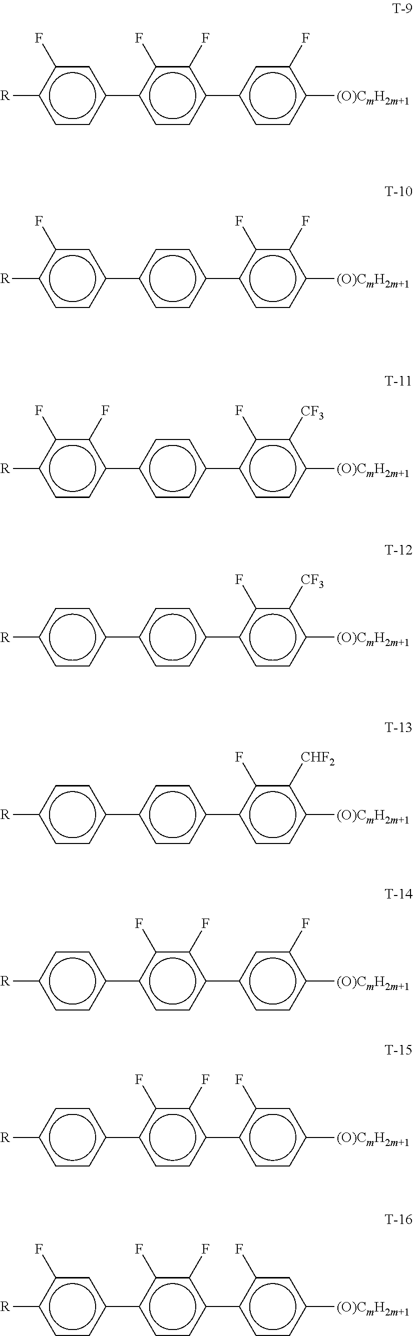 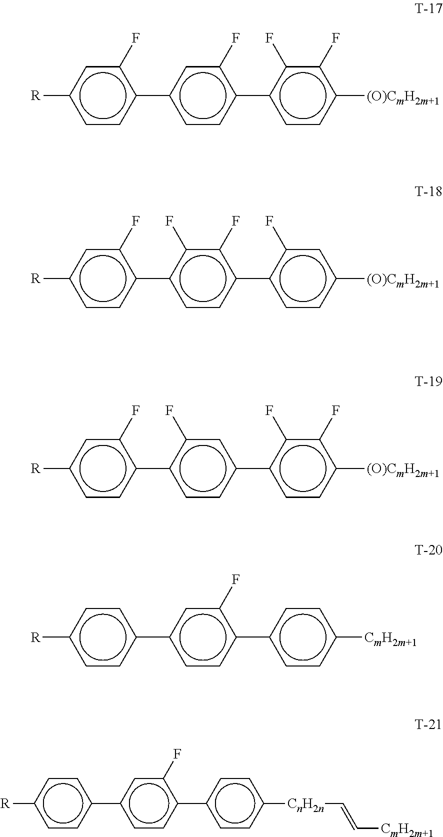
in which
R denotes a straight-chain alkyl or alkoxy radical having 1-7 C atoms or straight-chain alkenyl radical having 2-7 C atoms,
(O) denotes O or a single bond, and
m denotes 1-6.

6. The liquid-crystalline medium according to claim 1, wherein said medium additionally comprises one or more compounds of formulae O-1 to O-17, 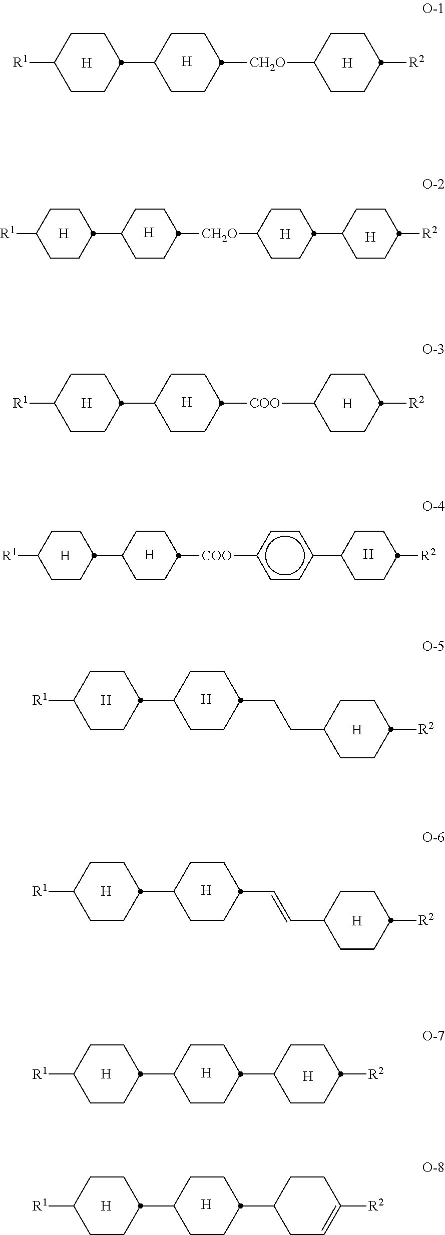 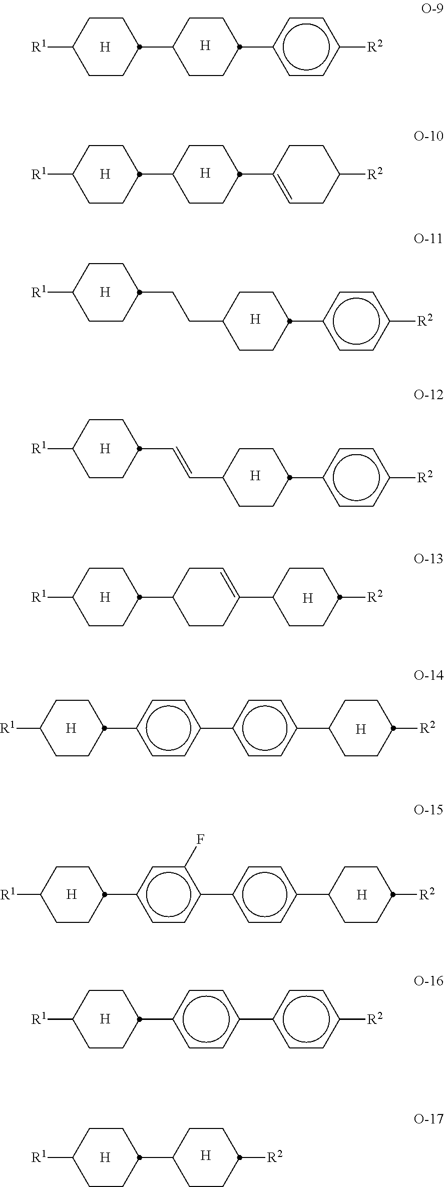
in which
R¹ and R² each, independently of one another, denote H, an alkyl or alkenyl radical having up to 15 C atoms which is unsubstituted, monosubstituted by CN or CF₃ or at least monosubstituted by halogen, where, in addition, one or more CH₂ groups in these radicals are each optionally replaced by —O—, —S—, 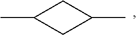
—C≡C—, —CF₂O—, —OCF₂—, —OC—O— or —O—CO— in such a way that O atoms are not linked directly to one another, and
the compound(s) of formula O-17 is (are) not identical with the compound of formula I.

7. The liquid-crystalline medium according to claim 1, wherein said medium additionally comprises one or more indane compounds of formula In, 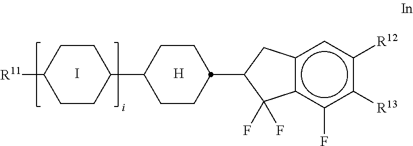
in which
R¹¹, R¹², R¹³ denote a straight-chain alkyl having 1-6 C atoms, alkoxy having 1-6 C atoms, alkoxyalkyl having 2-6 C atoms, or alkenyl having 2-6 C atoms,
R¹² and R¹³ additionally may also denote H or halogen, 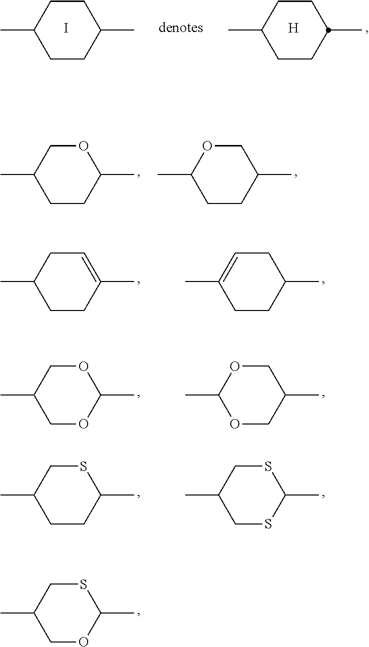
and
i denotes 0, 1 or 2.

8. The liquid-crystalline medium according to claim 1, wherein the proportion of compounds of formula I in the mixture as a whole is 1-30% by weight.

9. The liquid-crystalline medium according to claim 1, wherein said medium additionally comprises one or more compounds from the group of compounds of the formulae

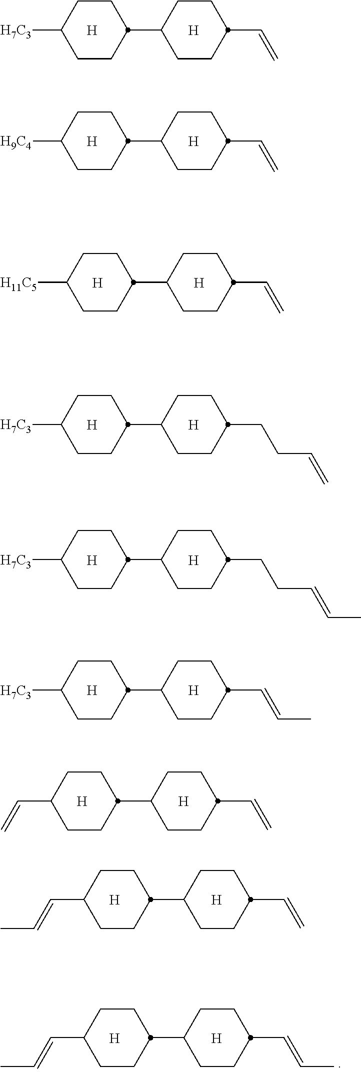

10. The liquid-crystalline medium according to claim 1, wherein said medium additionally comprises a compound selected from the following group:

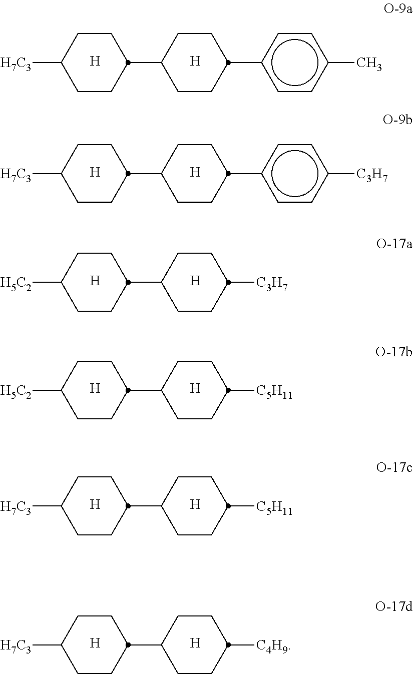

11. The liquid-crystalline medium according to claim 1, wherein said medium additionally comprises one or more compounds selected from the group of the compounds of formulae BC, CR, PH-1, PH-2, BF-1, BF-2, BS-1 and BS-2 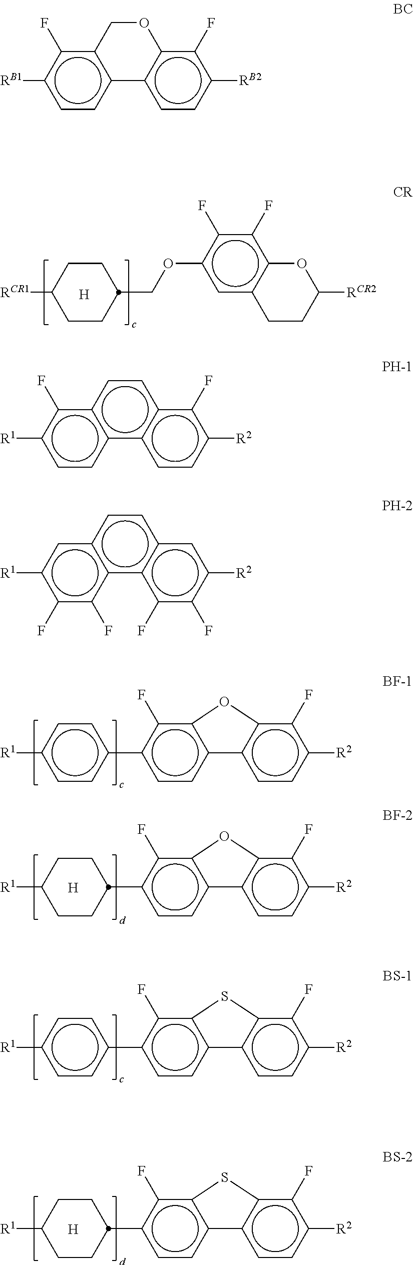
in which
R^{B1}, R^{B2}, R^{CR1}, R^{CR2}, R¹, R² each, independently of one another, denote H, an alkyl or alkenyl radical having up to 15 C atoms which is unsubstituted, monosubstituted by CN or CF₃ or at least monosubstituted by halogen, where, in addition, one or more CH₂ groups in these radicals are each optionally replaced by —O—, —S—, 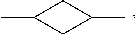
—C≡C—, —CF₂O—, —OCF₂—, —OC—O— or —O—CO— in such a way that O atoms are not linked directly to one another,
c denotes 0, 1 or 2,
d denotes 1 or 2, and
R¹ and R² each, independently of one another, denote alkyl having 1 to 6 C atoms, alkoxy having 1 to 6 C atoms, alkenyl having 2 to 6 C atoms, or alkenyloxy having 2 to 6 C atoms respectively.

12. The liquid-crystalline medium according to claim 1, wherein said medium comprises at least one polymerizable compound.

13. The liquid-crystalline medium according to claim 1, wherein said medium comprises one or more additives.

14. The liquid-crystalline medium according to claim 13, wherein said additive is selected from the group of free-radical scavengers, antioxidants and/or UV stabilizers.

15. The liquid-crystalline medium according to claim 1, wherein said medium further comprises one or more stabilizers selected from the following: 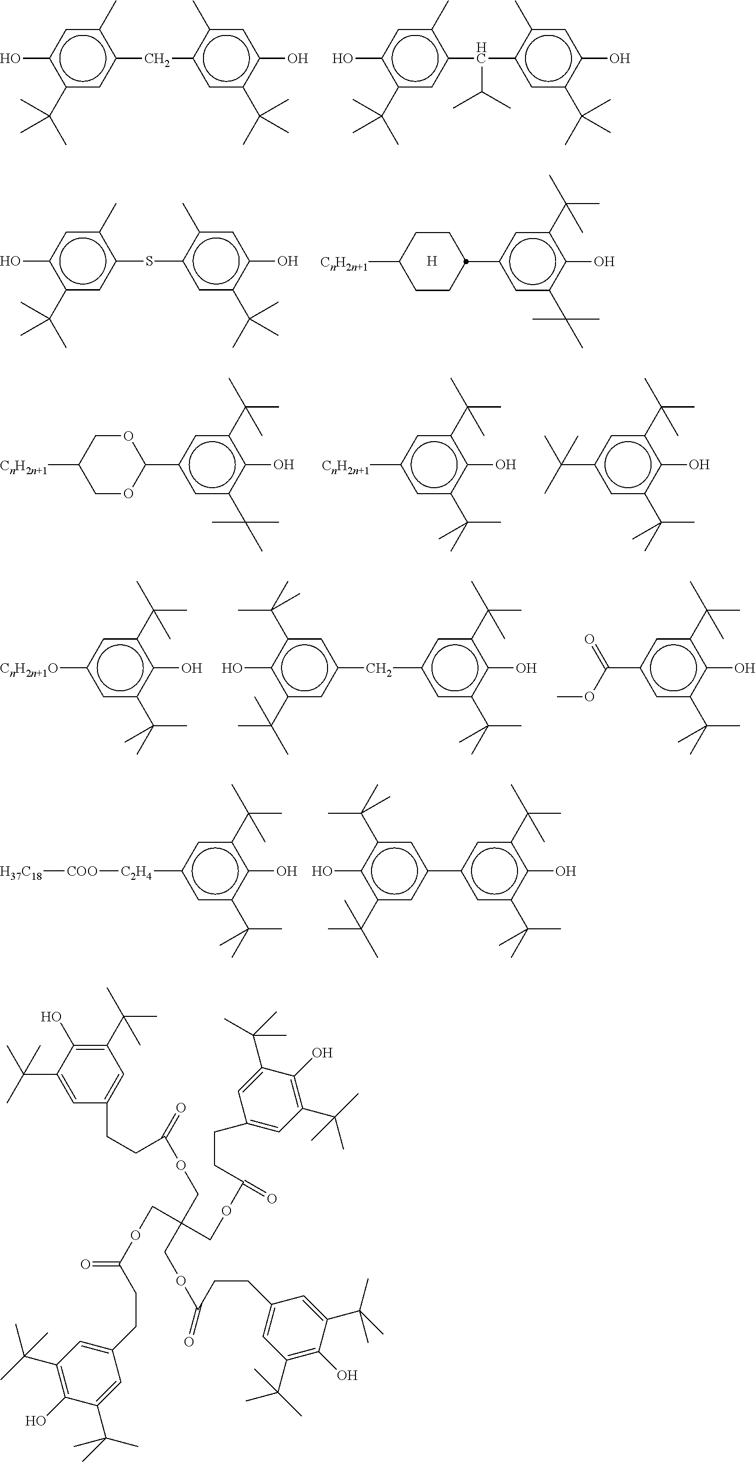 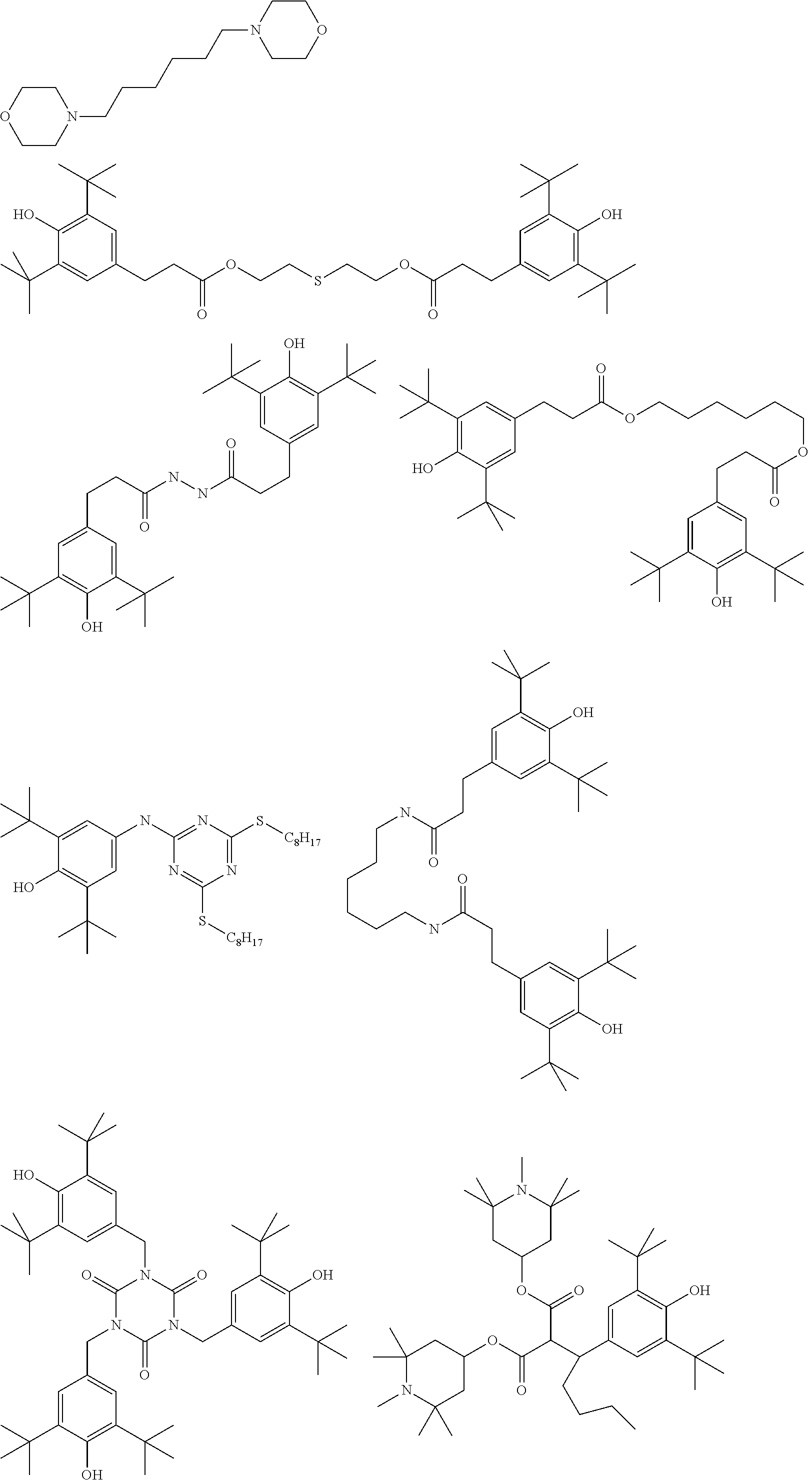 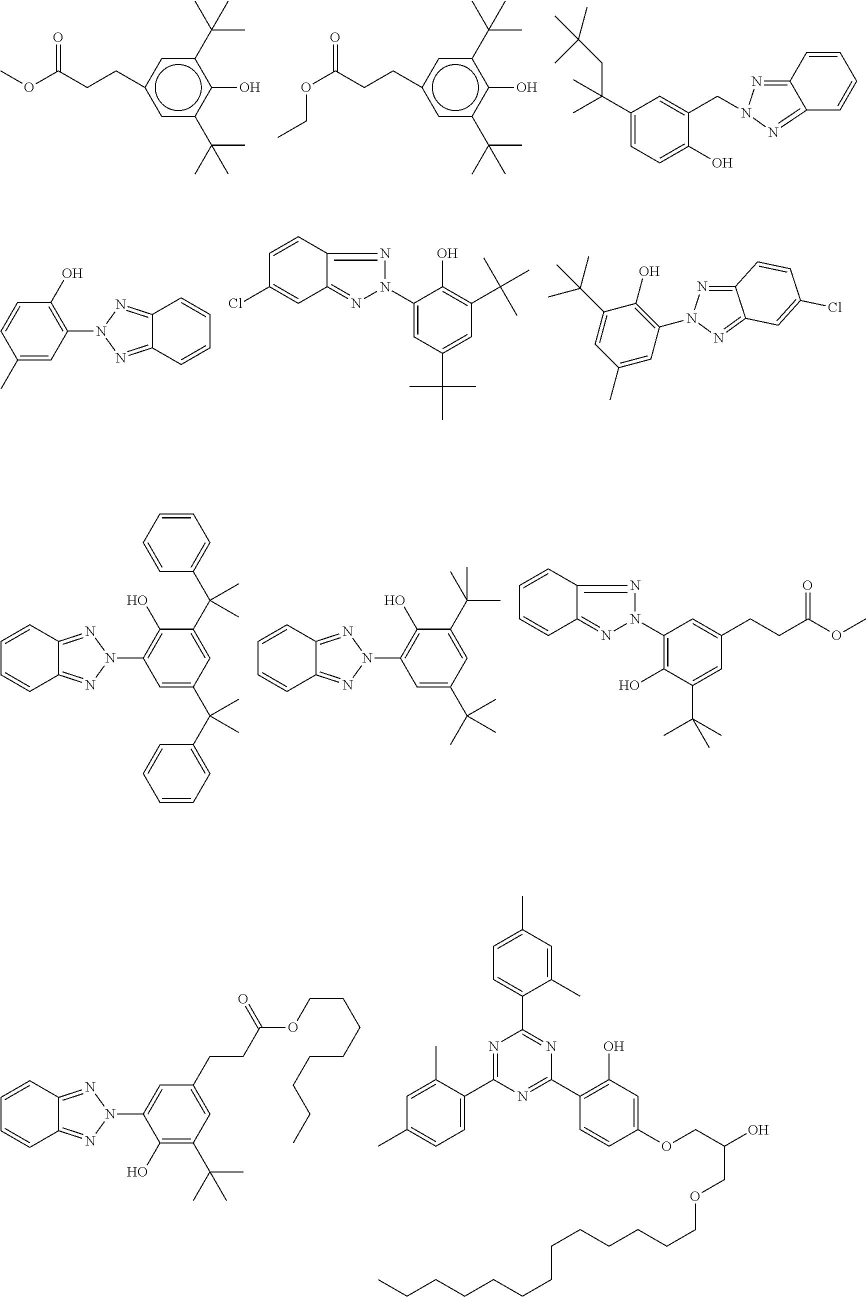 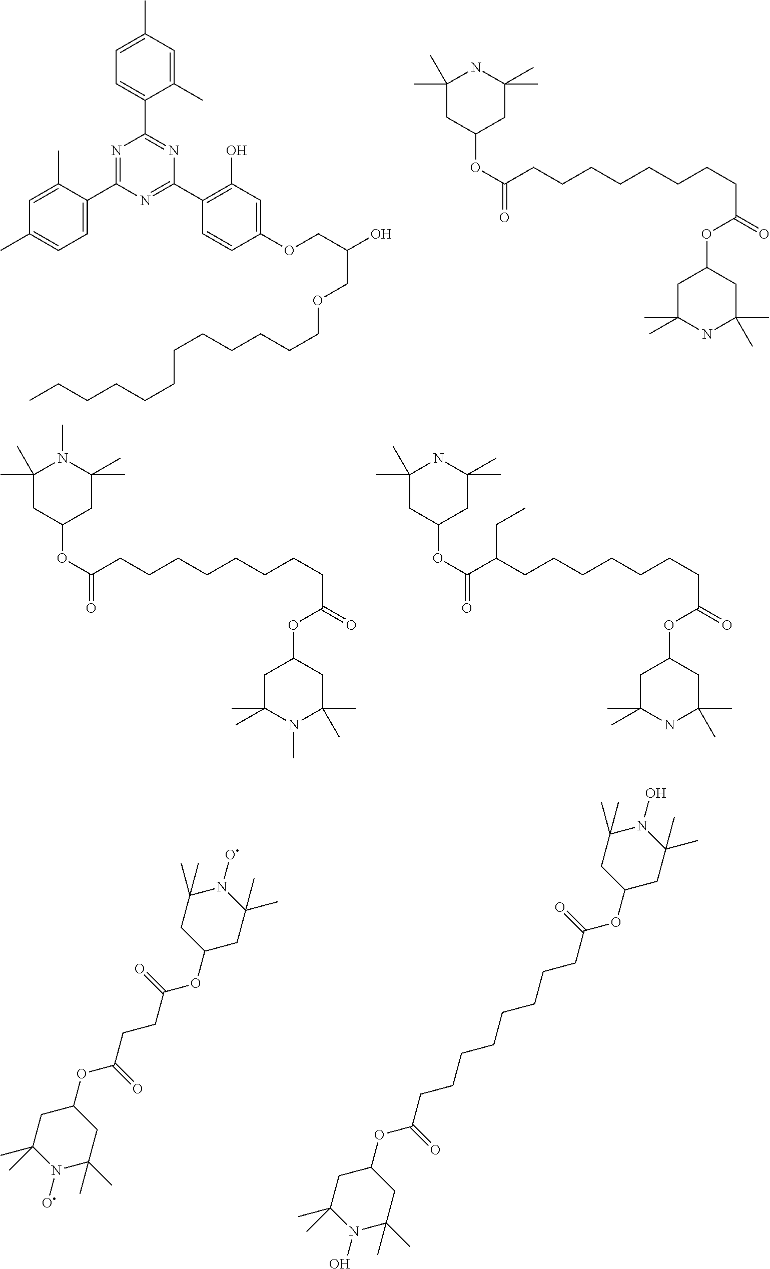
wherein n=1, 2, 3, 4, 5, 6 or 7.

16. The liquid-crystalline medium according to claim 1, wherein said medium further comprises one or more reactive mesogens selected from the following:

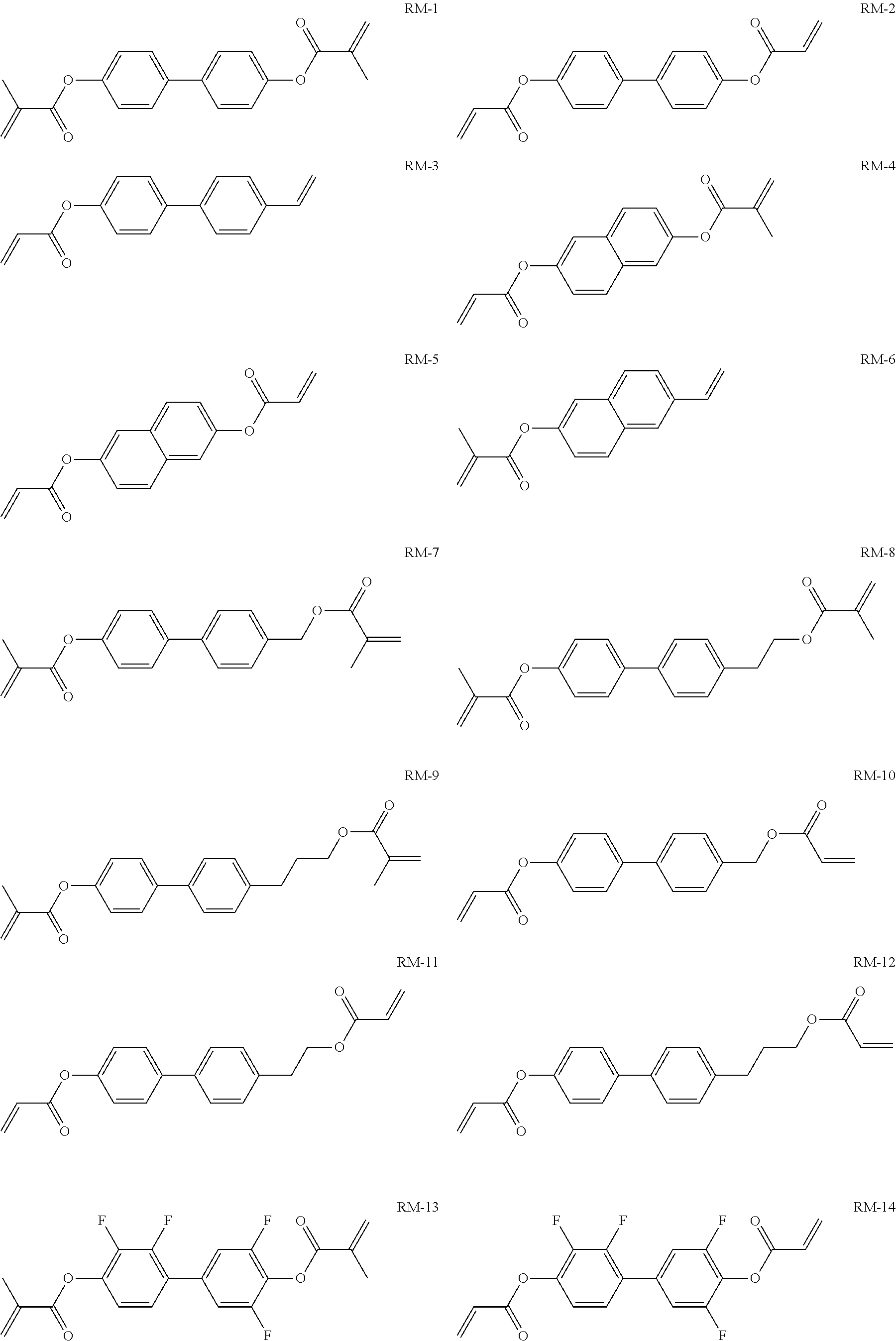

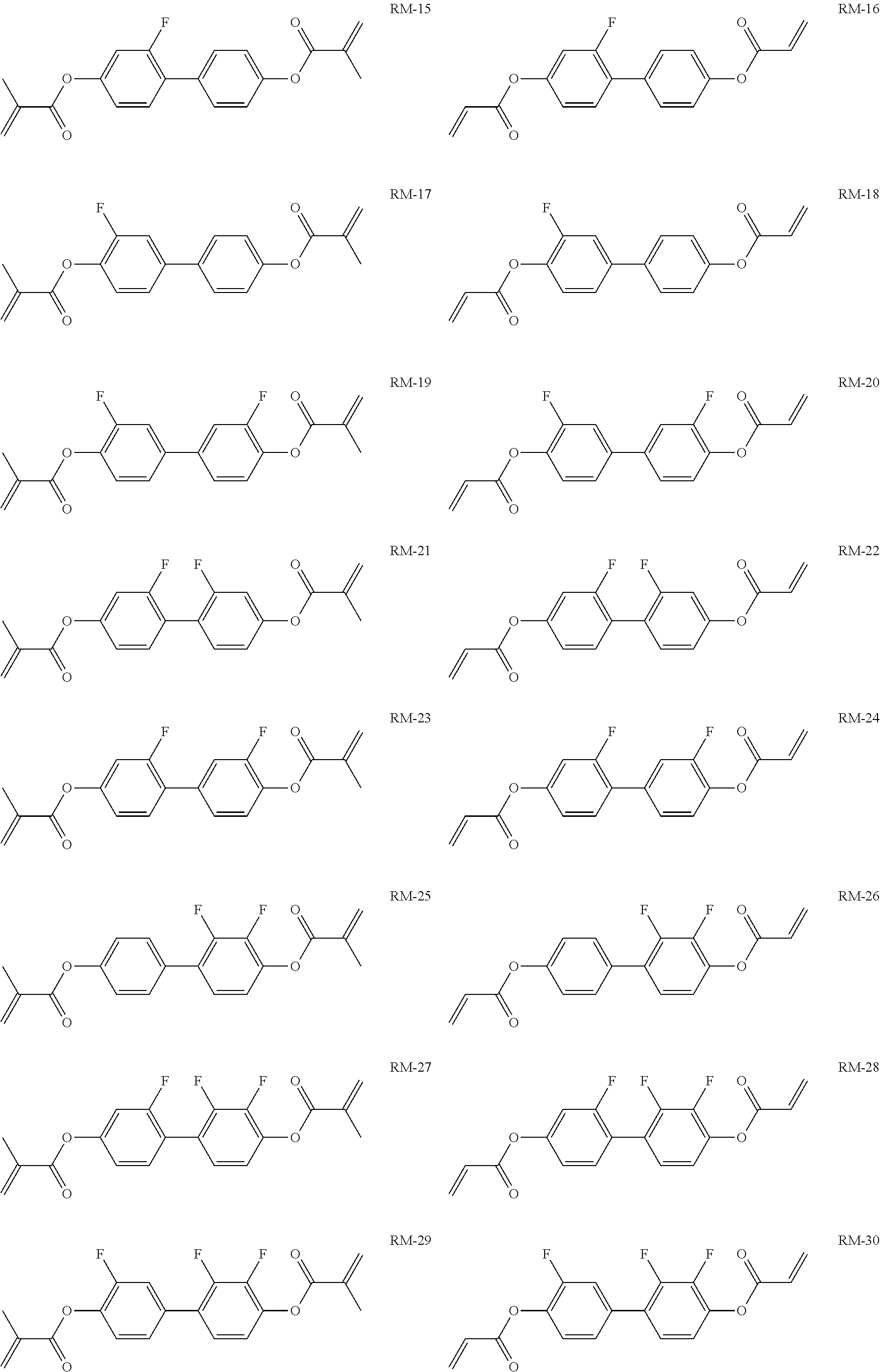

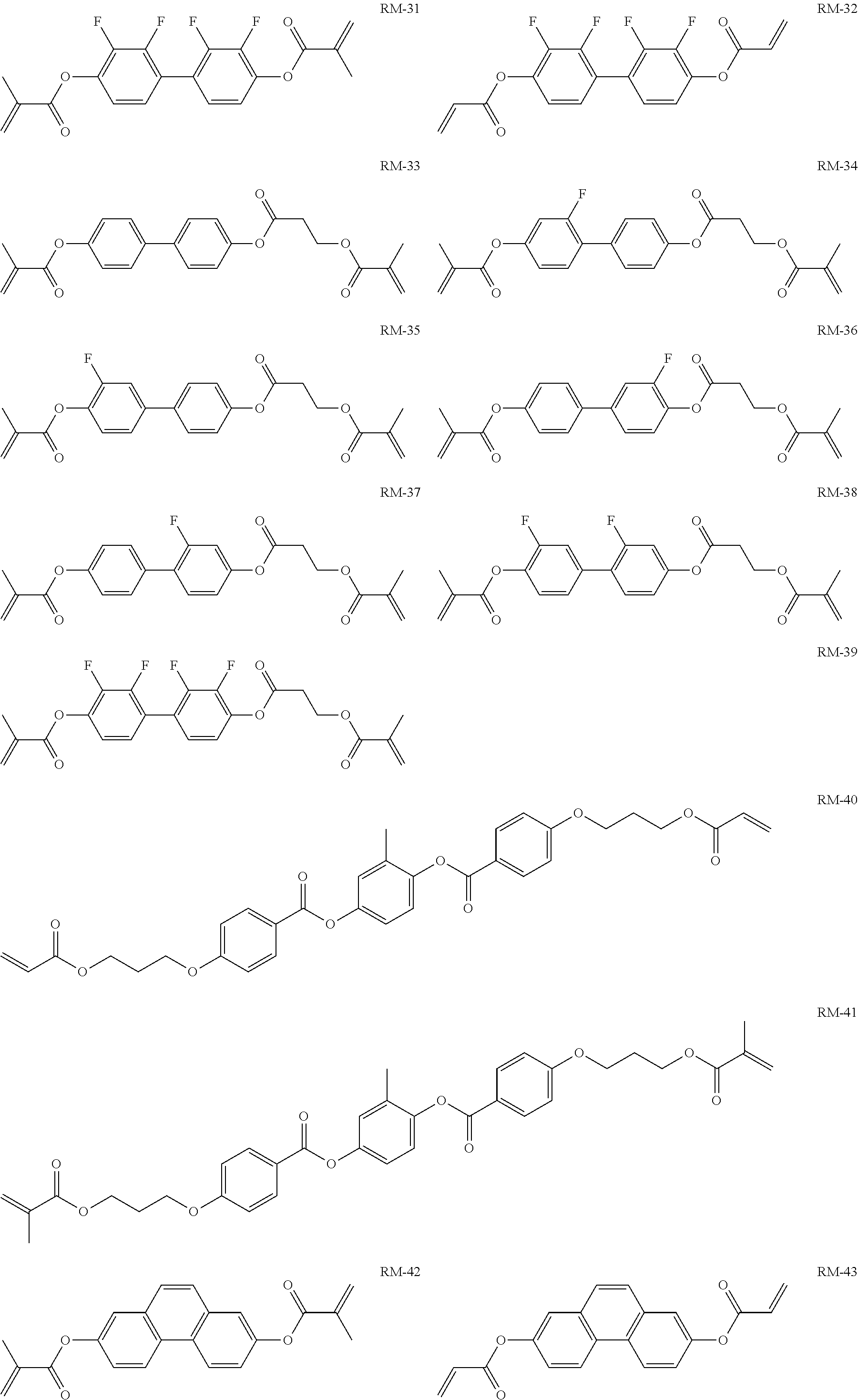

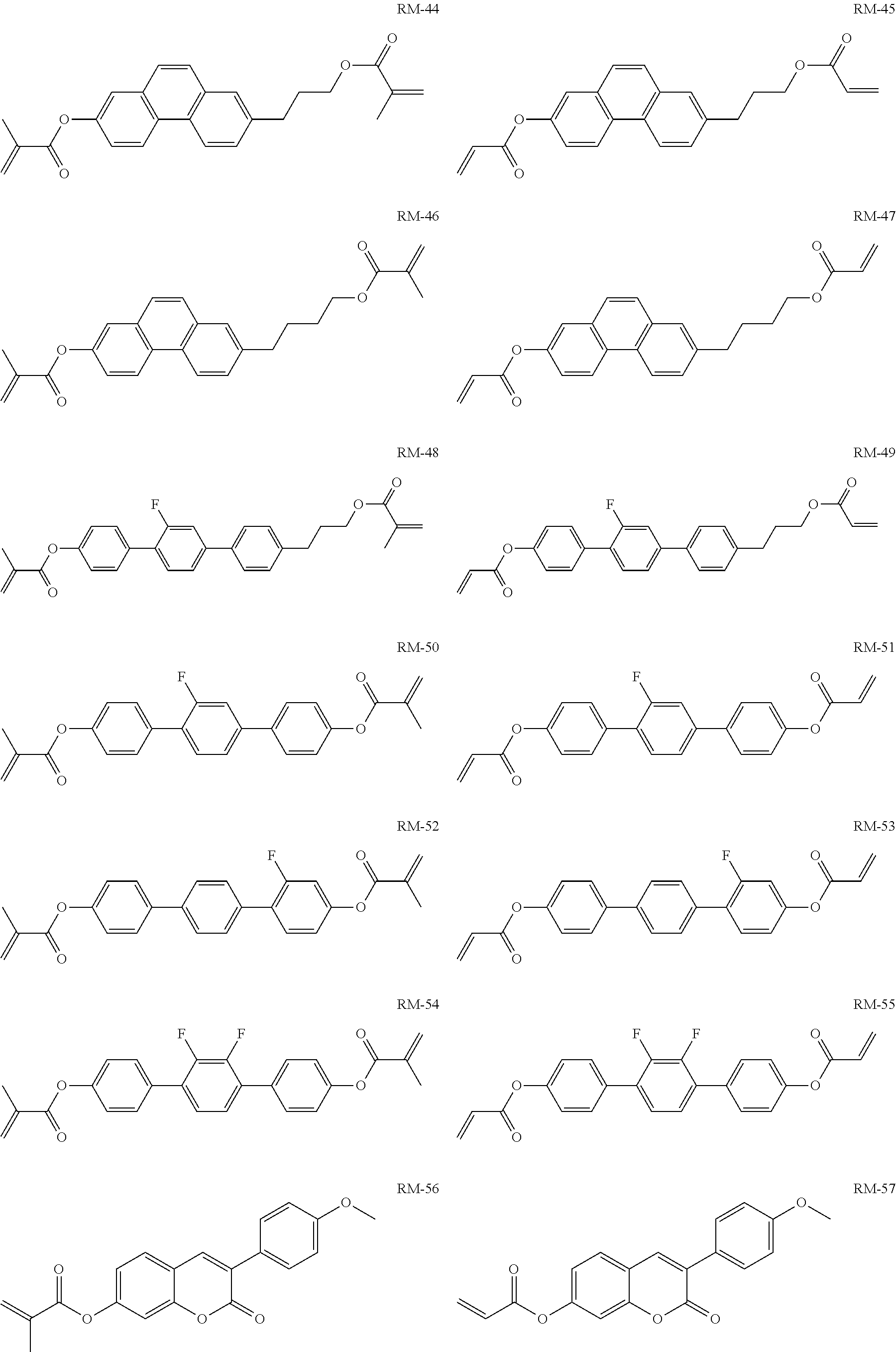

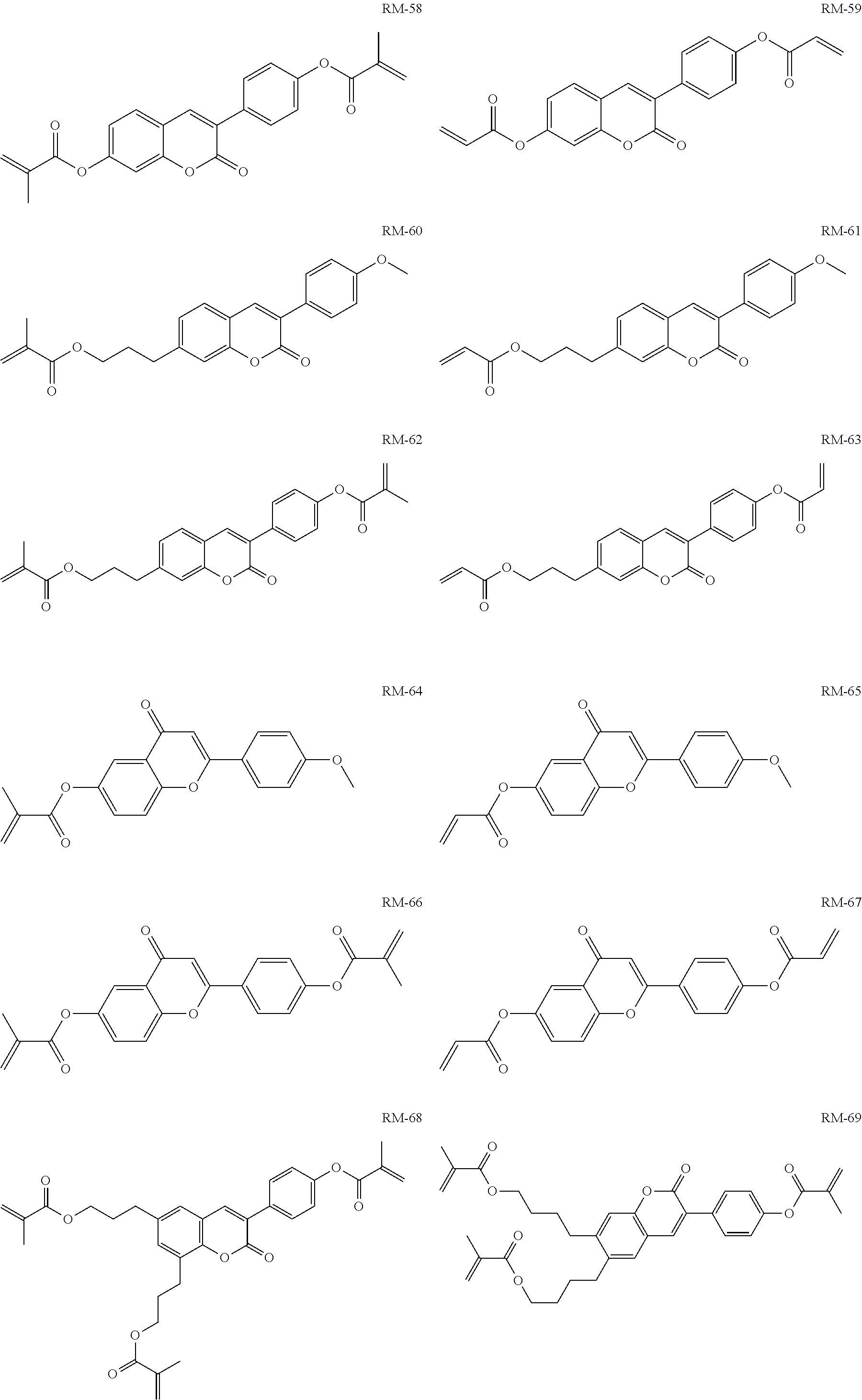

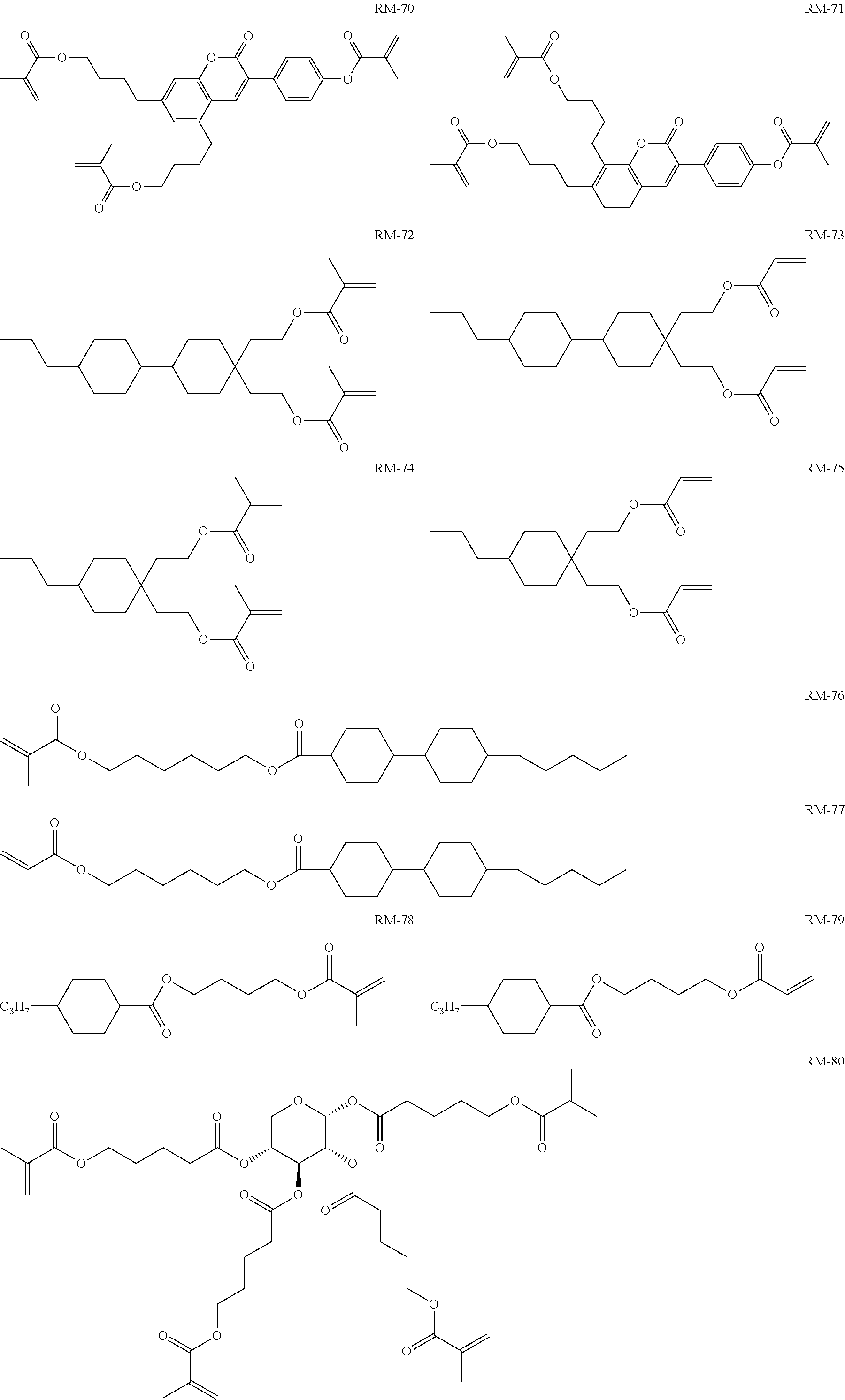

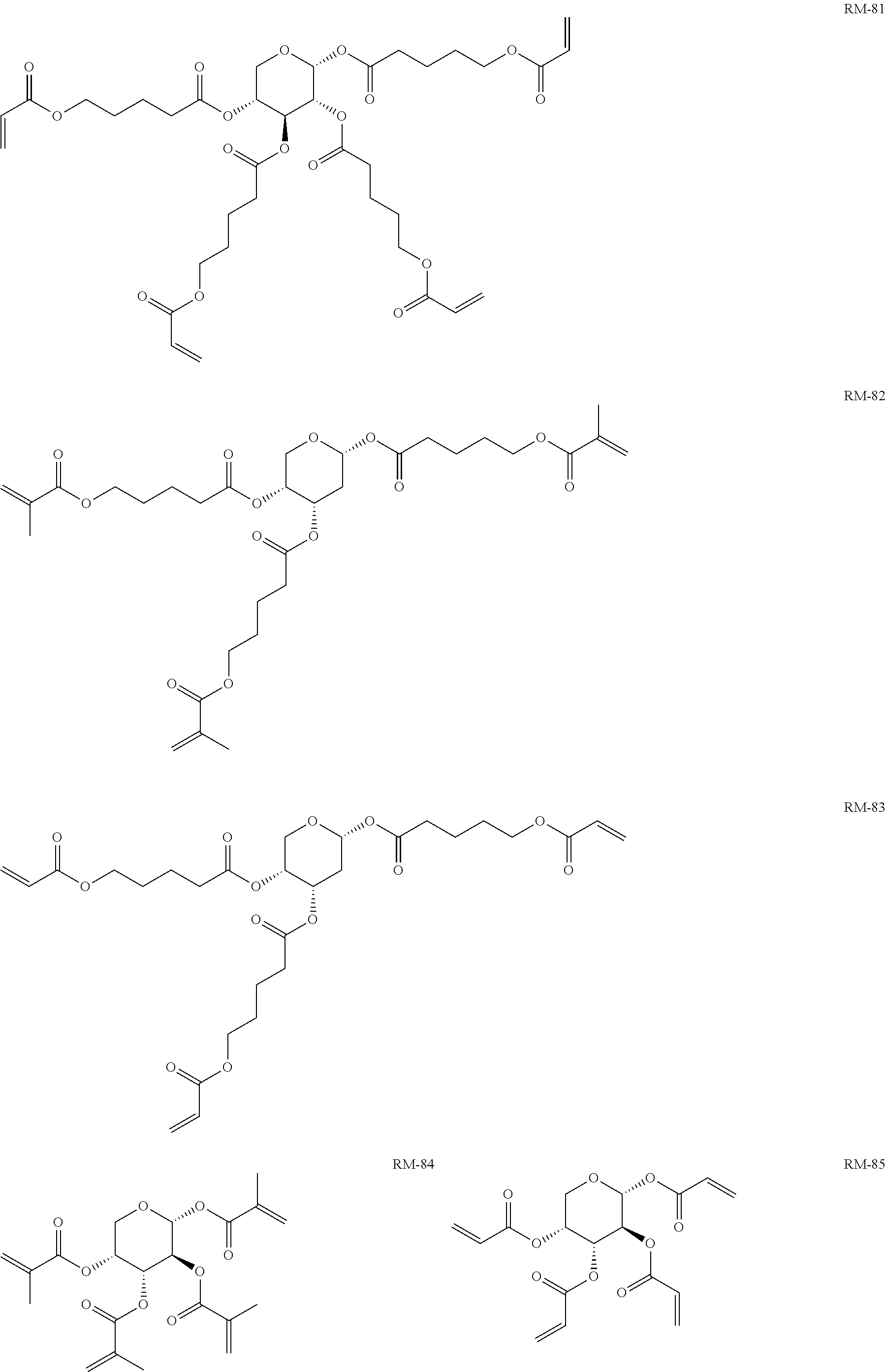

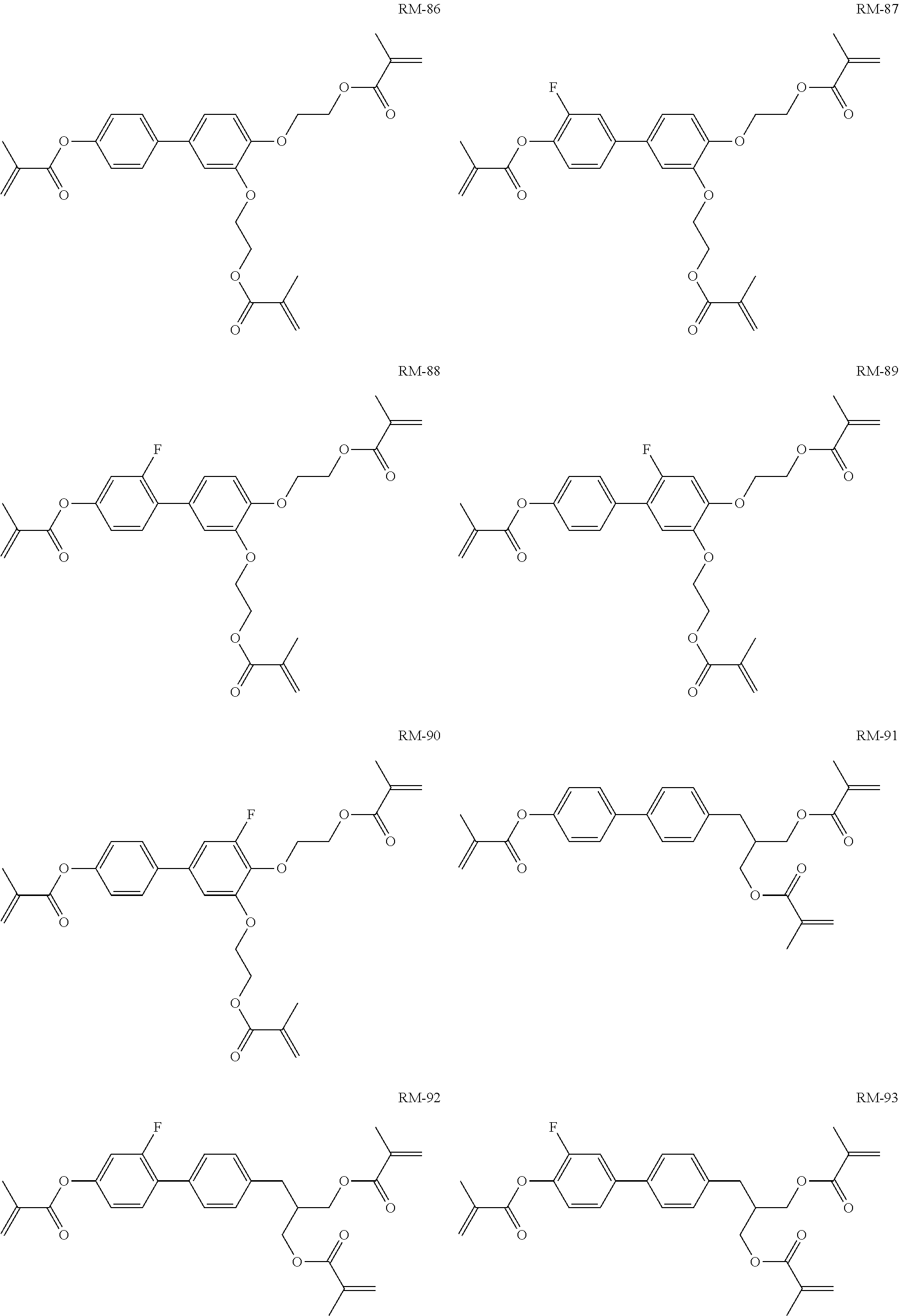

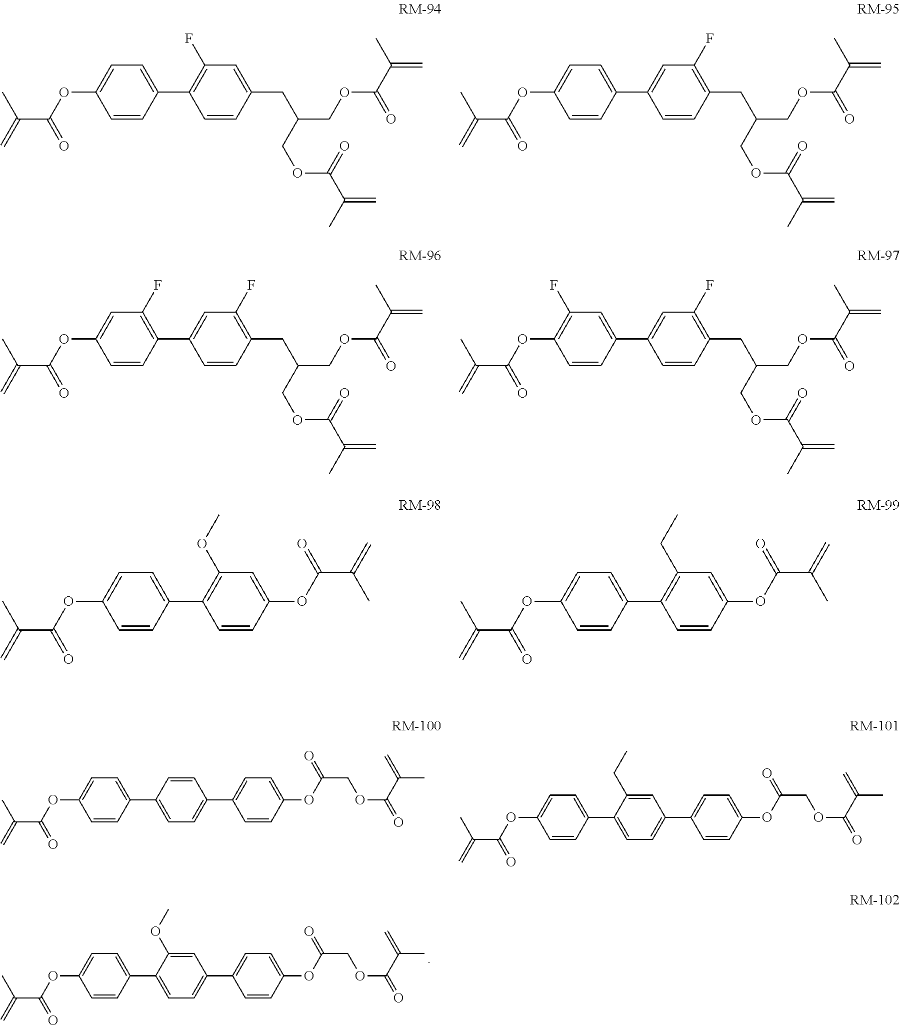

17. The liquid-crystalline medium according to claim 1, wherein said medium contains compounds of formulas CPY-n-Om and CY-n-Om, wherein n and m each, independently of one another, denote 1-6, in concentrations of 10-80%, based on the mixture as a whole:

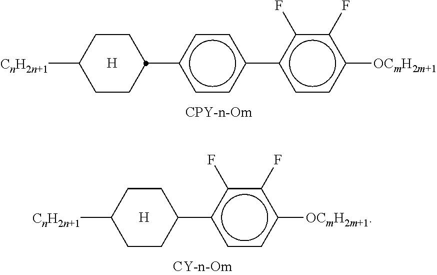

18. The liquid-crystalline medium according to claim 1, wherein said medium contains compounds of formulas CPY-n-Om and CK-n-F, wherein n and m each, independently of one another, denote 1-6, in concentrations of 10-70%, based on the mixture as a whole:

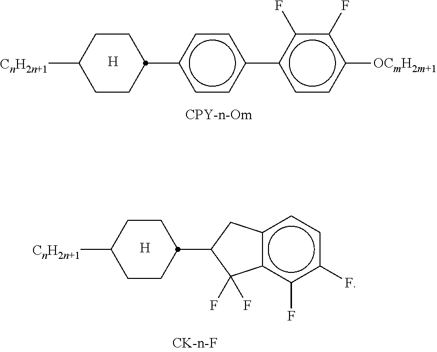

19. The liquid-crystalline medium according to claim 1, wherein said medium contains compounds of formulas CPY-n-Om and PY-n-Om, wherein n and m each, independently of one another, denote 1-6, in concentrations of 10-40%, based on the mixture as a whole:

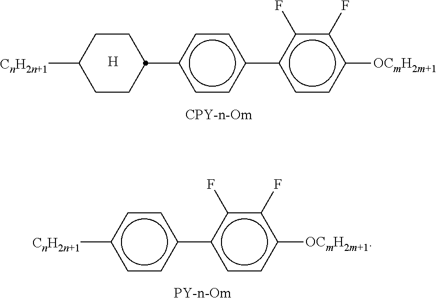

20. The liquid-crystalline medium according to claim 1, wherein said medium contains compounds of formulas CPY-n-Om and CLY-n-Om, wherein n and m each, independently of one another, denote 1-6, in concentrations of 10-80%, based on the mixture as a whole:

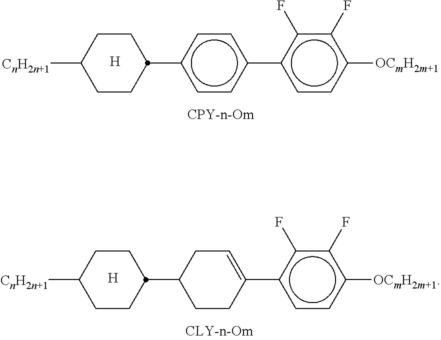

21. The liquid-crystalline medium according to claim 1, wherein said medium contains a compound of the formula CC-n-V1, wherein n is 3, in a concentration of 3-15%, based on the mixture as a whole:

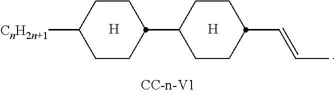

22. The liquid-crystalline medium according to claim 1, wherein said medium contains a compound of the formula CC-V-V, in a concentration of 5-60%, based on the mixture as a whole:

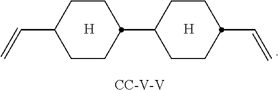

23. The liquid-crystalline medium according to claim 1, wherein said medium contains a compound of the formula CC-n-V, wherein n is 3, in a concentration of 5-60%, based on the mixture as a whole:

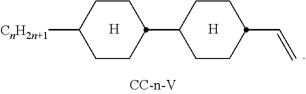

24. The liquid-crystalline medium according to claim 1, wherein said medium contains a compound of the formula PGIY-n-Om, wherein n and m each, independently of one another, denote 1-6, in a concentration of 3-15%, based on the mixture as a whole:

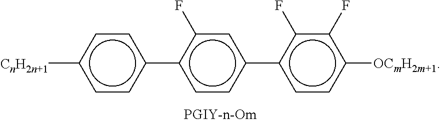

25. The liquid-crystalline medium according to claim 1, wherein said medium contains a compound of the formula CC-n-2V1, wherein n denotes 1-6, in a concentration of 3-20%, based on the mixture as a whole:

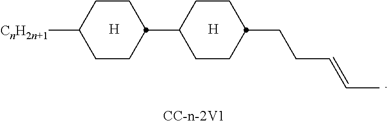

26. A process for preparation of a liquid-crystalline medium according to claim 1, said process comprising:
mixing at least compound of formula I with at least one further liquid-crystalline compound, and optionally one or more additives, and optionally at least one polymerizable compound.

27. A method of generating an electro-optical effect comprising applying a voltage across a liquid-crystalline medium according to claim 1.

28. An electro-optical display having active-matrix addressing, wherein said display contains, as dielectric, a liquid-crystalline medium according to claim 1.

29. The electro-optical display according to claim 28, wherein said display is a VA, PSA, PS-VA, PALC, IPS, PS-IPS, SS-VA, PM-VA, FFS, or PS-FFS display.
